(19) United States
(12) Patent Application Publication
(10) Pub. No.: US 2024/0172556 A1
(43) Pub. Date: May 23, 2024

(54) ORGANIC ELECTRIC ELEMENT USING COMPOUND FOR ORGANIC ELECTRIC ELEMENT, AND ELECTRONIC DEVICE THEREOF

(71) Applicant: DUK SAN NEOLUX CO., LTD., Chungcheongnam-do (KR)

(72) Inventors: Sun Hee LEE, Chungcheongnam-do (KR); Jung Geun LEE, Chungcheongnam-do (KR); Hyoung Keun PARK, Chungcheongnam-do (KR); Soung Yun MUN, Chungcheongnam-do (KR); Won Sam KIM, Chungcheongnam-do (KR); Hyun Jung BAEK, Chungcheongnam-do (KR)

(21) Appl. No.: 18/280,826

(22) PCT Filed: Feb. 21, 2022
(86) PCT No.: PCT/KR2022/002487

§ 371 Date: Sep. 7, 2023

(30) Foreign Application Priority Data
Mar. 8, 2021 (KR) 10-2021-0030243
Mar. 8, 2021 (KR) 1020210030243

(51) Int. Cl. H10K 85/60; C07D 251/24; C07D 307/91; C07D 333/76; C07D 401/04; C07D 403/14; C07D 405/04; C07D 405/12; C07D 407/12; C09K 11/06

(52) U.S. Cl. CPC H10K 85/636; C07D 251/24; C07D 307/91; C07D 333/76; C07D 401/04; C07D 403/14; C07D 405/04; C07D 405/12; C07D 407/12; C09K 11/06; H10K 85/615; H10K 85/633; H10K 85/654; H10K 85/6572; H10K 85/6574; H10K 50/15

(57) ABSTRACT

An organic electric element includes a hole transport band layer which includes compound represented by Formula 1 and an electron transport band layer includes compound represented by Formula 2. The driving voltage of an organic electric element including the organic electric element can be lowered and the luminous efficiency and lifespan can be improved.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/002487 filed on Feb. 21, 2022, which claims priority to the benefit of Korean Patent Application No. 10-2021-0030243 filed in the Korean Intellectual Property Office on Mar. 8, 2021, the entire contents of which are incorporated herein by reference.

### BACKGROUND

### 1. Technical Field

The present invention relates to organic electric element using organic compound for organic electric element and electronic device thereof.

### 2. Background Art

In general, an organic light emitting phenomenon refers to a phenomenon in which electric energy is converted into light energy of an organic material. An organic electric element utilizing the organic light emitting phenomenon usually has a structure including an anode, a cathode, and an organic material layer interposed therebetween. Here the organic material layer has a multi-layered structure having respectively different materials in order to improve efficiency and stability of an organic electric element, and for example, may include a hole injection layer, a hole transport layer, a light emitting layer, an electron transport layer, an electron injection layer, or the like.

The most important issues in organic electroluminescent element are life and efficiency, and as the display becomes larger, these efficiency and life problems must be solved.

Efficiency, life span, driving voltage, and the like are correlated with each other. If efficiency is increased, then driving voltage is relatively lowered, and the crystallization of an organic material due to Joule heating generated during operation is reduced as driving voltage is lowered, as a result of which life span shows a tendency to increase.

However, efficiency cannot be maximized only by simply improving the organic material layer. This is because long life span and high efficiency can be simultaneously achieved when energy levels and T1 values among the respective layers included in the organic material layer, inherent material properties (mobility, interfacial properties, etc.) and the like are optimal combination.

Therefore, the compound for a hole transport band layer and the compound for the electron transport band layer need to be used in proper combination in order to improve the luminous efficiency and lifetime while lowering the driving voltage of the organic electric element.

### SUMMARY

The object of the present invention is to provide an organic electric element using compounds used in a hole transport band layer and an electron transport band layer in an appropriate combination to lower the driving voltage of the element and improve the luminous efficiency and lifespan of the element, and an electronic device thereof.

In an aspect of the present invention, the present invention provides an organic electric element in which compound represented by Formula 1 is used for a hole transport band layer and compound represented by Formula 2 is used for an electron transport band layer.

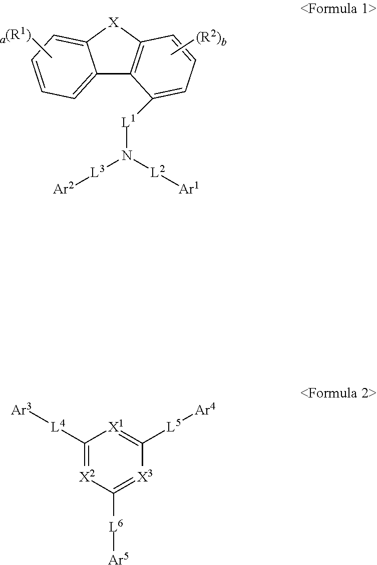

In another aspect of the present invention, the present invention provides an electronic device including the organic electric element.

By appropriately combining the compounds used in a hole transport band layer and an electron transport band layer, the driving voltage of an organic electric element can be lowered and the luminous efficiency and lifespan of the element can be improved.

### BRIEF DESCRIPTION OF THE DRAWINGS

FIGS. 1 to 3 illustrate an example of organic electroluminescent element according to the present invention.

### DETAILED DESCRIPTION

Unless otherwise stated, the term “aryl group” or “arylene group” as used herein has, but not limited to, 6 to 60 carbon atoms. The aryl group or arylene group in the present invention may comprise a monocyclic ring, ring assemblies, a fused polycyclic system, spiro compound and the like.

As used herein, the term “fluorenyl group” refers to a substituted or unsubstituted fluorenyl group, and “fluorenylene group” refers to a substituted or unsubstituted fluorenylene group. The fluorenyl group or fluorenylene group used in the present invention comprises a spiro compound formed by combining R and R′ with each other in the following structure, and also comprises compound formed by linking adjacent R″s to each other. “Substituted fluorenyl group”, “substituted fluorenylene group” means that at least one of R, R′, R″ in the following structure is a substituent other than hydrogen, and R″ may be 1 to 8 in the following formula. In this specification, a fluorene group and a fluorenylene group may be referred to as a fluorene group or fluorene regardless of the valence.

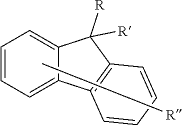

The term “spiro compound” as used herein has a spiro union which means union having one atom as the only common member of two rings. The common atom is designated as ‘spiro atom’. The compounds are defined as ‘monospiro-’, ‘dispiro-’ or ‘trispiro-’ depending on the number of spiro atoms in one compound.

The term “heterocyclic group” used in the specification comprises a non-aromatic ring as well as an aromatic ring like “heteroaryl group” or “heteroarylene group”. Unless otherwise stated, the term “heterocyclic group” means, but not limited to, a ring containing one or more heteroatoms and having 2 to 60 carbon atoms. Unless otherwise stated, the term “heteroatom” as used herein represents, for example, N, O, S, P or Si and may comprise a heteroatom group such as SO₂, P═O etc. instead of carbon forming a ring such as the following compound. In the specification, “heterocyclic group” comprises a monocyclic, ring assemblies, fused polycyclic system, a spiro-compound and the like.

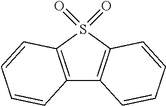 [SMILES: O=S1(=O)c2ccccc2-c2ccccc21]

The term “aliphatic ring group” as used herein refers to a cyclic hydrocarbon except for aromatic hydrocarbons, and comprises a monocyclic ring, ring assemblies, a fused polycyclic system, a spiro-compound and the like, and unless otherwise specified, it means a ring of 3 to 60 carbon atoms, but not limited thereto. For example, a fused ring of benzene and cyclohexane corresponds to aliphatic ring group, wherein benzene is an aromatic ring and cyclohexane is a non-aromatic ring.

In this specification, a ‘group name’ corresponding to an aryl group, an arylene group, a heterocyclic group, and the like exemplified for each symbol and its substituent may be written in the name of functional group reflecting the valence, and may also be described as the name of a parent compound. For example, in the case of phenanthrene which is a kind of aryl group, it may be described by distinguishing valence such as ‘phenanthryl (group)’ when it is ‘monovalent group’, and ‘phenanthrylene (group)’ when it is ‘divalent group’, and it may also be described as a parent compound name, ‘phenanthrene’, regardless of its valence. Similarly, in the case of pyrimidine, it may be described as ‘pyrimidine’ regardless of its valence, and it may also be described as the name of corresponding functional group such as pyrimidinyl (group) when it is ‘monovalent group’, and ‘pyrimidylene (group)’ when it is ‘divalent group’.

In addition, in the present specification, the numbers and alphabets indicating a position may be omitted when describing a compound name or a substituent name. For example, pyrido[4,3-d]pyrimidine, benzofuro[2,3-d]pyrimidine and 9,9-dimethyl-9H-fluorene can be described as pyridopyrimidine, benzofuropyrimidine and dimethylfluorene, respectively. Therefore, both benzo[g]quinoxaline and benzo[f]quinoxaline can be described as benzoquinoxaline.

In addition, unless otherwise expressed, where any formula of the present invention is represented by the following formula, the substituent according to the index may be defined as follows.

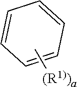

In the above formula, where a is an integer of zero, the substituent R¹ is absent, that is, hydrogen atoms are bonded to all the carbon constituting the benzene ring. Here, chemical formulas or compounds may be written without explicitly describing the hydrogen. In addition, one substituent R¹ is bonded to any carbon of the carbons forming the benzene ring when “a” is an integer of 1. Similarly, where “a” is an integer of 2 or 3, substituents R¹s may be bonded to the carbon of the benzene ring, for example, as followings. Also, where “a” is an integer of 4 to 6, substituents R¹s are bonded to the carbon of the benzene ring in a similar manner. Further, where “a” is an integer of 2 or more, R¹s may be the same or different from each other.

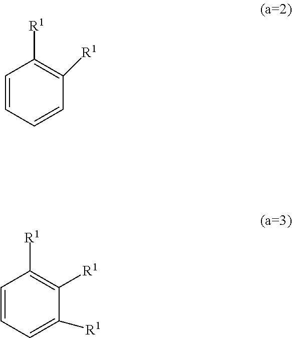

In addition, unless otherwise specified in the present specification, when referring to a condensed/fused ring, the number in the ‘number-condensed/fused ring’ indicates the number of condensed/fused rings. For example, a form in which three rings are condensed/fused with each other, such as anthracene, phenanthrene, and benzoquinazoline, may be represented by a 3-condensed/fused ring.

In addition, unless otherwise described herein, in the case of expressing a ring in the form of a ‘number-membered’ such as a 5-membered ring or a 6-membered ring, the number in the ‘number-membered’ represents the number of atoms forming the ring. For example, thiophene or furan may correspond to a 5-membered ring, and benzene or pyridine may correspond to a 6-membered ring.

In addition, unless otherwise specified in the present specification, when adjacent groups are linked to each other to form a ring, the ring may be selected from the group consisting of a C₆-C₆₀ aromatic ring group, a fluorenyl group, a C₂-C₆₀ heterocyclic group containing at least one heteroatom selected from the group consisting of O, N, S, Si and P, and a C₃-C₆₀ aliphatic ring.

Unless otherwise stated, the term “between adjacent groups”, for example, in case of the following Formulas, comprises not only “between R₁ and R₂”, “between R₂ and R₃”, “between R₃ and R₄”, “between R₅ and R₆”, but also “between R₇ and R₈” sharing one carbon, and may comprise “between substituents” attached to atom(carbon or nitrogen) consisting different ring, such as “between R₁ and R₇”, “between R₁ and R₈”, or “between R₄ and R₈” and the like. That is, where there are substituents bonded to adjacent elements constituting the same ring, the substituents may be correspond “adjacent groups”, and even if there are no adjacent substituents on the same ring, substituents attached to the adjacent ring may correspond to “adjacent groups”. In the following Formula, when the substituents bonded to the same carbon, such as R₇ and R₅, are linked to each other to form a ring, a compound containing a spiro-moiety may be formed.

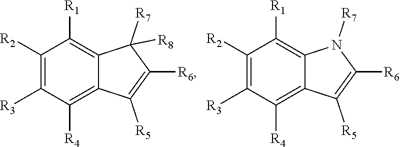

In addition, in the present specification, the expression ‘adjacent groups may be linked to each other to form a ring’ is used in the same sense as ‘adjacent groups are linked selectively to each other to form a ring’, and a case where at least one pair of adjacent groups may be bonded to each other to form a ring.

In addition, unless otherwise specified in the present specification, an aryl group, an arylene group, a fluorenyl group, a fluorenylene group, a heterocyclic group, an aliphatic ring group, an alkyl group, an alkenyl group, an alkynyl group, an alkoxyl group, an aryloxyl group, and a ring formed by adjacent groups may be each optionally substituted with one or more substituents selected from the group consisting of deuterium, halogen, an amino group unsubstituted or substituted with a C₁-C₂₀ alkyl group or a C₆-C₂₀ aryl group, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

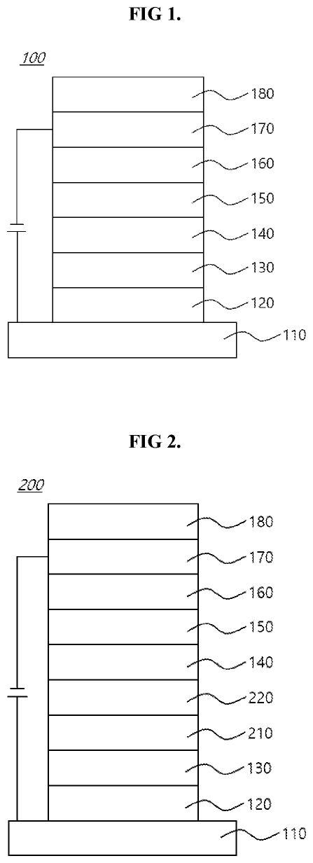

Hereinafter, referring to FIGS. 1 to 3, a lamination structure of an organic electric element including the compound of the present invention will be described.

In designation of reference numerals to components in respective drawings, it should be noted that the same elements will be designated by the same reference numerals although they are shown in different drawings. In addition, in the following description of the present invention, a detailed description of known functions and configurations incorporated herein will be omitted when it may make the subject matter of the present invention rather unclear.

Terms, such as first, second, A, B, (a), (b) or the like may be used herein when describing components of the present invention. Each of these terminologies is not used for defining an essence, order or sequence of a corresponding component but used merely to distinguish the corresponding component from other component(s). It will be understood that the expression ‘one component is “connected,” “coupled” or “joined” to another component’ comprises the case where a third component may be “connected,” “coupled” or “joined” between a first component and a second component as well as the case where the first component may be directly connected, coupled or joined to the second component.

In addition, it will be understood that when an element such as a layer, film, region or substrate is referred to as being “on” or “over” another element, it can be directly on the other element or intervening elements may also be present. In contrast, when an element is referred to as being “directly on” another element, there are no intervening elements present.

The FIGS. 1 to 3 show an example of an organic electric element according to an embodiment of the present invention, respectively.

Referring to the FIG. 1, an organic electric element 100 according to an embodiment of the present invention includes a first electrode 110 formed on a substrate (not shown), a second electrode 170, and an organic material layer between the first electrode 110 and the second electrode 170.

The first electrode 110 may be an anode (positive electrode), and the second electrode 170 may be a cathode (negative electrode). In the case of an inverted organic electric element, the first electrode may be a cathode, and the second electrode may be an anode.

The organic material layer may be comprised a hole injection layer 120, a hole transport layer 130, a light emitting layer 140, an electron transport layer 150, and an electron injection layer 160. Specifically, a hole injection layer 120, a hole transport layer 130, a light emitting layer 140, an electron transport layer 150, and an electron injection layer 160 may be formed on the first electrode 110 in sequence.

Preferably, a layer for improving the luminous efficiency 180 may be formed one side of sides of the first electrode 110 and the second electrode 170, wherein one side is not facing the organic material layer, as a result the luminous efficiency of an organic electric element can be improved.

For example, the light efficiency improving layer 180 may be formed on the second electrode 170, as a result, in the case of a top emission organic light emitting element, the optical energy loss due to Surface Plasmon Polaritons (SPPs) at the second electrode 170 may be reduced and in the case of a bottom emission organic light emitting element, the light efficiency improving layer 180 may serve as a buffer for the second electrode 170.

A buffer layer 210 or an emission-auxiliary layer 220 may be further formed between the hole transport layer 130 and the light emitting layer 140, which will be described with reference to FIG. 2.

Referring to FIG. 2, the organic electric element 200 according to another embodiment of the present invention may comprise a hole injection layer 120, a hole transport layer 130, a buffer layer 210, an emission-auxiliary layer 220, a light emitting layer 140, the electron transport layer 150, the electron injection layer 160, and a second electrode 170 formed on a first electrode 110 in sequence, and a layer for improving light efficiency 180 may be formed on the second electrode 170.

Although not shown in FIG. 2, an electron transport auxiliary layer may be further formed between the light emitting layer 140 and the electron transport layer 150.

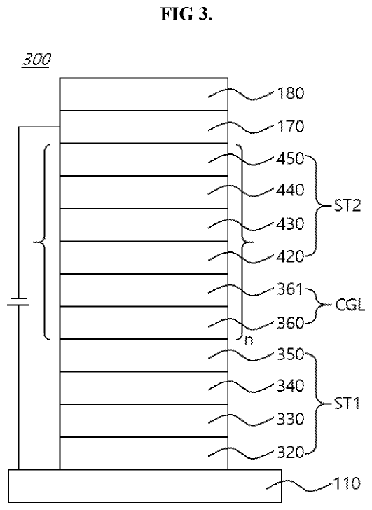

In addition, according to another embodiment of the present invention, the organic material layer may be a form consisting of multiple stacks, wherein the stacks comprise a hole transport layer, a light emitting layer, and an electron transport layer, respectively. This will be described with reference to FIG. 3.

Referring to FIG. 3, two or more sets of stacks of the organic material layers ST1 and ST2 may be formed between the first electrode 110 and the second electrode 170 in the organic electric element 300 according to another embodiment of the present invention, wherein the organic material layers are consisted of multiple layers, respectively, and the charge generation layer CGL may be formed between the stacks of the organic material layer.

Specifically, the organic electric element according to the embodiment of the present invention may comprise a first electrode 110, a first stack ST1, a charge generation layer CGL, a second stack ST2, and a second electrode 170 and a layer for improving light efficiency 180.

The first stack ST1 is an organic layer formed on the first electrode 110, and the first stack ST1 may comprise the first hole injection layer 320, the first hole transport layer 330, the first light emitting layer 340 and the first electron transport layer 350 and the second stack ST2 may comprise a second hole injection layer 420, a second hole transport layer 430, a second light emitting layer 440 and a second electron transport layer 450. As such, the first stack and the second stack may be the organic layers having the same or different stacked structures.

The charge generation layer CGL may be formed between the first stack ST1 and the second stack ST2. The charge generation layer CGL may comprise a first charge generation layer 360 and a second charge generation layer 361. The charge generating layer CGL is formed between the first light emitting layer 340 and the second light emitting layer 440 to increase the current efficiency generated in each light emitting layer and to smoothly distribute charges.

The first light emitting layer 340 may comprise a light emitting material comprising a blue host doped with a blue fluorescent dopant and the second light emitting layer 440 may comprise a light emitting material comprising a green host doped with a greenish yellow dopant and a red dopant together, but the material of the first light emitting layer 340 and the second light emitting layer 440 according to an embodiment of the present invention is not limited thereto.

In FIG. 3, n may be an integer of 1 to 5 and the charge generation layer CGL and the third stack may be further stacked on the second stack ST2 when n is 2.

When multiple light emitting layers are formed in a multi-layer stack structure as shown in FIG. 3, it is possible to manufacture an organic electroluminescent element that emits not only white light but also various colors, wherein the white light is emitted by the mixing effect of light emitted from each light emitting layer.

Compound represented by Formula 1 may be used for a hole transport band layer and compound represented by Formula 2 is used for an electron transport band layer. Here, a hole transport band layer is an organic layer between a first electrode and a light-emitting layer and includes a hole injection layer 120, 320, 420, a hole transport layer 130, 330, 430, a buffer layer 210, and an emission-auxiliary layer 220, and an electron transport band layer is an organic material layer between a second electrode and a light emitting layer and may include an electron transport auxiliary layer(a hole blocking layer), an electron transport layer 150, 350, 450, and an electron injection layer 160.

Preferably, compound represented by Formula 1 of the present invention is included in a hole transport layer 130, 330, 430 or an emission-auxiliary layer 220, and more preferably is included in an emission-auxiliary layer 220, and compound represented by Formula 2 can be used in the electron transport auxiliary layer or electron transport layer 150, 350, 450.

Even if the cores of compounds are identical or similar to each other, the band gap, the electrical characteristics, the interface characteristics and the like may be different depending on which substituent is bonded at which position. Therefore, it is necessary to study the selection of the core and the combination with sub-substituent bonded to the core. In particular, long life span and high efficiency can be simultaneously achieved when the optimal combination of energy levels and T₁ values, inherent material properties (mobility, interfacial properties, etc.), and the like among the respective layers of an organic material layer is achieved.

Therefore, the energy level and T₁ value between the respective layers of the organic material layer, inherent material properties (mobility, interfacial properties, etc.) and the like can be optimized by using compound represented by Formula 1 for a hole transport band layer and compound represented by Formula 2 for an electron transport band layer.

The organic electric element according to an embodiment of the present invention may be manufactured using various deposition methods. The organic electric element according to an embodiment of the present invention may be manufactured using a PVD (physical vapor deposition) method or CVD (chemical vapor deposition) method. For example, the organic electric element may be manufactured by depositing a metal, a conductive metal oxide, or a mixture thereof on the substrate to form the anode 110, forming the organic material layer comprising the hole injection layer 120, the hole transport layer 130, the light emitting layer 140, the electron transport layer 150, and the electron injection layer 160 thereon, and then depositing a material, which can be used as the cathode 170, thereon. Also, an emission-auxiliary layer 220 may be formed between a hole transport layer 130 and a light emitting layer 140, and an electron transport auxiliary layer(not shown) may be further formed between a light emitting layer 140 and an electron transport layer 150 and, as described above, a stack structure may be formed.

Also, the organic material layer may be manufactured in such a manner that the fewer layers are formed using various polymer materials by a soluble process or solvent process, for example, spin coating, nozzle printing, inkjet printing, slot coating, dip coating, roll-to-roll, doctor blading, screen printing, or thermal transfer, instead of deposition. Since the organic material layer according to the present invention may be formed in various ways, the scope of protection of the present invention is not limited by a method of forming the organic material layer.

The organic electric element according to an embodiment of the present invention may be of a top emission type, a bottom emission type, or a dual emission type depending on the material used.

In addition, the organic electric element according to an embodiment of the present invention may be selected from the group consisting of an organic electroluminescent element, an organic solar cell, an organic photo conductor, an organic transistor, an element for monochromatic illumination and an element for a quantum dot display.

Another embodiment of the present invention provides an electronic device including a display device which includes the above described organic electric element, and a control unit for controlling the display device. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, an organic electric element according to one aspect of the present invention will be described.

An organic electric element according to one aspect of the present invention includes a first electrode, a second electrode, and an organic material layer formed between the first electrode and the second electrode, the organic material layer includes a light-emitting layer, a hole transport band layer formed between the light-emitting layer and the first electrode, and an electron transport band layer formed between the light-emitting layer and the second electrode, the hole transport band layer includes compound represented by the following Formula 1, and the electron transport band layer includes compound represented by the following Formula 2.

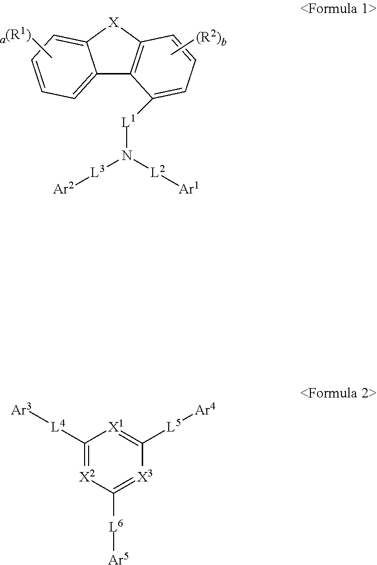

In Formulas, each of symbols may be defined as follows.
X is O or S.
X¹ to X³ are N or C(R¹) and at least one of X¹ to X³ is N. At least one of X¹ to X³ may be N, and the ring containing X¹ to X³ may be, for example, pyridine, pyrimidine, or triazine, preferably, triazine.
R¹, R² and R₁ are each independently selected from the group consisting of hydrogen, deuterium, halogen, a cyano group, a nitro group, a C₆-C₆₀ aryl group, a fluorenyl group, a C₂-C₆₀ heterocyclic group comprising at least one heteroatom selected from the group consisting of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, a C₆-C₂₀ aryloxy group and -L′-N(Rₐ)(R_{b}), and adjacent groups may be bonded to each other to form a ring, with a proviso that at least one of R¹ and R² is deuterium.

The term ‘adjacent groups’ may mean, for example, adjacent R¹s or adjacent R²s. When a ring is formed by at least one pair of adjacent groups, the ring may be selected from the group consisting of a C₆-C₆₀ aromatic ring group, a fluorene group, a C₂-C₆₀ heterocyclic group containing at least one heteroatom of O, N, S, Si and P, and a C₃-C₆₀ aliphatic ring group.

When an aromatic ring group is formed by adjacent groups, the aromatic ring group may be a C₆-C₃₀, a C₆-C₂₀, a C₆-C₁₆, a C₆-C₁₄, a C₆-C₁₀ or a C₆ aromatic ring group, specifically, benzene, naphthalene, phenanthrene or the like.
a is an integer of 1 to 4, b is an integer of 1 to 3, and when each of these is an integer of 2 or more, each of R¹s, each of R²s are the same as or different from each other, and when there are multiple R₁s, each of these is the same or different from each other.
L¹ to L⁶ are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P.
Ar¹ to Ar⁵ are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group. At least one of Ar¹ and Ar² may be a heterocyclic group, and at least one of Ar⁴ to Ar⁶ may be an aryl group substituted with a cyano group or a heterocyclic group substituted with a cyano group.
L′ is selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P.
Rₐ and R_{b} are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group.

When at least one of Ar¹ to Ar⁵, R¹, R², R₁, Rₐ and R_{b} is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group, specifically, phenyl, biphenyl, naphthyl, terphenyl, phenanthrene, triphenylene, or the like.

When at least one of L¹ to L⁶ and L′ is an arylene group, the arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ aryl group, specifically, phenylene, biphenyl, naphthylene, terphenyl, phenanthrene, triphenylene, or the like.

When at least one of Ar¹ to Ar⁵, R¹, R², R₁, Rₐ, R_{b}, L¹ to L⁶ and L′ is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, or a C₂₉ heterocyclic group, specifically, pyridine, pyrimidine, pyrazine, pyridazine, triazine, furan, pyrrole, silole, indene, indole, phenyl-indole, benzoindole, phenyl-benzoindole, pyrazinoindol, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, benzofuran, naphthobenzofuran, dibenzofuran, dinaphthofuran, thiophene, benzothiophene, dibenzothiophene, naphthobenzothiophene, dinaphthothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzofuropyrimidine, benzothienopyrazine, benzofuropyrazine, benzoimidazole, benzothiazole, benzooxazole, benzosiloe, phenanthroline, dihydro-phenylphenazine, 10-phenyl-10H-phenoxazine, phenoxazine, phenothiazine, dibenzodioxin, benzodibenzodioxin, thianthrene, 9,9-dimethyl-9H-xantene, 9,9-dimethyl-9H-thioxantene, dihydrodimethylphenylacridine, spiro[fluorene-9,9′-xanthene] and the like.

When at least one of Ar¹ to Ar⁵, R¹, R², R₁, Rₐ and R_{b} is a fluorenyl group or at least one of L¹ to L⁶ and L′ is a fluorenylene group, the fluorenyl group or the fluorenylene group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of Ar¹ to Ar⁵, R¹, R², R₁, Rₐ, R_{b}, L¹ to L⁶ and L′ is an aliphatic ring group, the aliphatic ring group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ aliphatic ring group, specifically, cyclohexane, adamantyl group, bicyclo[2.2.1]hept-2-ene, etc.

When at least one of R¹ and R₂ is an alkyl group, the alkyl group may be, for example, a C₁-C₂₀, a C₁-C₁₀, a C₁-C₄, a C₁, a C₂, a C₃ and a C₄ alkyl group, specifically, methyl, ethyl, propyl, t-butyl, etc.

When at least one of R¹ and R₂ is an alkenyl group, the alkenyl group may be, for example, a C₂-C₂₀, a C₂-C₁₀, a C₂-C₄, a C₂, a C₃, a C₄ alkenyl group.

When at least one of R¹ and R₂ is an alkoxy group, the alkoxy group may be, for example, a C₁-C₂₀, a C₁-C₁₀, a C₁-C₄, a C₁, a C₂, a C₃, a C₄ alkoxy group, specifically, a methoxy group, ethoxy group, butoxy group, etc.

The aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent groups may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

When at least one of the aryl group, the fluorenyl group, the heterocyclic group, the aliphatic ring group, the aromatic ring group, the fluorene group, the alkyl group, the alkenyl group, the alkynyl group, the alkoxyl group, the aryloxyl group, the arylene group, the fluorenylene group, and the ring formed by adjacent groups is substituted with an aryl group, the aryl group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇ or a C₁₈ aryl group.

When at least one of the aryl group, the fluorenyl group, the heterocyclic group, the aliphatic ring group, the aromatic ring group, the fluorene group, the alkyl group, the alkenyl group, the alkynyl group, the alkoxyl group, the aryloxyl group, the arylene group, the fluorenylene group, and the ring formed by adjacent groups is substituted with a heterocyclic group, the heterocyclic group may be, for example, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉ or a C₂₀ heterocyclic group.

When at least one of the aryl group, the fluorenyl group, the heterocyclic group, the aliphatic ring group, the aromatic ring group, the fluorene group, the alkyl group, the alkenyl group, the alkynyl group, the alkoxyl group, the aryloxyl group, the arylene group, the fluorenylene group, and the ring formed by adjacent groups is substituted with a fluorenyl group, the fluorenyl group may be 9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of the aryl group, the fluorenyl group, the heterocyclic group, the aliphatic ring group, the aromatic ring group, the fluorene group, the alkyl group, the alkenyl group, the alkynyl group, the alkoxyl group, the aryloxyl group, the arylene group, the fluorenylene group, and the ring formed by adjacent groups is substituted with an alkyl group, the alkyl group may be, for example, a C₁-C₂₀, a C₁-C₁₀, a C₁-C₄, a C₁, a C₂, a C₃, a C₄ alkoxy group.

When at least one of the aryl group, the fluorenyl group, the heterocyclic group, the aliphatic ring group, the aromatic ring group, the fluorene group, the alkyl group, the alkenyl group, the alkynyl group, the alkoxyl group, the aryloxyl group, the arylene group, the fluorenylene group, and the ring formed by adjacent groups is substituted with an alkoxy group, the alkoxy group may be, for example, a C₁-C₂₀, a C₁-C₁₀, a C₁-C₄, a C₁, a C₂, a C₃, a C₄ alkoxy group.

When at least one of the aryl group, the fluorenyl group, the heterocyclic group, the aliphatic ring group, the aromatic ring group, the fluorene group, the alkyl group, the alkenyl group, the alkynyl group, the alkoxyl group, the aryloxyl group, the arylene group, the fluorenylene group, and the ring formed by adjacent groups is substituted with an aliphatic ring group, the aliphatic ring group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇ or a C₁₈ aliphatic ring group.

In Formulas 1 and 2, at least one of Ar¹ to Ar⁵ may be represented by one of the following Formulas Ar-1 to Ar-5.

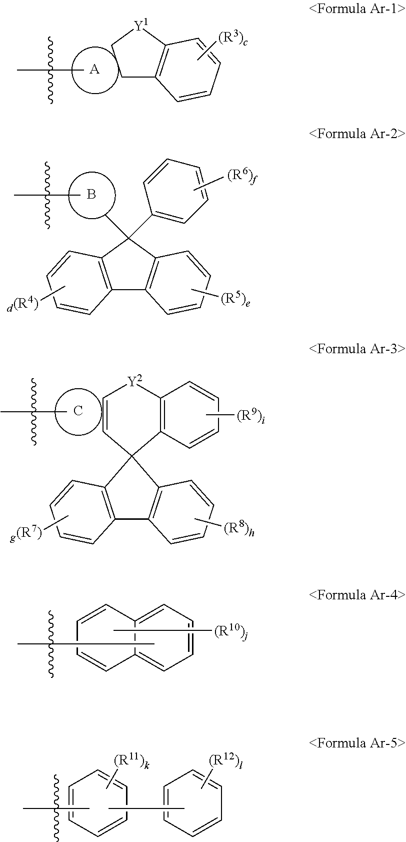

In Formulas Ar-1 to Ar-5, each symbol may be defined as follows.
Y¹ is O, S, C(R′)(R″) or N(Ar₁).
Y² is a single bond, O, S, C(R′)(R″) or N(Ar2).
Rings A to C are each independently selected from the group consisting of a C₆-C₆₀ aromatic ring group, a fluorenylene group, a C₃-C₆₀ aliphatic ring group, and a C₂-C₆₀ heterocyclic group containing at least one heteroatom of O, N, S, Si and P, rings A to C may be each independently substituted with R¹³.

When the rings A to C are an aromatic ring group, the aromatic ring group may be, for example, C₆-C₃₀, C₆-C₂₉, C₆-C₂₈, C₆-C₂₇, C₆-C₂₆, C₆-C₂₅, C₆-C₂₄, C₆-C₂₃, C₆-C₂₂, C₆-C₂₁, C₆-C₂₀, C₆-C₁₉, C₆-C₁₈, C₆-C₁₇, C₆-C₁₆, C₆-C₁₅, C₆-C₁₄, C₆-C₁₃, C₆-C₁₂, C₆-C₁₁, C₆-C₁₀, C₆, C₁₀, C₁₂, C₁₃, C₁₄, C₁₅, C₁₆, C₁₇ or C₁₈ aromatic ring group, specifically, phenylene, biphenyl, naphthylene, terphenyl, phenanthrene, triphenylene, etc.
R³ to R¹³, R′ and R″ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and adjacent groups may be bonded to each other to form a ring, R′ and R″ may be bonded to each other to form a ring, and multiple R¹³s are the same or different from each other. When R′ and R″ are bonded to each other to form a ring, a spiro compound can be formed.
c to e, g to i, and k are each an integer of 0 to 4, f and 1 are each an integer of 0 to 5, j is an integer of 0 to 7, and when each of these is an integer of 2 or more, each of R³, each of R⁴, each of R⁵, each of R⁶, each of R⁷, each of R⁸, each of R⁹, each of R¹⁰, each of R¹¹ and each of R¹² are the same as or different from each other.
Ar₁ and Ar₂ are each independently selected from the group consisting of a C₆-C₂₀ aryl group, a fluorenyl group, a C₂-C₂₀ heterocyclic group containing at least one heteroatom of O, N, S, Si and P, and a C₃-C₂₀ aliphatic ring group.

When at least one of R³ to R¹³, R′, R″, Ar₁, Ar₂ is an aryl group, the aryl group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇ or a C₁₈ aryl group.

When at least one of R³ to R¹³, R′, R″, Ar₁, Ar₂ is a heterocyclic group, the heterocyclic group may be, for example, a C₂-C₂₀, a C₂-C₁₉, a C₂-C₁₈, a C₂-C₁₇, a C₂-C₁₆, a

C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉ or a C₂₀ heterocyclic group.

When at least one of R³ to R¹³, R′, R″, Ar₁, Ar₂ is a fluorenyl group, the fluorenyl group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of R³ to R¹³, R′, R″, Ar₁, Ar₂ is an aliphatic ring group, the aliphatic ring group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ aliphatic ring group.

When at least one of R¹ and R² is an alkyl group, the alkyl group may be, for example, a C₁-C₂₀, a C₁-C₁₀, a C₁-C₄, a C₁, a C₂, a C₃, a C₄ alkyl group.

When at least one of R³ to R¹³, R′, R″ is an alkenyl group, the alkenyl group may be, for example, a C₂-C₂₀, a C₂-C₁₀, a C₂-C₄, a C₂, a C₃, a C₄ alkenyl group.

When at least one of R³ to R¹³, R′, R″ is an alkoxy group, the alkoxy group may be, for example, a C₁-C₂₀, a C₁-C₁₀, a C₁-C₄, a C₁, C₂, a C₃, a C₄ alkoxy group.

At least one of the rings A to C may be selected from the group consisting of Formula F-1 to Formula F-7 below.

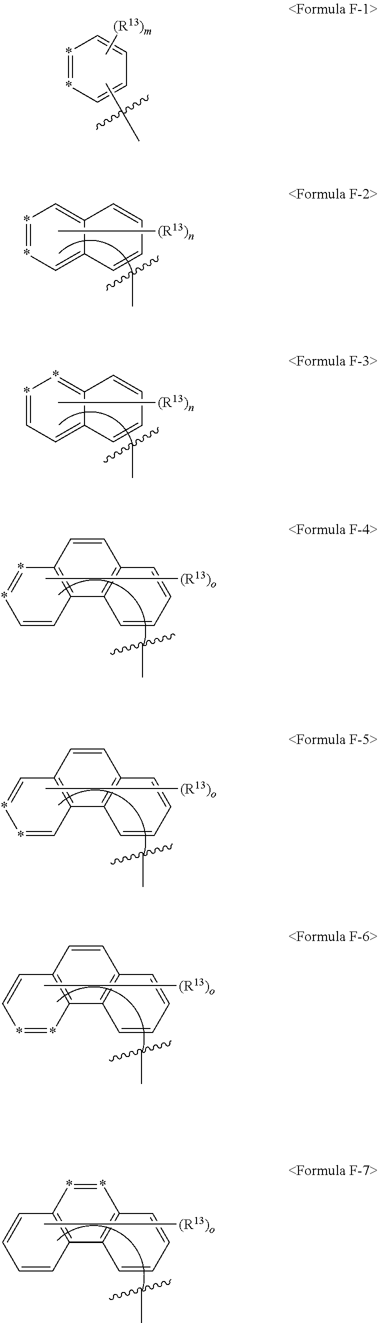

The Formulas, * indicates the condensation position, R¹³ is the same as defined above, m is an integer of 0 to 3, n is an integer of 0 to 5, and o is an integer of 0 to 7.

In Formulas above, at least one of L¹ to L⁶ may be selected from the group consisting of Formulas b-1 to b-13 below.

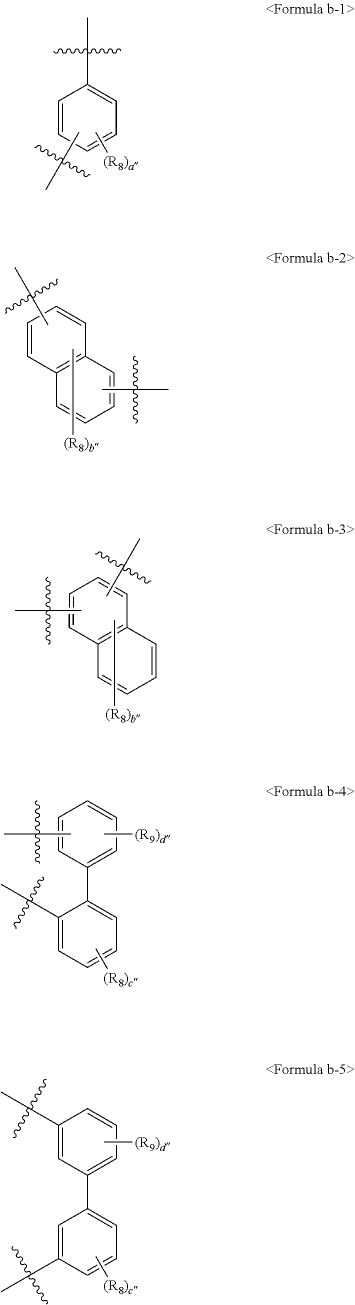

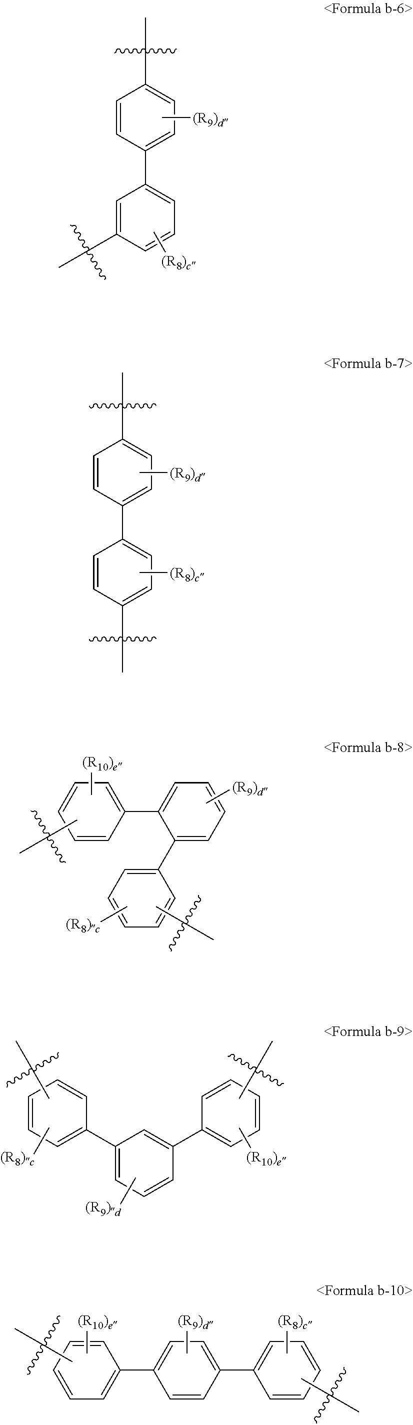

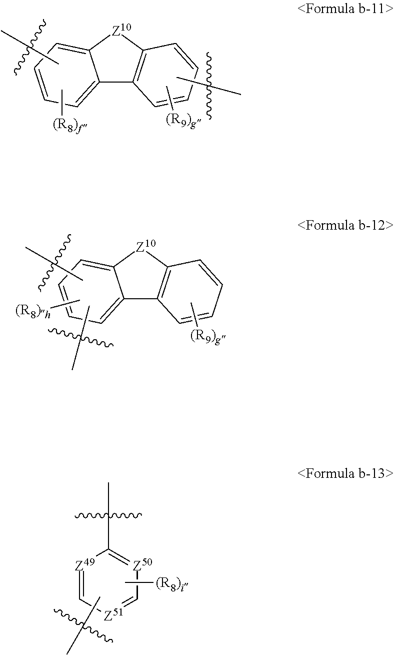

In Formula b-1 to Formula b-13, each symbol can be defined as follows.
Z¹⁰ is O, S, C(R′)(R″) or N(Ar₃).
Z⁴⁰ to Z⁵¹ are each independently N or C(R′), and at least one of Z⁴⁹ to Z⁵¹ is N.
R₈ to R₁₀, R′ and R″ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and adjacent groups may be bonded to each other to form a ring and R′ and R″ may be bonded to each other to form a ring.
a″, c″, d″, e″ and g″ in Formula b-12 are each integer of 0 to 4, f′ and g″ in Formula b-11 are integers of 0 to 3, h″ is 0 is an integer of 0 to 2, and i″ is an integer of 0 or 1, and when each of these is an integer of 2 or more, each of R₈, each of R₉, and each of R₁₀ are the same as or different from each other.
Ar₃ is selected from the group consisting of a C₆-C₂₀ aryl group, a fluorenyl group, a C₂-C₂₀ heterocyclic group containing at least one heteroatom of O, N, S, Si and P, and a C₃-C₂₀ aliphatic ring group.

When at least one of R₈ to R₁₀, R′, R″, Ar₃ is an aryl group, the aryl group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-Cii, a C₆-C₁₀, a C₆, a C₁₀, a C₁₂, a C₁₃, a C₁₄, a C₁₅, a C₁₆, a C₁₇ or a C₁₈ aryl group.

When at least one of R₈ to R₁₀, R′, R″, Ar₃ is a heterocyclic group, the heterocyclic group may be, for example, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉ or a C₂₀ heterocyclic group.

When at least one of R₈ to R₁₀, R′, R″, Ar₃ is a fluorenyl group, the fluorenyl group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of R₈ to R₁₀, R′, R″, Ar₃ is an aliphatic ring group, the aliphatic ring group may be, for example, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈ aliphatic ring group.

When at least one of R₈ to R₁₀, R′, R″ is an alkyl group, the alkyl group may be, for example, a C₁-C₂₀, a C₁-C₁₀, a C₁-C₄, a C₁, a C₂, a C₃, a C₄ alkyl group.

When at least one of R₈ to R₁₀, R′, R″ is an alkenyl group, the alkenyl group may be, for example, a C₂-C₂₀, a C₂-C₁₀, a C₂-C₄, a C₂, a C₃, a C₄ alkenyl group.

When at least one of R₈ to R¹⁰, R′, R″ is an alkoxy group, the alkoxy group may be, for example, a C₁-C₂₀, a C₁-C₁₀, a C₁-C₄, a C₁, a C₂, a C₃, a C₄ alkoxy group.

Compound represented by Formula 1 may be represented by Formula 1-1 or Formula 1-2 below.

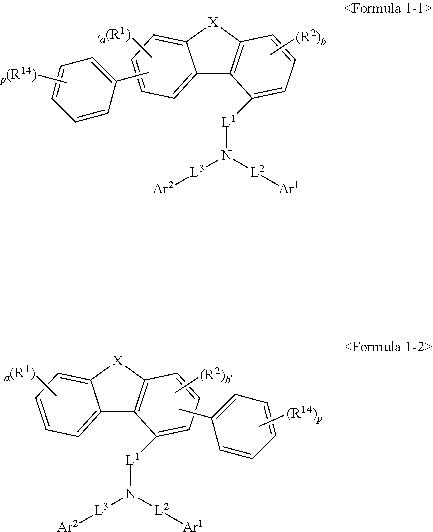

In Formula 1-1 and Formula 1-2, R¹, R², a, b, L¹-L³, Ar¹ and Ar² are the same as defined in Formula 1.
R¹⁴ is selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and adjacent groups may be bonded to each other to form a ring.
a′ is an integer of 0 to 3, b′ is an integer of 0 to 2, and p is an integer of 0 to 5, and when each of these is an integer of 2 or more, each of R¹, each of R² and each of R H are the same or different from each other.

Compound represented by Formula 1 may be one of the following compounds, but there is no limitation thereto.

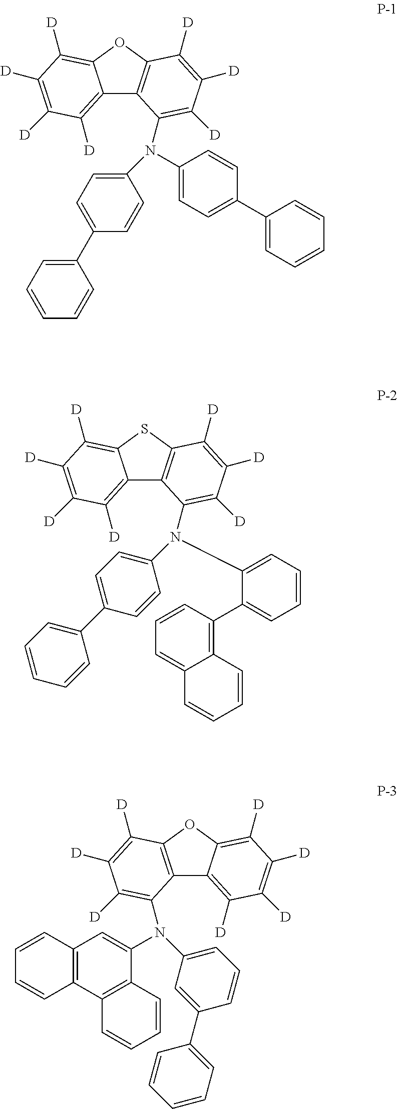 [SMILES: [2H]c1c([2H])c([2H])c2c(oc3c([2H])c([2H])c([2H])c(N(c4ccc(-c5ccccc5)cc4)c4ccc(-c5ccccc5)cc4)c32)c1[2H] ; [2H]c1c([2H])c([2H])c2c(sc3c([2H])c([2H])c([2H])c(N(c4ccc(-c5ccccc5)cc4)c4ccccc4-c4cccc5ccccc45)c32)c1[2H] ; [2H]c1c([2H])c([2H])c2c(oc3c([2H])c([2H])c([2H])c(N(c4cccc(-c5ccccc5)c4)c4cc5ccccc5c5ccccc45)c32)c1[2H]]

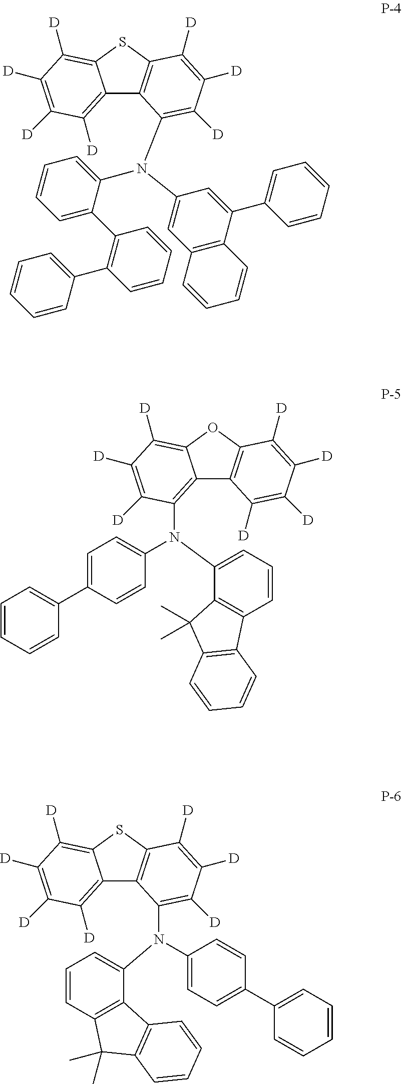 [SMILES: [2H]c1c([2H])c([2H])c2c(sc3c([2H])c([2H])c([2H])c(N(c4cc(-c5ccccc5)c5ccccc5c4)c4ccccc4-c4ccccc4-c4ccccc4)c32)c1[2H] ; [2H]c1c([2H])c([2H])c2c(oc3c([2H])c([2H])c([2H])c(N(c4ccc(-c5ccccc5)cc4)c4cccc5c4C(C)(C)c4ccccc4-5)c32)c1[2H] ; [2H]c1c([2H])c([2H])c2c(sc3c([2H])c([2H])c([2H])c(N(c4ccc(-c5ccccc5)cc4)c4cccc5c4-c4ccccc4C5(C)C)c32)c1[2H]]

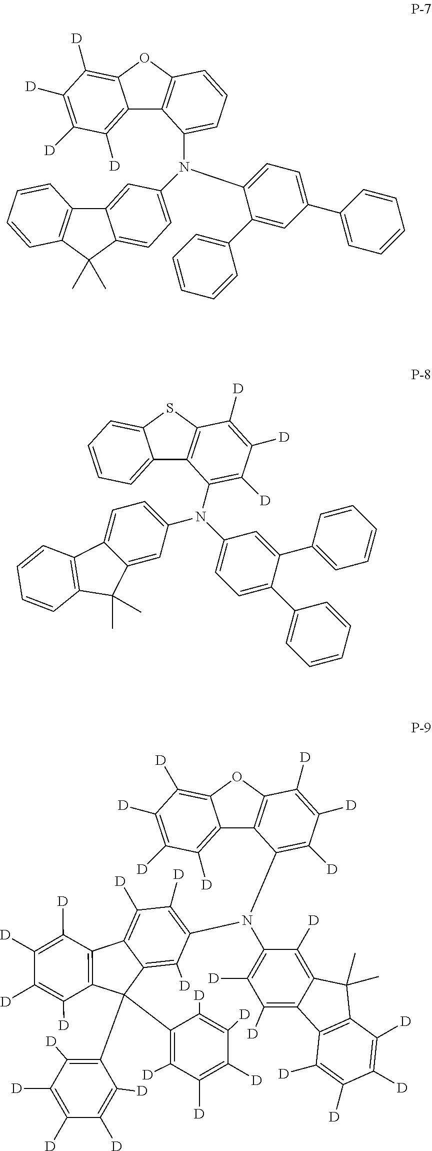 [SMILES: [2H]c1c([2H])c([2H])c2c(oc3cccc(N(c4ccc5c(c4)-c4ccccc4C5(C)C)c4ccc(-c5ccccc5)cc4-c4ccccc4)c32)c1[2H] ; [2H]c1c([2H])c(N(c2ccc(-c3ccccc3)c(-c3ccccc3)c2)c2ccc3c(c2)C(C)(C)c2ccccc2-3)c2c(sc3ccccc32)c1[2H] ; [2H]c1c([2H])c([2H])c(C2(c3c([2H])c([2H])c([2H])c([2H])c3[2H])c3c([2H])c([2H])c([2H])c([2H])c3-c3c([2H])c([2H])c(N(c4c([2H])c([2H])c5c(c4[2H])C(C)(C)c4c([2H])c([2H])c([2H])c([2H])c4-5)c4c([2H])c([2H])c([2H])c5oc6c([2H])c([2H])c([2H])c([2H])c6c45)c([2H])c32)c([2H])c1[2H]]

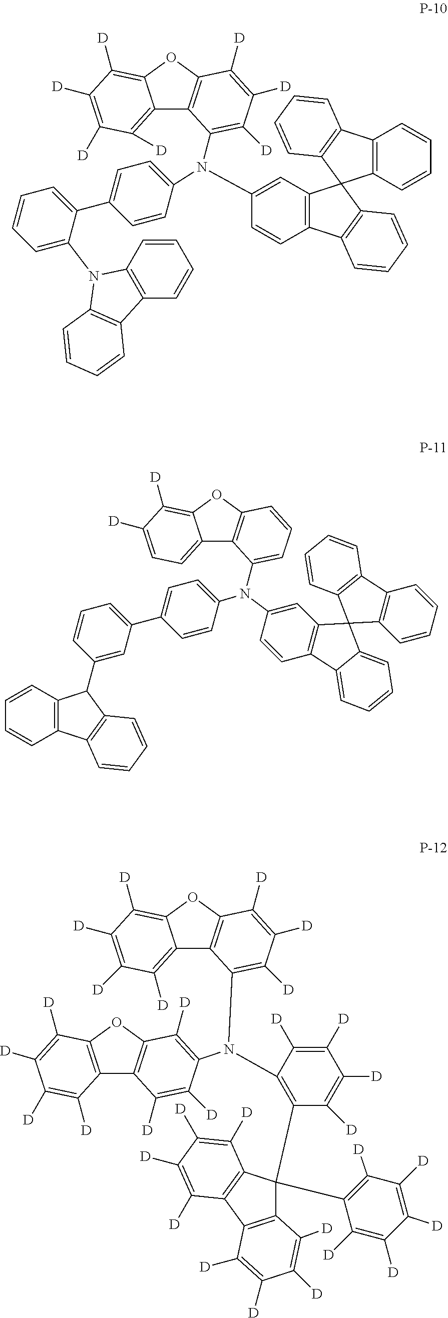

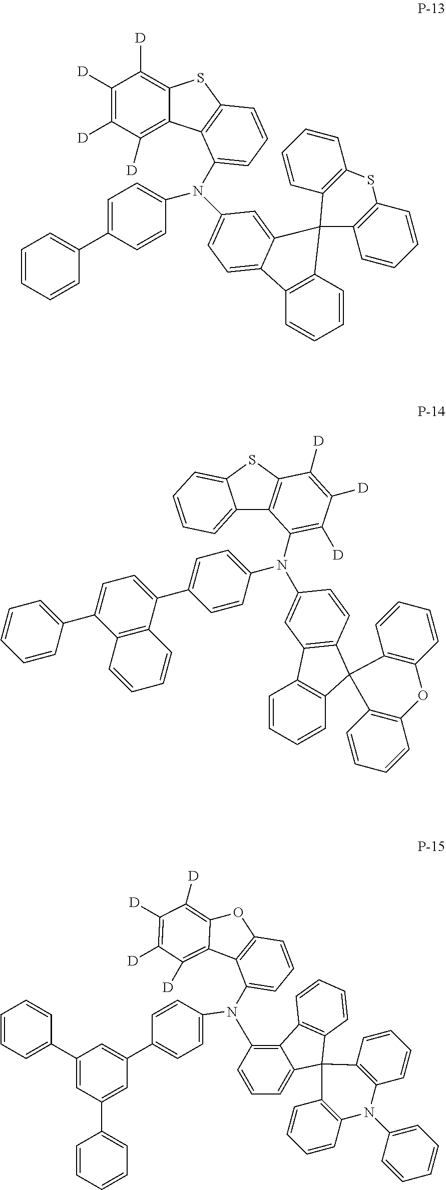 [SMILES: [2H]c1c([2H])c([2H])c2c(sc3cccc(N(c4ccc(-c5ccccc5)cc4)c4ccc5c(c4)C4(c6ccccc6Sc6ccccc64)c4ccccc4-5)c32)c1[2H] ; [2H]c1c([2H])c(N(c2ccc(-c3ccc(-c4ccccc4)c4ccccc34)cc2)c2ccc3c(c2)-c2ccccc2C32c3ccccc3Oc3ccccc32)c2c(sc3ccccc32)c1[2H] ; [2H]c1c([2H])c([2H])c2c(oc3cccc(N(c4ccc(-c5cc(-c6ccccc6)cc(-c6ccccc6)c5)cc4)c4cccc5c4-c4ccccc4C54c5ccccc5N(c5ccccc5)c5ccccc54)c32)c1[2H]]

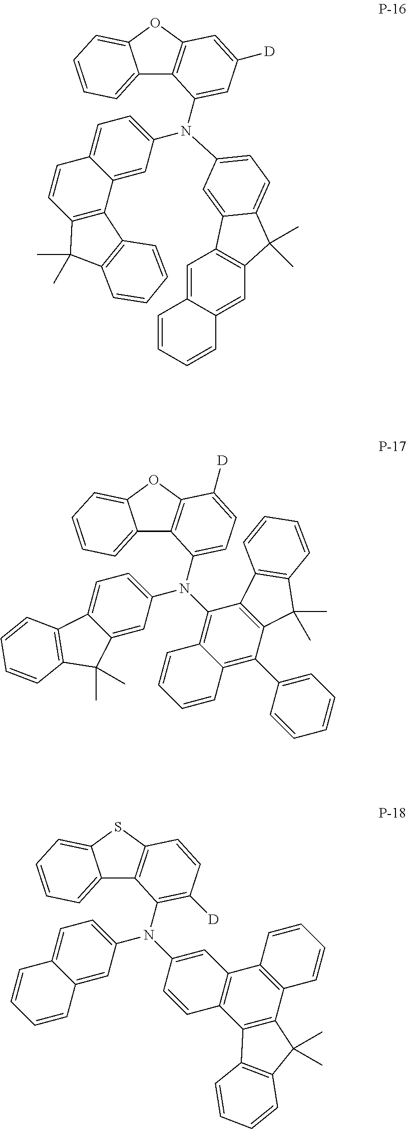

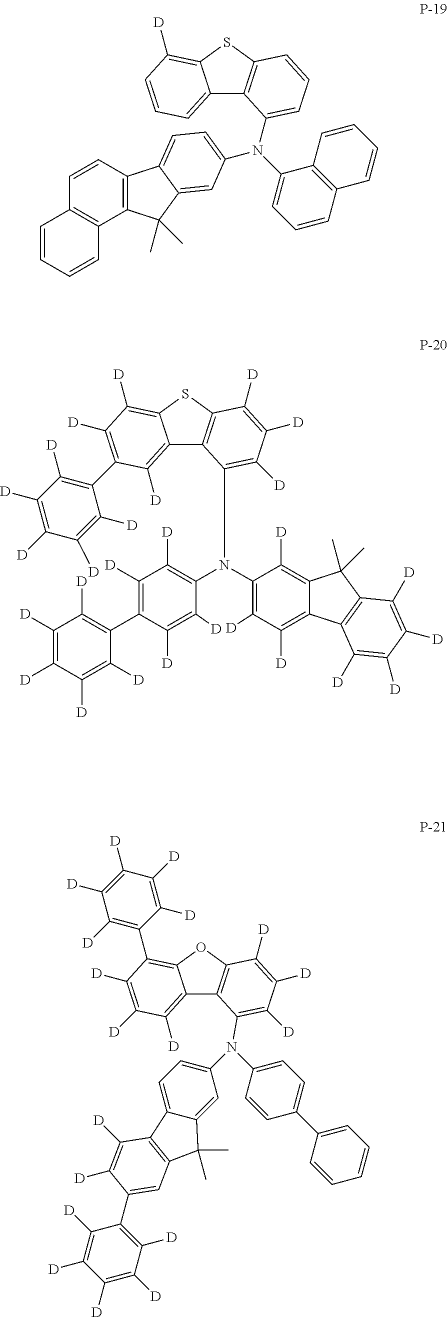

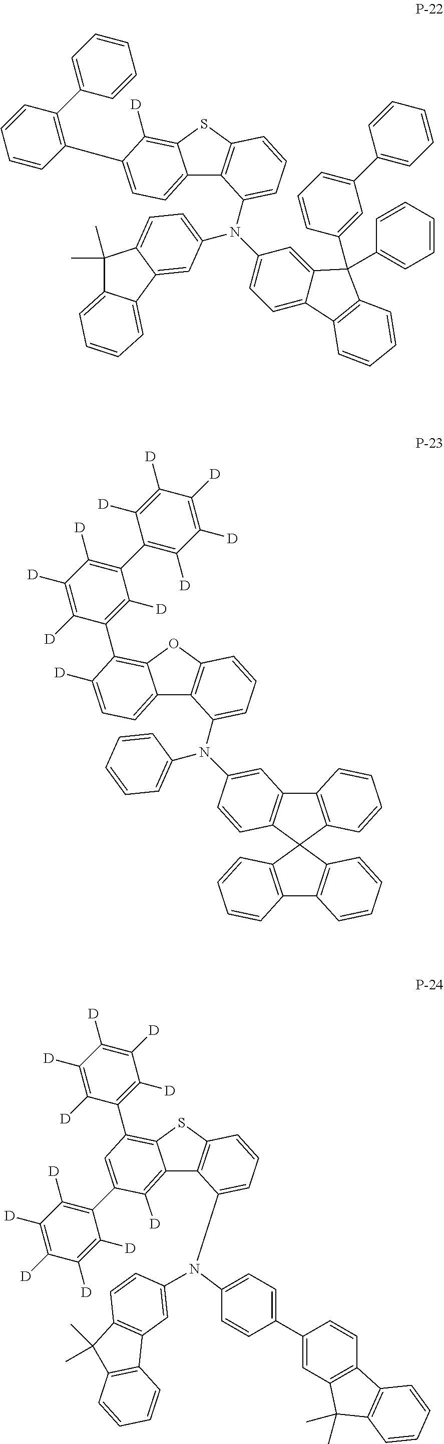 [SMILES: [2H]c1c(-c2ccccc2-c2ccccc2)ccc2c1sc1cccc(N(c3ccc4c(c3)-c3ccccc3C4(C)C)c3ccc4c(c3)C(c3ccccc3)(c3cccc(-c5ccccc5)c3)c3ccccc3-4)c12 ; [2H]c1ccc2c(oc3cccc(N(c4ccccc4)c4ccc5c(c4)-c4ccccc4C54c5ccccc5-c5ccccc54)c32)c1-c1c([2H])c([2H])c([2H])c(-c2c([2H])c([2H])c([2H])c([2H])c2[2H])c1[2H] ; [2H]c1c([2H])c([2H])c(-c2cc(-c3c([2H])c([2H])c([2H])c([2H])c3[2H])c3sc4cccc(N(c5ccc(-c6ccc7c(c6)C(C)(C)c6ccccc6-7)cc5)c5ccc6c(c5)-c5ccccc5C6(C)C)c4c3c2[2H])c([2H])c1[2H]]

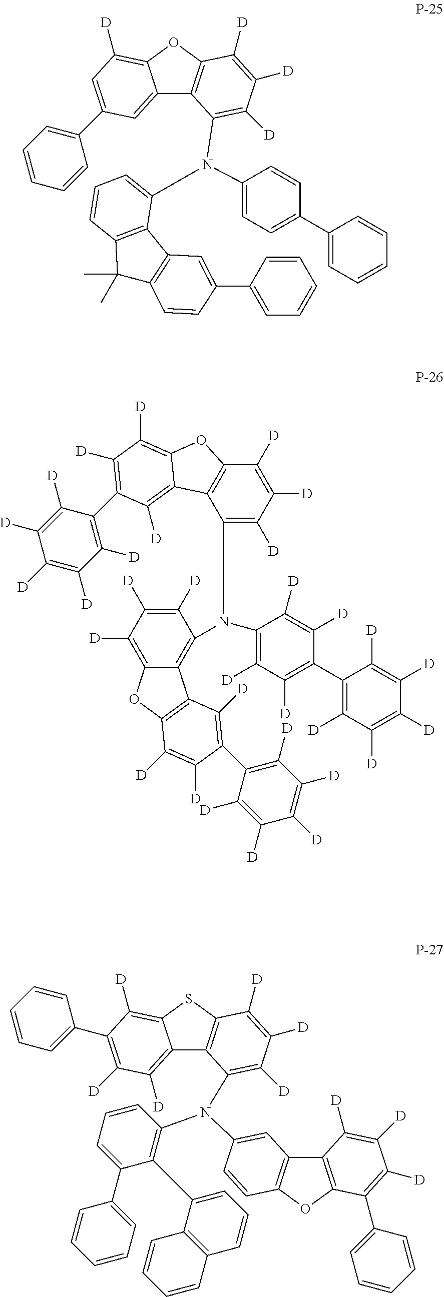

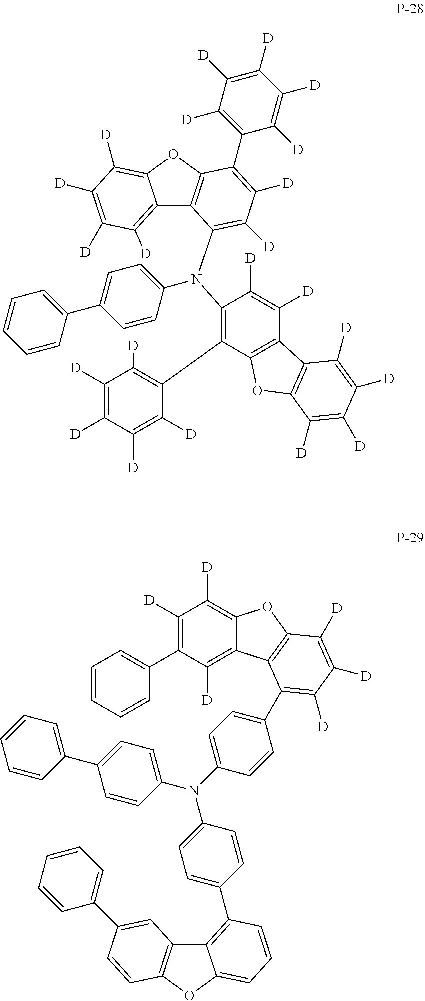 [SMILES: [2H]c1c([2H])c([2H])c(-c2c(N(c3ccc(-c4ccccc4)cc3)c3c([2H])c([2H])c(-c4c([2H])c([2H])c([2H])c([2H])c4[2H])c4oc5c([2H])c([2H])c([2H])c([2H])c5c34)c([2H])c([2H])c3c2oc2c([2H])c([2H])c([2H])c([2H])c23)c([2H])c1[2H] ; [2H]c1c(-c2ccccc2)c([2H])c2c(oc3c([2H])c([2H])c([2H])c(-c4ccc(N(c5ccc(-c6ccccc6)cc5)c5ccc(-c6cccc7oc8ccc(-c9ccccc9)cc8c67)cc5)cc4)c32)c1[2H]]

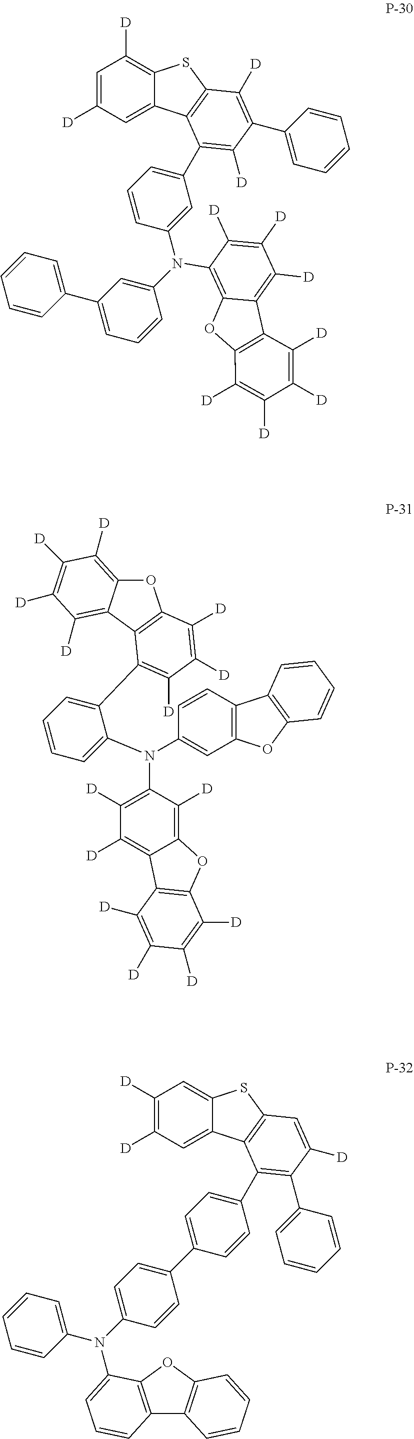 [SMILES: [2H]c1cc([2H])c2sc3c([2H])c(-c4ccccc4)c([2H])c(-c4cccc(N(c5cccc(-c6ccccc6)c5)c5c([2H])c([2H])c([2H])c6c5oc5c([2H])c([2H])c([2H])c([2H])c56)c4)c3c2c1 ; [2H]c1c([2H])c([2H])c2c(oc3c([2H])c(N(c4ccc5c(c4)oc4ccccc45)c4ccccc4-c4c([2H])c([2H])c([2H])c5oc6c([2H])c([2H])c([2H])c([2H])c6c45)c([2H])c([2H])c32)c1[2H] ; [2H]c1cc2sc3cc([2H])c(-c4ccccc4)c(-c4ccc(-c5ccc(N(c6ccccc6)c6cccc7c6oc6ccccc67)cc5)cc4)c3c2cc1[2H]]

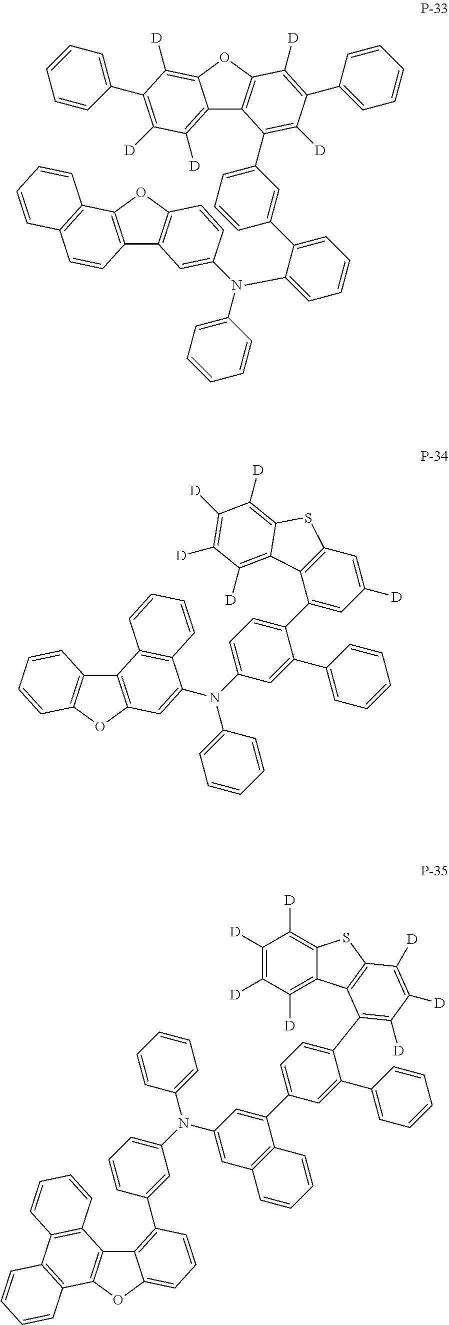 [SMILES: [2H]c1c(-c2ccccc2)c([2H])c2oc3c([2H])c(-c4ccccc4)c([2H])c(-c4cccc(-c5ccccc5N(c5ccccc5)c5ccc6oc7c8ccccc8ccc7c6c5)c4)c3c2c1[2H] ; [2H]c1cc(-c2ccc(N(c3ccccc3)c3cc4oc5ccccc5c4c4ccccc34)cc2-c2ccccc2)c2c(c1)sc1c([2H])c([2H])c([2H])c([2H])c12 ; [2H]c1c([2H])c([2H])c2c(sc3c([2H])c([2H])c([2H])c(-c4ccc(-c5cc(N(c6ccccc6)c6cccc(-c7cccc8oc9c%10ccccc%10c%10ccccc%10c9c78)c6)cc6ccccc56)cc4-c4ccccc4)c32)c1[2H]]

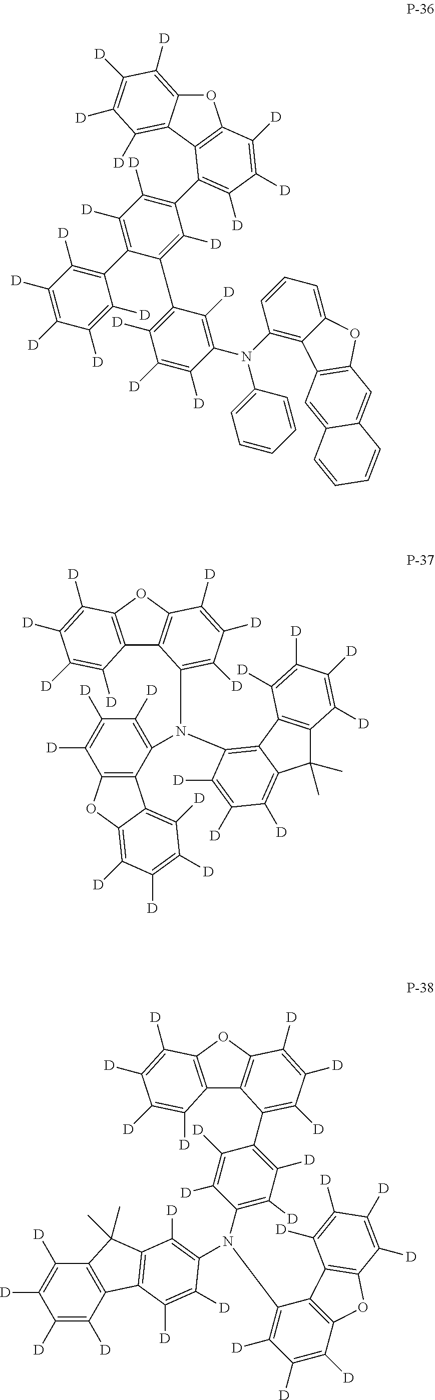

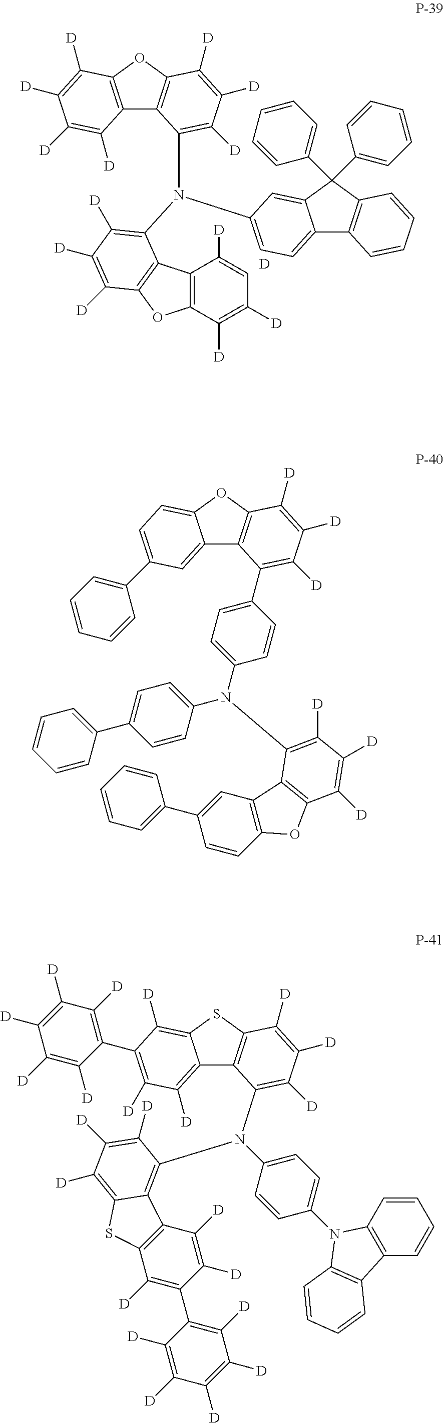

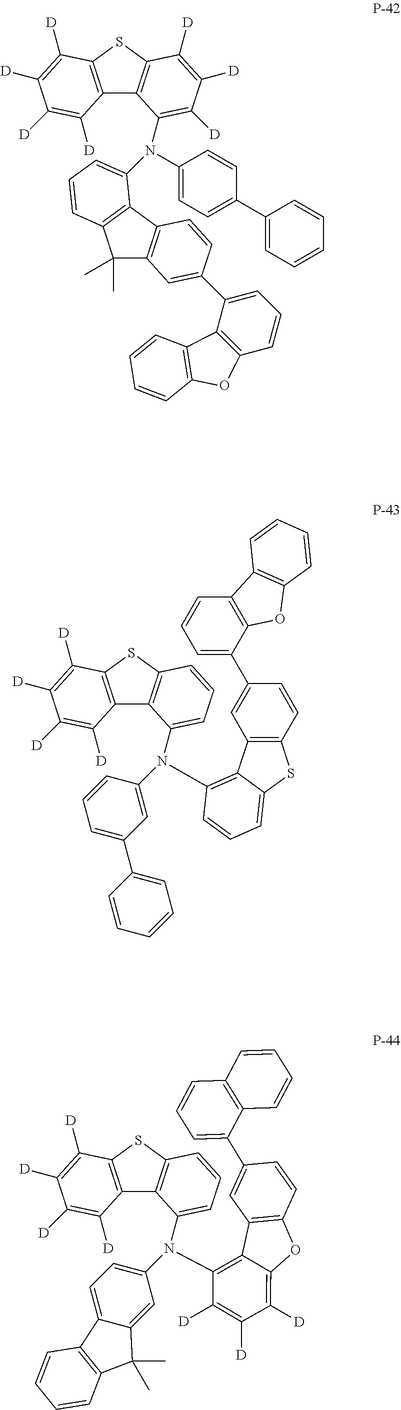 [SMILES: [2H]c1c([2H])c([2H])c2c(sc3c([2H])c([2H])c([2H])c(N(c4ccc(-c5ccccc5)cc4)c4cccc5c4-c4ccc(-c6cccc7oc8ccccc8c67)cc4C5(C)C)c32)c1[2H] ; [2H]c1c([2H])c([2H])c2c(sc3cccc(N(c4cccc(-c5ccccc5)c4)c4cccc5sc6ccc(-c7cccc8c7oc7ccccc78)cc6c45)c32)c1[2H] ; [2H]c1c([2H])c(N(c2ccc3c(c2)C(C)(C)c2ccccc2-3)c2cccc3sc4c([2H])c([2H])c([2H])c([2H])c4c23)c2c(oc3ccc(-c4cccc5ccccc45)cc32)c1[2H]]

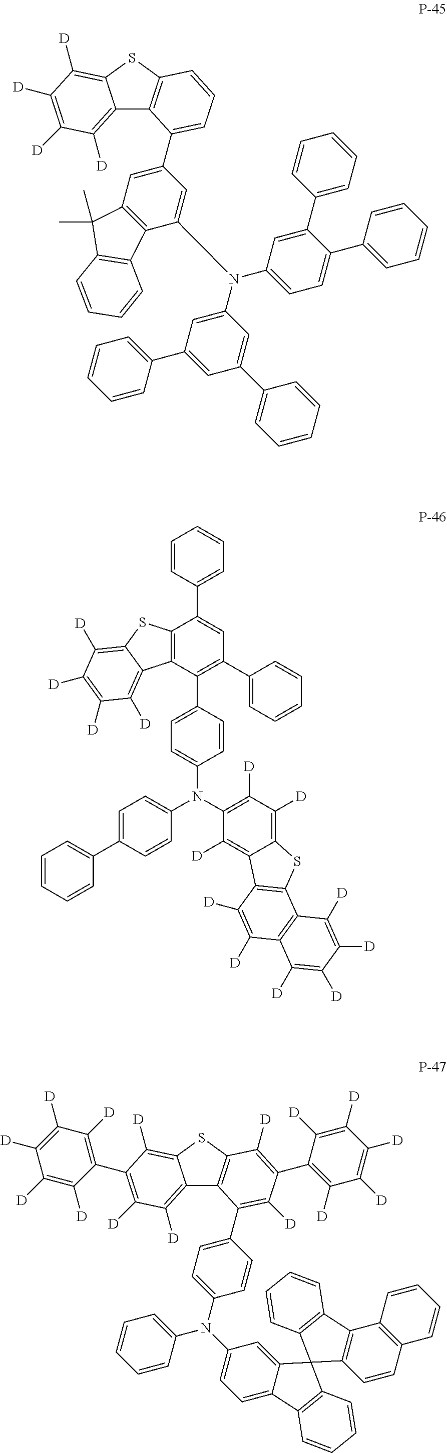

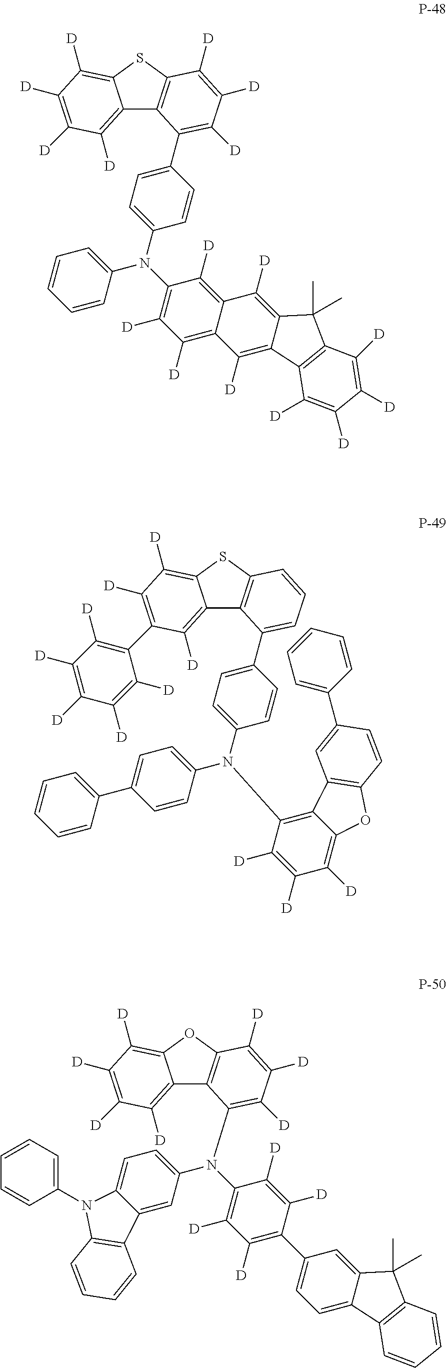 [SMILES: [2H]c1c([2H])c([2H])c2c(c1[2H])-c1c(c([2H])c3c([2H])c(N(c4ccccc4)c4ccc(-c5c([2H])c([2H])c([2H])c6sc7c([2H])c([2H])c([2H])c([2H])c7c56)cc4)c([2H])c([2H])c3c1[2H])C2(C)C ; [2H]c1c([2H])c([2H])c(-c2c([2H])c([2H])c3sc4cccc(-c5ccc(N(c6ccc(-c7ccccc7)cc6)c6c([2H])c([2H])c([2H])c7oc8ccc(-c9ccccc9)cc8c67)cc5)c4c3c2[2H])c([2H])c1[2H] ; [2H]c1c([2H])c(N(c2ccc3c(c2)c2ccccc2n3-c2ccccc2)c2c([2H])c([2H])c([2H])c3oc4c([2H])c([2H])c([2H])c([2H])c4c23)c([2H])c([2H])c1-c1ccc2c(c1)C(C)(C)c1ccccc1-2]

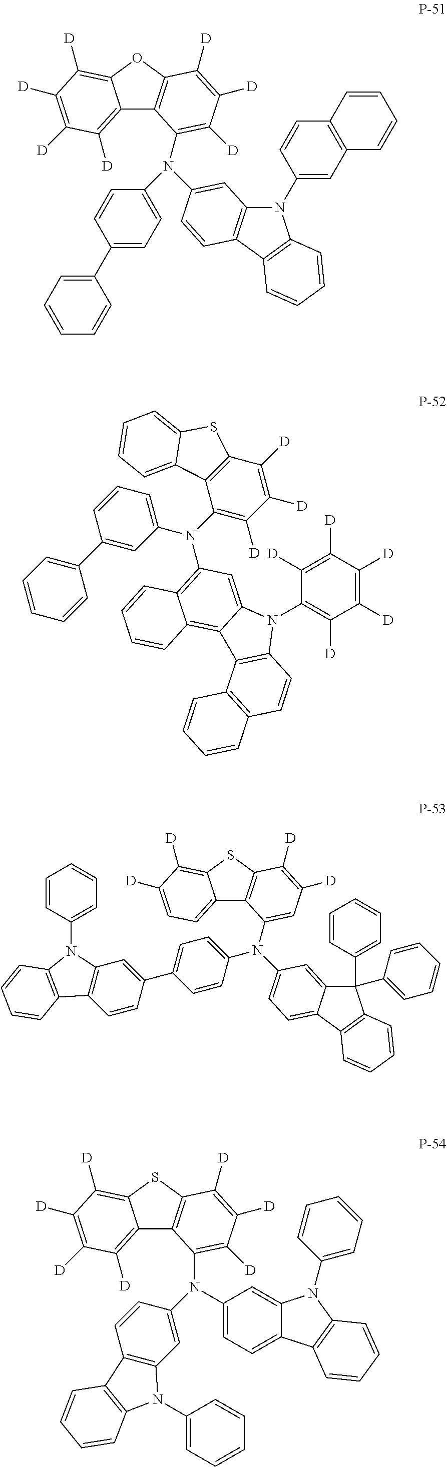

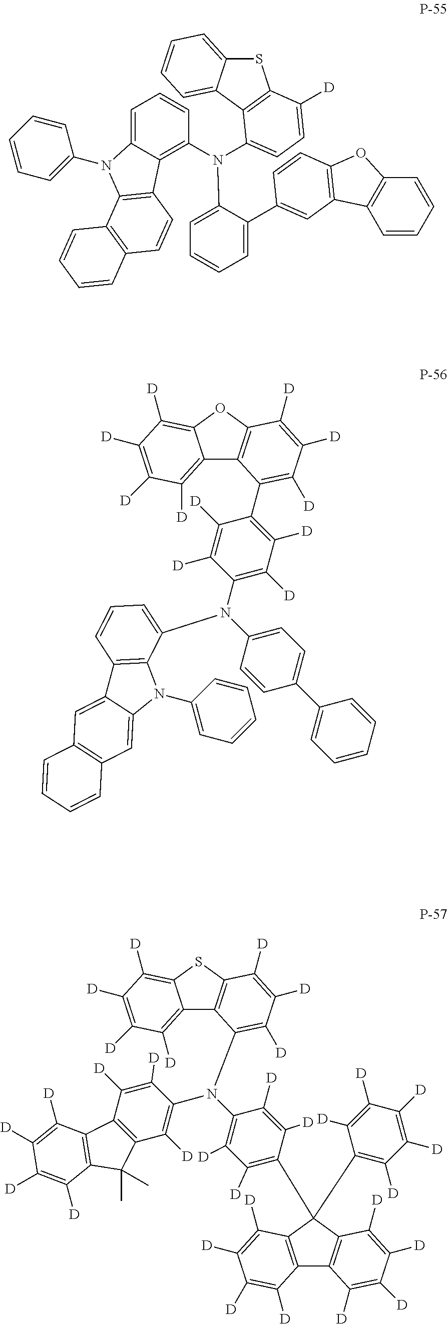

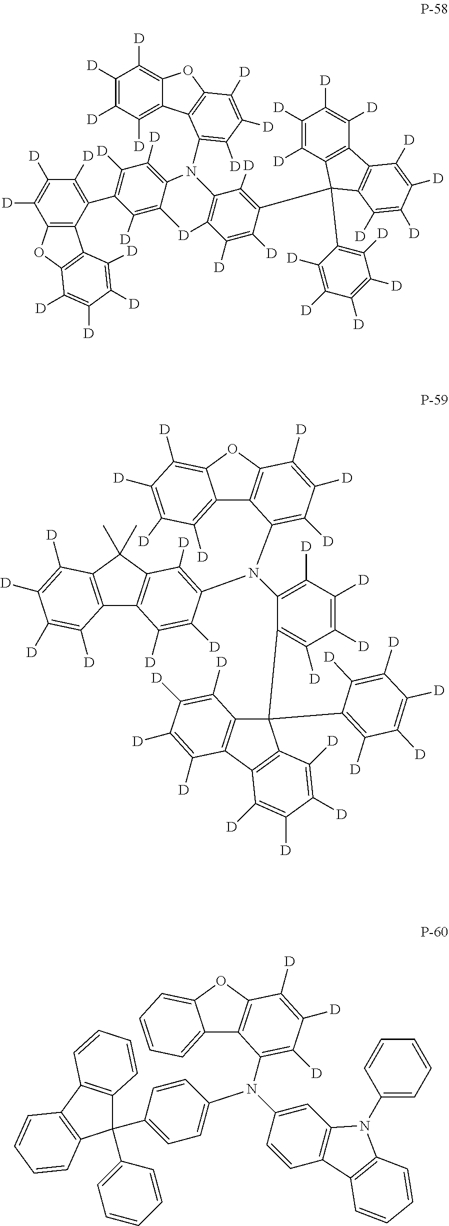

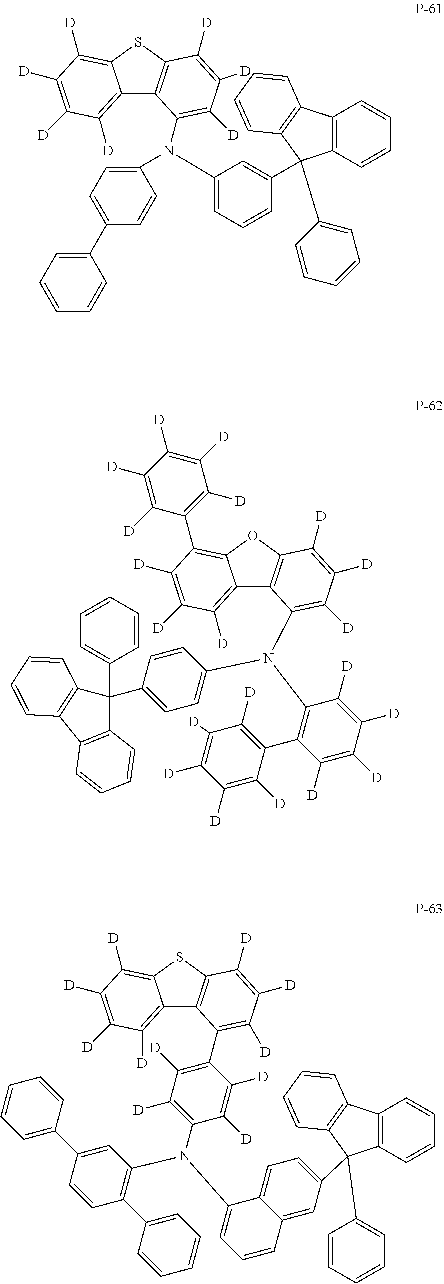

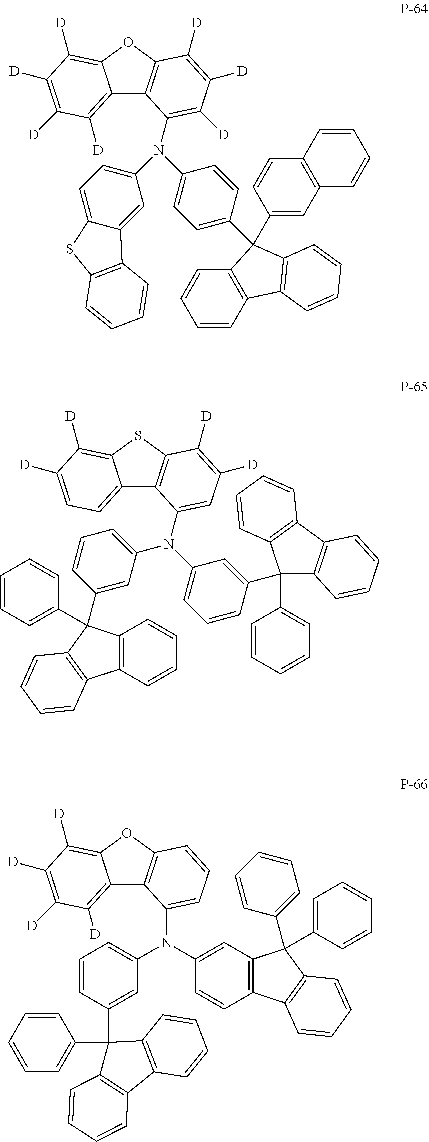 [SMILES: [2H]c1c([2H])c([2H])c2c(oc3c([2H])c([2H])c([2H])c(N(c4ccc(C5(c6ccc7ccccc7c6)c6ccccc6-c6ccccc65)cc4)c4ccc5sc6ccccc6c5c4)c32)c1[2H] ; [2H]c1ccc2c(sc3c([2H])c([2H])cc(N(c4cccc(C5(c6ccccc6)c6ccccc6-c6ccccc65)c4)c4cccc(C5(c6ccccc6)c6ccccc6-c6ccccc65)c4)c32)c1[2H] ; [2H]c1c([2H])c([2H])c2c(oc3cccc(N(c4cccc(C5(c6ccccc6)c6ccccc6-c6ccccc65)c4)c4ccc5c(c4)C(c4ccccc4)(c4ccccc4)c4ccccc4-5)c32)c1[2H]]

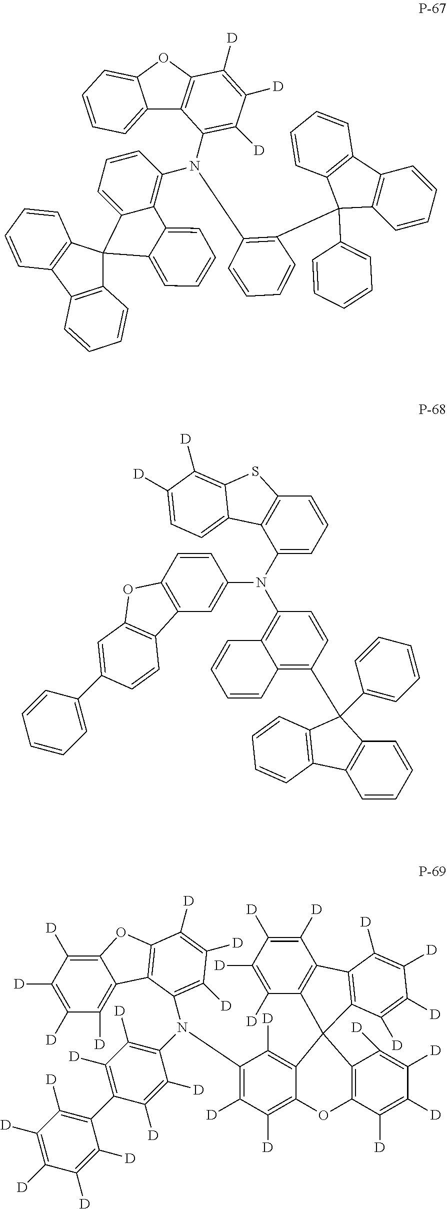 [SMILES: [2H]c1c([2H])c(N(c2ccccc2C2(c3ccccc3)c3ccccc3-c3ccccc32)c2cccc3c2-c2ccccc2C32c3ccccc3-c3ccccc32)c2c(oc3ccccc32)c1[2H] ; [2H]c1ccc2c(sc3cccc(N(c4ccc5oc6cc(-c7ccccc7)ccc6c5c4)c4ccc(C5(c6ccccc6)c6ccccc6-c6ccccc65)c5ccccc45)c32)c1[2H] ; [2H]c1c([2H])c([2H])c(-c2c([2H])c([2H])c(N(c3c([2H])c([2H])c4c(c3[2H])C3(c5c([2H])c([2H])c([2H])c([2H])c5O4)c4c([2H])c([2H])c([2H])c([2H])c4-c4c([2H])c([2H])c([2H])c([2H])c43)c3c([2H])c([2H])c([2H])c4oc5c([2H])c([2H])c([2H])c([2H])c5c34)c([2H])c2[2H])c([2H])c1[2H]]

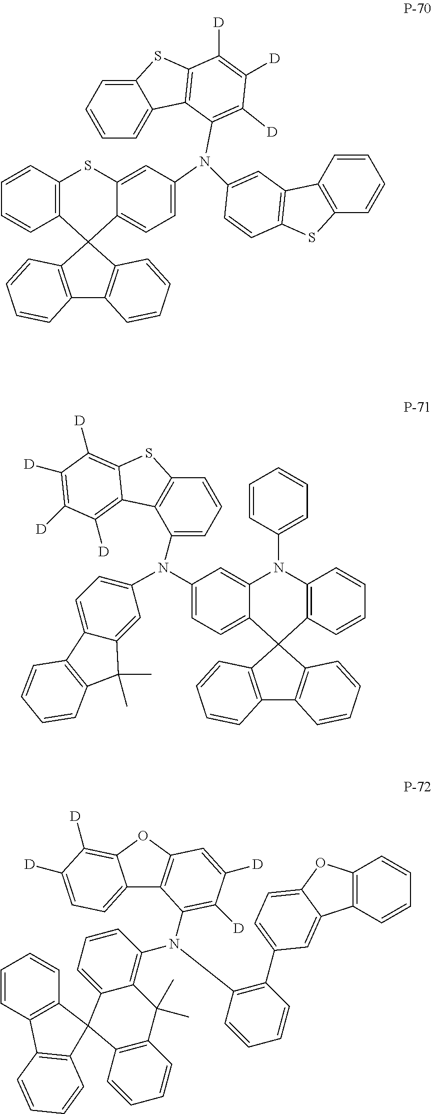 [SMILES: [2H]c1c([2H])c(N(c2ccc3c(c2)Sc2ccccc2C32c3ccccc3-c3ccccc32)c2ccc3sc4ccccc4c3c2)c2c(sc3ccccc32)c1[2H] ; [2H]c1c([2H])c([2H])c2c(sc3cccc(N(c4ccc5c(c4)N(c4ccccc4)c4ccccc4C54c5ccccc5-c5ccccc54)c4ccc5c(c4)C(C)(C)c4ccccc4-5)c32)c1[2H] ; [2H]c1cc2oc3c([2H])c([2H])ccc3c2c(N(c2ccccc2-c2ccc3oc4ccccc4c3c2)c2cccc3c2C(C)(C)c2ccccc2C32c3ccccc3-c3ccccc32)c1[2H]]

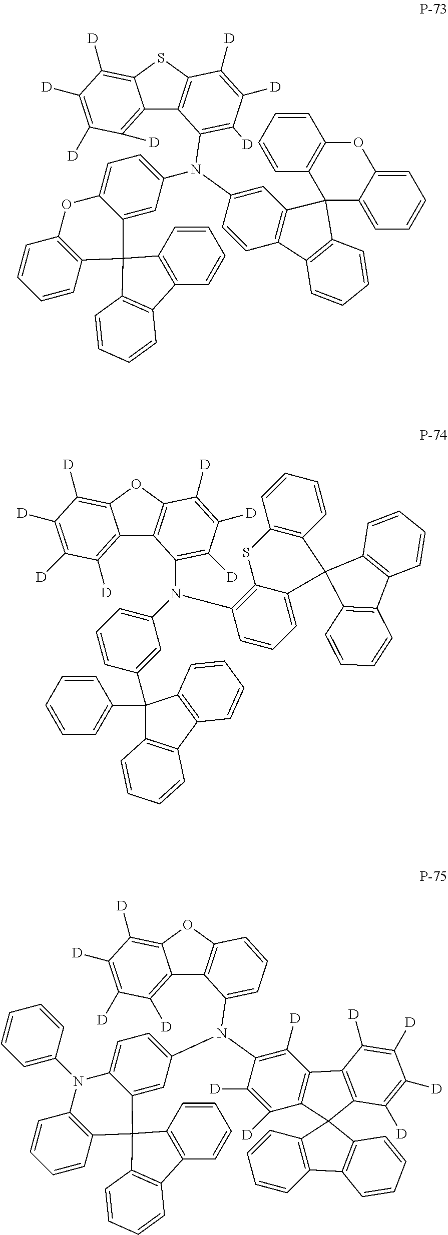 [SMILES: [2H]c1c([2H])c([2H])c2c(sc3c([2H])c([2H])c([2H])c(N(c4ccc5c(c4)C4(c6ccccc6O5)c5ccccc5-c5ccccc54)c4ccc5c(c4)C4(c6ccccc6Oc6ccccc64)c4ccccc4-5)c32)c1[2H] ; [2H]c1c([2H])c([2H])c2c(oc3c([2H])c([2H])c([2H])c(N(c4cccc(C5(c6ccccc6)c6ccccc6-c6ccccc65)c4)c4cccc5c4Sc4ccccc4C54c5ccccc5-c5ccccc54)c32)c1[2H] ; [2H]c1c([2H])c([2H])c2c(c1[2H])-c1c([2H])c(N(c3ccc4c(c3)C3(c5ccccc5-c5ccccc53)c3ccccc3N4c3ccccc3)c3cccc4oc5c([2H])c([2H])c([2H])c([2H])c5c34)c([2H])c([2H])c1C21c2ccccc2-c2ccccc21]

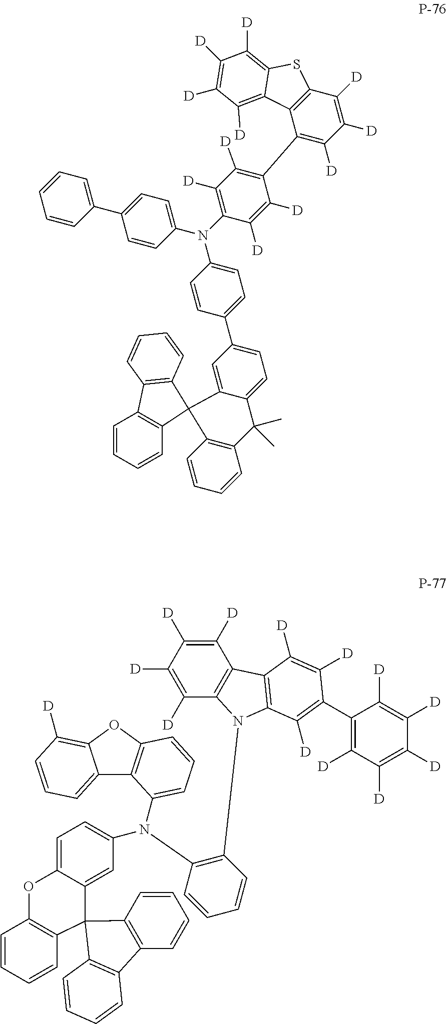 [SMILES: [2H]c1c([2H])c(N(c2ccc(-c3ccccc3)cc2)c2ccc(-c3ccc4c(c3)C3(c5ccccc5-c5ccccc53)c3ccccc3C4(C)C)cc2)c([2H])c([2H])c1-c1c([2H])c([2H])c([2H])c2sc3c([2H])c([2H])c([2H])c([2H])c3c12 ; [2H]c1c([2H])c([2H])c(-c2c([2H])c([2H])c3c4c([2H])c([2H])c([2H])c([2H])c4n(-c4ccccc4N(c4ccc5c(c4)C4(c6ccccc6O5)c5ccccc5-c5ccccc54)c4cccc5oc6c([2H])cccc6c45)c3c2[2H])c([2H])c1[2H]]

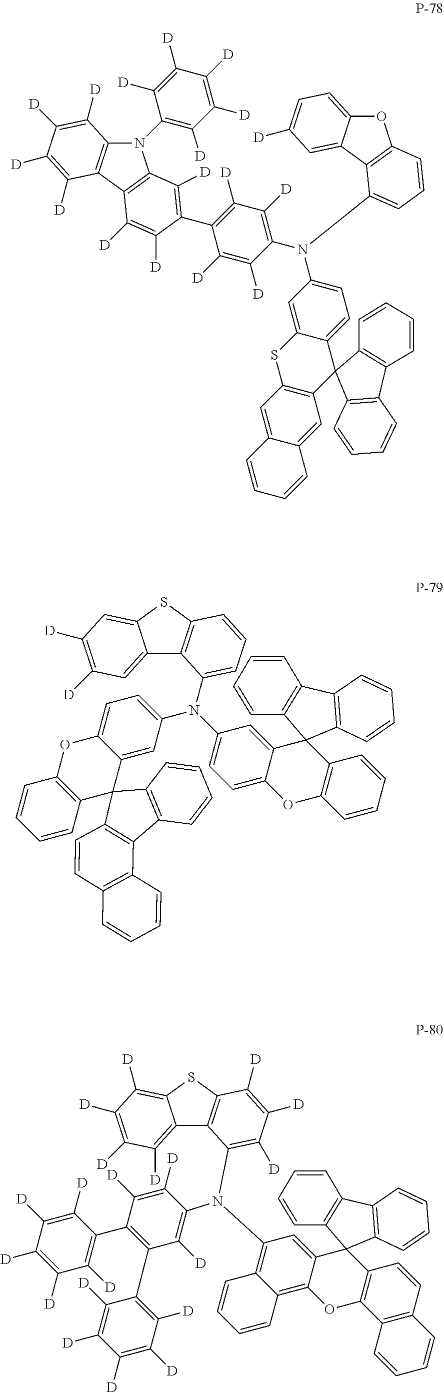

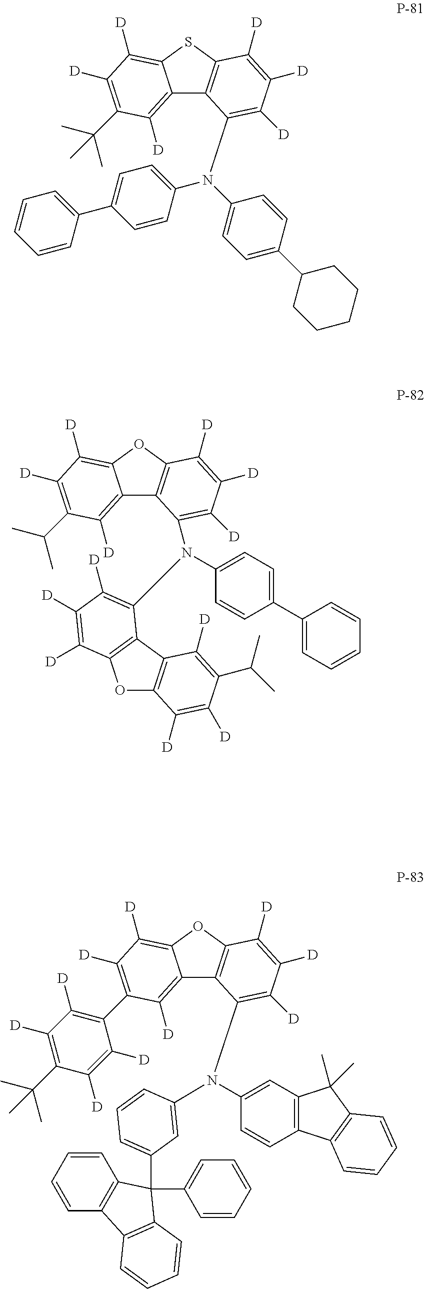 [SMILES: [2H]c1c(C(C)(C)C)c([2H])c2c(sc3c([2H])c([2H])c([2H])c(N(c4ccc(-c5ccccc5)cc4)c4ccc(C5CCCCC5)cc4)c32)c1[2H] ; [2H]c1c(C(C)C)c([2H])c2c(oc3c([2H])c([2H])c([2H])c(N(c4ccc(-c5ccccc5)cc4)c4c([2H])c([2H])c([2H])c5oc6c([2H])c([2H])c(C(C)C)c([2H])c6c45)c32)c1[2H] ; [2H]c1c([2H])c(C(C)(C)C)c([2H])c([2H])c1-c1c([2H])c([2H])c2oc3c([2H])c([2H])c([2H])c(N(c4cccc(C5(c6ccccc6)c6ccccc6-c6ccccc65)c4)c4ccc5c(c4)C(C)(C)c4ccccc4-5)c3c2c1[2H]]

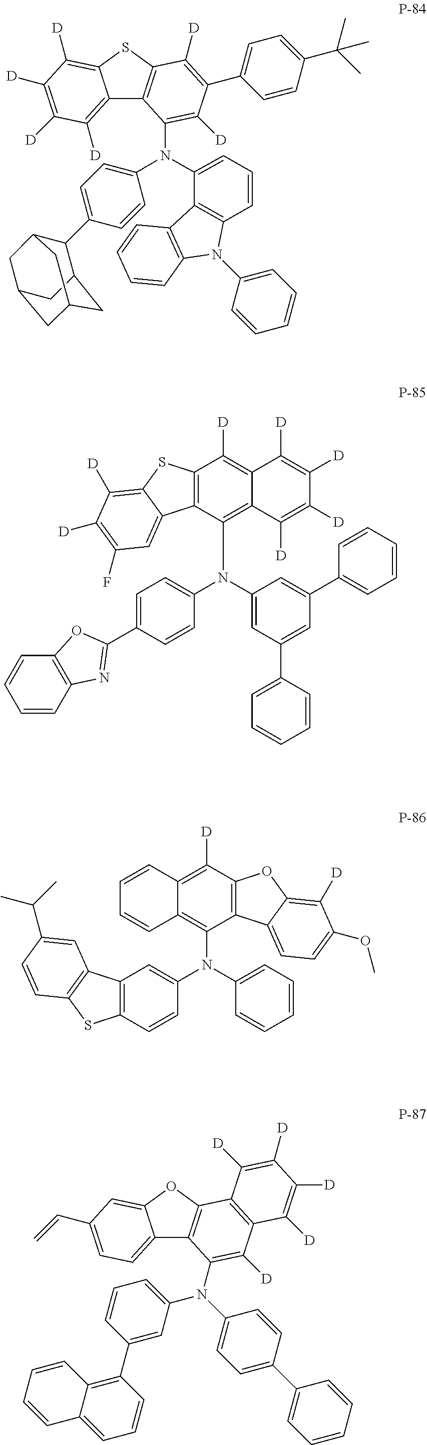

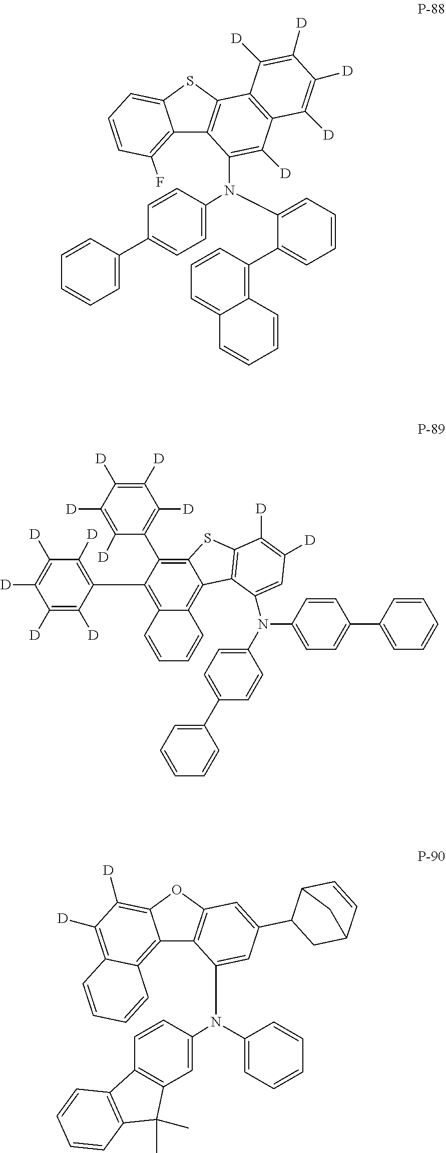 [SMILES: [2H]c1c([2H])c([2H])c2c(c1[2H])c([2H])c(N(c1ccc(-c3ccccc3)cc1)c1ccccc1-c1cccc3ccccc13)c1c2sc2cccc(F)c21 ; [2H]c1cc(N(c2ccc(-c3ccccc3)cc2)c2ccc(-c3ccccc3)cc2)c2c(sc3c(-c4c([2H])c([2H])c([2H])c([2H])c4[2H])c(-c4c([2H])c([2H])c([2H])c([2H])c4[2H])c4ccccc4c32)c1[2H] ; [2H]c1c([2H])c2oc3cc(C4CC5C=CC4C5)cc(N(c4ccccc4)c4ccc5c(c4)C(C)(C)c4ccccc4-5)c3c2c2ccccc12]

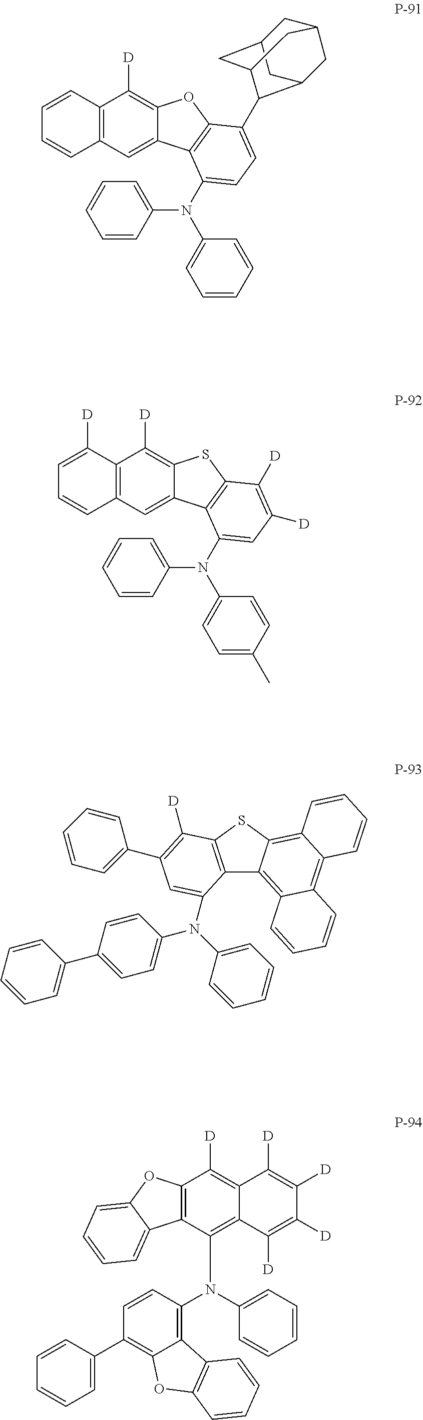 [SMILES: [2H]c1c2ccccc2cc2c1oc1c(C3C4CC5CC(C4)CC3C5)ccc(N(c3ccccc3)c3ccccc3)c12 ; [2H]c1cc(N(c2ccccc2)c2ccc(C)cc2)c2c(sc3c([2H])c4c([2H])cccc4cc32)c1[2H] ; [2H]c1c(-c2ccccc2)cc(N(c2ccccc2)c2ccc(-c3ccccc3)cc2)c2c1sc1c3ccccc3c3ccccc3c12 ; [2H]c1c([2H])c([2H])c2c(N(c3ccccc3)c3ccc(-c4ccccc4)c4oc5ccccc5c34)c3c(oc4ccccc43)c([2H])c2c1[2H]]

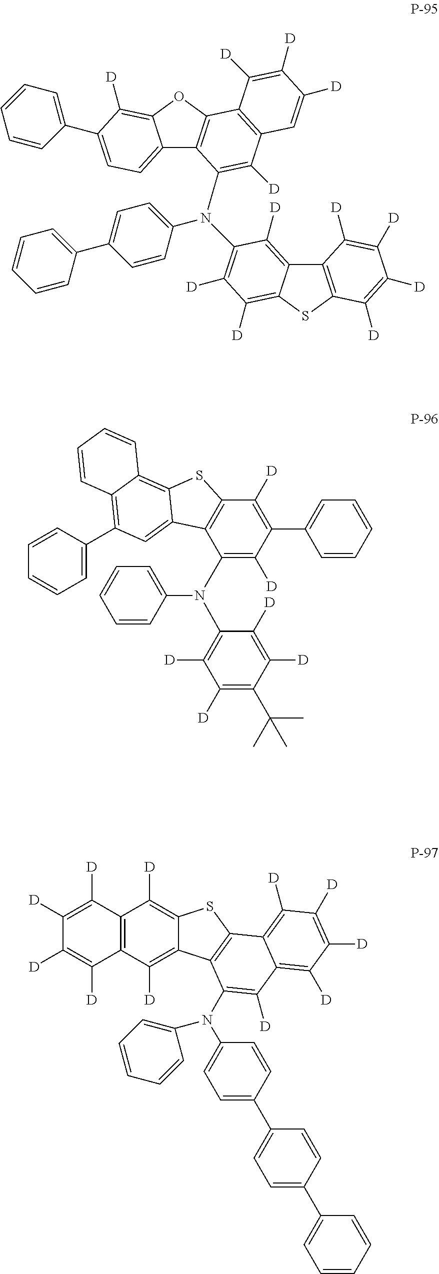 [SMILES: [2H]c1cc2c([2H])c(N(c3ccc(-c4ccccc4)cc3)c3c([2H])c([2H])c4sc5c([2H])c([2H])c([2H])c([2H])c5c4c3[2H])c3c4ccc(-c5ccccc5)c([2H])c4oc3c2c([2H])c1[2H] ; [2H]c1c([2H])c(C(C)(C)C)c([2H])c([2H])c1N(c1ccccc1)c1c([2H])c(-c2ccccc2)c([2H])c2sc3c4ccccc4c(-c4ccccc4)cc3c12 ; [2H]c1c([2H])c([2H])c2c([2H])c3c(sc4c5c([2H])c([2H])c([2H])c([2H])c5c([2H])c(N(c5ccccc5)c5ccc(-c6ccc(-c7ccccc7)cc6)cc5)c43)c([2H])c2c1[2H]]

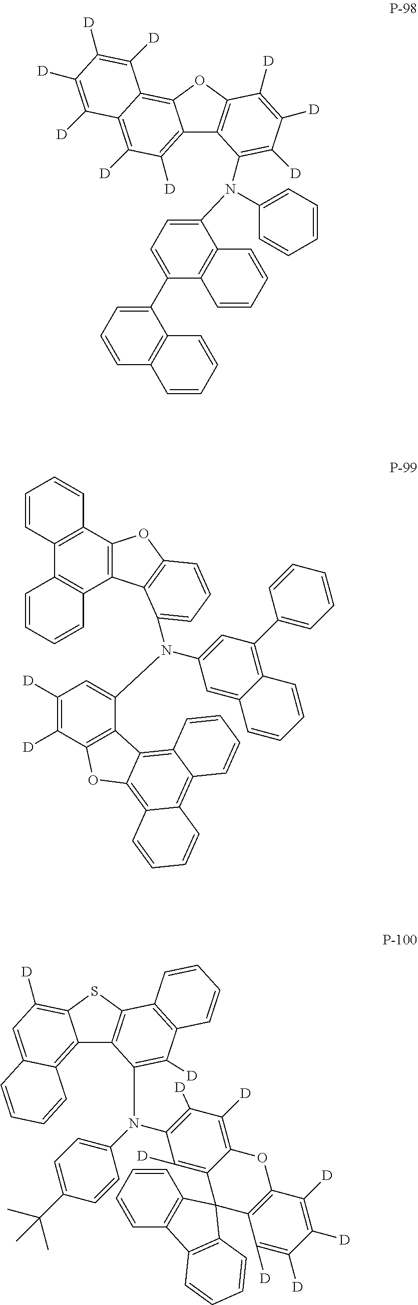 [SMILES: [2H]c1c([2H])c(N(c2ccccc2)c2ccc(-c3cccc4ccccc34)c3ccccc23)c2c(oc3c4c([2H])c([2H])c([2H])c([2H])c4c([2H])c([2H])c32)c1[2H] ; [2H]c1cc(N(c2cc(-c3ccccc3)c3ccccc3c2)c2cccc3oc4c5ccccc5c5ccccc5c4c23)c2c(oc3c4ccccc4c4ccccc4c32)c1[2H] ; [2H]c1c([2H])c([2H])c2c(c1[2H])Oc1c([2H])c([2H])c(N(c3ccc(C(C)(C)C)cc3)c3c([2H])c4ccccc4c4sc5c([2H])cc6ccccc6c5c34)c([2H])c1C21c2ccccc2-c2ccccc21]

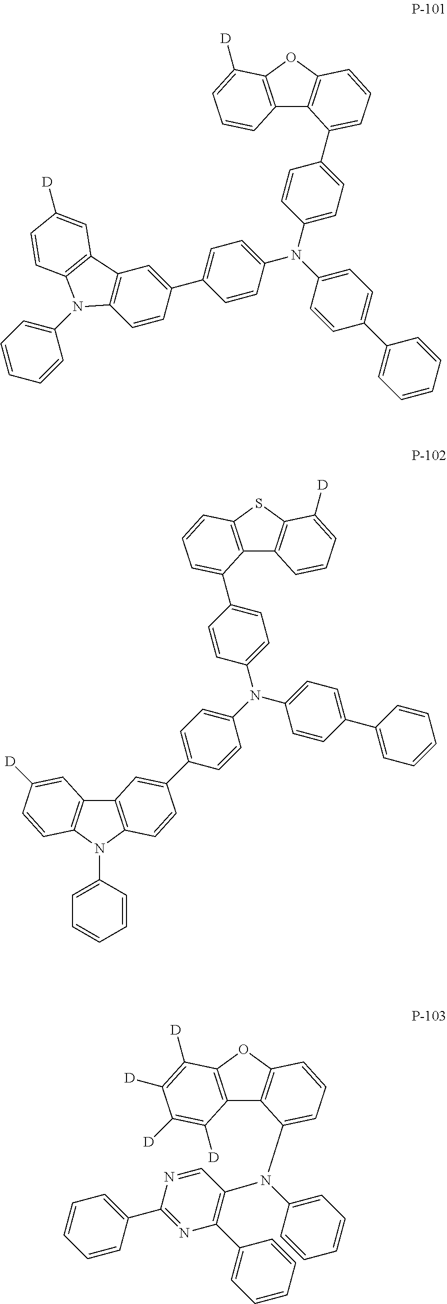 [SMILES: [2H]c1ccc2c(c1)c1cc(-c3ccc(N(c4ccc(-c5ccccc5)cc4)c4ccc(-c5cccc6oc7c([2H])cccc7c56)cc4)cc3)ccc1n2-c1ccccc1 ; [2H]c1ccc2c(c1)c1cc(-c3ccc(N(c4ccc(-c5ccccc5)cc4)c4ccc(-c5cccc6sc7c([2H])cccc7c56)cc4)cc3)ccc1n2-c1ccccc1 ; [2H]c1c([2H])c([2H])c2c(oc3cccc(N(c4ccccc4)c4cnc(-c5ccccc5)nc4-c4ccccc4)c32)c1[2H]]

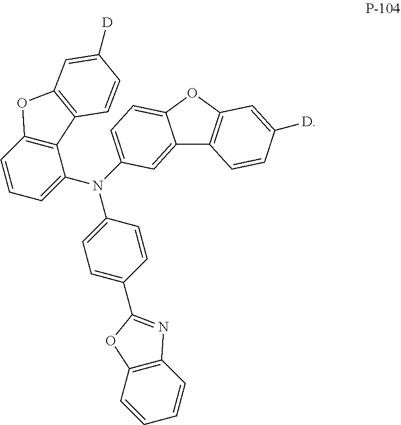

Compound represented by Formula 2 may be one of the following compounds, but there is no limitation thereto.

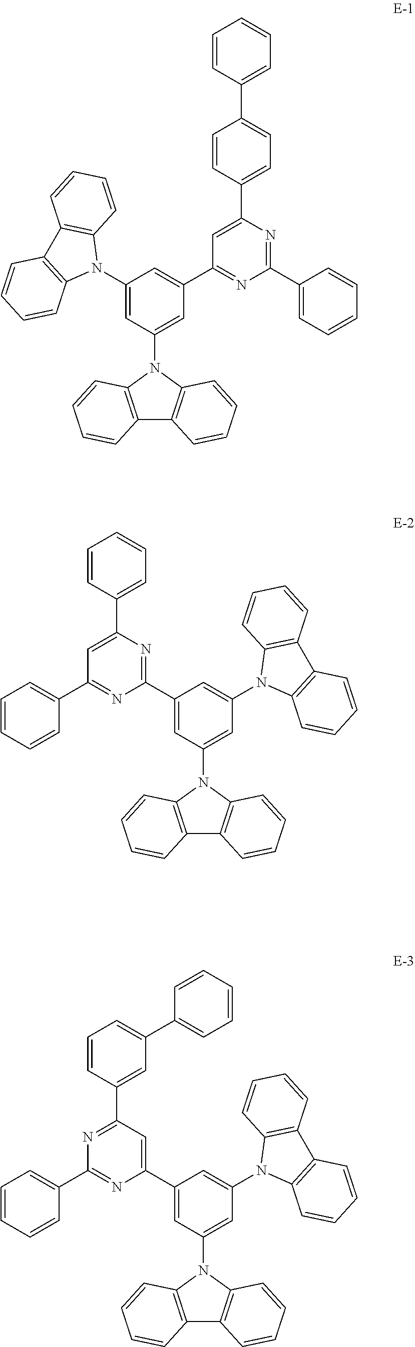 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cc(-c3ccccc3)nc(-c3cc(-n4c5ccccc5c5ccccc54)cc(-n4c5ccccc5c5ccccc54)c3)n2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

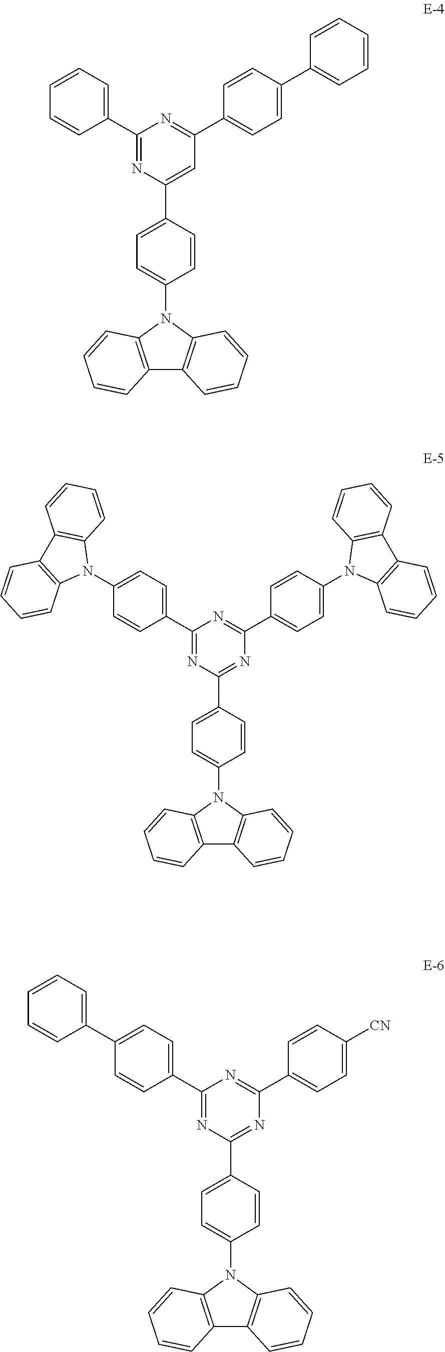 [SMILES: c1ccc(-c2ccc(-c3cc(-c4ccc(-n5c6ccccc6c6ccccc65)cc4)nc(-c4ccccc4)n3)cc2)cc1 ; c1ccc2c(c1)c1ccccc1n2-c1ccc(-c2nc(-c3ccc(-n4c5ccccc5c5ccccc54)cc3)nc(-c3ccc(-n4c5ccccc5c5ccccc54)cc3)n2)cc1 ; N#Cc1ccc(-c2nc(-c3ccc(-c4ccccc4)cc3)nc(-c3ccc(-n4c5ccccc5c5ccccc54)cc3)n2)cc1]

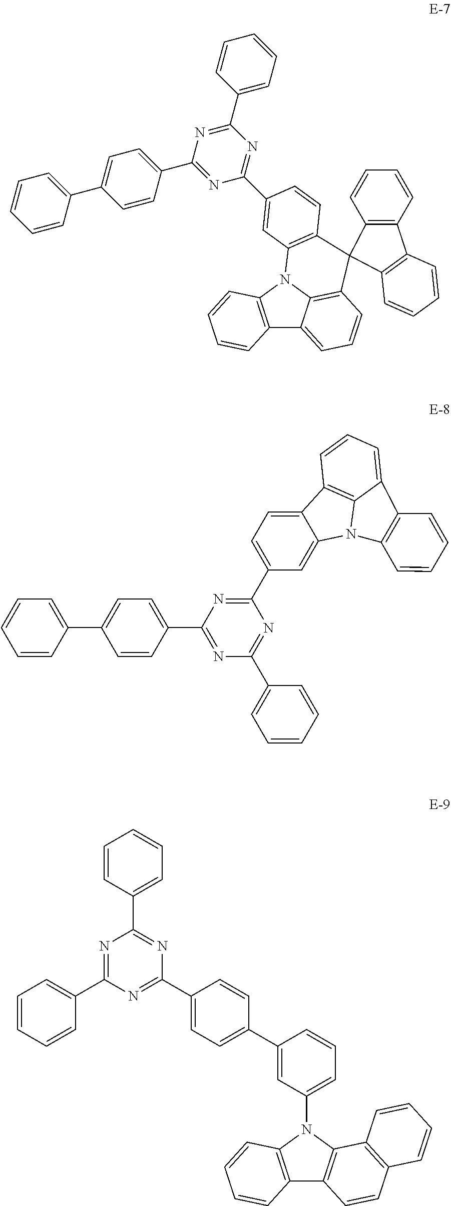 [SMILES: c1ccc(-c2ccc(-c3nc(-c4ccccc4)nc(-c4ccc5c(c4)-n4c6ccccc6c6cccc(c64)C54c5ccccc5-c5ccccc54)n3)cc2)cc1 ; c1ccc(-c2ccc(-c3nc(-c4ccccc4)nc(-c4ccc5c6cccc7c8ccccc8n(c5c4)c76)n3)cc2)cc1 ; c1ccc(-c2nc(-c3ccccc3)nc(-c3ccc(-c4cccc(-n5c6ccccc6c6ccc7ccccc7c65)c4)cc3)n2)cc1]

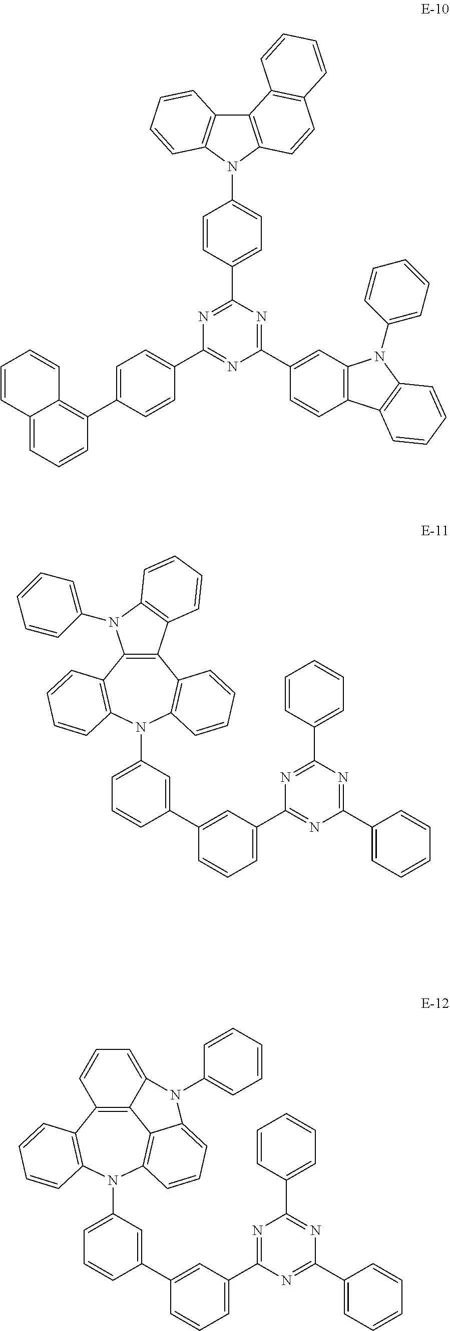 [SMILES: c1ccc(-n2c3ccccc3c3ccc(-c4nc(-c5ccc(-c6cccc7ccccc67)cc5)nc(-c5ccc(-n6c7ccccc7c7c8ccccc8ccc76)cc5)n4)cc32)cc1 ; c1ccc(-c2nc(-c3ccccc3)nc(-c3cccc(-c4cccc(N5c6ccccc6-c6c(n(-c7ccccc7)c7ccccc67)-c6ccccc65)c4)c3)n2)cc1 ; c1ccc(-c2nc(-c3ccccc3)nc(-c3cccc(-c4cccc(-n5c6ccccc6c6cccc7c6c6c5cccc6n7-c5ccccc5)c4)c3)n2)cc1]

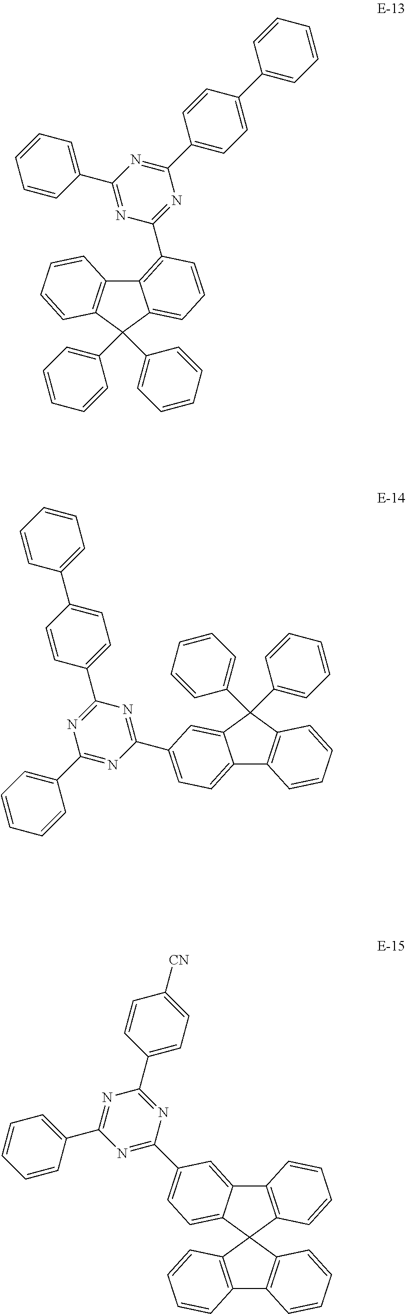 [SMILES: c1ccc(-c2ccc(-c3nc(-c4ccccc4)nc(-c4cccc5c4-c4ccccc4C5(c4ccccc4)c4ccccc4)n3)cc2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1 ; N#Cc1ccc(-c2nc(-c3ccccc3)nc(-c3ccc4c(c3)-c3ccccc3C43c4ccccc4-c4ccccc43)n2)cc1]

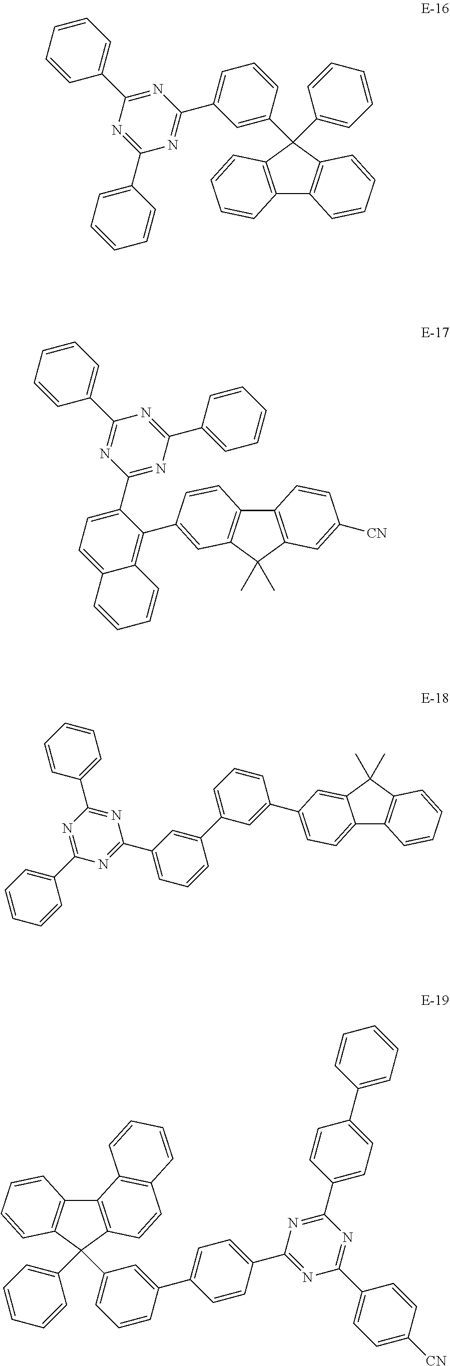 [SMILES: c1ccc(-c2nc(-c3ccccc3)nc(-c3cccc(C4(c5ccccc5)c5ccccc5-c5ccccc54)c3)n2)cc1 ; CC1(C)c2cc(C#N)ccc2-c2ccc(-c3c(-c4nc(-c5ccccc5)nc(-c5ccccc5)n4)ccc4ccccc34)cc21 ; CC1(C)c2ccccc2-c2ccc(-c3cccc(-c4cccc(-c5nc(-c6ccccc6)nc(-c6ccccc6)n5)c4)c3)cc21 ; N#Cc1ccc(-c2nc(-c3ccc(-c4ccccc4)cc3)nc(-c3ccc(-c4cccc(C5(c6ccccc6)c6ccccc6-c6c5ccc5ccccc65)c4)cc3)n2)cc1]

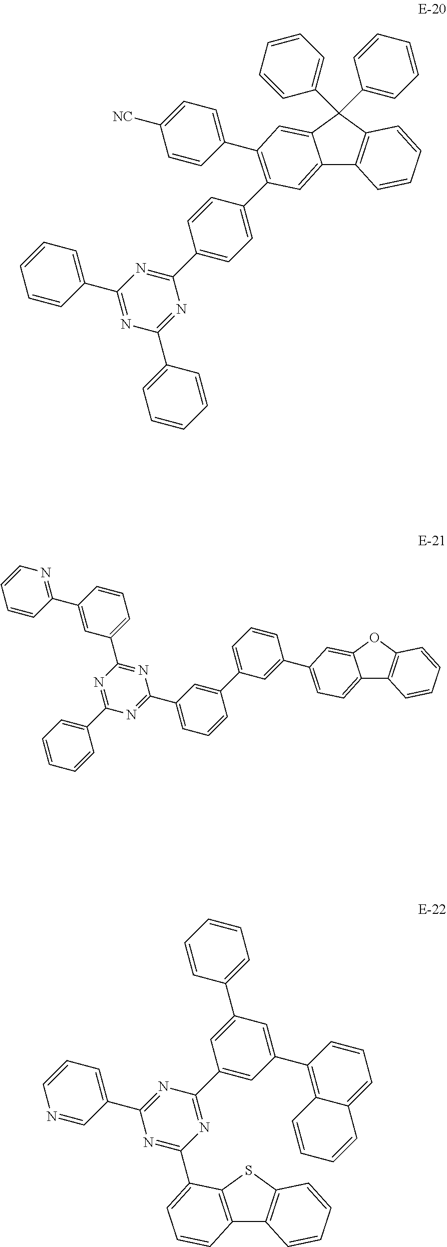 [SMILES: N#Cc1ccc(-c2cc3c(cc2-c2ccc(-c4nc(-c5ccccc5)nc(-c5ccccc5)n4)cc2)-c2ccccc2C3(c2ccccc2)c2ccccc2)cc1 ; c1ccc(-c2nc(-c3cccc(-c4cccc(-c5ccc6c(c5)oc5ccccc56)c4)c3)nc(-c3cccc(-c4ccccn4)c3)n2)cc1 ; c1ccc(-c2cc(-c3nc(-c4cccnc4)nc(-c4cccc5c4sc4ccccc45)n3)cc(-c3cccc4ccccc34)c2)cc1]

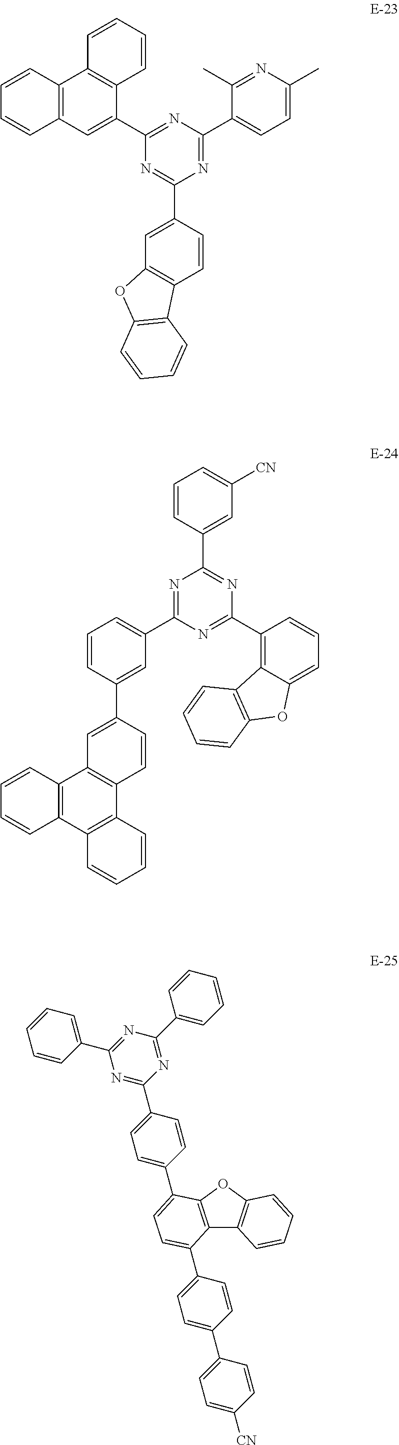 [SMILES: Cc1ccc(-c2nc(-c3ccc4c(c3)oc3ccccc34)nc(-c3cc4ccccc4c4ccccc34)n2)c(C)n1 ; N#Cc1cccc(-c2nc(-c3cccc(-c4ccc5c6ccccc6c6ccccc6c5c4)c3)nc(-c3cccc4oc5ccccc5c34)n2)c1 ; N#Cc1ccc(-c2ccc(-c3ccc(-c4ccc(-c5nc(-c6ccccc6)nc(-c6ccccc6)n5)cc4)c4oc5ccccc5c34)cc2)cc1]

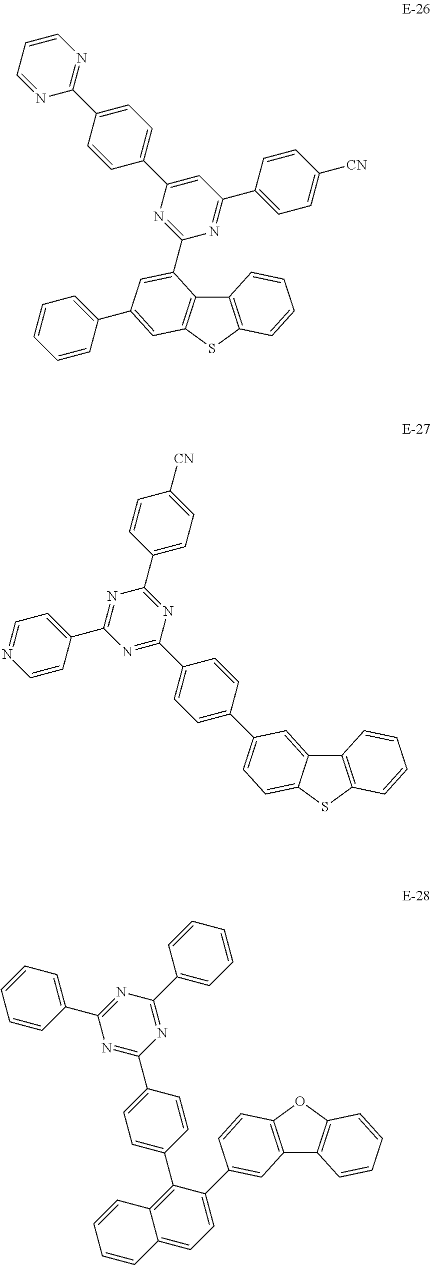 [SMILES: N#Cc1ccc(-c2cc(-c3ccc(-c4ncccn4)cc3)nc(-c3cc(-c4ccccc4)cc4sc5ccccc5c34)n2)cc1 ; N#Cc1ccc(-c2nc(-c3ccncc3)nc(-c3ccc(-c4ccc5sc6ccccc6c5c4)cc3)n2)cc1 ; c1ccc(-c2nc(-c3ccccc3)nc(-c3ccc(-c4c(-c5ccc6oc7ccccc7c6c5)ccc5ccccc45)cc3)n2)cc1]

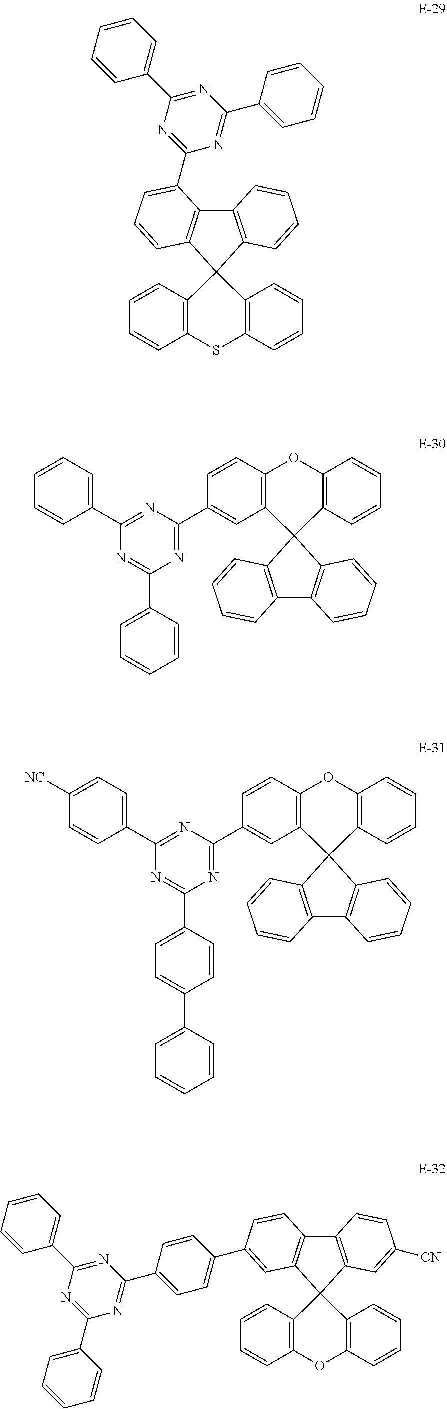 [SMILES: c1ccc(-c2nc(-c3ccccc3)nc(-c3cccc4c3-c3ccccc3C43c4ccccc4Sc4ccccc43)n2)cc1 ; c1ccc(-c2nc(-c3ccccc3)nc(-c3ccc4c(c3)C3(c5ccccc5O4)c4ccccc4-c4ccccc43)n2)cc1 ; N#Cc1ccc(-c2nc(-c3ccc(-c4ccccc4)cc3)nc(-c3ccc4c(c3)C3(c5ccccc5O4)c4ccccc4-c4ccccc43)n2)cc1 ; N#Cc1ccc2c(c1)C1(c3ccccc3Oc3ccccc31)c1cc(-c3ccc(-c4nc(-c5ccccc5)nc(-c5ccccc5)n4)cc3)ccc1-2]

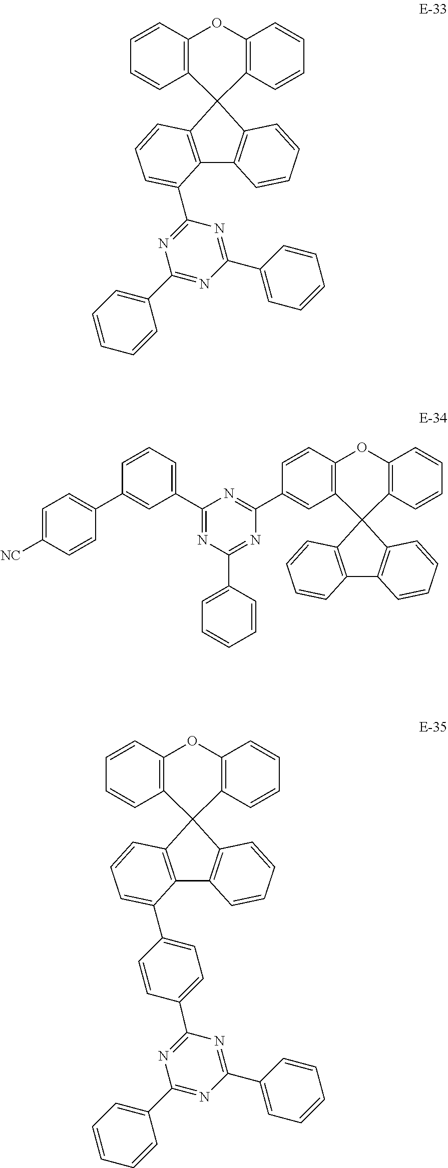 [SMILES: c1ccc(-c2nc(-c3ccccc3)nc(-c3cccc4c3-c3ccccc3C43c4ccccc4Oc4ccccc43)n2)cc1 ; N#Cc1ccc(-c2cccc(-c3nc(-c4ccccc4)nc(-c4ccc5c(c4)C4(c6ccccc6O5)c5ccccc5-c5ccccc54)n3)c2)cc1 ; c1ccc(-c2nc(-c3ccccc3)nc(-c3ccc(-c4cccc5c4-c4ccccc4C54c5ccccc5Oc5ccccc54)cc3)n2)cc1]

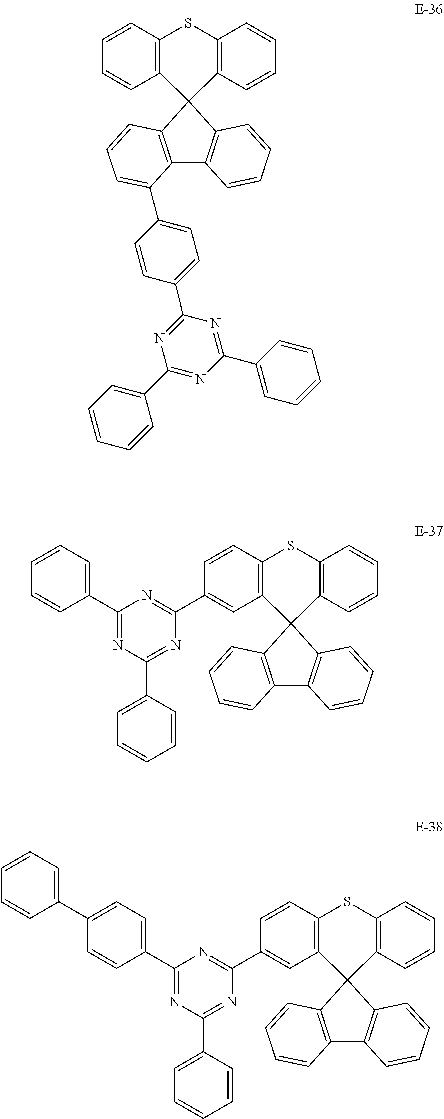 [SMILES: c1ccc(-c2nc(-c3ccccc3)nc(-c3ccc(-c4cccc5c4-c4ccccc4C54c5ccccc5Sc5ccccc54)cc3)n2)cc1 ; c1ccc(-c2nc(-c3ccccc3)nc(-c3ccc4c(c3)C3(c5ccccc5S4)c4ccccc4-c4ccccc43)n2)cc1 ; c1ccc(-c2ccc(-c3nc(-c4ccccc4)nc(-c4ccc5c(c4)C4(c6ccccc6S5)c5ccccc5-c5ccccc54)n3)cc2)cc1]

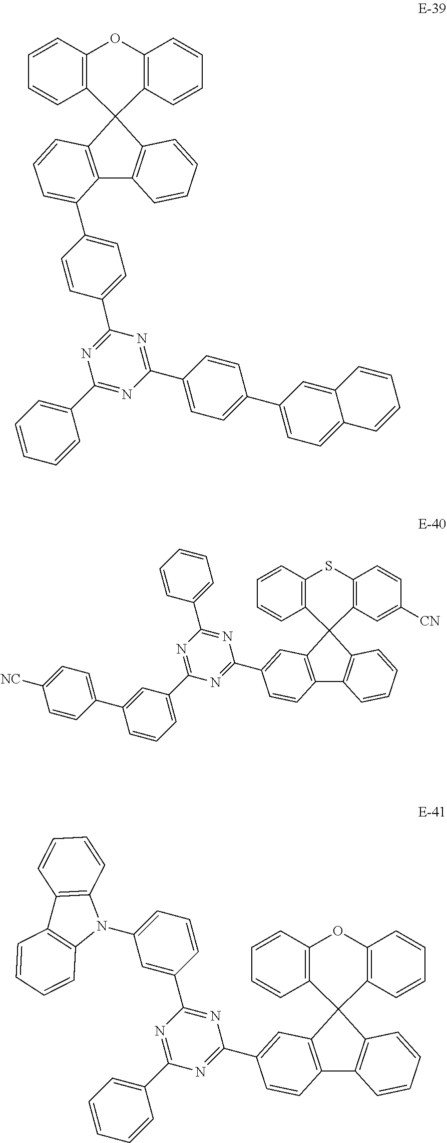 [SMILES: c1ccc(-c2nc(-c3ccc(-c4ccc5ccccc5c4)cc3)nc(-c3ccc(-c4cccc5c4-c4ccccc4C54c5ccccc5Oc5ccccc54)cc3)n2)cc1 ; N#Cc1ccc(-c2cccc(-c3nc(-c4ccccc4)nc(-c4ccc5c(c4)C4(c6ccccc6Sc6ccc(C#N)cc64)c4ccccc4-5)n3)c2)cc1 ; c1ccc(-c2nc(-c3cccc(-n4c5ccccc5c5ccccc54)c3)nc(-c3ccc4c(c3)C3(c5ccccc5Oc5ccccc53)c3ccccc3-4)n2)cc1]

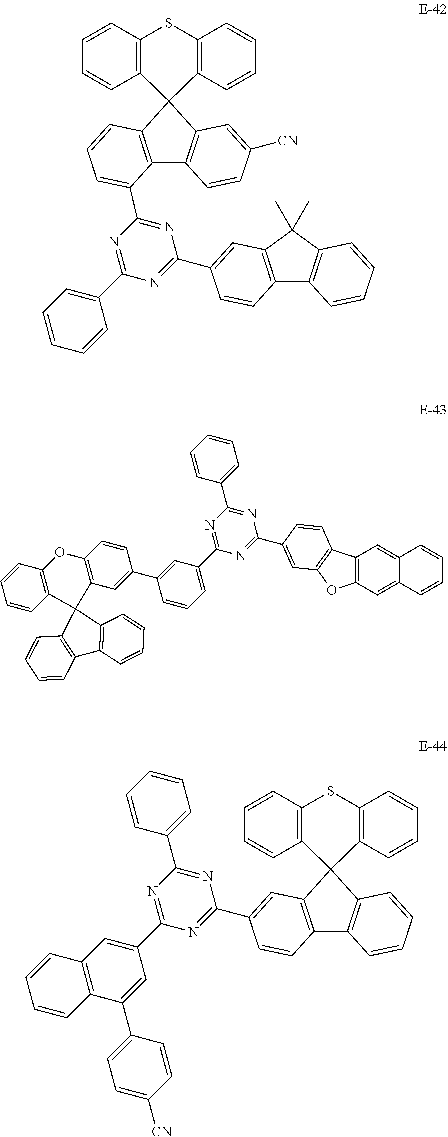 [SMILES: CC1(C)c2ccccc2-c2ccc(-c3nc(-c4ccccc4)nc(-c4cccc5c4-c4ccc(C#N)cc4C54c5ccccc5Sc5ccccc54)n3)cc21 ; c1ccc(-c2nc(-c3cccc(-c4ccc5c(c4)C4(c6ccccc6O5)c5ccccc5-c5ccccc54)c3)nc(-c3ccc4c(c3)oc3cc5ccccc5cc34)n2)cc1 ; N#Cc1ccc(-c2cc(-c3nc(-c4ccccc4)nc(-c4ccc5c(c4)C4(c6ccccc6Sc6ccccc64)c4ccccc4-5)n3)cc3ccccc23)cc1]

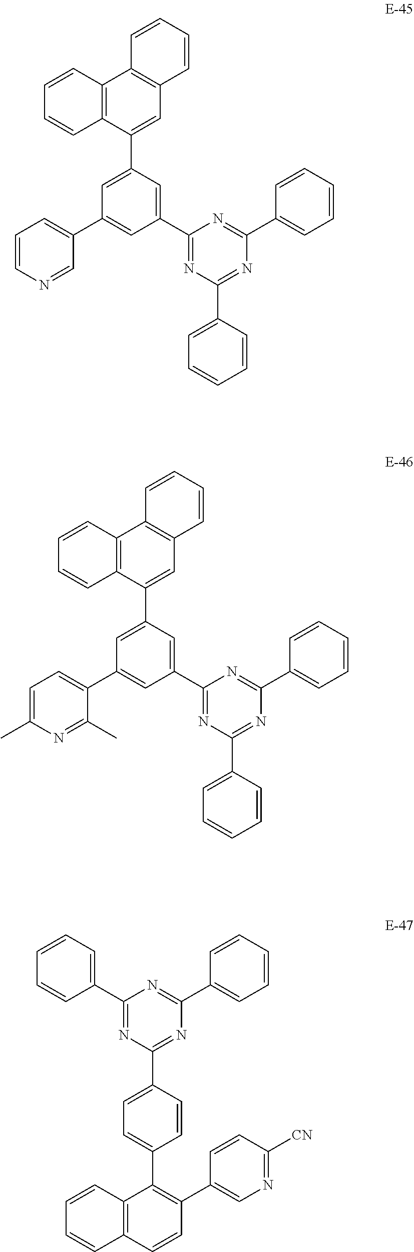 [SMILES: c1ccc(-c2nc(-c3ccccc3)nc(-c3cc(-c4cccnc4)cc(-c4cc5ccccc5c5ccccc45)c3)n2)cc1 ; Cc1ccc(-c2cc(-c3nc(-c4ccccc4)nc(-c4ccccc4)n3)cc(-c3cc4ccccc4c4ccccc34)c2)c(C)n1 ; N#Cc1ccc(-c2ccc3ccccc3c2-c2ccc(-c3nc(-c4ccccc4)nc(-c4ccccc4)n3)cc2)cn1]

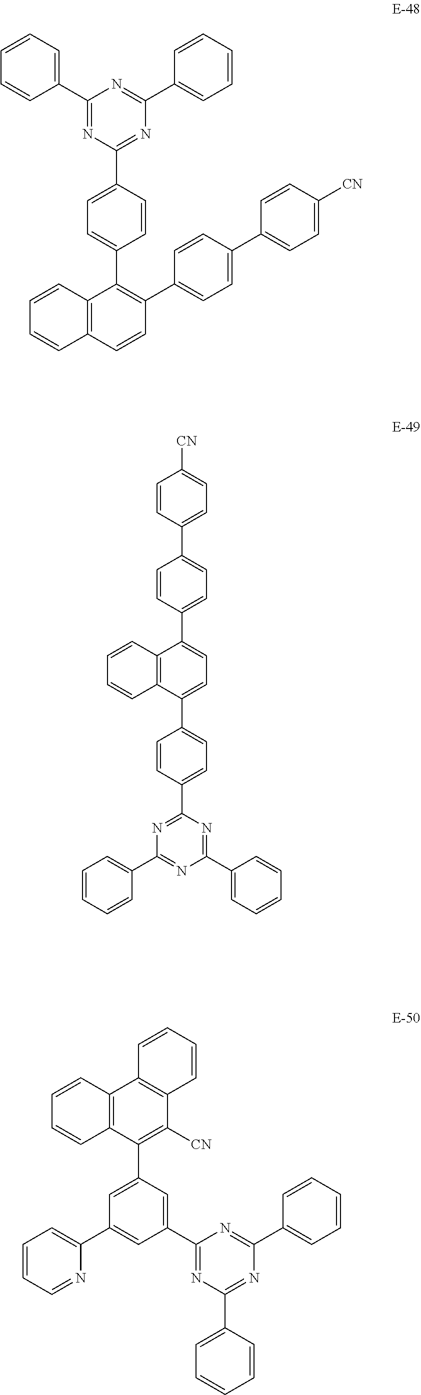 [SMILES: N#Cc1ccc(-c2ccc(-c3ccc4ccccc4c3-c3ccc(-c4nc(-c5ccccc5)nc(-c5ccccc5)n4)cc3)cc2)cc1 ; N#Cc1ccc(-c2ccc(-c3ccc(-c4ccc(-c5nc(-c6ccccc6)nc(-c6ccccc6)n5)cc4)c4ccccc34)cc2)cc1 ; N#Cc1c(-c2cc(-c3ccccn3)cc(-c3nc(-c4ccccc4)nc(-c4ccccc4)n3)c2)c2ccccc2c2ccccc12]

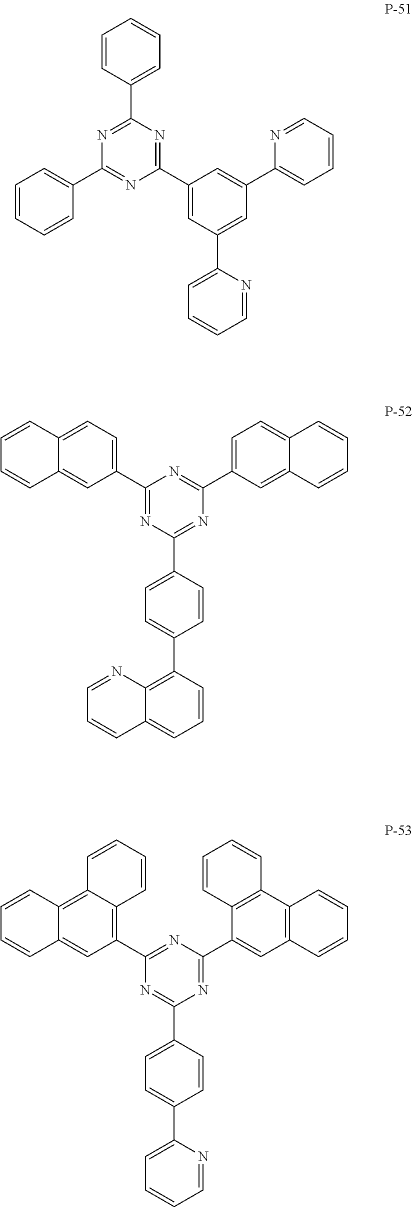 [SMILES: c1ccc(-c2nc(-c3ccccc3)nc(-c3cc(-c4ccccn4)cc(-c4ccccn4)c3)n2)cc1 ; c1ccc2cc(-c3nc(-c4ccc(-c5cccc6cccnc56)cc4)nc(-c4ccc5ccccc5c4)n3)ccc2c1 ; c1ccc(-c2ccc(-c3nc(-c4cc5ccccc5c5ccccc45)nc(-c4cc5ccccc5c5ccccc45)n3)cc2)nc1]

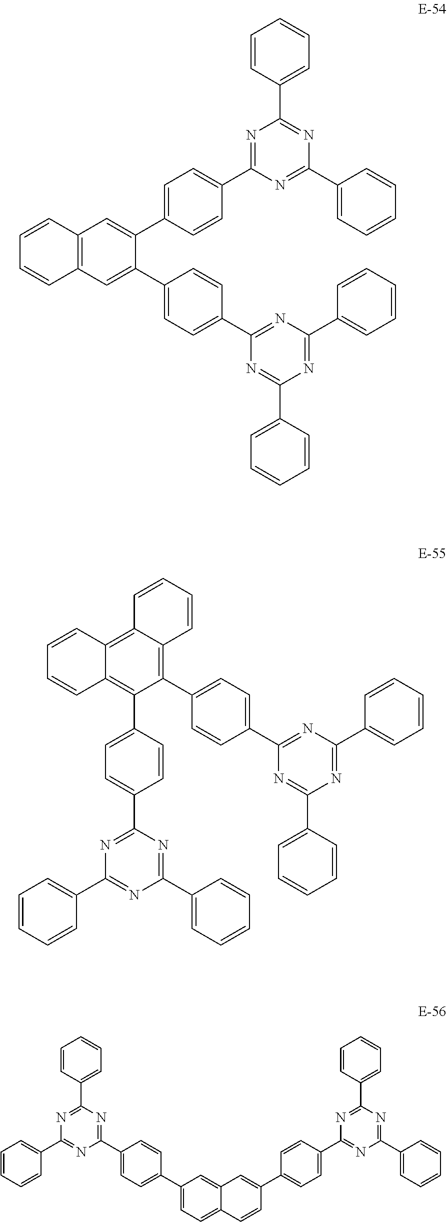 [SMILES: c1ccc(-c2nc(-c3ccccc3)nc(-c3ccc(-c4cc5ccccc5cc4-c4ccc(-c5nc(-c6ccccc6)nc(-c6ccccc6)n5)cc4)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

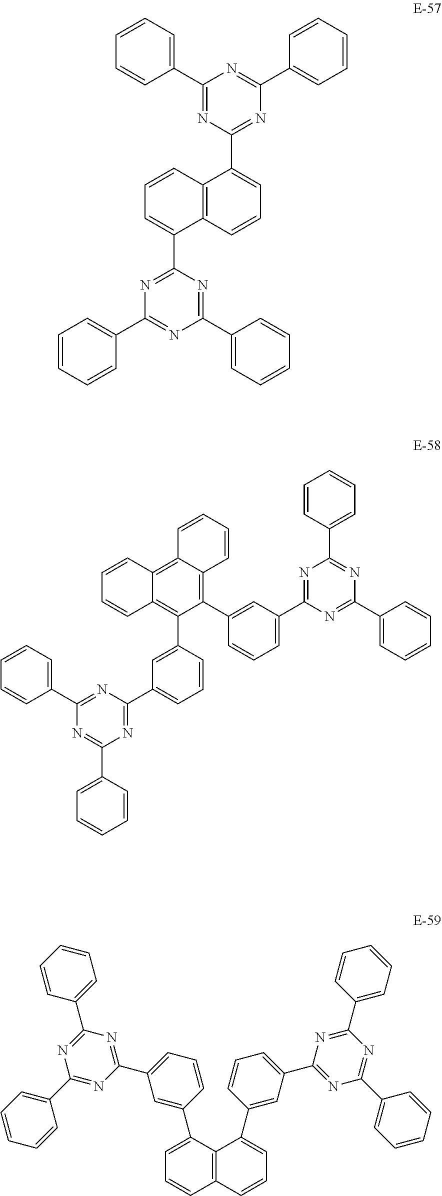 [SMILES: c1ccc(-c2nc(-c3ccccc3)nc(-c3cccc4c(-c5nc(-c6ccccc6)nc(-c6ccccc6)n5)cccc34)n2)cc1 ; c1ccc(-c2nc(-c3ccccc3)nc(-c3cccc(-c4c(-c5cccc(-c6nc(-c7ccccc7)nc(-c7ccccc7)n6)c5)c5ccccc5c5ccccc45)c3)n2)cc1 ; c1ccc(-c2nc(-c3ccccc3)nc(-c3cccc(-c4cccc5cccc(-c6cccc(-c7nc(-c8ccccc8)nc(-c8ccccc8)n7)c6)c45)c3)n2)cc1]

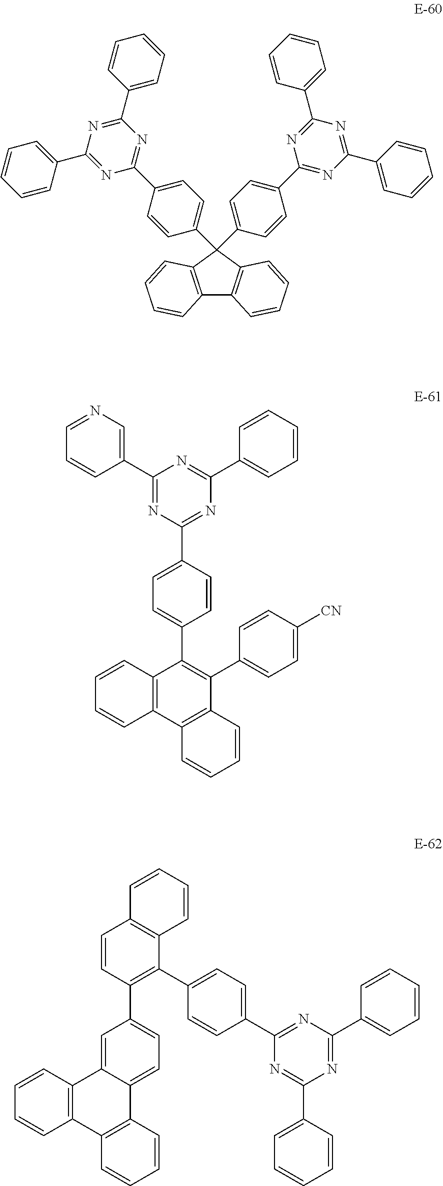 [SMILES: c1ccc(-c2nc(-c3ccccc3)nc(-c3ccc(C4(c5ccc(-c6nc(-c7ccccc7)nc(-c7ccccc7)n6)cc5)c5ccccc5-c5ccccc54)cc3)n2)cc1 ; N#Cc1ccc(-c2c(-c3ccc(-c4nc(-c5ccccc5)nc(-c5cccnc5)n4)cc3)c3ccccc3c3ccccc23)cc1 ; c1ccc(-c2nc(-c3ccccc3)nc(-c3ccc(-c4c(-c5ccc6c7ccccc7c7ccccc7c6c5)ccc5ccccc45)cc3)n2)cc1]

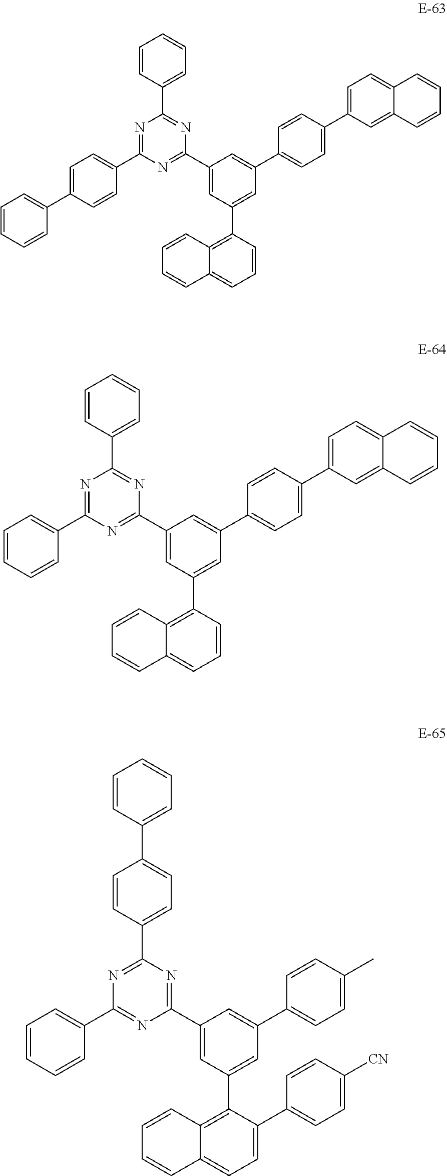 [SMILES: c1ccc(-c2ccc(-c3nc(-c4ccccc4)nc(-c4cc(-c5ccc(-c6ccc7ccccc7c6)cc5)cc(-c5cccc6ccccc56)c4)n3)cc2)cc1 ; c1ccc(-c2nc(-c3ccccc3)nc(-c3cc(-c4ccc(-c5ccc6ccccc6c5)cc4)cc(-c4cccc5ccccc45)c3)n2)cc1 ; Cc1ccc(-c2cc(-c3nc(-c4ccccc4)nc(-c4ccc(-c5ccccc5)cc4)n3)cc(-c3c(-c4ccc(C#N)cc4)ccc4ccccc34)c2)cc1]

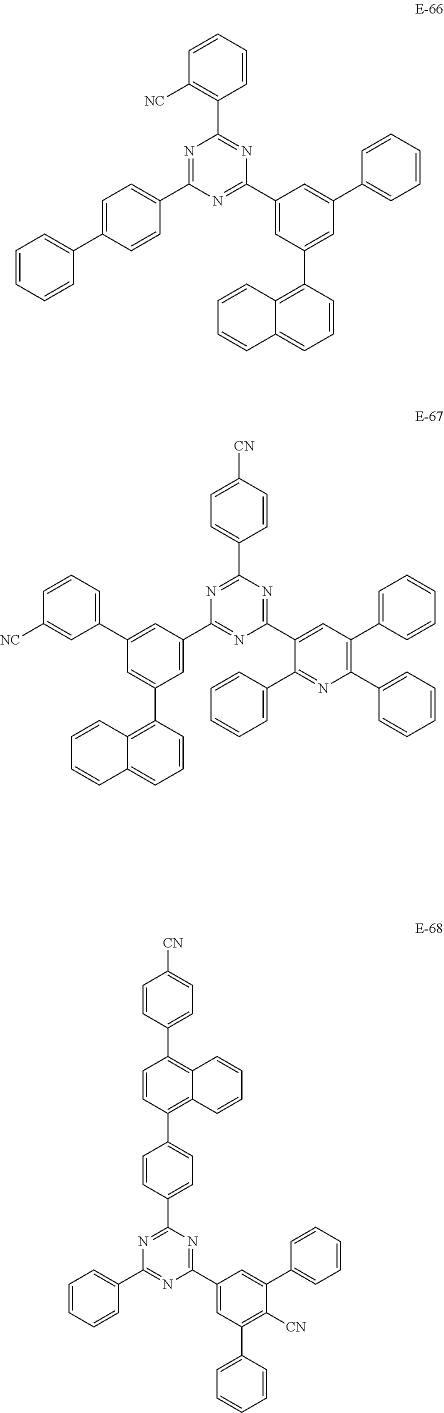 [SMILES: N#Cc1ccccc1-c1nc(-c2ccc(-c3ccccc3)cc2)nc(-c2cc(-c3ccccc3)cc(-c3cccc4ccccc34)c2)n1 ; N#Cc1ccc(-c2nc(-c3cc(-c4cccc(C#N)c4)cc(-c4cccc5ccccc45)c3)nc(-c3cc(-c4ccccc4)c(-c4ccccc4)nc3-c3ccccc3)n2)cc1 ; N#Cc1ccc(-c2ccc(-c3ccc(-c4nc(-c5ccccc5)nc(-c5cc(-c6ccccc6)c(C#N)c(-c6ccccc6)c5)n4)cc3)c3ccccc23)cc1]

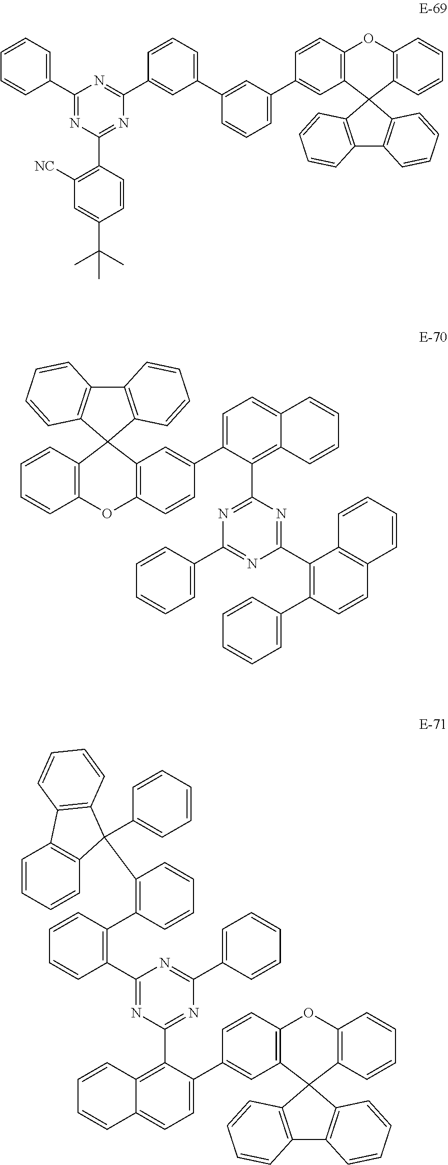 [SMILES: CC(C)(C)c1ccc(-c2nc(-c3ccccc3)nc(-c3cccc(-c4cccc(-c5ccc6c(c5)C5(c7ccccc7O6)c6ccccc6-c6ccccc65)c4)c3)n2)c(C#N)c1 ; c1ccc(-c2nc(-c3c(-c4ccccc4)ccc4ccccc34)nc(-c3c(-c4ccc5c(c4)C4(c6ccccc6O5)c5ccccc5-c5ccccc54)ccc4ccccc34)n2)cc1 ; c1ccc(-c2nc(-c3ccccc3-c3ccccc3C3(c4ccccc4)c4ccccc4-c4ccccc43)nc(-c3c(-c4ccc5c(c4)C4(c6ccccc6O5)c5ccccc5-c5ccccc54)ccc4ccccc34)n2)cc1]

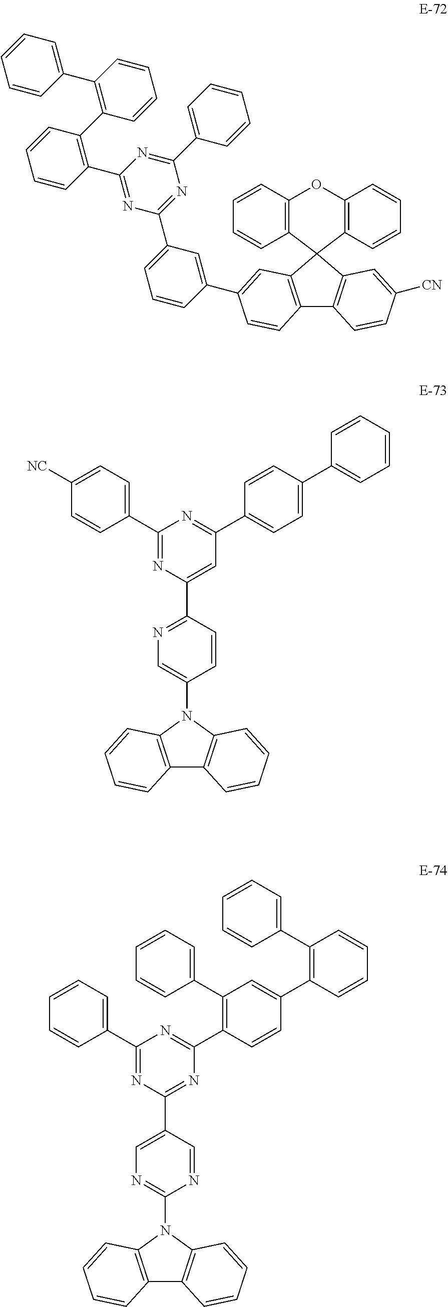 [SMILES: N#Cc1ccc2c(c1)C1(c3ccccc3Oc3ccccc31)c1cc(-c3cccc(-c4nc(-c5ccccc5)nc(-c5ccccc5-c5ccccc5-c5ccccc5)n4)c3)ccc1-2 ; N#Cc1ccc(-c2nc(-c3ccc(-c4ccccc4)cc3)cc(-c3ccc(-n4c5ccccc5c5ccccc54)cn3)n2)cc1 ; c1ccc(-c2nc(-c3cnc(-n4c5ccccc5c5ccccc54)nc3)nc(-c3ccc(-c4ccccc4-c4ccccc4)cc3-c3ccccc3)n2)cc1]

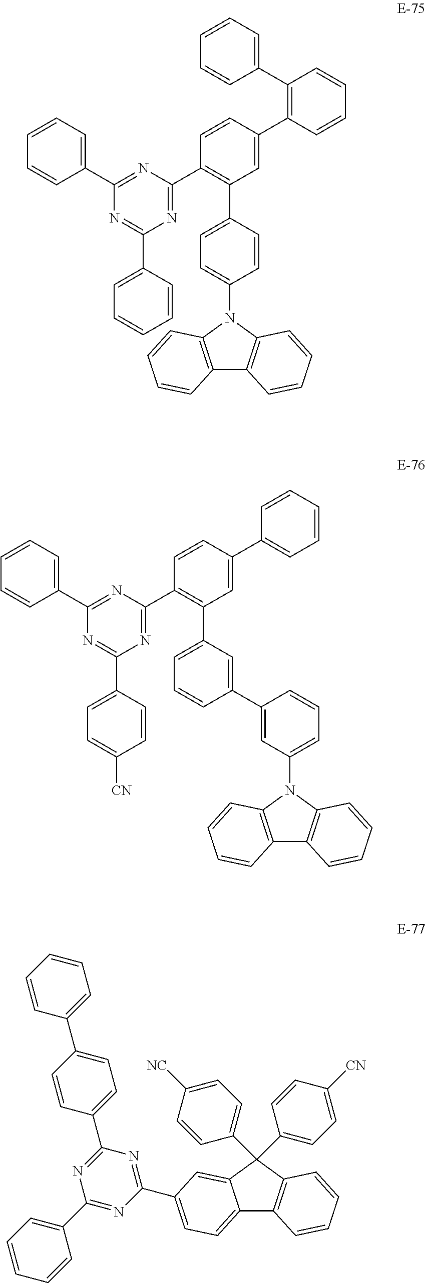 [SMILES: c1ccc(-c2nc(-c3ccccc3)nc(-c3ccc(-c4ccccc4-c4ccccc4)cc3-c3ccc(-n4c5ccccc5c5ccccc54)cc3)n2)cc1 ; N#Cc1ccc(-c2nc(-c3ccccc3)nc(-c3ccc(-c4ccccc4)cc3-c3cccc(-c4cccc(-n5c6ccccc6c6ccccc65)c4)c3)n2)cc1 ; N#Cc1ccc(C2(c3ccc(C#N)cc3)c3ccccc3-c3ccc(-c4nc(-c5ccccc5)nc(-c5ccc(-c6ccccc6)cc5)n4)cc32)cc1]

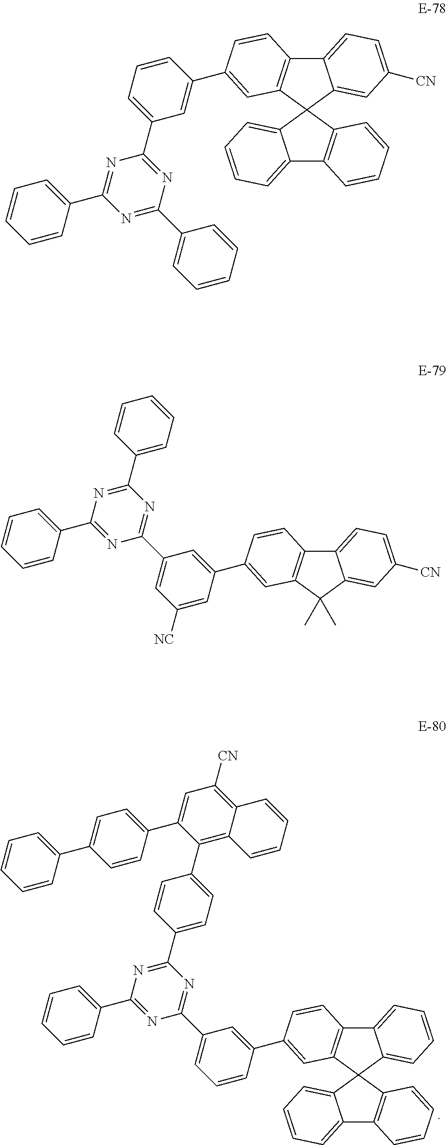 [SMILES: N#Cc1ccc2c(c1)C1(c3ccccc3-c3ccccc31)c1cc(-c3cccc(-c4nc(-c5ccccc5)nc(-c5ccccc5)n4)c3)ccc1-2 ; CC1(C)c2cc(C#N)ccc2-c2ccc(-c3cc(C#N)cc(-c4nc(-c5ccccc5)nc(-c5ccccc5)n4)c3)cc21 ; N#Cc1cc(-c2ccc(-c3ccccc3)cc2)c(-c2ccc(-c3nc(-c4ccccc4)nc(-c4cccc(-c5ccc6c(c5)C5(c7ccccc7-c7ccccc75)c5ccccc5-6)c4)n3)cc2)c2ccccc12]

Hereinafter, synthesis examples of compound represented by Formulas 1 and 2 according to the present invention, and preparation methods of an organic electric element will be described in detail by way of examples. However, the present invention is not limited to the following examples.

### SYNTHESIS EXAMPLE

### Synthesis Example of Formula 1

Compound (final product 1) represented by Formula 1 according to the present invention can be synthesized by reacting Sub 1A and Sub 1B as shown in Reaction Scheme 1 below, but is not limited thereto.

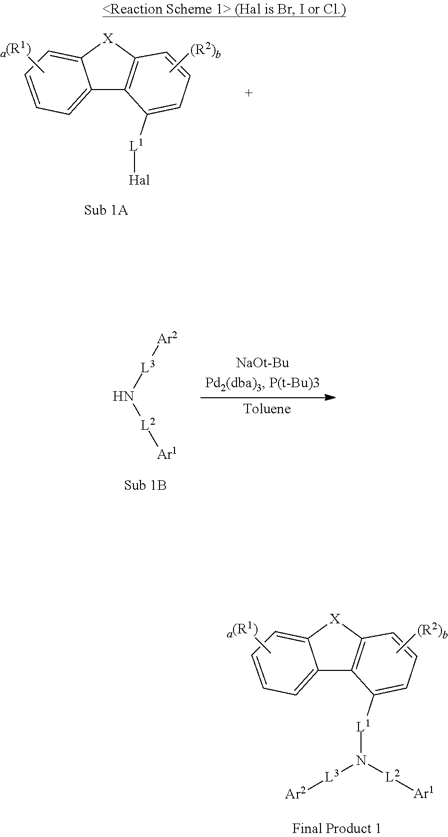

### Synthesis Example of Sub 1A

### 1. Synthesis Example of Sub 1-1

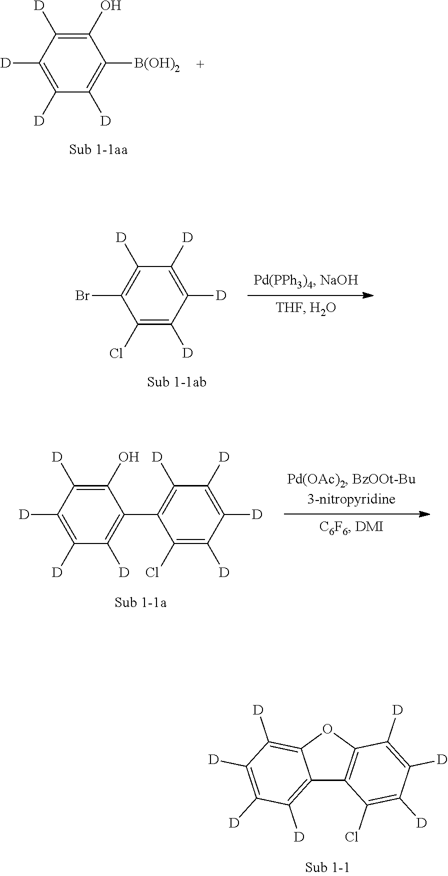

### (1) Synthesis of Sub 1-1a

After Sub 2-1aa (50 g, 352.2 mmol) is dissolved in THF (1761 ml), Sub 1-1ab (68.9 g, 352.2 mmol), Pd(PPh₃)₄ (24.4 g, 21.1 mmol), NaOH (42.3 g, 1056.7 mmol) and water (881 ml) are added thereto and the reaction is carried out at 80° C. When the reaction is completed, the reaction product is extracted with CH₂Cl₂ and water, and then the organic layer is dried with MgSO₄ and concentrated. Then, the concentrate is separated through a silica gel column and recrystallized to obtain 61.1 g (yield: 81.5%) of the product.

### (2) Synthesis of Sub

After dissolving Sub 1-1a (61.1 g, 287.1 mmol) by adding C₆F₆ (428 ml) and DMI (287 ml), Pd(OAc)₂ (3.2 g, 14.4 mmol), 3-nitropyridine (1.8 g, 14.4 mmol) and tent-butyl peroxybenzoate (111.5 g, 574.1 mmol) are added thereto and the reaction is carried out at 90° C. When the reaction is completed, the reaction product is extracted with CH₂Cl₂ and water, and then the organic layer is dried with MgSO₄ and concentrated. Then, the concentrate is separated through a silica gel column and recrystallized to obtain 42.0 g (yield: 69.8%) of the product.

### 2. Synthesis Example of Sub 1-2

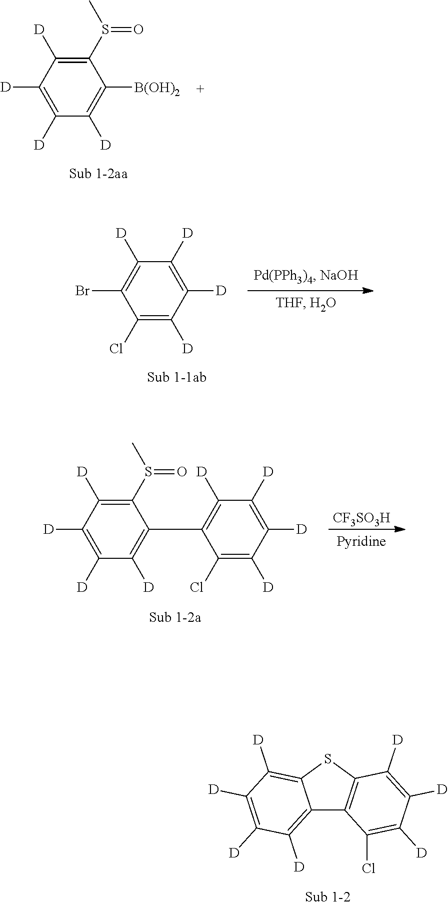

### (1) Synthesis of Sub 1-2a

After dissolving Sub 1-2aa (50.0 g, 265.9 mmol) in THF (1330 ml), Sub 1-1ab (52.0 g, 265.9 mmol), Pd(PPh₃)₄ (18.4 g, 16.0 mmol), NaOH (31.9 g, 797.7 mmol) and water (665 ml) are added thereto, and the synthesis was carried out in the same manner as the synthesis of Sub 1-1a to obtain 56.6 g (yield: 82.2%) of product.

### (2) Synthesis of Sub 1-2

After adding an excess amount of trifluoromethane-sulfonic acid to Sub 1-2a (56.6 g, 218.6 mmol), the solution is stirred at room temperature for 24 hours, then water and pyridine (8:1) are added thereto and the mixture is refluxed for 30 minutes. After cooling the resultant to room temperature, the resultant is extracted with CH₂Cl₂ and water, and then the organic layer is dried with MgSO₄ and concentrated. Then, the concentrate is separated through a silica gel column and recrystallized to obtain 46.2 g (yield: 93.7%) of the product.

### 3. Synthesis Example of Sub 1-11

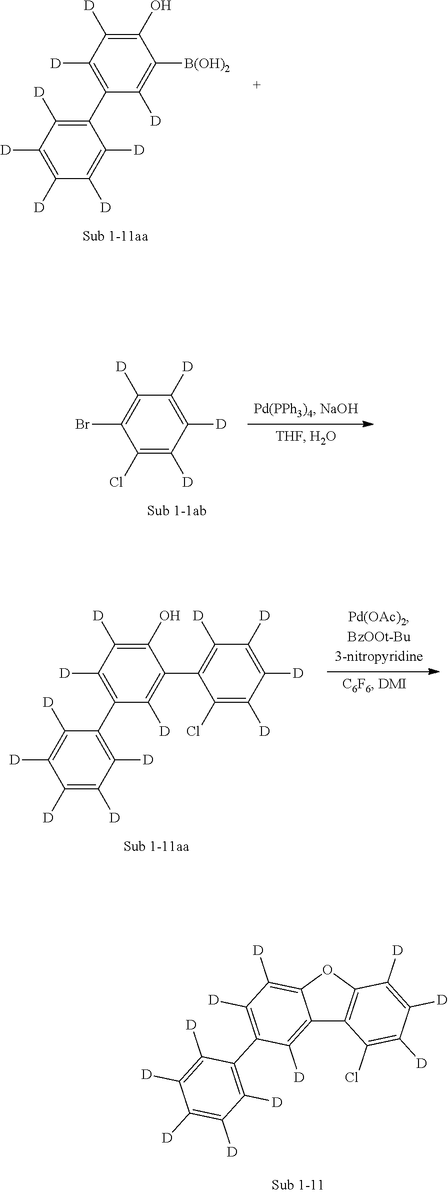

### (1) Synthesis of Sub 1-11a

After dissolving Sub 1-11aa (50.0 g, 225.1 mmol) in THF (1126 ml), Sub 1-1ab (44.0 g, 225.1 mmol), Pd(PPh₃)₄ (15.6 g, 13.5 mmol), NaOH (27.0 g, 675.4 mmol) and water (563 ml) are added thereto, and the synthesis is carried out in the same manner as the synthesis of Sub 1-1a to obtain 53.3 g (yield: 80.8%) of product.

### (2) Synthesis of Sub 1-11

After dissolving Sub 1-11a (53.3 g, 181.9 mmol) by adding C₆F₆ (272 ml) and DMI (182 ml), Pd(OAc)₂ (2.0 g, 9.1 mmol), 3-nitropyridine (1.1 g, 9.1 mmol) and tent-butyl peroxybenzoate (70.7 g, 363.8 mmol) are added thereto. Afterwards, the synthesis is carried out in the same manner as the synthesis of Sub 1-1 to obtain 37.5 g (yield: 71.1%) of product.

### 4. Synthesis Example of Sub 1-63

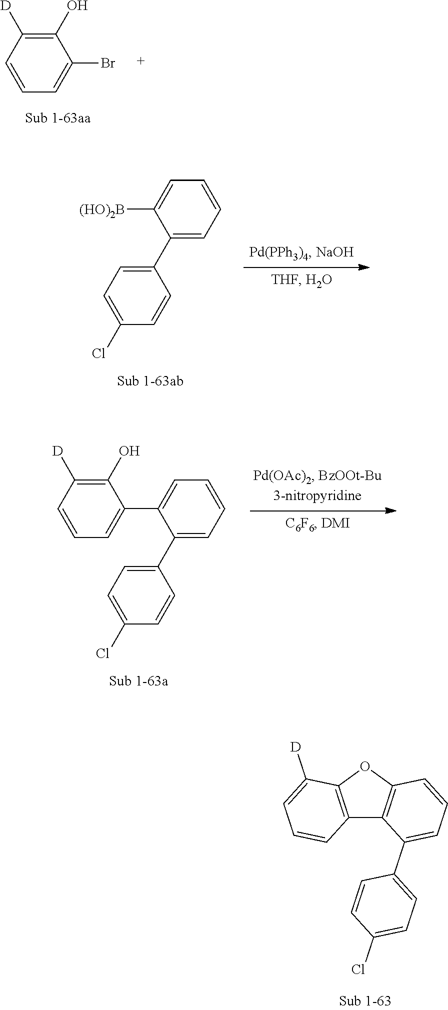 [SMILES: [2H]c1cccc(Br)c1O ; OB(O)c1ccccc1-c1ccc(Cl)cc1 ; [2H]c1cccc(-c2ccccc2-c2ccc(Cl)cc2)c1O ; [2H]c1cccc2c1oc1cccc(-c3ccc(Cl)cc3)c12]

### (1) Synthesis of Sub 1-63a

After dissolving Sub 1-63aa (50.0 g, 287.3 mmol) in THF (1437 ml), Sub 1-63ab (66.8 g, 287.3 mmol), Pd(PPh₃)₄ (19.9 g, 17.2 mmol), NaOH (34.5 g, 862.0 mmol) and water (718 ml) are added thereto, and the synthesis is carried out in the same manner as the synthesis of Sub 1-1a to obtain 57.5 g (yield: 83.4%) of product.

### (2) Synthesis of Sub 1-63

After dissolving Sub 1-63a (67.5 g, 239.6 mmol) by adding C₆F₆ (358 ml) and DMI (240 ml), Pd(OAc)₂ (2.7 g, 12.0 mmol), 3-nitropyridine (1.5 g, 12.0 mmol), tent-butyl peroxybenzoate (93.1 g, 479.3 mmol) are added thereto. Afterwards, the synthesis is carried out in the same manner as the synthesis of Sub 1-1 to obtain 45.8 g (yield: 68.3%) of product.

Compounds belonging to Sub 1 may be, but not limited to, the following compounds, and Table 1 shows FD-MS (Field Desorption-Mass Spectrometry) values of the following compounds.

TABLE 1

| Compound | FD-MS |
|---|---|
| Sub 1-1 | m/z = 209.06(C₁₂D₇ClO = 209.68) |
| Sub 1-2 | m/z = 225.04(C₁₂D₇ClS = 225.74) |
| Sub 1-3 | m/z = 206.04(C₁₂H₃D₄ClO = 206.66) |
| Sub 1-4 | m/z = 221.01(C₁₂H₄D₃ClS = 221.72) |
| Sub 1-5 | m/z = 204.03(C₁₂H₅D₂ClO = 204.65) |
| Sub 1-6 | m/z = 222.02(C₁₂H₃D₄ClS = 222.72) |
| Sub 1-7 | m/z = 203.02(C₁₂H₆DClO = 203.64) |
| Sub 1-8 | m/z = 203.02(C₁₂H₆DClO = 203.64) |
| Sub 1-9 | m/z = 219.00(C₁₂H₆DClS = 219.70) |
| Sub 1-10 | m/z = 219.00(C₁₂H₆DClS = 219.70) |
| Sub 1-11 | m/z = 289.12(C₁₈D₁₁ClO = 289.80) |
| Sub 1-12 | m/z = 289.12(C₁₈D₁₁ClO = 289.80) |
| Sub 1-13 | m/z = 371.06(C₂₄H₁₄DClS = 371.90) |
| Sub 1-14 | m/z = 364.14(C₂₄H₅D₁₀ClO = 364.89) |
| Sub 1-15 | m/z = 381.13(C₂₄H₄D₁₁ClS = 381.96) |
| Sub 1-16 | m/z = 282.07(C₁₈H₇D₄ClO = 282.76) |
| Sub 1-17 | m/z = 300.06(C₁₈H₅D₆ClS = 300.83) |
| Sub 1-18 | m/z = 289.12(C₁₈D₁₁ClO = 289.80) |
| Sub 1-19 | m/z = 360.12(C₂₄H₉D₆ClO = 360.87) |
| Sub 1-20 | m/z = 374.08(C₂₄H₁₁D₄ClS = 374.92) |
| Sub 1-21 | m/z = 285.09(C₁₈H₄D₇ClO = 285.78) |
| Sub 1-22 | m/z = 449.11(C₃₀H₁₆D₃ClS = 450.01) |
| Sub 1-23 | m/z = 511.18(C₃₆H₁₈D₅ClO = 512.06) |
| Sub 1-24 | m/z = 375.09(C₂₄H₁₀D₅ClS = 375.92) |
| Sub 1-25 | m/z = 503.15(C₃₄H₁₄D₇ClS = 504.09) |
| Sub 1-26 | m/z = 449.23(C₃₀D₁₉ClO = 450.05) |
| Sub 1-27 | m/z = 289.12(C₁₈D₁₁ClO = 289.80) |
| Sub 1-28 | m/z = 357.10(C₂₄H₁₂D₃ClO = 357.85) |
| Sub 1-29 | m/z = 305.10(C₁₈D₁₁ClS = 305.86) |
| Sub 1-30 | m/z = 414.11(C₂₇H₁₅D₄ClS = 414.98) |
| Sub 1-31 | m/z = 450.11(C₃₀H₁₅D₄ClS = 451.02) |
| Sub 1-32 | m/z = 461.18(C₃₀H₄D₁₅ClS = 462.08) |
| Sub 1-33 | m/z = 301.07(C₁₈H₄D₇ClS = 301.84) |
| Sub 1-34 | m/z = 378.11(C₂₄H₇D₈ClS = 378.94) |
| Sub 1-35 | m/z = 222.02(C₁₂H₃D₄ClS = 222.72) |
| Sub 1-36 | m/z = 219.00(C₁₂H₆DClS = 219.70) |
| Sub 1-37 | m/z = 305.10(C₁₈D₁₁ClS = 305.86) |
| Sub 1-38 | m/z = 220.01(C₁₂H₅D₂ClS = 220.71) |
| Sub 1-39 | m/z = 206.04(C₁₂H₃D₄ClO = 206.66) |
| Sub 1-40 | m/z = 203.02(C₁₂H₆DClO = 203.64) |
| Sub 1-41 | m/z = 203.02(C₁₂H₆DClO = 203.64) |
| Sub 1-42 | m/z = 220.01(C₁₂H₅D₂ClS = 220.71) |
| Sub 1-43 | m/z = 280.10(C₁₆H₉D₆ClS = 280.84) |
| Sub 1-44 | m/z = 250.10(C₁₅H₇D₆ClO = 250.75) |
| Sub 1-45 | m/z = 344.18(C₂₂H₉D₁₀ClO = 344.90) |
| Sub 1-46 | m/z = 356.13(C₂₂H₁₃D₆ClS = 356.94) |
| Sub 1-47 | m/z = 293.05(C₁₆HD₇ClFS = 293.79) |
| Sub 1-48 | m/z = 284.06(C₁₇H₉D₂ClO₂ = 284.74) |
| Sub 1-49 | m/z = 283.08(C₁₈H₆D₅ClO = 283.77) |
| Sub 1-50 | m/z = 291.03(C₁₆H₃D₅ClFS = 291.78) |
| Sub 1-51 | m/z = 432.15(C₂₈H₅D₁₂ClS = 433.03) |
| Sub 1-52 | m/z = 346.11(C₂₃H₁₅D₂ClO = 346.85) |
| Sub 1-53 | m/z = 387.15(C₂₆H₂₂DClO = 387.93) |
| Sub 1-54 | m/z = 362.08(C₂₃H₁₁D₄ClS = 362.91) |
| Sub 1-55 | m/z = 395.06(C₂₆H₁₄DClS = 395.92) |
| Sub 1-56 | m/z = 257.07(C₁₆H₄D₅ClO = 257.73) |
| Sub 1-57 | m/z = 333.10(C₂₂H₈D₅ClO = 333.83) |
| Sub 1-58 | m/z = 422.09(C₂₈H₁₅D₂ClS = 422.97) |
| Sub 1-59 | m/z = 329.10(C₂₀D₁₁ClS = 329.89) |
| Sub 1-60 | m/z = 261.09(C₁₆D₉ClO = 261.75) |
| Sub 1-61 | m/z = 304.06(C₂₀H₉D₂ClO = 304.77) |
| Sub 1-62 | m/z = 320.04(C₂₀H₉D₂ClS = 320.83) |
| Sub 1-63 | m/z = 279.06(C₁₈H₁₀DClO = 279.74) |
| Sub 1-64 | m/z = 295.03(C₁₈H₁₀DClS = 295.80) |
| Sub 1-65 | m/z = 203.02(C₁₂H₆DClO = 203.64) |

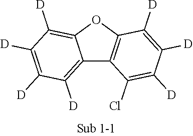 [SMILES: [2H]c1c([2H])c([2H])c2c(oc3c([2H])c([2H])c([2H])c(Cl)c32)c1[2H]]

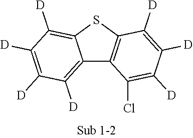 [SMILES: [2H]c1c([2H])c([2H])c2c(sc3c([2H])c([2H])c([2H])c(Cl)c32)c1[2H]]

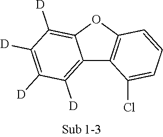 [SMILES: [2H]c1c([2H])c([2H])c2c(oc3cccc(Cl)c32)c1[2H]]

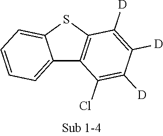 [SMILES: [2H]c1c([2H])c(Cl)c2c(sc3ccccc32)c1[2H]]

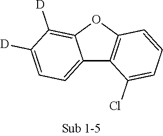 [SMILES: [2H]c1ccc2c(oc3cccc(Cl)c32)c1[2H]]

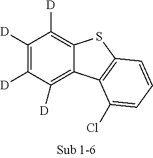 [SMILES: [2H]c1c([2H])c([2H])c2c(sc3cccc(Cl)c32)c1[2H]]

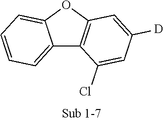 [SMILES: [2H]c1cc(Cl)c2c(c1)oc1ccccc12]

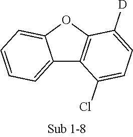 [SMILES: [2H]c1ccc(Cl)c2c1oc1ccccc12]

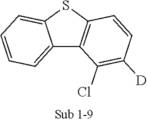 [SMILES: [2H]c1ccc2sc3ccccc3c2c1Cl]

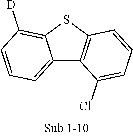 [SMILES: [2H]c1cccc2c1sc1cccc(Cl)c12]

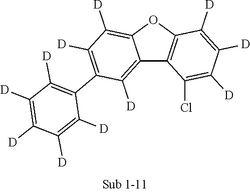 [SMILES: [2H]c1c([2H])c([2H])c(-c2c([2H])c([2H])c3oc4c([2H])c([2H])c([2H])c(Cl)c4c3c2[2H])c([2H])c1[2H]]

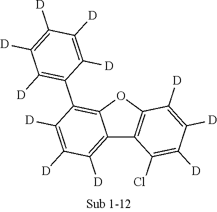 [SMILES: [2H]c1c([2H])c([2H])c(-c2c([2H])c([2H])c([2H])c3c2oc2c([2H])c([2H])c([2H])c(Cl)c23)c([2H])c1[2H]]

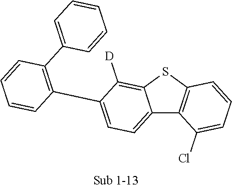 [SMILES: [2H]c1c(-c2ccccc2-c2ccccc2)ccc2c1sc1cccc(Cl)c12]

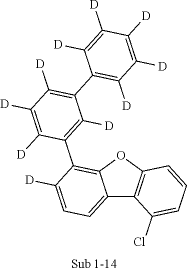 [SMILES: [2H]c1ccc2c(oc3cccc(Cl)c32)c1-c1c([2H])c([2H])c([2H])c(-c2c([2H])c([2H])c([2H])c([2H])c2[2H])c1[2H]]

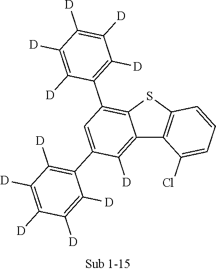 [SMILES: [2H]c1c([2H])c([2H])c(-c2cc(-c3c([2H])c([2H])c([2H])c([2H])c3[2H])c3sc4cccc(Cl)c4c3c2[2H])c([2H])c1[2H]]

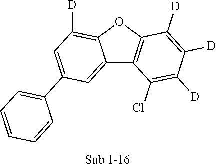 [SMILES: [2H]c1c([2H])c(Cl)c2c(oc3c([2H])cc(-c4ccccc4)cc32)c1[2H]]

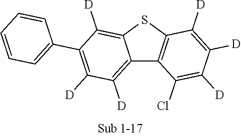 [SMILES: [2H]c1c([2H])c(Cl)c2c(sc3c([2H])c(-c4ccccc4)c([2H])c([2H])c32)c1[2H]]

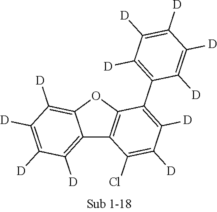 [SMILES: [2H]c1c([2H])c([2H])c(-c2c([2H])c([2H])c(Cl)c3c2oc2c([2H])c([2H])c([2H])c([2H])c23)c([2H])c1[2H]]

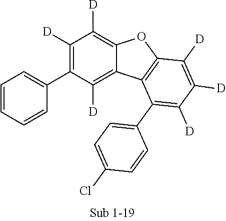 [SMILES: [2H]c1c(-c2ccccc2)c([2H])c2c(oc3c([2H])c([2H])c([2H])c(-c4ccc(Cl)cc4)c32)c1[2H]]

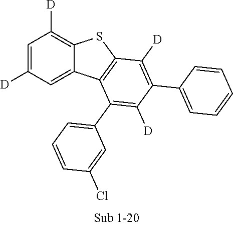 [SMILES: [2H]c1cc([2H])c2sc3c([2H])c(-c4ccccc4)c([2H])c(-c4cccc(Cl)c4)c3c2c1]

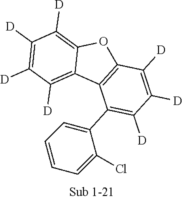 [SMILES: [2H]c1c([2H])c([2H])c2c(oc3c([2H])c([2H])c([2H])c(-c4ccccc4Cl)c32)c1[2H]]

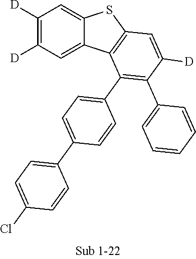 [SMILES: [2H]c1cc2sc3cc([2H])c(-c4ccccc4)c(-c4ccc(-c5ccc(Cl)cc5)cc4)c3c2cc1[2H]]

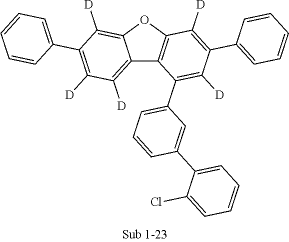 [SMILES: [2H]c1c(-c2ccccc2)c([2H])c2oc3c([2H])c(-c4ccccc4)c([2H])c(-c4cccc(-c5ccccc5Cl)c4)c3c2c1[2H]]

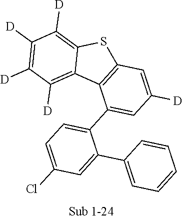 [SMILES: [2H]c1cc(-c2ccc(Cl)cc2-c2ccccc2)c2c(c1)sc1c([2H])c([2H])c([2H])c([2H])c12]

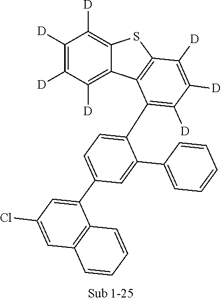 [SMILES: [2H]c1c([2H])c([2H])c2c(sc3c([2H])c([2H])c([2H])c(-c4ccc(-c5cc(Cl)cc6ccccc56)cc4-c4ccccc4)c32)c1[2H]]

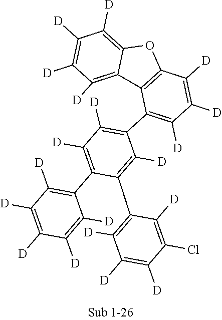 [SMILES: [2H]c1c([2H])c([2H])c(-c2c([2H])c([2H])c(-c3c([2H])c([2H])c([2H])c4oc5c([2H])c([2H])c([2H])c([2H])c5c34)c([2H])c2-c2c([2H])c([2H])c([2H])c(Cl)c2[2H])c([2H])c1[2H]]

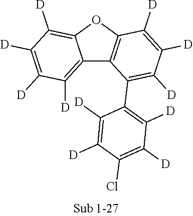 [SMILES: [2H]c1c([2H])c(-c2c([2H])c([2H])c([2H])c3oc4c([2H])c([2H])c([2H])c([2H])c4c23)c([2H])c([2H])c1Cl]

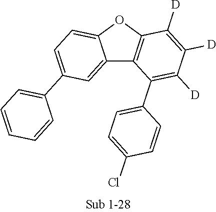 [SMILES: [2H]c1c([2H])c(-c2ccc(Cl)cc2)c2c(oc3ccc(-c4ccccc4)cc32)c1[2H]]

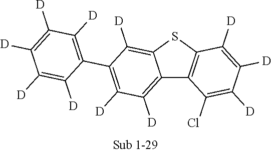 [SMILES: [2H]c1c([2H])c([2H])c(-c2c([2H])c([2H])c3c(sc4c([2H])c([2H])c([2H])c(Cl)c43)c2[2H])c([2H])c1[2H]]

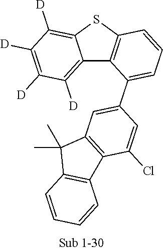 [SMILES: [2H]c1c([2H])c([2H])c2c(sc3cccc(-c4cc(Cl)c5c(c4)C(C)(C)c4ccccc4-5)c32)c1[2H]]

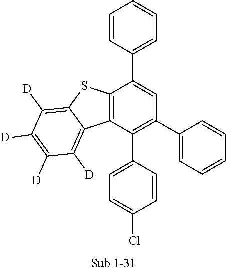 [SMILES: [2H]c1c([2H])c([2H])c2c(sc3c(-c4ccccc4)cc(-c4ccccc4)c(-c4ccc(Cl)cc4)c32)c1[2H]]

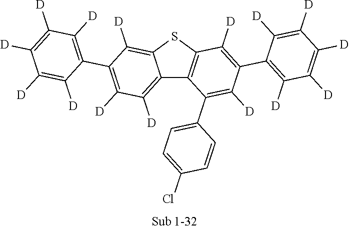 [SMILES: [2H]c1c([2H])c([2H])c(-c2c([2H])c([2H])c3c(sc4c([2H])c(-c5c([2H])c([2H])c([2H])c([2H])c5[2H])c([2H])c(-c5ccc(Cl)cc5)c43)c2[2H])c([2H])c1[2H]]

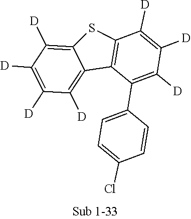 [SMILES: [2H]c1c([2H])c([2H])c2c(sc3c([2H])c([2H])c([2H])c(-c4ccc(Cl)cc4)c32)c1[2H]]

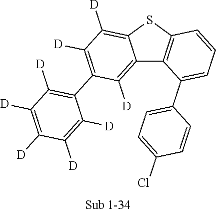 [SMILES: [2H]c1c([2H])c([2H])c(-c2c([2H])c([2H])c3sc4cccc(-c5ccc(Cl)cc5)c4c3c2[2H])c([2H])c1[2H]]

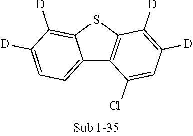 [SMILES: [2H]c1ccc2c(sc3c([2H])c([2H])cc(Cl)c32)c1[2H]]

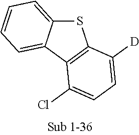 [SMILES: [2H]c1ccc(Cl)c2c1sc1ccccc12]

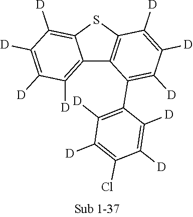 [SMILES: [2H]c1c([2H])c(-c2c([2H])c([2H])c([2H])c3sc4c([2H])c([2H])c([2H])c([2H])c4c23)c([2H])c([2H])c1Cl]

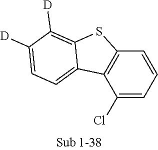 [SMILES: [2H]c1ccc2c(sc3cccc(Cl)c32)c1[2H]]

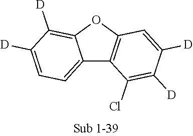 [SMILES: [2H]c1cc2oc3c([2H])c([2H])ccc3c2c(Cl)c1[2H]]

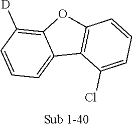 [SMILES: [2H]c1cccc2c1oc1cccc(Cl)c12]

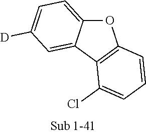 [SMILES: [2H]c1ccc2oc3cccc(Cl)c3c2c1]

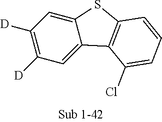 [SMILES: [2H]c1cc2sc3cccc(Cl)c3c2cc1[2H]]

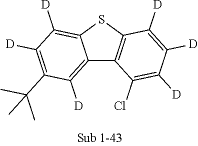 [SMILES: [2H]c1c(C(C)(C)C)c([2H])c2c(sc3c([2H])c([2H])c([2H])c(Cl)c32)c1[2H]]

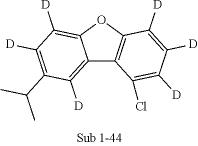 [SMILES: [2H]c1c(C(C)C)c([2H])c2c(oc3c([2H])c([2H])c([2H])c(Cl)c32)c1[2H]]

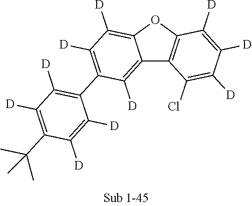 [SMILES: [2H]c1c([2H])c(C(C)(C)C)c([2H])c([2H])c1-c1c([2H])c([2H])c2oc3c([2H])c([2H])c([2H])c(Cl)c3c2c1[2H]]

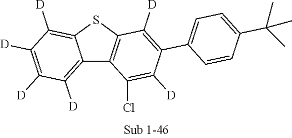 [SMILES: [2H]c1c([2H])c([2H])c2c(sc3c([2H])c(-c4ccc(C(C)(C)C)cc4)c([2H])c(Cl)c32)c1[2H]]

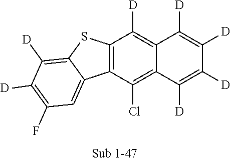 [SMILES: [2H]c1c(F)cc2c(sc3c([2H])c4c([2H])c([2H])c([2H])c([2H])c4c(Cl)c32)c1[2H]]

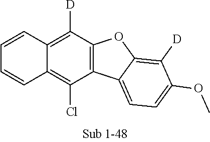 [SMILES: [2H]c1c(OC)ccc2c1oc1c([2H])c3ccccc3c(Cl)c12]

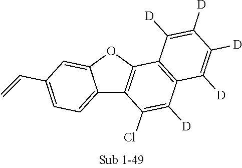 [SMILES: [2H]c1c([2H])c([2H])c2c(c1[2H])c([2H])c(Cl)c1c3ccc(C=C)cc3oc21]

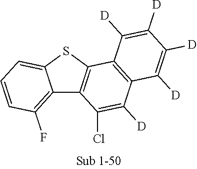 [SMILES: [2H]c1c([2H])c([2H])c2c(c1[2H])c([2H])c(Cl)c1c2sc2cccc(F)c21]

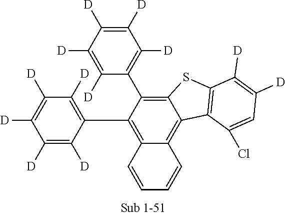 [SMILES: [2H]c1cc(Cl)c2c(sc3c(-c4c([2H])c([2H])c([2H])c([2H])c4[2H])c(-c4c([2H])c([2H])c([2H])c([2H])c4[2H])c4ccccc4c32)c1[2H]]

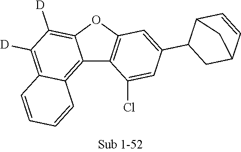 [SMILES: [2H]c1c([2H])c2oc3cc(C4CC5C=CC4C5)cc(Cl)c3c2c2ccccc12]

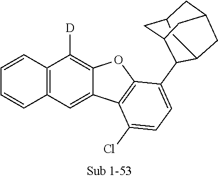 [SMILES: [2H]c1c2ccccc2cc2c1oc1c(C3C4CC5CC(C4)CC3C5)ccc(Cl)c12]

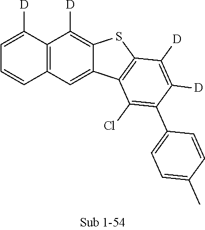 [SMILES: [2H]c1c(-c2ccc(C)cc2)c(Cl)c2c(sc3c([2H])c4c([2H])cccc4cc32)c1[2H]]

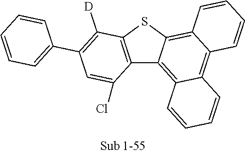 [SMILES: [2H]c1c(-c2ccccc2)cc(Cl)c2c1sc1c3ccccc3c3ccccc3c12]

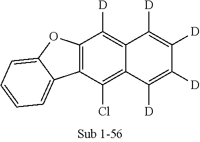 [SMILES: [2H]c1c([2H])c([2H])c2c(Cl)c3c(oc4ccccc43)c([2H])c2c1[2H]]

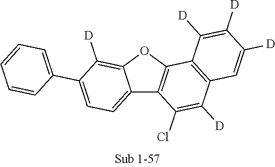 [SMILES: [2H]c1cc2c([2H])c(Cl)c3c4ccc(-c5ccccc5)c([2H])c4oc3c2c([2H])c1[2H]]

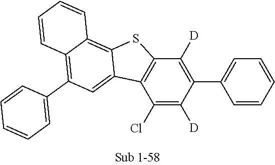 [SMILES: [2H]c1c(-c2ccccc2)c([2H])c2sc3c4ccccc4c(-c4ccccc4)cc3c2c1Cl]

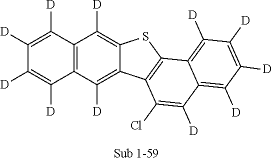 [SMILES: [2H]c1c([2H])c([2H])c2c([2H])c3c(sc4c5c([2H])c([2H])c([2H])c([2H])c5c([2H])c(Cl)c43)c([2H])c2c1[2H]]

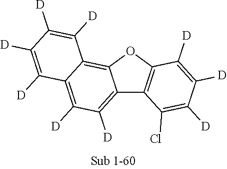 [SMILES: [2H]c1c([2H])c(Cl)c2c(oc3c4c([2H])c([2H])c([2H])c([2H])c4c([2H])c([2H])c32)c1[2H]]

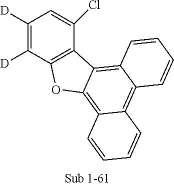 [SMILES: [2H]c1cc(Cl)c2c(oc3c4ccccc4c4ccccc4c32)c1[2H]]

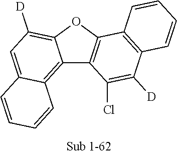 [SMILES: [2H]c1c(Cl)c2c(oc3c([2H])cc4ccccc4c32)c2ccccc12]

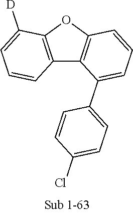 [SMILES: [2H]c1cccc2c1oc1cccc(-c3ccc(Cl)cc3)c12]

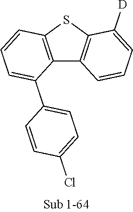 [SMILES: [2H]c1cccc2c1sc1cccc(-c3ccc(Cl)cc3)c12]

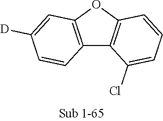 [SMILES: [2H]c1ccc2c(c1)oc1cccc(Cl)c12]

### Synthesis example of Sub 1B

Sub 1B of the Reaction Scheme 1 can be synthesized by the reaction route of Reaction Scheme 2, but there is no limitation thereto.

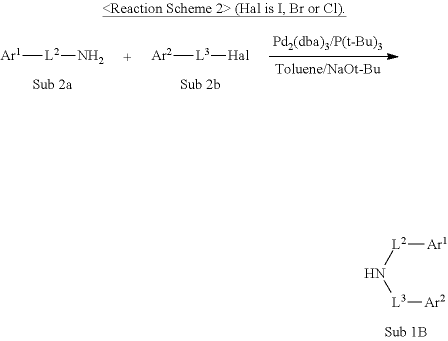

### 1. Synthesis Example of Sub 2-6

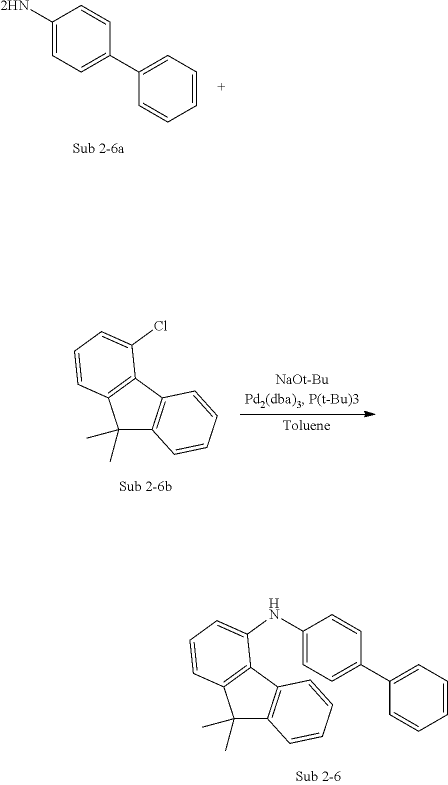

After dissolving Sub 2-6a (50.0 g, 295.5 mmol) in toluene (1477 ml), Sub 2-6b (65.3 g, 295.5 mmol), Pd₂(dba)₃ (8.1 g, 8.9 mmol), P(t-Bu)₃ (3.6 g, 17.7 mmol) and NaOt-Bu (56.8 g, 590.9 mmol) are added thereto and the reaction is carried out at 80° C. When the reaction is completed, the reaction product is extracted with CH₂Cl₂ and water, and then the organic layer is dried with MgSO₄ and concentrated. Then, the concentrate is separated through a silica gel column and recrystallized to obtain 78.6 g (yield: 73.6%) of the product.

### 2. Synthesis Example of Sub 2-9

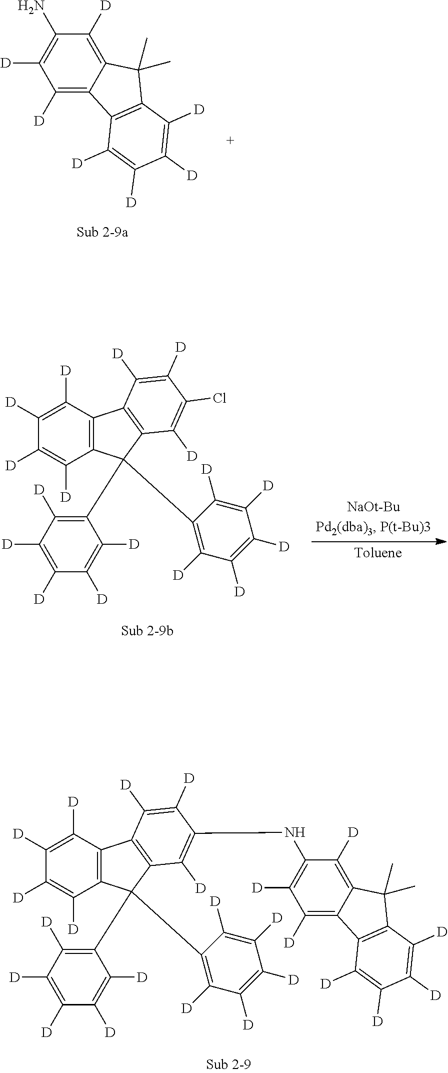 [SMILES: [2H]c1c([2H])c([2H])c2c(c1[2H])-c1c([2H])c([2H])c(N)c([2H])c1C2(C)C ; [2H]c1c([2H])c([2H])c(C2(c3c([2H])c([2H])c([2H])c([2H])c3[2H])c3c([2H])c([2H])c([2H])c([2H])c3-c3c([2H])c([2H])c(Cl)c([2H])c32)c([2H])c1[2H] ; [2H]c1c([2H])c([2H])c(C2(c3c([2H])c([2H])c([2H])c([2H])c3[2H])c3c([2H])c([2H])c([2H])c([2H])c3-c3c([2H])c([2H])c(Nc4c([2H])c([2H])c5c(c4[2H])C(C)(C)c4c([2H])c([2H])c([2H])c([2H])c4-5)c([2H])c32)c([2H])c1[2H]]

After dissolving Sub 2-9a (50.0 g, 216.3 mmol) in toluene (1156 ml), Sub 2-9b (82.6 g, 231.1 mmol), Pd₂(dba)₃ (6.4 g, 6.9 mmol), P(t-Bu)₃ (2.8 g, 13.9 mmol) and NaOt-Bu (44.4 g, 462.3 mmol) are thereto, and the synthesis is carried out in the same manner as the synthesis of Sub 2-6 to obtain 92.0 g (yield: 72.4%) of product.

### 3. Synthesis of Sub 2-26

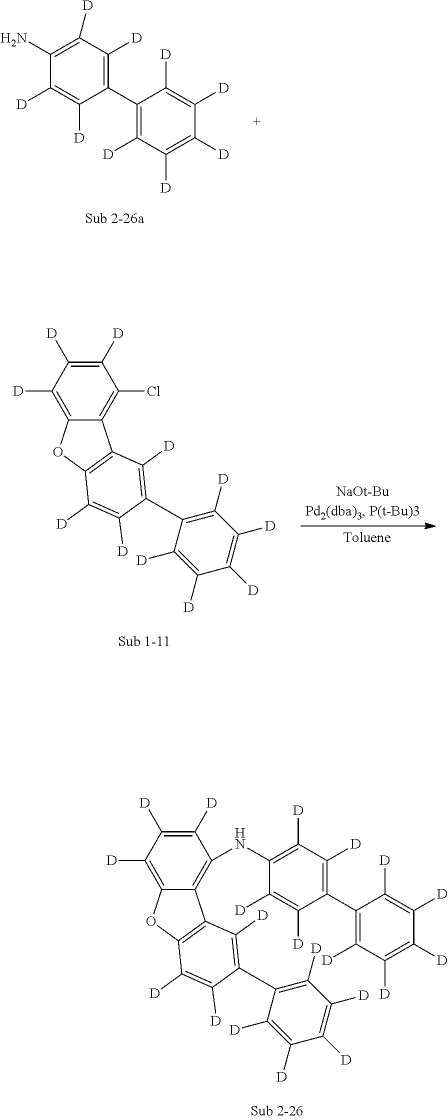 [SMILES: [2H]c1c([2H])c([2H])c(-c2c([2H])c([2H])c(N)c([2H])c2[2H])c([2H])c1[2H] ; [2H]c1c([2H])c([2H])c(-c2c([2H])c([2H])c3oc4c([2H])c([2H])c([2H])c(Cl)c4c3c2[2H])c([2H])c1[2H] ; [2H]c1c([2H])c([2H])c(-c2c([2H])c([2H])c(Nc3c([2H])c([2H])c([2H])c4oc5c([2H])c([2H])c(-c6c([2H])c([2H])c([2H])c([2H])c6[2H])c([2H])c5c34)c([2H])c2[2H])c([2H])c1[2H]]

After dissolving Sub 2-26a (50.0 g, 280.5 mmol) in toluene (1402 ml), Sub 1-11 (78.5 g, 280.5 mmol), Pd₂(dba)₃ (7.7 g, 8.4 mmol), P(t-Bu)₃ (3.4 g, 16.8 mmol), NaOt-Bu (53.9 g, 560.9 mmol) are thereto, and the synthesis is carried out in the same manner as the synthesis of Sub 2-6 to obtain 83.2 g (yield: 68.7%) of product.

### 4. Synthesis of Sub 2-68

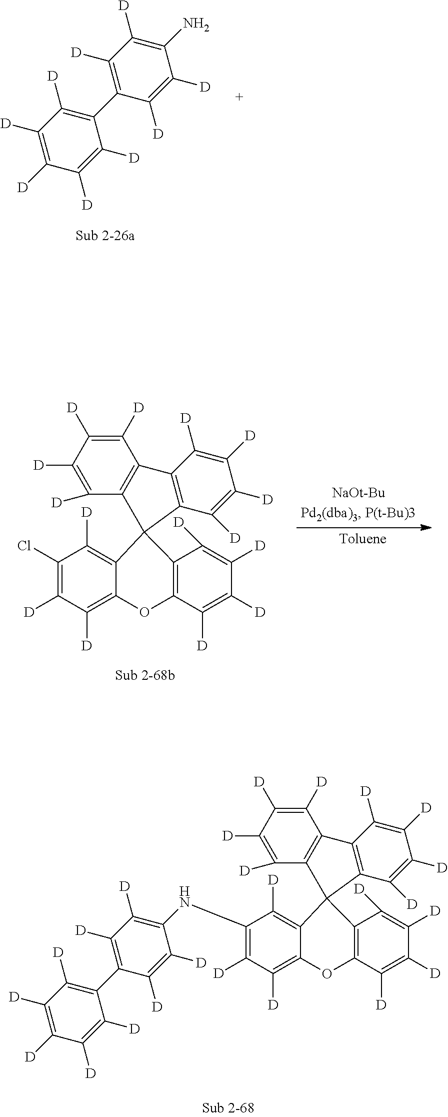 [SMILES: [2H]c1c([2H])c([2H])c(-c2c([2H])c([2H])c(N)c([2H])c2[2H])c([2H])c1[2H] ; [2H]c1c([2H])c([2H])c2c(c1[2H])Oc1c([2H])c([2H])c(Cl)c([2H])c1C21c2c([2H])c([2H])c([2H])c([2H])c2-c2c([2H])c([2H])c([2H])c([2H])c21 ; [2H]c1c([2H])c([2H])c(-c2c([2H])c([2H])c(Nc3c([2H])c([2H])c4c(c3[2H])C3(c5c([2H])c([2H])c([2H])c([2H])c5O4)c4c([2H])c([2H])c([2H])c([2H])c4-c4c([2H])c([2H])c([2H])c([2H])c43)c([2H])c2[2H])c([2H])c1[2H]]

After dissolving Sub 2-26a (50.0 g, 280.5 mmol) in toluene (1402 ml), Sub 2-68b (103.5 g, 280.5 mmol), Pd₂(dba)₃ (7.7 g, 8.4 mmol), P(t-Bu)₃ (3.4 g, 16.8 mmol), NaOt-Bu (53.9 g, 560.9 mmol) are thereto, and the synthesis is carried out in the same manner as the synthesis of Sub 2-6 to obtain 102.8 g (yield: 70.0%) of product.

### 5. Synthesis of Sub 2-97

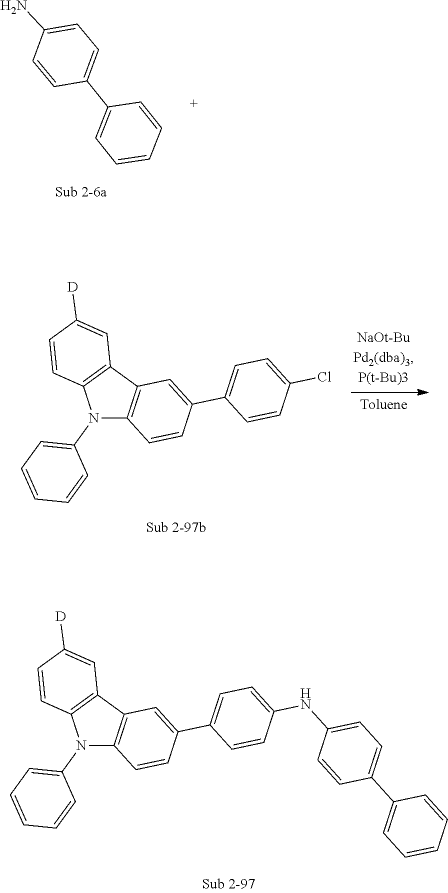 [SMILES: Nc1ccc(-c2ccccc2)cc1 ; [2H]c1ccc2c(c1)c1cc(-c3ccc(Cl)cc3)ccc1n2-c1ccccc1 ; [2H]c1ccc2c(c1)c1cc(-c3ccc(Nc4ccc(-c5ccccc5)cc4)cc3)ccc1n2-c1ccccc1]

After dissolving Sub 2-6a (50.0 g, 295.5 mmol) in toluene (1477 ml), Sub 2-97b (101.3 g, 295.5 mmol), Pd₂(dba)₃ (8.1 g, 8.9 mmol), P(t-Bu)₃ (3.6 g, 17.7 mmol), NaOt-Bu (56.8 g, 590.9 mmol) are thereto, and the synthesis is carried out in the same manner as the synthesis of Sub 2-6 to obtain 102.6 g (yield: 71.2%) of product.

Compounds belonging to Sub 2 may be, but not limited to, the following compounds, and Table 2 shows FD-MS (Field Desorption-Mass Spectrometry) values of the following compounds.

TABLE 2

| Compound | FD-MS |
|---|---|
| Sub 2-1 | m/z = 321.15(C₂₄H₁₉N = 321.42) |
| Sub 2-2 | m/z = 371.17(C₂₈H₂₁N = 371.48) |
| Sub 2-3 | m/z = 345.15(C₂₆H₁₉N = 345.45) |
| Sub 2-4 | m/z = 447.20(C₃₄H₂₅N = 447.58) |
| Sub 2-5 | m/z = 361.18(C₂₇H₂₃N = 361.49) |
| Sub 2-6 | m/z = 361.18(C₂₇H₂₃N = 361.49) |
| Sub 2-7 | m/z = 437.21(C₃₃H₂₇N = 437.59) |
| Sub 2-8 | m/z = 437.21(C₃₃H₂₇N = 437.59) |
| Sub 2-9 | m/z = 549.40(C₄₀H₇D₂₄N = 549.84) |
| Sub 2-10 | m/z = 648.26(C₄₉H₃₂N₂ = 648.81) |
| Sub 2-11 | m/z = 648.26(C₄₉H₃₂N₂ = 648.81) |
| Sub 2-12 | m/z = 523.34(C₃₇HD₂₄NO = 523.76) |
| Sub 2-13 | m/z = 515.17(C₃₇H₂₅NS = 515.67) |
| Sub 2-14 | m/z = 625.24(C₄₇H₃₁NO = 625.77) |
| Sub 2-15 | m/z = 726.30(C₅₅H₃₈N₂ = 726.92) |
| Sub 2-16 | m/z = 501.25(C₃₈H₃₁N = 501.67) |
| Sub 2-17 | m/z = 527.26(C₄₀H₃₃N = 527.71) |
| Sub 2-18 | m/z = 435.20(C₃₃H₂₅N = 435.57) |
| Sub 2-19 | m/z = 385.18(C₂₉H₂₃N = 385.51) |
| Sub 2-20 | m/z = 377.28(C₂₇H₇D₁₆N = 377.59) |
| Sub 2-21 | m/z = 444.26(C₃₃H₂₀D₇N = 444.63) |
| Sub 2-22 | m/z = 601.28(C₄₆H₃₅N = 601.79) |
| Sub 2-23 | m/z = 407.17(C₃₁H₂₁N = 407.52) |
| Sub 2-24 | m/z = 477.25(C₃₆H₃₁N = 477.65) |
| Sub 2-25 | m/z = 437.21(C₃₃H₂₇N = 437.59) |
| Sub 2-26 | m/z = 431.29(C₃₀HD₂₀NO = 431.63) |
| Sub 2-27 | m/z = 540.23(C₄₀H₂₄D₃NO = 540.68) |
| Sub 2-28 | m/z = 422.23(C₃₀H₁₀D₁₁NO = 422.57) |
| Sub 2-29 | m/z = 487.19(C₃₆H₂₅NO = 487.60) |
| Sub 2-30 | m/z = 342.17(C₂₄H₁₀D₇NO = 342.45) |
| Sub 2-31 | m/z = 356.15(C₂₄H₈D₇NO₂ = 356.43) |
| Sub 2-32 | m/z = 259.10(C₁₈H₁₃NO = 259.31) |
| Sub 2-33 | m/z = 309.12(C₂₂H₁₅NO = 309.37) |
| Sub 2-34 | m/z = 309.12(C₂₂H₁₅NO = 309.37) |
| Sub 2-35 | m/z = 435.16(C₃₂H₂₁NO = 435.53) |
| Sub 2-36 | m/z = 309.12(C₂₂H₁₅NO = 309.37) |
| Sub 2-37 | m/z = 389.25(C₂₇H₇D₁₄NO = 389.56) |
| Sub 2-38 | m/z = 389.25(C₂₇H₇D₁₄NO = 389.56) |
| Sub 2-39 | m/z = 506.24(C₃₇H₁₈D₇NO = 506.66) |
| Sub 2-40 | m/z = 414.18(C₃₀H₁₈D₃NO = 414.52) |
| Sub 2-41 | m/z = 527.24(C₃₆H₁₃D₁₁N₂S = 527.73) |
| Sub 2-42 | m/z = 527.22(C₃₉H₂₉NO = 527.67) |
| Sub 2-43 | m/z = 517.15(C₃₆H₂₃NOS = 517.65) |
| Sub 2-44 | m/z = 504.23(C₃₇H₂₄D₃NO = 504.65) |
| Sub 2-45 | m/z = 473.21(C₃₆H₂₇N = 473.62) |
| Sub 2-46 | m/z = 410.18(C₂₈H₁₀D₉NS = 410.58) |
| Sub 2-47 | m/z = 457.18(C₃₅H₂₃N = 457.58) |
| Sub 2-48 | m/z = 344.22(C₂₅H₁₂D₉N = 344.50) |
| Sub 2-49 | m/z = 530.27(C₃₉H₂₆D₄N₂ = 530.71) |
| Sub 2-50 | m/z = 460.19(C₃₄H₂₄N₂ = 460.58) |
| Sub 2-51 | m/z = 515.24(C₃₈H₂₁D₅N₂ = 515.67) |
| Sub 2-52 | m/z = 650.27(C₄₉H₃₄N₂ = 650.83) |
| Sub 2-53 | m/z = 499.20(C₃₆H₂₅N₃ = 499.62) |
| Sub 2-54 | m/z = 550.20(C₄₀H₂₆N₂O = 550.66) |
| Sub 2-55 | m/z = 460.19(C₃₄H₂₄N₂ = 460.58) |
| Sub 2-56 | m/z = 549.40(C₄₀H₇D₂₄N = 549.84) |
| Sub 2-57 | m/z = 616.41(C₄₄H₄D₂₇NO = 616.90) |
| Sub 2-58 | m/z = 549.40(C₄₀H₇D₂₄N = 549.84) |
| Sub 2-59 | m/z = 574.24(C₄₃H₃₀N₂ = 574.73) |
| Sub 2-60 | m/z = 485.21(C₃₇H₂₇N = 485.63) |
| Sub 2-61 | m/z = 494.27(C₃₇H₁₈D₉N = 494.68) |
| Sub 2-62 | m/z = 611.26(C₄₇H₃₃N = 611.79) |
| Sub 2-63 | m/z = 565.19(C₄₁H₂₇NS = 565.73) |
| Sub 2-64 | m/z = 649.28(C₅₀H₃₅N = 649.84) |
| Sub 2-65 | m/z = 649.28(C₅₀H₃₅N = 649.84) |
| Sub 2-66 | m/z = 647.26(C₅₀H₃₃N = 647.82) |
| Sub 2-67 | m/z = 625.24(C₄₇H₃₁NO = 625.77) |
| Sub 2-68 | m/z = 523.34(C₃₇HD₂₄NO = 523.76) |
| Sub 2-69 | m/z = 545.13(C₃₇H₂₃NS₂ = 545.72) |
| Sub 2-70 | m/z = 614.27(C₄₆H₃₄N₂ = 614.79) |
| Sub 2-71 | m/z = 615.26(C₄₆H₃₃NO = 615.78) |
| Sub 2-72 | m/z = 677.24(C₅₀H₃₁NO₂ = 677.80) |
| Sub 2-73 | m/z = 679.23(C₅₀H₃₃NS = 679.88) |
| Sub 2-74 | m/z = 743.33(C₅₆H₂₉D₇N₂ = 743.96) |
| Sub 2-75 | m/z = 601.28(C₄₆H₃₅N = 601.79) |
| Sub 2-76 | m/z = 676.33(C₄₉H₂₀D₁₂N₂O = 676.88) |
| Sub 2-77 | m/z = 746.34(C₅₃H₁₈D₁₆N₂S = 747.03) |
| Sub 2-78 | m/z = 727.25(C₅₄H₃₃NO₂ = 727.86) |
| Sub 2-79 | m/z = 688.34(C₅₁H₂₀D₁₃NO = 688.91) |
| Sub 2-80 | m/z = 327.20(C₂₄H₂₅N = 327.47) |
| Sub 2-81 | m/z = 383.22(C₂₇H₁₇D₆NO = 383.52) |
| Sub 2-82 | m/z = 525.25(C₄₀H₃₁N = 525.70) |
| Sub 2-83 | m/z = 468.26(C₃₄H₃₂N₂ = 468.64) |
| Sub 2-84 | m/z = 438.17(C₃₁H₂₂N₂O = 438.53) |
| Sub 2-85 | m/z = 317.12(C₂₁H₁₉NS = 317.45) |
| Sub 2-86 | m/z = 371.17(C₂₈H₂₁N = 371.48) |
| Sub 2-87 | m/z = 285.15(C₂₁H₁₉N = 285.39) |
| Sub 2-88 | m/z = 169.09(C₁₂H₁₁N = 169.23) |
| Sub 2-89 | m/z = 245.12(C₁₈H₁₅N = 245.33) |
| Sub 2-90 | m/z = 335.13(C₂₄H₁₇NO = 335.41) |
| Sub 2-91 | m/z = 358.15(C₂₄H₁₀D₇NS = 358.51) |
| Sub 2-92 | m/z = 229.18(C₁₆H₁₅D₄N = 229.36) |
| Sub 2-93 | m/z = 321.15(C₂₄H₁₉N = 321.42) |
| Sub 2-94 | m/z = 345.15(C₂₆H₁₉N = 345.45) |
| Sub 2-95 | m/z = 485.18(C₃₆H₂₃NO = 485.59) |
| Sub 2-96 | m/z = 486.27(C₃₅H₂₂D₇NO = 486.67) |
| Sub 2-97 | m/z = 487.22(C₃₆H₂₅DN₂ = 487.62) |
| Sub 2-98 | m/z = 323.14(C₂₂H₁₇N₃ = 323.40) |
| Sub 2-99 | m/z = 377.13(C₂₅H₁₅DN₂O₂ = 377.42) |

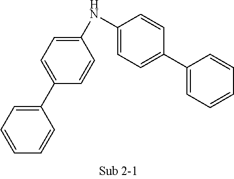 [SMILES: c1ccc(-c2ccc(Nc3ccc(-c4ccccc4)cc3)cc2)cc1]

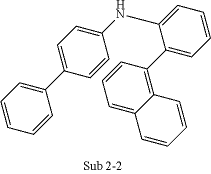 [SMILES: c1ccc(-c2ccc(Nc3ccccc3-c3cccc4ccccc34)cc2)cc1]

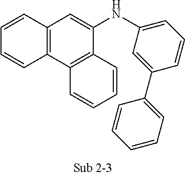 [SMILES: c1ccc(-c2cccc(Nc3cc4ccccc4c4ccccc34)c2)cc1]

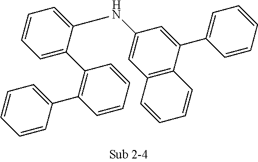 [SMILES: c1ccc(-c2ccccc2-c2ccccc2Nc2cc(-c3ccccc3)c3ccccc3c2)cc1]

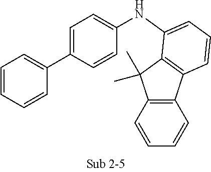 [SMILES: CC1(C)c2ccccc2-c2cccc(Nc3ccc(-c4ccccc4)cc3)c21]

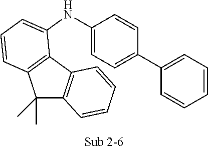 [SMILES: CC1(C)c2ccccc2-c2c(Nc3ccc(-c4ccccc4)cc3)cccc21]

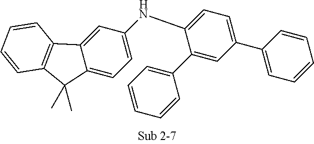 [SMILES: CC1(C)c2ccccc2-c2cc(Nc3ccc(-c4ccccc4)cc3-c3ccccc3)ccc21]

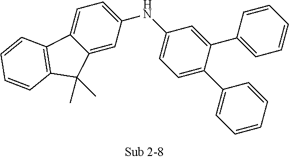 [SMILES: CC1(C)c2ccccc2-c2ccc(Nc3ccc(-c4ccccc4)c(-c4ccccc4)c3)cc21]

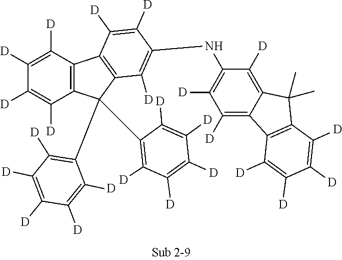 [SMILES: [2H]c1c([2H])c([2H])c(C2(c3c([2H])c([2H])c([2H])c([2H])c3[2H])c3c([2H])c([2H])c([2H])c([2H])c3-c3c([2H])c([2H])c(Nc4c([2H])c([2H])c5c(c4[2H])C(C)(C)c4c([2H])c([2H])c([2H])c([2H])c4-5)c([2H])c32)c([2H])c1[2H]]

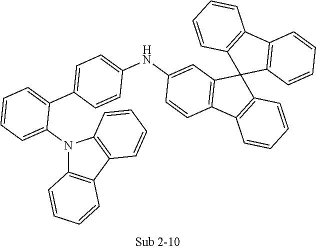 [SMILES: c1ccc(-n2c3ccccc3c3ccccc32)c(-c2ccc(Nc3ccc4c(c3)C3(c5ccccc5-c5ccccc53)c3ccccc3-4)cc2)c1]

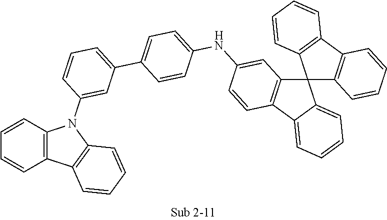 [SMILES: c1cc(-c2ccc(Nc3ccc4c(c3)C3(c5ccccc5-c5ccccc53)c3ccccc3-4)cc2)cc(-n2c3ccccc3c3ccccc32)c1]

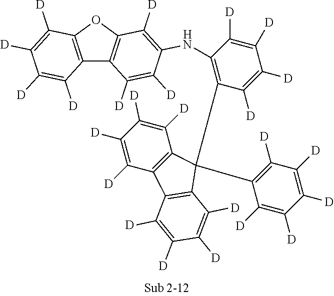 [SMILES: [2H]c1c([2H])c([2H])c(C2(c3c([2H])c([2H])c([2H])c([2H])c3Nc3c([2H])c([2H])c4c(oc5c([2H])c([2H])c([2H])c([2H])c54)c3[2H])c3c([2H])c([2H])c([2H])c([2H])c3-c3c([2H])c([2H])c([2H])c([2H])c32)c([2H])c1[2H]]

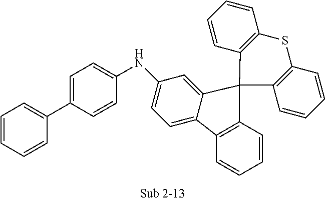 [SMILES: c1ccc(-c2ccc(Nc3ccc4c(c3)C3(c5ccccc5Sc5ccccc53)c3ccccc3-4)cc2)cc1]

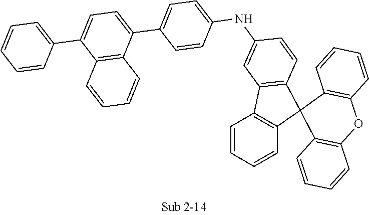 [SMILES: c1ccc(-c2ccc(-c3ccc(Nc4ccc5c(c4)-c4ccccc4C54c5ccccc5Oc5ccccc54)cc3)c3ccccc23)cc1]

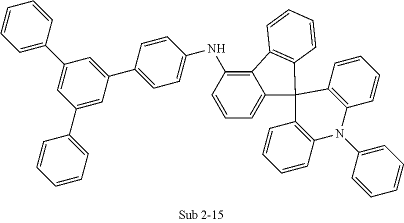 [SMILES: c1ccc(-c2cc(-c3ccccc3)cc(-c3ccc(Nc4cccc5c4-c4ccccc4C54c5ccccc5N(c5ccccc5)c5ccccc54)cc3)c2)cc1]

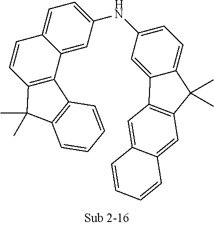

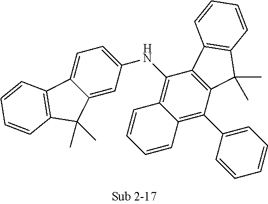 [SMILES: CC1(C)c2ccccc2-c2ccc(Nc3c4c(c(-c5ccccc5)c5ccccc35)C(C)(C)c3ccccc3-4)cc21]

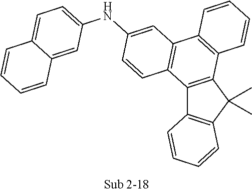 [SMILES: CC1(C)c2ccccc2-c2c1c1ccccc1c1cc(Nc3ccc4ccccc4c3)ccc21]

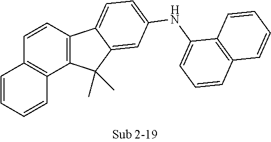 [SMILES: CC1(C)c2cc(Nc3cccc4ccccc34)ccc2-c2ccc3ccccc3c21]

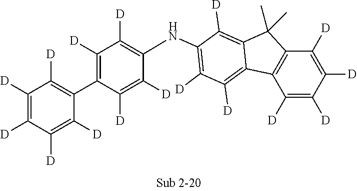 [SMILES: [2H]c1c([2H])c([2H])c(-c2c([2H])c([2H])c(Nc3c([2H])c([2H])c4c(c3[2H])C(C)(C)c3c([2H])c([2H])c([2H])c([2H])c3-4)c([2H])c2[2H])c([2H])c1[2H]]

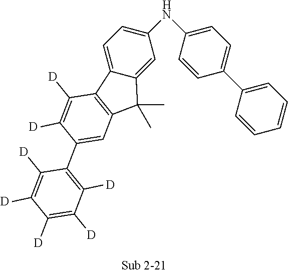 [SMILES: [2H]c1c(-c2c([2H])c([2H])c([2H])c([2H])c2[2H])cc2c(c1[2H])-c1ccc(Nc3ccc(-c4ccccc4)cc3)cc1C2(C)C]

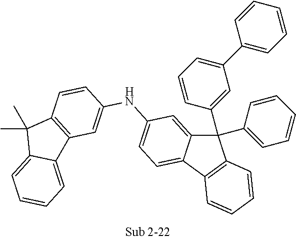 [SMILES: CC1(C)c2ccccc2-c2cc(Nc3ccc4c(c3)C(c3ccccc3)(c3cccc(-c5ccccc5)c3)c3ccccc3-4)ccc21]

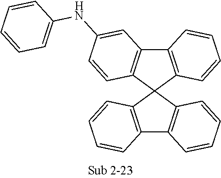 [SMILES: c1ccc(Nc2ccc3c(c2)-c2ccccc2C32c3ccccc3-c3ccccc32)cc1]

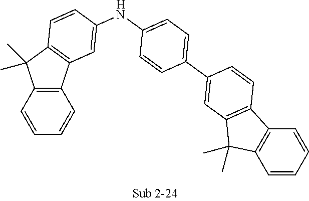 [SMILES: CC1(C)c2ccccc2-c2cc(Nc3ccc(-c4ccc5c(c4)C(C)(C)c4ccccc4-5)cc3)ccc21]

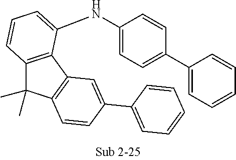 [SMILES: CC1(C)c2ccc(-c3ccccc3)cc2-c2c(Nc3ccc(-c4ccccc4)cc3)cccc21]

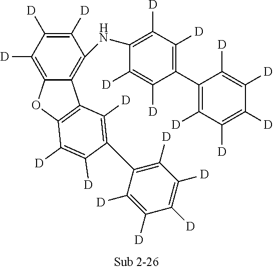 [SMILES: [2H]c1c([2H])c([2H])c(-c2c([2H])c([2H])c(Nc3c([2H])c([2H])c([2H])c4oc5c([2H])c([2H])c(-c6c([2H])c([2H])c([2H])c([2H])c6[2H])c([2H])c5c34)c([2H])c2[2H])c([2H])c1[2H]]

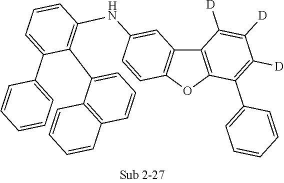 [SMILES: [2H]c1c([2H])c([2H])c2c(oc3ccc(Nc4cccc(-c5ccccc5)c4-c4cccc5ccccc45)cc32)c1-c1ccccc1]

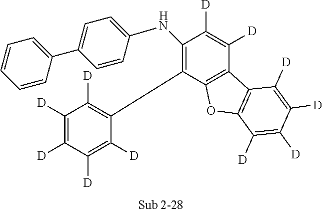 [SMILES: [2H]c1c([2H])c([2H])c(-c2c(Nc3ccc(-c4ccccc4)cc3)c([2H])c([2H])c3c2oc2c([2H])c([2H])c([2H])c([2H])c23)c([2H])c1[2H]]

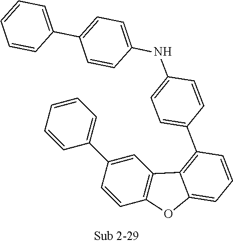 [SMILES: c1ccc(-c2ccc(Nc3ccc(-c4cccc5oc6ccc(-c7ccccc7)cc6c45)cc3)cc2)cc1]

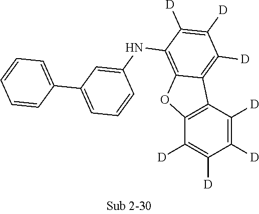 [SMILES: [2H]c1c([2H])c([2H])c2c(oc3c(Nc4cccc(-c5ccccc5)c4)c([2H])c([2H])c([2H])c32)c1[2H]]

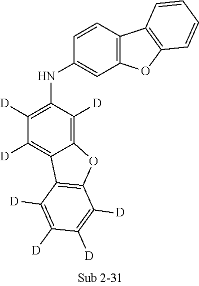 [SMILES: [2H]c1c([2H])c([2H])c2c(oc3c([2H])c(Nc4ccc5c(c4)oc4ccccc45)c([2H])c([2H])c32)c1[2H]]

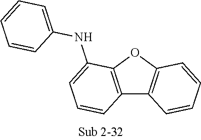 [SMILES: c1ccc(Nc2cccc3c2oc2ccccc23)cc1]

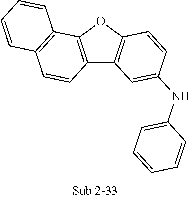 [SMILES: c1ccc(Nc2ccc3oc4c5ccccc5ccc4c3c2)cc1]

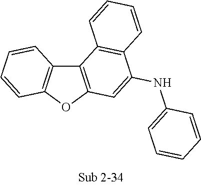 [SMILES: c1ccc(Nc2cc3oc4ccccc4c3c3ccccc23)cc1]

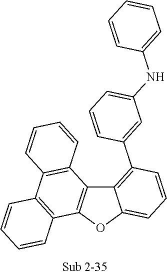 [SMILES: c1ccc(Nc2cccc(-c3cccc4oc5c6ccccc6c6ccccc6c5c34)c2)cc1]

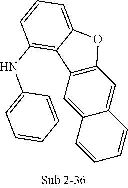 [SMILES: c1ccc(Nc2cccc3oc4cc5ccccc5cc4c23)cc1]

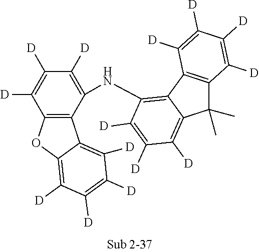 [SMILES: [2H]c1c([2H])c([2H])c2c(c1[2H])-c1c(Nc3c([2H])c([2H])c([2H])c4oc5c([2H])c([2H])c([2H])c([2H])c5c34)c([2H])c([2H])c([2H])c1C2(C)C]

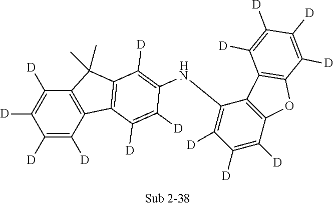 [SMILES: [2H]c1c([2H])c([2H])c2c(c1[2H])-c1c([2H])c([2H])c(Nc3c([2H])c([2H])c([2H])c4oc5c([2H])c([2H])c([2H])c([2H])c5c34)c([2H])c1C2(C)C]

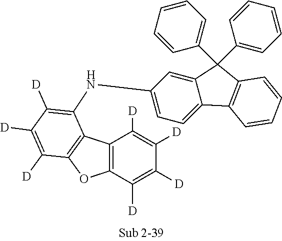 [SMILES: [2H]c1c([2H])c([2H])c2c(oc3c([2H])c([2H])c([2H])c(Nc4ccc5c(c4)C(c4ccccc4)(c4ccccc4)c4ccccc4-5)c32)c1[2H]]

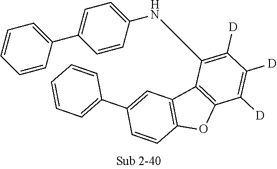 [SMILES: [2H]c1c([2H])c(Nc2ccc(-c3ccccc3)cc2)c2c(oc3ccc(-c4ccccc4)cc32)c1[2H]]

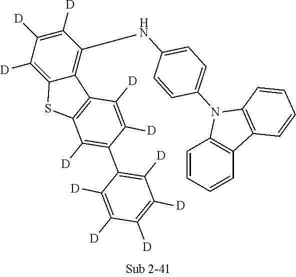 [SMILES: [2H]c1c([2H])c([2H])c(-c2c([2H])c([2H])c3c(sc4c([2H])c([2H])c([2H])c(Nc5ccc(-n6c7ccccc7c7ccccc76)cc5)c43)c2[2H])c([2H])c1[2H]]

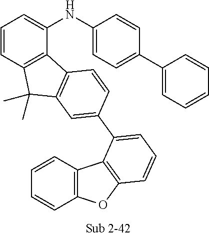 [SMILES: CC1(C)c2cc(-c3cccc4oc5ccccc5c34)ccc2-c2c(Nc3ccc(-c4ccccc4)cc3)cccc21]

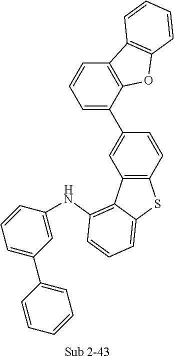 [SMILES: c1ccc(-c2cccc(Nc3cccc4sc5ccc(-c6cccc7c6oc6ccccc67)cc5c34)c2)cc1]

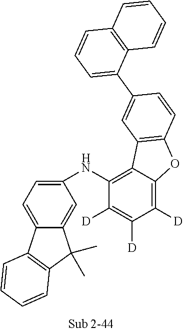 [SMILES: [2H]c1c([2H])c(Nc2ccc3c(c2)C(C)(C)c2ccccc2-3)c2c(oc3ccc(-c4cccc5ccccc45)cc32)c1[2H]]

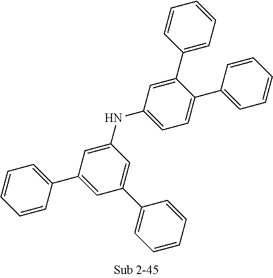 [SMILES: c1ccc(-c2cc(Nc3ccc(-c4ccccc4)c(-c4ccccc4)c3)cc(-c3ccccc3)c2)cc1]

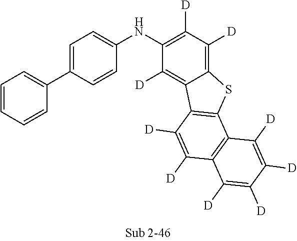 [SMILES: [2H]c1c(Nc2ccc(-c3ccccc3)cc2)c([2H])c2c(sc3c4c([2H])c([2H])c([2H])c([2H])c4c([2H])c([2H])c32)c1[2H]]

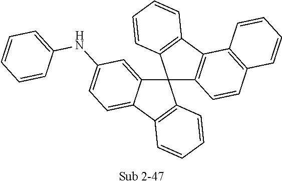 [SMILES: c1ccc(Nc2ccc3c(c2)C2(c4ccccc4-3)c3ccccc3-c3c2ccc2ccccc32)cc1]

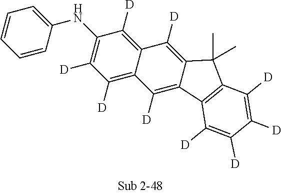 [SMILES: [2H]c1c([2H])c([2H])c2c(c1[2H])-c1c(c([2H])c3c([2H])c(Nc4ccccc4)c([2H])c([2H])c3c1[2H])C2(C)C]

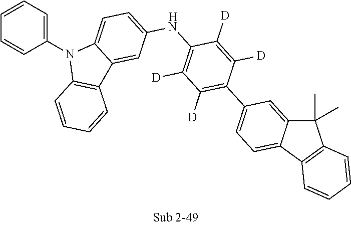 [SMILES: [2H]c1c([2H])c(-c2ccc3c(c2)C(C)(C)c2ccccc2-3)c([2H])c([2H])c1Nc1ccc2c(c1)c1ccccc1n2-c1ccccc1]

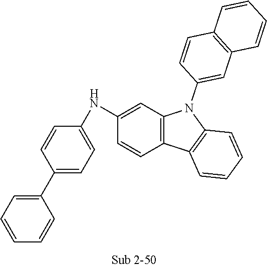 [SMILES: c1ccc(-c2ccc(Nc3ccc4c5ccccc5n(-c5ccc6ccccc6c5)c4c3)cc2)cc1]

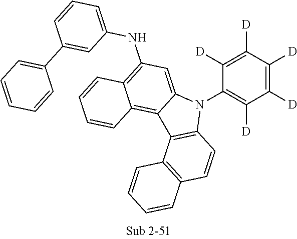 [SMILES: [2H]c1c([2H])c([2H])c(-n2c3ccc4ccccc4c3c3c4ccccc4c(Nc4cccc(-c5ccccc5)c4)cc32)c([2H])c1[2H]]

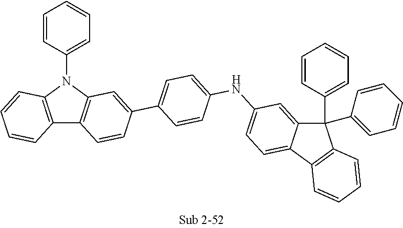 [SMILES: c1ccc(-n2c3ccccc3c3ccc(-c4ccc(Nc5ccc6c(c5)C(c5ccccc5)(c5ccccc5)c5ccccc5-6)cc4)cc32)cc1]

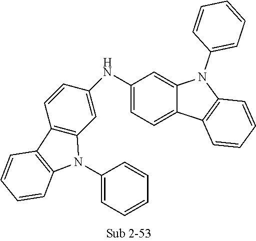 [SMILES: c1ccc(-n2c3ccccc3c3ccc(Nc4ccc5c6ccccc6n(-c6ccccc6)c5c4)cc32)cc1]

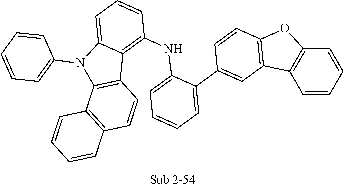 [SMILES: c1ccc(-n2c3cccc(Nc4ccccc4-c4ccc5oc6ccccc6c5c4)c3c3ccc4ccccc4c32)cc1]

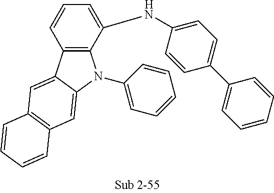 [SMILES: c1ccc(-c2ccc(Nc3cccc4c5cc6ccccc6cc5n(-c5ccccc5)c34)cc2)cc1]

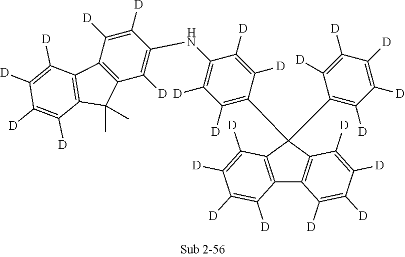 [SMILES: [2H]c1c([2H])c([2H])c(C2(c3c([2H])c([2H])c(Nc4c([2H])c([2H])c5c(c4[2H])C(C)(C)c4c([2H])c([2H])c([2H])c([2H])c4-5)c([2H])c3[2H])c3c([2H])c([2H])c([2H])c([2H])c3-c3c([2H])c([2H])c([2H])c([2H])c32)c([2H])c1[2H]]

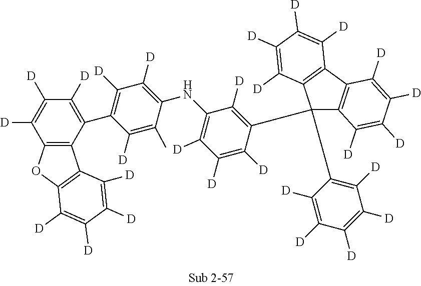 [SMILES: [2H]c1c([2H])c([2H])c(C2(c3c([2H])c([2H])c([2H])c(Nc4c([2H])c([2H])c(-c5c([2H])c([2H])c([2H])c6oc7c([2H])c([2H])c([2H])c([2H])c7c56)c([2H])c4C)c3[2H])c3c([2H])c([2H])c([2H])c([2H])c3-c3c([2H])c([2H])c([2H])c([2H])c32)c([2H])c1[2H]]

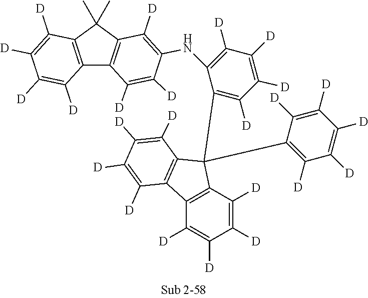 [SMILES: [2H]c1c([2H])c([2H])c(C2(c3c([2H])c([2H])c([2H])c([2H])c3Nc3c([2H])c([2H])c4c(c3[2H])C(C)(C)c3c([2H])c([2H])c([2H])c([2H])c3-4)c3c([2H])c([2H])c([2H])c([2H])c3-c3c([2H])c([2H])c([2H])c([2H])c32)c([2H])c1[2H]]

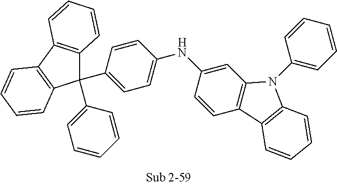 [SMILES: c1ccc(-n2c3ccccc3c3ccc(Nc4ccc(C5(c6ccccc6)c6ccccc6-c6ccccc65)cc4)cc32)cc1]

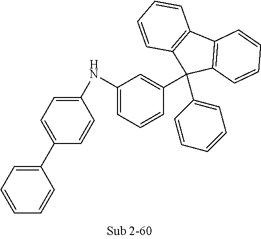 [SMILES: c1ccc(-c2ccc(Nc3cccc(C4(c5ccccc5)c5ccccc5-c5ccccc54)c3)cc2)cc1]

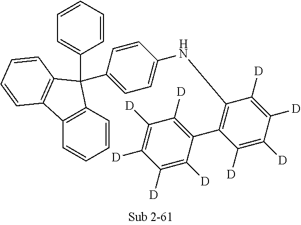 [SMILES: [2H]c1c([2H])c([2H])c(-c2c([2H])c([2H])c([2H])c([2H])c2Nc2ccc(C3(c4ccccc4)c4ccccc4-c4ccccc43)cc2)c([2H])c1[2H]]

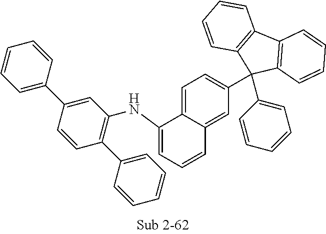 [SMILES: c1ccc(-c2ccc(-c3ccccc3)c(Nc3cccc4cc(C5(c6ccccc6)c6ccccc6-c6ccccc65)ccc34)c2)cc1]

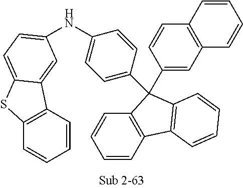 [SMILES: c1ccc2c(c1)-c1ccccc1C2(c1ccc(Nc2ccc3sc4ccccc4c3c2)cc1)c1ccc2ccccc2c1]

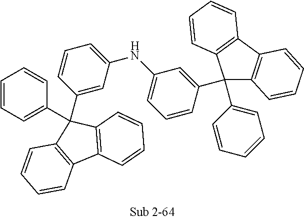 [SMILES: c1ccc(C2(c3cccc(Nc4cccc(C5(c6ccccc6)c6ccccc6-c6ccccc65)c4)c3)c3ccccc3-c3ccccc32)cc1]

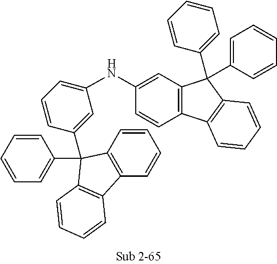 [SMILES: c1ccc(C2(c3cccc(Nc4ccc5c(c4)C(c4ccccc4)(c4ccccc4)c4ccccc4-5)c3)c3ccccc3-c3ccccc32)cc1]

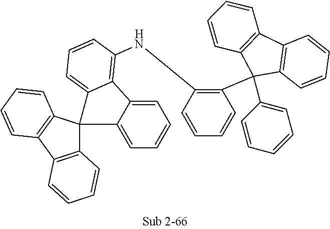 [SMILES: c1ccc(C2(c3ccccc3Nc3cccc4c3-c3ccccc3C43c4ccccc4-c4ccccc43)c3ccccc3-c3ccccc32)cc1]

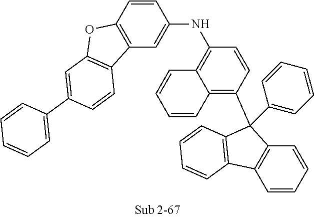 [SMILES: c1ccc(-c2ccc3c(c2)oc2ccc(Nc4ccc(C5(c6ccccc6)c6ccccc6-c6ccccc65)c5ccccc45)cc23)cc1]

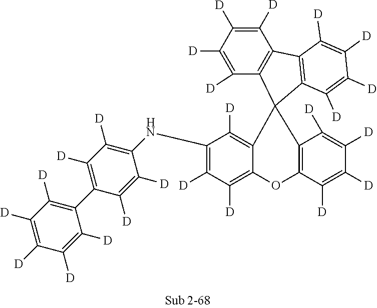 [SMILES: [2H]c1c([2H])c([2H])c(-c2c([2H])c([2H])c(Nc3c([2H])c([2H])c4c(c3[2H])C3(c5c([2H])c([2H])c([2H])c([2H])c5O4)c4c([2H])c([2H])c([2H])c([2H])c4-c4c([2H])c([2H])c([2H])c([2H])c43)c([2H])c2[2H])c([2H])c1[2H]]

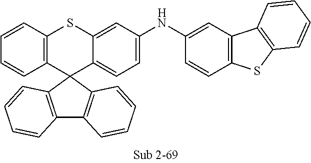 [SMILES: c1ccc2c(c1)Sc1cc(Nc3ccc4sc5ccccc5c4c3)ccc1C21c2ccccc2-c2ccccc21]

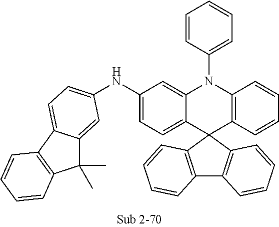 [SMILES: CC1(C)c2ccccc2-c2ccc(Nc3ccc4c(c3)N(c3ccccc3)c3ccccc3C43c4ccccc4-c4ccccc43)cc21]

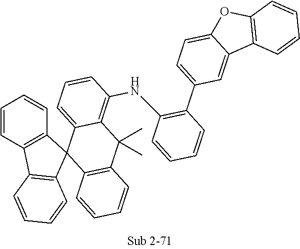 [SMILES: CC1(C)c2ccccc2C2(c3ccccc3-c3ccccc32)c2cccc(Nc3ccccc3-c3ccc4oc5ccccc5c4c3)c21]

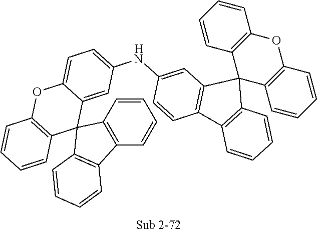 [SMILES: c1ccc2c(c1)Oc1ccc(Nc3ccc4c(c3)C3(c5ccccc5Oc5ccccc53)c3ccccc3-4)cc1C21c2ccccc2-c2ccccc21]

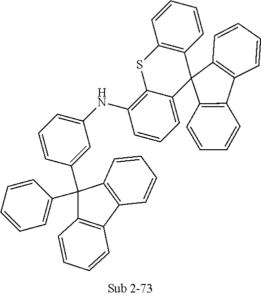 [SMILES: c1ccc(C2(c3cccc(Nc4cccc5c4Sc4ccccc4C54c5ccccc5-c5ccccc54)c3)c3ccccc3-c3ccccc32)cc1]

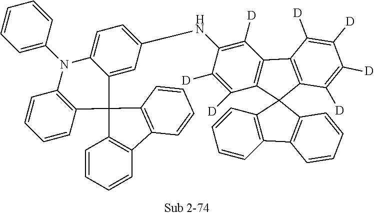 [SMILES: [2H]c1c([2H])c([2H])c2c(c1[2H])-c1c([2H])c(Nc3ccc4c(c3)C3(c5ccccc5-c5ccccc53)c3ccccc3N4c3ccccc3)c([2H])c([2H])c1C21c2ccccc2-c2ccccc21]

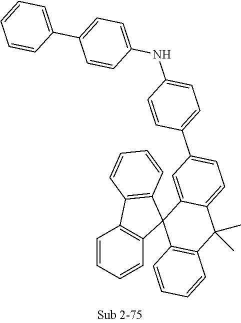 [SMILES: CC1(C)c2ccccc2C2(c3ccccc3-c3ccccc32)c2cc(-c3ccc(Nc4ccc(-c5ccccc5)cc4)cc3)ccc21]

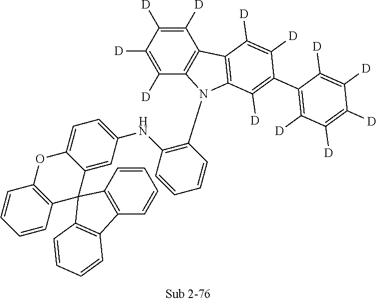 [SMILES: [2H]c1c([2H])c([2H])c(-c2c([2H])c([2H])c3c4c([2H])c([2H])c([2H])c([2H])c4n(-c4ccccc4Nc4ccc5c(c4)C4(c6ccccc6O5)c5ccccc5-c5ccccc54)c3c2[2H])c([2H])c1[2H]]

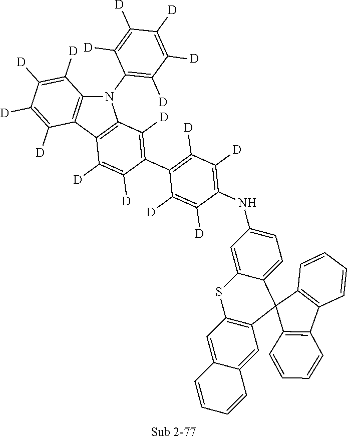 [SMILES: [2H]c1c([2H])c([2H])c(-n2c3c([2H])c([2H])c([2H])c([2H])c3c3c([2H])c([2H])c(-c4c([2H])c([2H])c(Nc5ccc6c(c5)Sc5cc7ccccc7cc5C65c6ccccc6-c6ccccc65)c([2H])c4[2H])c([2H])c32)c([2H])c1[2H]]

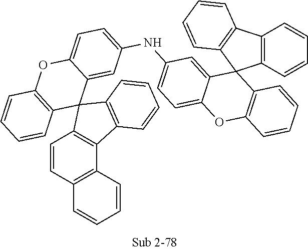 [SMILES: c1ccc2c(c1)Oc1ccc(Nc3ccc4c(c3)C3(c5ccccc5O4)c4ccccc4-c4c3ccc3ccccc43)cc1C21c2ccccc2-c2ccccc21]

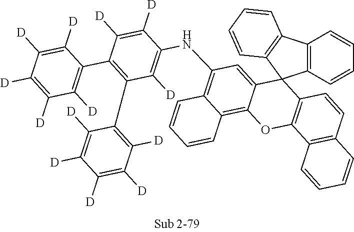 [SMILES: [2H]c1c([2H])c([2H])c(-c2c([2H])c([2H])c(Nc3cc4c(c5ccccc35)Oc3c(ccc5ccccc35)C43c4ccccc4-c4ccccc43)c([2H])c2-c2c([2H])c([2H])c([2H])c([2H])c2[2H])c([2H])c1[2H]]

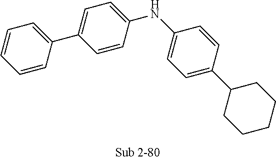 [SMILES: c1ccc(-c2ccc(Nc3ccc(C4CCCCC4)cc3)cc2)cc1]

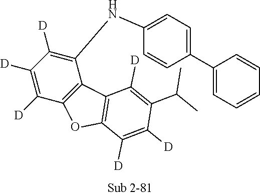 [SMILES: [2H]c1c(C(C)C)c([2H])c2c(oc3c([2H])c([2H])c([2H])c(Nc4ccc(-c5ccccc5)cc4)c32)c1[2H]]

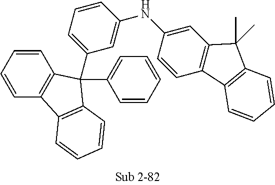 [SMILES: CC1(C)c2ccccc2-c2ccc(Nc3cccc(C4(c5ccccc5)c5ccccc5-c5ccccc54)c3)cc21]

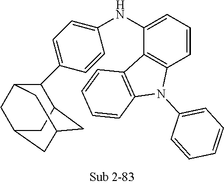 [SMILES: c1ccc(-n2c3ccccc3c3c(Nc4ccc(C5C6CC7CC(C6)CC5C7)cc4)cccc32)cc1]

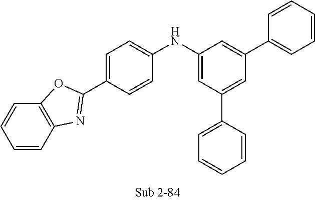 [SMILES: c1ccc(-c2cc(Nc3ccc(-c4nc5ccccc5o4)cc3)cc(-c3ccccc3)c2)cc1]

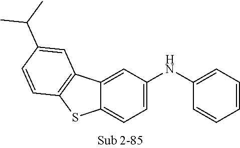 [SMILES: CC(C)c1ccc2sc3ccc(Nc4ccccc4)cc3c2c1]

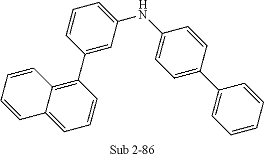 [SMILES: c1ccc(-c2ccc(Nc3cccc(-c4cccc5ccccc45)c3)cc2)cc1]

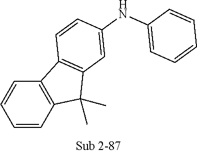 [SMILES: CC1(C)c2ccccc2-c2ccc(Nc3ccccc3)cc21]

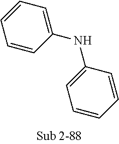 [SMILES: c1ccc(Nc2ccccc2)cc1]

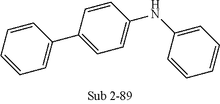 [SMILES: c1ccc(Nc2ccc(-c3ccccc3)cc2)cc1]

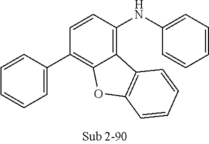 [SMILES: c1ccc(Nc2ccc(-c3ccccc3)c3oc4ccccc4c23)cc1]

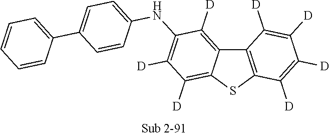 [SMILES: [2H]c1c([2H])c([2H])c2c(sc3c([2H])c([2H])c(Nc4ccc(-c5ccccc5)cc4)c([2H])c32)c1[2H]]

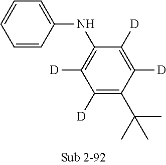 [SMILES: [2H]c1c([2H])c(C(C)(C)C)c([2H])c([2H])c1Nc1ccccc1]

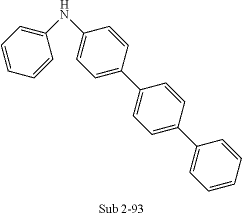 [SMILES: c1ccc(Nc2ccc(-c3ccc(-c4ccccc4)cc3)cc2)cc1]

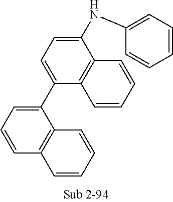 [SMILES: c1ccc(Nc2ccc(-c3cccc4ccccc34)c3ccccc23)cc1]

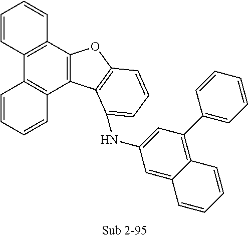 [SMILES: c1ccc(-c2cc(Nc3cccc4oc5c6ccccc6c6ccccc6c5c34)cc3ccccc23)cc1]

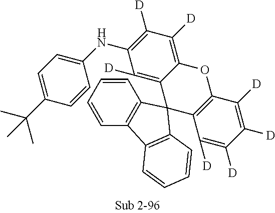 [SMILES: [2H]c1c([2H])c([2H])c2c(c1[2H])Oc1c([2H])c([2H])c(Nc3ccc(C(C)(C)C)cc3)c([2H])c1C21c2ccccc2-c2ccccc21]

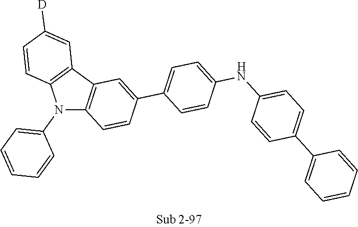 [SMILES: [2H]c1ccc2c(c1)c1cc(-c3ccc(Nc4ccc(-c5ccccc5)cc4)cc3)ccc1n2-c1ccccc1]

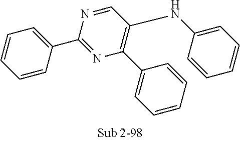 [SMILES: c1ccc(Nc2cnc(-c3ccccc3)nc2-c2ccccc2)cc1]

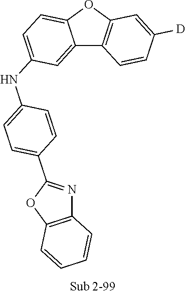 [SMILES: [2H]c1ccc2c(c1)oc1ccc(Nc3ccc(-c4nc5ccccc5o4)cc3)cc12]

### Synthesis Example of the Final Compound

### 1. Synthesis Example of P-6

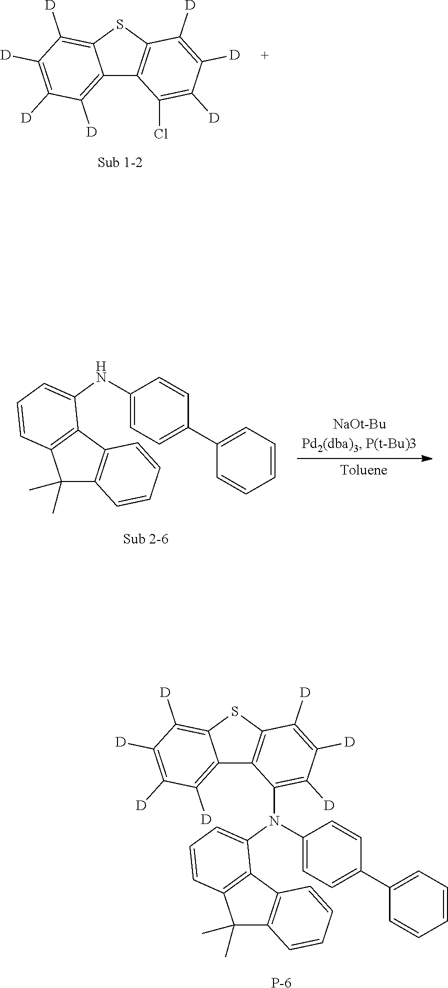

After dissolving Sub 1-2 (10.0 g, 44.3 mmol) in toluene (221 ml), Sub 2-6 (15.5 g, 44.3 mmol), Pd₂(dba)₃ (1.2 g, 1.3 mmol), P(t-Bu)₃ (0.5 g, 2.7 mmol), NaOt-Bu (8.5 g, 88.6 mmol) are added thereto and the reaction is carried out at 80° C. When the reaction is completed, the reaction product is extracted with CH₂Cl₂ and water, and then the organic layer is dried with MgSO₄ and concentrated. Then, the concentrate is separated through a silica gel column and recrystallized to obtain 18.0 g (yield: 73.8%) of the product.

### 2. Synthesis Example of P-9

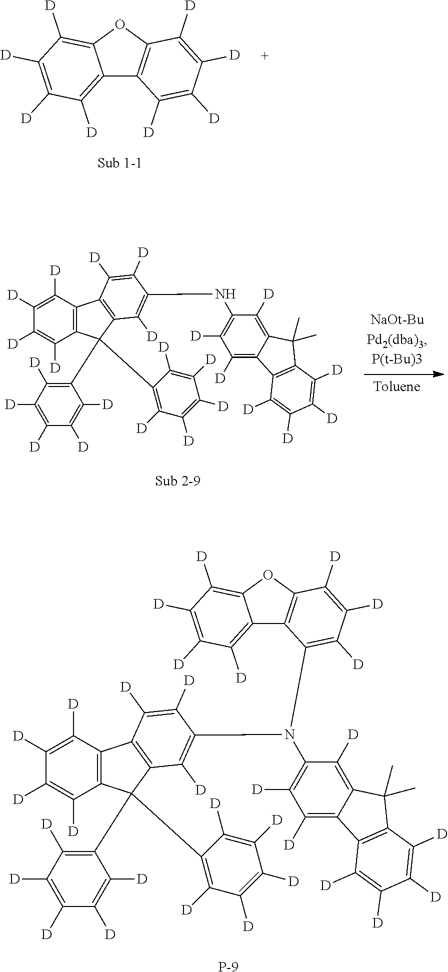

After dissolving Sub 1-1 (10.0 g, 47.7 mmol) in toluene (238 ml), Sub 2-9 (25.3 g, 47.7 mmol), Pd₂(dba)₃ (1.3 g, 1.4 mmol), P(t-Bu)₃ (0.6 g, 2.9 mmol), NaOt-Bu (9.2 g, 95.4 mmol) are thereto, and the synthesis is carried out in the same manner as the synthesis of P-6 to obtain 24.3 g (yield: 70.5%) of product.

### 3. Synthesis Example of P-26

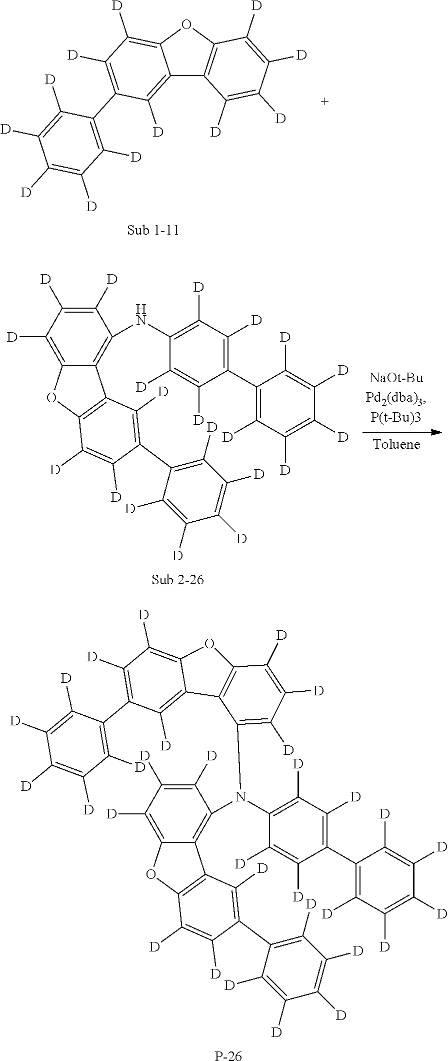

After dissolving Sub 1-11 (10.0 g, 34.5 mmol) in toluene (173 ml), Sub 2-26 (14.4 g, 34.5 mmol), Pd₂(dba)₃ (1.0 g, 1.0 mmol), P(t-Bu)₃ (0.4 g, 2.1 mmol), NaOt-Bu (6.6 g, 69.0 mmol) are thereto, and the synthesis is carried out in the same manner as the synthesis of P-6 to obtain 17.1 g (yield: 72.4%) of product.

### 4. Synthesis Example of P-37

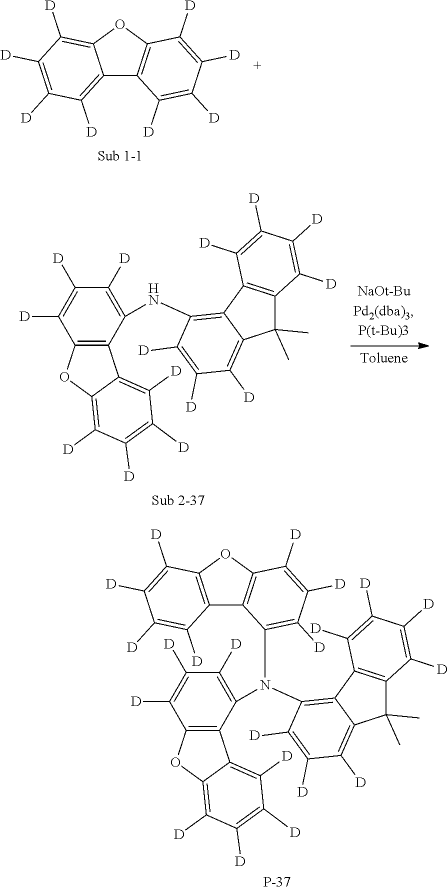

After dissolving Sub 1-1 (10.0 g, 47.7 mmol) in toluene (238 ml), Sub 2-37 (18.0 g, 47.7 mmol), Pd₂(dba)₃ (1.3 g, 1.4 mmol), P(t-Bu)₃ (0.6 g, 2.9 mmol), NaOt-Bu (9.2 g, 95.4 mmol) are thereto, and the synthesis is carried out in the same manner as the synthesis of P-6 to obtain 18.8 g (yield: 70.2%) of product.

### 5. Synthesis Example of P-57

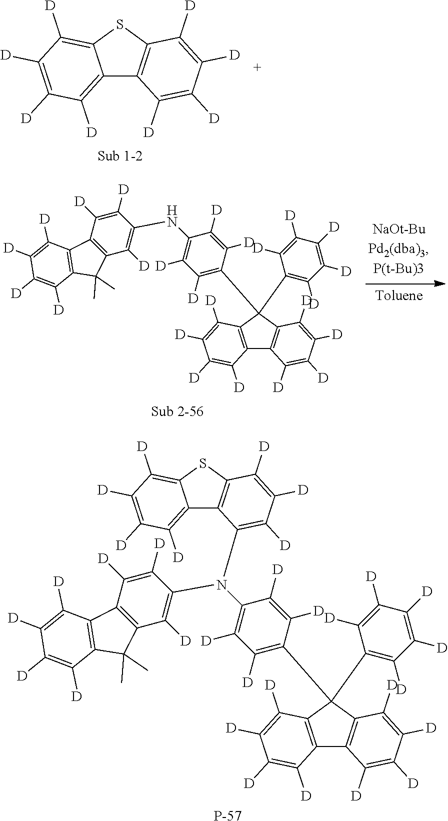

After dissolving Sub 1-2 (10.0 g, 44.3 mmol) in toluene (221 ml), Sub 2-56 (23.5 g, 44.3 mmol), Pd₂(dba)₃ (1.2 g, 1.3 mmol), P(t-Bu)₃ (0.5 g, 2.7 mmol), NaOt-Bu (8.5 g, 88.6 mmol) are thereto, and the synthesis is carried out in the same manner as the synthesis of P-6 to obtain 23.8 g (yield: 72.6%) of product.

### 5. Synthesis Example of P-69

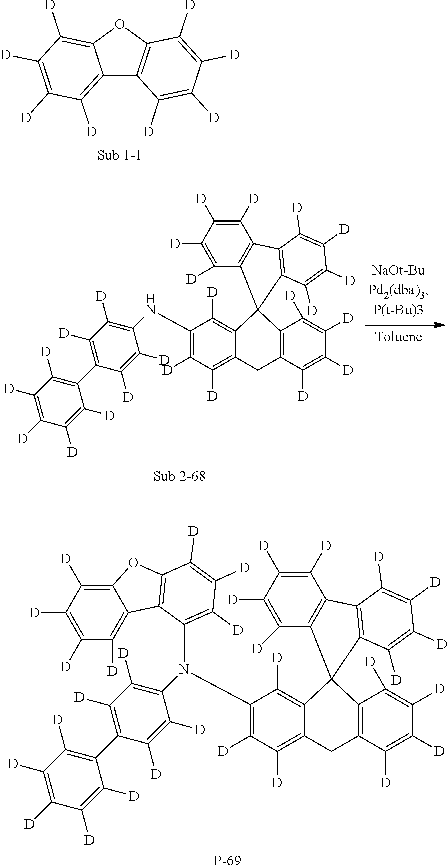

After dissolving Sub 1-1 (10.0 g, 47.7 mmol) in toluene (238 ml), Sub 2-68 (24.1 g, 47.7 mmol), Pd₂(dba)₃ (1.3 g, 1.4 mmol), P(t-Bu)₃ (0.6 g, 2.9 mmol), NaOt-Bu (9.2 g, 95.4 mmol) are thereto, and the synthesis is carried out in the same manner as the synthesis of P-6 to obtain 23.2 g (yield: 69.8%) of product.

### 6. Synthesis Example of P-101

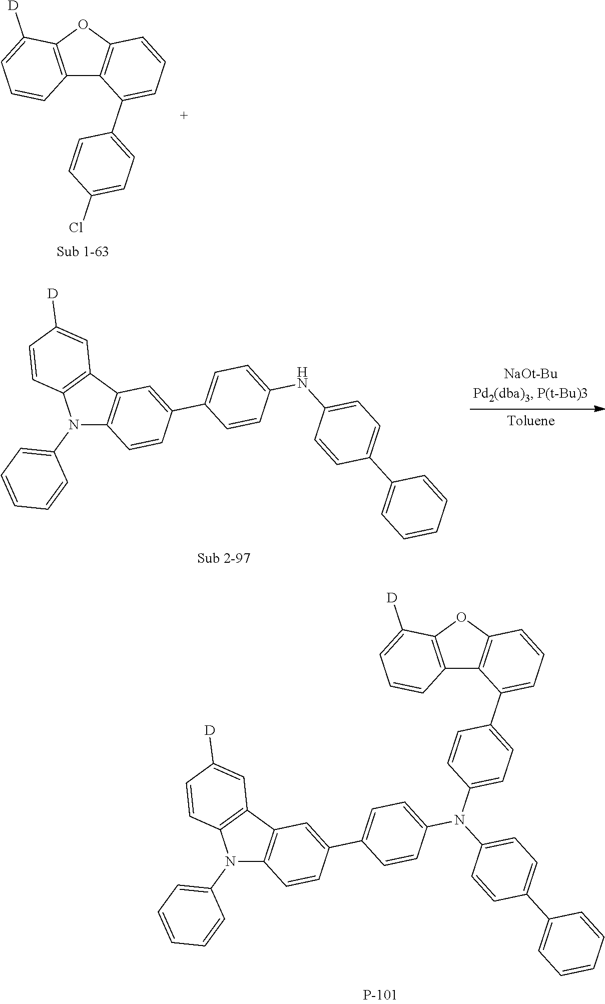 [SMILES: [2H]c1cccc2c1oc1cccc(-c3ccc(Cl)cc3)c12 ; [2H]c1ccc2c(c1)c1cc(-c3ccc(Nc4ccc(-c5ccccc5)cc4)cc3)ccc1n2-c1ccccc1 ; [2H]c1ccc2c(c1)c1cc(-c3ccc(N(c4ccc(-c5ccccc5)cc4)c4ccc(-c5cccc6oc7c([2H])cccc7c56)cc4)cc3)ccc1n2-c1ccccc1]

After dissolving Sub 1-63 (10.0 g, 35.7 mmol) in toluene (179 ml), Sub 2-97 (16.8 g, 35.7 mmol), Pd₂(dba)₃ (1.0 g, 1.1 mmol), P(t-Bu)₃ (0.4 g, 2.1 mmol), NaOt-Bu (6.9 g, 71.5 mmol) are thereto, and the synthesis is carried out in the same manner as the synthesis of P-6 to obtain 18.6 g (yield: 71.1%) of product.

The FD-MS values of the compounds P-1 to P-104 of the present invention prepared according to the above synthesis examples are shown in Table 3 below.

TABLE 3

| Compound | FD-MS | Compound | FD-MS |
|---|---|---|---|
| P-1 | m/z = 494.24(C₃₆H₁₈D₇NO = 494.64) | P-2 | m/z = 560.23(C₄₀H₂₀D₇NS = 560.77) |
| P-3 | m/z = 518.24(C₃₈H₁₈D₇NO = 518.67) | P-4 | m/z = 636.26(C₄₆H₂₄D₇NS = 636.86) |
| P-5 | m/z = 534.27(C₃₉H₂₂D₇NO = 534.71) | P-6 | m/z = 550.25(C₃₉H₂₂D₇NS = 550.77) |
| P-7 | m/z = 607.28(C₄₅H₂₉D₄NO = 607.79) | P-8 | m/z = 622.25(C₄₅H₃₀D₃NS = 622.84) |
| P-9 | m/z = 722.48(C₅₂H₆D₃₁NO = 723.06) | P-10 | m/z = 821.34(C₆₁H₃₁D₇N₂O = 822.03) |
| P-11 | m/z = 816.31(C₆₁H₃₆D₂N₂O = 817) | P-12 | m/z = 696.43(C₄₉D₃₁NO₂ = 696.98) |
| P-13 | m/z = 701.21(C₄₉H₂₇D₄NS₂ = 701.94) | P-14 | m/z = 810.28(C₅₉H₃₄D₃NOS = 811.03) |
| P-15 | m/z = 896.37(C₆₇H₄₀D₄N₂O = 897.13) | P-16 | m/z = 668.29(C₅₀H₃₆DNO = 668.86) |
| P-17 | m/z = 694.31(C₅₂H₃₈DNO = 694.9) | P-18 | m/z = 618.22(C₄₅H₃₀DNS = 618.82) |
| P-19 | m/z = 568.21(C₄₁H₂₈DNS = 568.76) | P-20 | m/z = 630.43(C₄₅H₆D₂₇NO = 630.93) |
| P-21 | m/z = 697.4(C₅₁H₁₉D₁₈NO = 697.97) | P-22 | m/z = 936.36(C₇₀H₄₈DNS = 937.24) |
| P-23 | m/z = 735.33(C₅₅H₂₅D₁₀NO = 735.95) | P-24 | m/z = 822.4(C₆₀H₃₄D₁₁NS = 823.15) |
| P-25 | m/z = 683.31(C₅₁H₃₃D₄NO = 683.89) | P-26 | m/z = 684.43(C₄₈D₃₁NO₂ = 684.97) |
| P-27 | m/z = 804.32(C₅₈H₂₈D₉NOS = 805.05) | P-28 | m/z = 675.37(C₄₈H₉D₂₂NO₂ = 675.92) |
| P-29 | m/z = 811.34(C₆₀H₃₃D₆NO₂ = 812.01) | P-30 | m/z = 680.28(C₄₈H₂₀D₁₁NOS = 680.91) |
| P-31 | m/z = 605.27(C₄₂H₁₁D₁₄NO₃ = 605.75) | P-32 | m/z = 672.23(C₄₈H₂₈D₃NOS = 672.86) |
| P-33 | m/z = 784.31(C₅₈H₃₂D₅NO₂ = 784.97) | P-34 | m/z = 648.23(C₄₆H₂₄D₅NOS = 648.83) |
| P-35 | m/z = 902.33(C₆₆H₃₄D₇NOS = 903.16) | P-36 | m/z = 722.37(C₅₂H₁₄D₁₉NO₂ = 722.96) |
| P-37 | m/z = 562.34(C₃₉H₆D₂₁NO₂ = 562.78) | P-38 | m/z = 642.39(C₄₅H₆D₂₅NO₂ = 642.9) |
| P-39 | m/z = 679.32(C₄₉H₁₇D₁₄NO₂ = 679.88) | P-40 | m/z = 735.3(C₅₄H₂₉D₆NO₂ = 735.92) |
| P-41 | m/z = 796.35(C₅₄H₁₂D₂₂N₂S₂ = 797.13) | P-42 | m/z = 716.29(C₅₁H₂₈D₇NOS = 716.95) |
| P-43 | m/z = 703.19(C₄₈H₂₅D₄NOS₂ = 703.91) | P-44 | m/z = 690.27(C₄₉H₂₆D₇NOS = 690.91) |
| P-45 | m/z = 851.35(C₆₃H₄₁D₄NS = 852.14) | P-46 | m/z = 824.32(C₅₈H₂₄D₁₃NS₂ = 825.14) |
| P-47 | m/z = 882.39(C₆₅H₂₆D₁₅NS = 883.2) | P-48 | m/z = 609.32(C₄₃H₁₅D₁₆NS = 609.89) |
| P-49 | m/z = 756.31(C₅₄H₂₄D₁₁NOS = 757.01) | P-50 | m/z = 703.35(C₅₁H₂₅D₁₁N₂O = 703.93) |
| P-51 | m/z = 633.28(C₄₆H₂₃D₇N₂O = 633.8) | P-52 | m/z = 700.28(C₅₀H₂₄D₈N₂S = 700.93) |
| P-53 | m/z = 836.32(C₆₁H₃₆D₄N₂S = 837.09) | P-54 | m/z = 688.27(C₄₈H₂₄D₇N₃S = 688.9) |
| P-55 | m/z = 733.23(C₅₂H₃₁DN₂OS = 733.91) | P-56 | m/z = 713.34(C₅₂H₂₃D₁₁N₂O = 713.92) |
| P-57 | m/z = 738.46(C₅₂H₆D₃₁NS = 739.12) | P-58 | m/z = 789.5(C₅₆H₃D₃₄NO₂ = 790.12) |
| P-59 | m/z = 722.48(C₅₂H₆D₃₁NO = 723.06) | P-60 | m/z = 743.3(C₅₅H₃₃D₃N₂O = 743.92) |
| P-61 | m/z = 674.28(C₄₉H₂₆D₇NS = 674.91) | P-62 | m/z = 747.41(C₅₅H₁₇D₂₀NO = 748.03) |
| P-63 | m/z = 880.38(C₆₅H₃₂D₁₁NS = 881.19) | P-64 | m/z = 738.27(C₅₃H₂₆D₇NOS = 738.96) |
| P-65 | m/z = 835.32(C₆₂H₃₇D₄NS = 836.1) | P-66 | m/z = 819.34(C₆₂H₃₇D₄NO = 820.04) |
| P-67 | m/z = 816.32(C₆₂H₃₆D₃NO = 817.02) | P-68 | m/z = 809.27(C₅₉H₃₅D₂NOS = 810.02) |
| P-69 | m/z = 696.43(C₄₉D₃₁NO₂ = 696.98) | P-70 | m/z = 730.17(C₄₉H₂₆D₃NS₃ = 730.98) |
| P-71 | m/z = 800.32(C₅₈H₃₆D₄N₂S = 801.06) | P-72 | m/z = 785.32(C₅₈H₃₅D₄NO₂ = 785.98) |
| P-73 | m/z = 866.3(C₆₂H₃₀D₇NO₂S = 867.09) | P-74 | m/z = 852.32(C₆₂H₃₂D₇NOS = 853.1) |
| P-75 | m/z = 913.4(C₆₈H₃₁D₁₁N₂O = 914.16) | P-76 | m/z = 870.4(C₆₄H₃₄D₁₁NS = 871.2) |
| P-77 | m/z = 843.37(C₆₁H₂₅D₁₃N₂O₂ = 844.07) | P-78 | m/z = 913.39(C₆₅H₂₃D₁₇N₂OS = 914.21) |
| P-79 | m/z = 911.28(C₆₆H₃₇D₂NO₂S = 912.12) | P-80 | m/z = 877.4(C₆₃H₁₉D₂₀NOS = 878.19) |
| P-81 | m/z = 571.32(C₄₀H₃₃D₆NS = 571.86) | P-82 | m/z = 597.34(C₄₂H₂₃D₁₂NO₂ = 597.82) |
| P-83 | m/z = 833.44(C₆₂H₃₉D₁₀NO = 834.14) | P-84 | m/z = 788.41(C₅₆H₄₄D₆N₂S = 789.13) |
| P-85 | m/z = 695.24(C₄₇H₂₂D₇FN₂OS = 695.86) | P-86 | m/z = 565.2(C₃₈H₂₇D₂NO₂S = 565.73) |
| P-87 | m/z = 618.27(C₄₆H₂₆D₅NO = 618.79) | P-88 | m/z = 626.22(C₄₄H₂₃D₅FNS = 626.8) |
| P-89 | m/z = 717.32(C₅₂H₂₃D₁₂NS = 717.99) | P-90 | m/z = 595.28(C₄₄H₃₃D₂NO = 595.78) |
| P-91 | m/z = 520.26(C₃₈H₃₂DNO = 520.69) | P-92 | m/z = 495.2(C₃₅H₂₁D₄NS = 495.68) |
| P-93 | m/z = 604.21(C₄₄H₂₈DNS = 604.79) | P-94 | m/z = 556.22(C₄₀H₂₀D₅NO₂ = 556.68) |
| P-95 | m/z = 655.27(C₄₆H₁₇D₁₂NOS = 655.88) | P-96 | m/z = 615.29(C₄₄H₂₉D₆NS = 615.87) |
| P-97 | m/z = 614.27(C₄₄H₁₈D₁₁NS = 614.85) | P-98 | m/z = 570.27(C₄₂H₁₈D₉NO = 570.74) |
| P-99 | m/z = 753.26(C₅₆H₃₁D₂NO₂ = 753.9) | P-100 | m/z = 770.33(C₅₅H₃₀D₉NOS = 771.04) |
| P-101 | m/z = 730.3(C₅₄H₃₄D₂N₂O = 730.91) | P-102 | m/z = 746.27(C₅₄H₃₄D₂N₂S = 746.97) |
| P-103 | m/z = 493.21(C₃₄H₁₉D₄N₃O = 493.6) | P-104 | m/z = 544.18(C₃₇H₂₀D₂N₂O₃ = 544.61) |

### Synthesis Example of Formula 2

Compound (final product 2) represented by Formula 2 of the present invention can be synthesized by the reaction route of Reaction Scheme 3 below, but is not limited thereto.

In the following Reaction Scheme 3, G¹ is L⁵ or L⁶ of Formula 2, and G² is Ar⁴ or Ar⁵.

### <Reaction Scheme 3>(Hal is I, Br or Cl.)

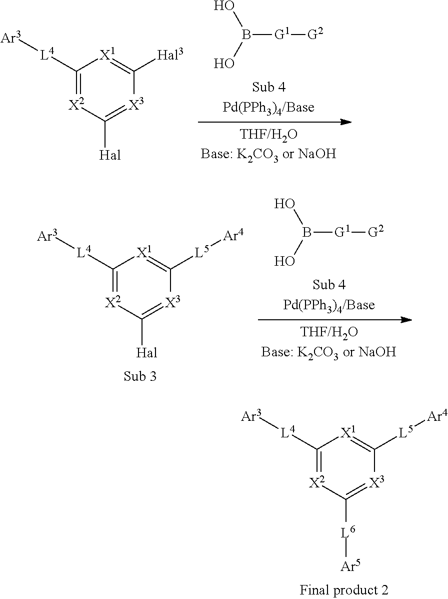

### Synthesis Example of Sub 3

A synthesis example of Sub 3 of Reaction Scheme 3 is as follows, but is not limited thereto.

### 1. Synthesis Example of Sub 3-7

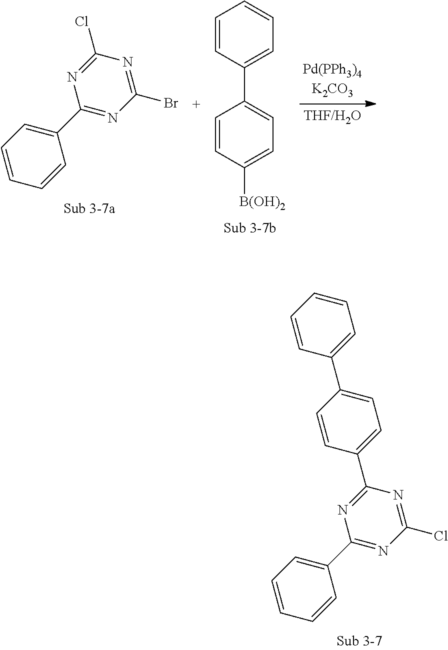 [SMILES: Clc1nc(Br)nc(-c2ccccc2)n1 ; OB(O)c1ccc(-c2ccccc2)cc1 ; Clc1nc(-c2ccccc2)nc(-c2ccc(-c3ccccc3)cc2)n1]

After dissolving Sub 3-7a (50.0 g, 184.8 mmol) in THF (924 ml), Sub 3-7b (36.6 g, 184.8 mmol), Pd(PPh₃)₄ (12.8 g, 11.1 mmol), NaOH (22.2 g, 554.5 mmol) and water (462 ml) are added thereto and the reaction is carried out at 80° C. When the reaction is completed, the reaction product is extracted with CH₂Cl₂ and water, and then the organic layer is dried with MgSO₄ and concentrated. Then, the concentrate is separated through a silica gel column and recrystallized to obtain 51.7 g (yield: 81.4%) of the product.

### 2. Synthesis Example of Sub 3-27

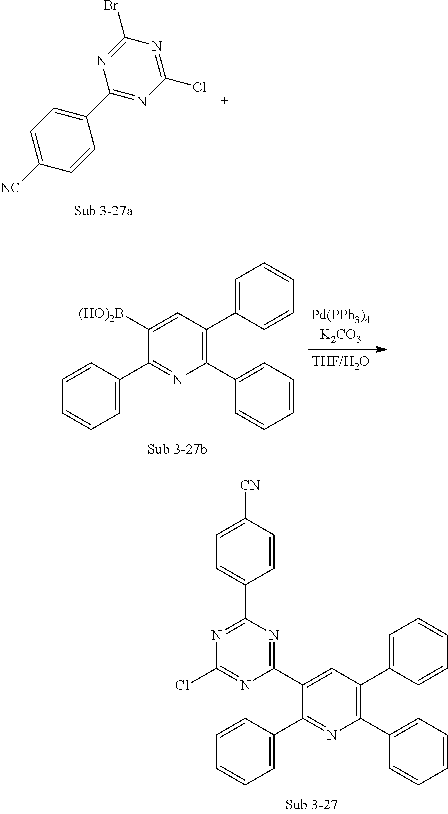 [SMILES: N#Cc1ccc(-c2nc(Cl)nc(Br)n2)cc1 ; OB(O)c1cc(-c2ccccc2)c(-c2ccccc2)nc1-c1ccccc1 ; N#Cc1ccc(-c2nc(Cl)nc(-c3cc(-c4ccccc4)c(-c4ccccc4)nc3-c3ccccc3)n2)cc1]

After dissolving Sub 3-27a (50.0 g, 169.2 mmol) in THF (846 ml), Sub 3-27b (59.4 g, 169.2 mmol), Pd(PPh₃)₄ (11.7 g, 10.2 mmol), NaOH (20.3 g, 507.6 mmol) and water (423 ml) are thereto, and the synthesis is carried out in the same manner as the synthesis of Sub 3-7 to obtain 71.1 g (yield: 80.5%) of product.

Compounds belonging to Sub 3 may be, but not limited to, the following compounds, and Table 4 shows FD-MS (Field Desorption-Mass Spectrometry) values of the following compounds.

TABLE 4

| Compound | FD-MS |
|---|---|
| Sub 3-1 | m/z = 342.09(C₂₂H₁₅ClN₂ = 342.83) |
| Sub 3-2 | m/z = 266.06(C₁₆H₁₁ClN₂ = 266.73) |
| Sub 3-3 | m/z = 342.09(C₂₂H₁₅ClN₂ = 342.83) |
| Sub 3-4 | m/z = 342.09(C₂₂H₁₅ClN₂ = 342.83) |
| Sub 3-5 | m/z = 597.17(C₃₉H₂₄ClN₅ = 598.11) |
| Sub 3-6 | m/z = 368.08(C₂₂H₁₃ClN₄ = 368.82) |
| Sub 3-7 | m/z = 343.09(C₂₁H₁₄ClN₃ = 343.81) |
| Sub 3-8 | m/z = 267.06(C₁₅H₁₀ClN₃ = 267.72) |
| Sub 3-9 | m/z = 558.16(C₃₇H₂₃ClN₄ = 559.07) |
| Sub 3-10 | m/z = 292.05(C₁₆H₉ClN₄ = 292.73) |
| Sub 3-11 | m/z = 344.08(C₂₀H₁₃ClN₄ = 344.80) |
| Sub 3-12 | m/z = 470.13(C₃₀H₁₉ClN₄ = 470.96) |
| Sub 3-13 | m/z = 396.11(C₂₄H₁₇ClN₄ = 396.88) |
| Sub 3-14 | m/z = 518.13(C₃₄H₁₉ClN₄ = 519.00) |
| Sub 3-15 | m/z = 369.08(C₂₁H₁₂ClN₅ = 369.81) |
| Sub 3-16 | m/z = 293.05(C₁₅H₈ClN₅ = 293.71) |
| Sub 3-17 | m/z = 368.08(C₂₂H₁₃ClN₄ = 368.82) |
| Sub 3-18 | m/z = 393.10(C₂₅H₁₆ClN₃ = 393.87) |
| Sub 3-19 | m/z = 432.11(C₂₇H₁₇ClN₄ = 432.91) |
| Sub 3-20 | m/z = 383.12(C₂₄H₁₈ClN₃ = 383.88) |
| Sub 3-21 | m/z = 407.08(C₂₅H₁₄ClN₃O = 407.86) |
| Sub 3-22 | m/z = 418.10(C₂₆H₁₅ClN₄ = 418.88) |
| Sub 3-23 | m/z = 367.09(C₂₃H₁₄ClN₃ = 367.84) |
| Sub 3-24 | m/z = 467.12(C₃₁H₁₈ClN₃ = 467.96) |
| Sub 3-25 | m/z = 268.05(C₁₄H₉ClN₄ = 268.70) |
| Sub 3-26 | m/z = 368.08(C₂₂H₁₃ClN₄ = 368.82) |
| Sub 3-27 | m/z = 521.14(C₃₃H₂₀ClN₅ = 522.01) |
| Sub 3-28 | m/z = 444.11(C₂₈H₁₇ClN₄ = 444.92) |
| Sub 3-29 | m/z = 348.11(C₂₀H₁₇ClN₄ = 348.83) |
| Sub 3-30 | m/z = 393.10(C₂₅H₁₆ClN₃ = 393.87) |
| Sub 3-31 | m/z = 583.18(C₄₀H₂₆ClN₃ = 584.12) |
| Sub 3-32 | m/z = 419.12(C₂₇H₁₈ClN₃ = 419.91) |
| Sub 3-33 | m/z = 367.09(C₂₃H₁₄ClN₃ = 367.84) |
| Sub 3-34 | m/z = 495.15(C₃₃H₂₂ClN₃ = 496.01) |
| Sub 3-35 | m/z = 570.16(C₃₈H₂₃ClN₄ = 571.08) |

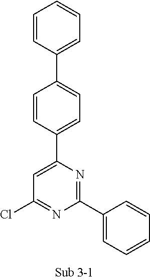 [SMILES: Clc1cc(-c2ccc(-c3ccccc3)cc2)nc(-c2ccccc2)n1]

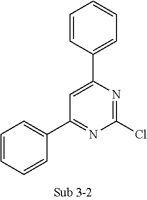 [SMILES: Clc1nc(-c2ccccc2)cc(-c2ccccc2)n1]

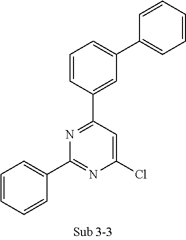 [SMILES: Clc1cc(-c2cccc(-c3ccccc3)c2)nc(-c2ccccc2)n1]

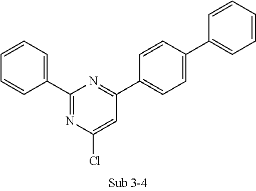 [SMILES: Clc1cc(-c2ccc(-c3ccccc3)cc2)nc(-c2ccccc2)n1]

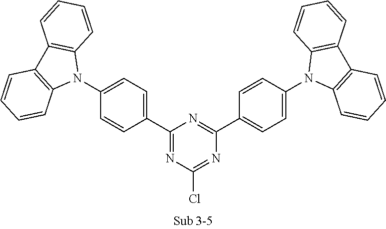 [SMILES: Clc1nc(-c2ccc(-n3c4ccccc4c4ccccc43)cc2)nc(-c2ccc(-n3c4ccccc4c4ccccc43)cc2)n1]

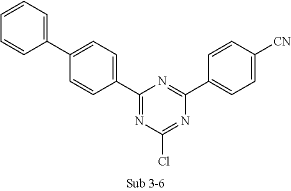 [SMILES: N#Cc1ccc(-c2nc(Cl)nc(-c3ccc(-c4ccccc4)cc3)n2)cc1]

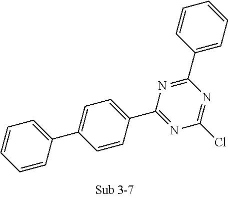 [SMILES: Clc1nc(-c2ccccc2)nc(-c2ccc(-c3ccccc3)cc2)n1]

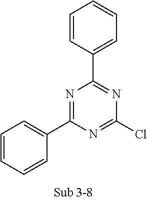 [SMILES: Clc1nc(-c2ccccc2)nc(-c2ccccc2)n1]

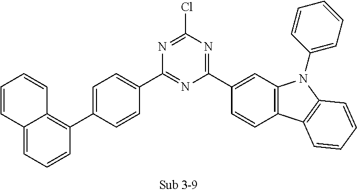 [SMILES: Clc1nc(-c2ccc(-c3cccc4ccccc34)cc2)nc(-c2ccc3c4ccccc4n(-c4ccccc4)c3c2)n1]

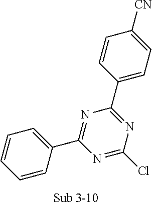 [SMILES: N#Cc1ccc(-c2nc(Cl)nc(-c3ccccc3)n2)cc1]

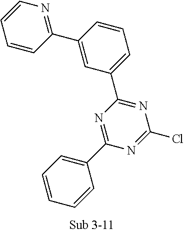 [SMILES: Clc1nc(-c2ccccc2)nc(-c2cccc(-c3ccccn3)c2)n1]

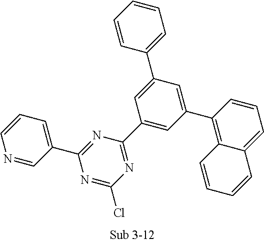 [SMILES: Clc1nc(-c2cccnc2)nc(-c2cc(-c3ccccc3)cc(-c3cccc4ccccc34)c2)n1]

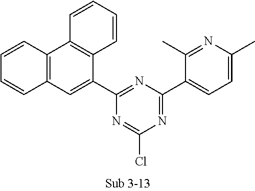 [SMILES: Cc1ccc(-c2nc(Cl)nc(-c3cc4ccccc4c4ccccc34)n2)c(C)n1]

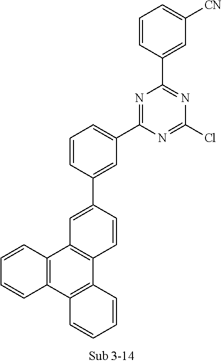 [SMILES: N#Cc1cccc(-c2nc(Cl)nc(-c3cccc(-c4ccc5c6ccccc6c6ccccc6c5c4)c3)n2)c1]

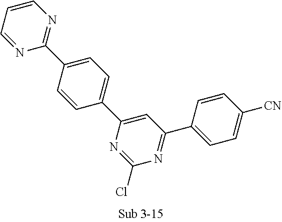 [SMILES: N#Cc1ccc(-c2cc(-c3ccc(-c4ncccn4)cc3)nc(Cl)n2)cc1]

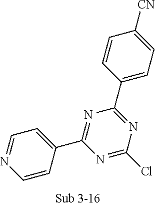 [SMILES: N#Cc1ccc(-c2nc(Cl)nc(-c3ccncc3)n2)cc1]

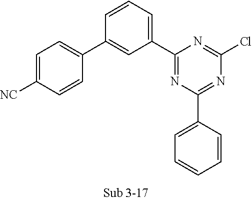 [SMILES: N#Cc1ccc(-c2cccc(-c3nc(Cl)nc(-c4ccccc4)n3)c2)cc1]

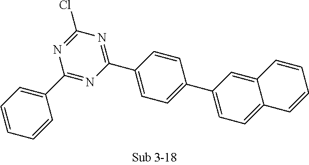 [SMILES: Clc1nc(-c2ccccc2)nc(-c2ccc(-c3ccc4ccccc4c3)cc2)n1]

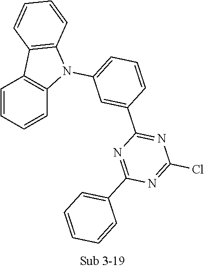 [SMILES: Clc1nc(-c2ccccc2)nc(-c2cccc(-n3c4ccccc4c4ccccc43)c2)n1]

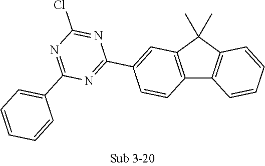 [SMILES: CC1(C)c2ccccc2-c2ccc(-c3nc(Cl)nc(-c4ccccc4)n3)cc21]

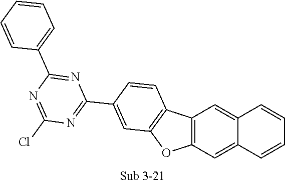 [SMILES: Clc1nc(-c2ccccc2)nc(-c2ccc3c(c2)oc2cc4ccccc4cc23)n1]

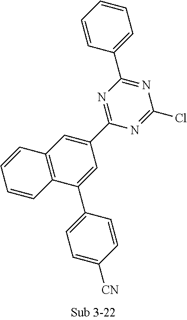 [SMILES: N#Cc1ccc(-c2cc(-c3nc(Cl)nc(-c4ccccc4)n3)cc3ccccc23)cc1]

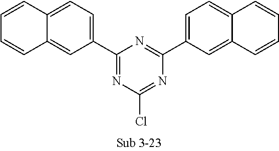 [SMILES: Clc1nc(-c2ccc3ccccc3c2)nc(-c2ccc3ccccc3c2)n1]

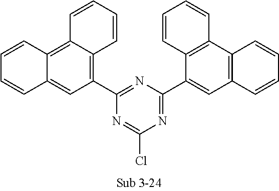 [SMILES: Clc1nc(-c2cc3ccccc3c3ccccc23)nc(-c2cc3ccccc3c3ccccc23)n1]

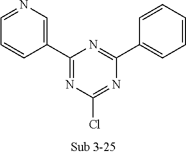 [SMILES: Clc1nc(-c2ccccc2)nc(-c2cccnc2)n1]

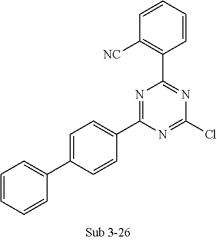 [SMILES: N#Cc1ccccc1-c1nc(Cl)nc(-c2ccc(-c3ccccc3)cc2)n1]

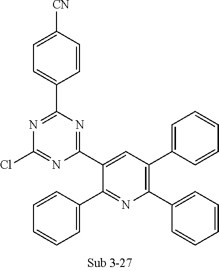 [SMILES: N#Cc1ccc(-c2nc(Cl)nc(-c3cc(-c4ccccc4)c(-c4ccccc4)nc3-c3ccccc3)n2)cc1]

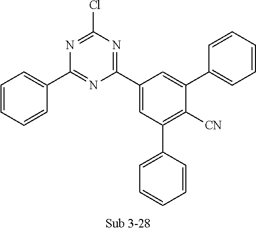 [SMILES: N#Cc1c(-c2ccccc2)cc(-c2nc(Cl)nc(-c3ccccc3)n2)cc1-c1ccccc1]

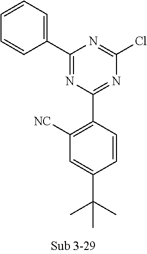 [SMILES: CC(C)(C)c1ccc(-c2nc(Cl)nc(-c3ccccc3)n2)c(C#N)c1]

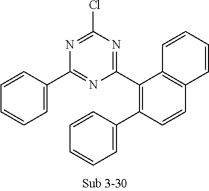 [SMILES: Clc1nc(-c2ccccc2)nc(-c2c(-c3ccccc3)ccc3ccccc23)n1]

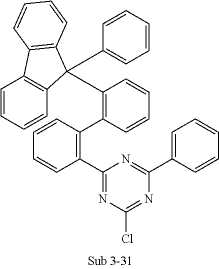 [SMILES: Clc1nc(-c2ccccc2)nc(-c2ccccc2-c2ccccc2C2(c3ccccc3)c3ccccc3-c3ccccc32)n1]

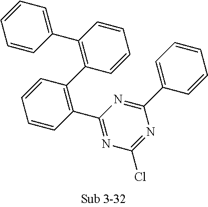 [SMILES: Clc1nc(-c2ccccc2)nc(-c2ccccc2-c2ccccc2-c2ccccc2)n1]

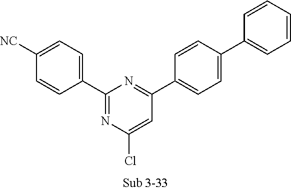 [SMILES: N#Cc1ccc(-c2nc(Cl)cc(-c3ccc(-c4ccccc4)cc3)n2)cc1]

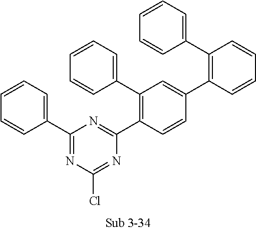 [SMILES: Clc1nc(-c2ccccc2)nc(-c2ccc(-c3ccccc3-c3ccccc3)cc2-c2ccccc2)n1]

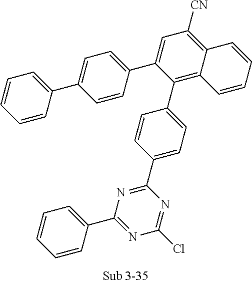 [SMILES: N#Cc1cc(-c2ccc(-c3ccccc3)cc2)c(-c2ccc(-c3nc(Cl)nc(-c4ccccc4)n3)cc2)c2ccccc12]

### Synthesis Example of Sub 4

### Synthesis Example of Sub 4-26

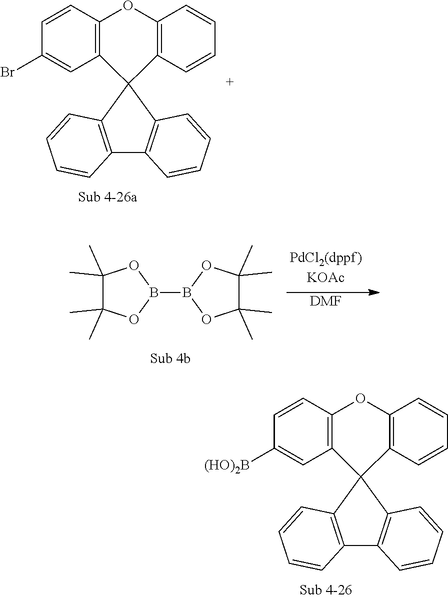

After adding Sub 4b (34.0 g, 133.7 mmol), PdCl₂(dppf) (2.7 g, 3.7 mmol), KOAc (35.8 g, 364.7 mmol) and DMF (608 ml) to Sub 4-26a (50.0 g, 121.6 mmol), the mixture is stirred and refluxed at 120° C. When the reaction is completed, the temperature of the reactant is cooled to room temperature, extracted with MC (methylene chloride), and washed with water. The organic layer was dried over MgSO₄ and concentrated, and the concentrate was separated with a silica gel column to obtain 32.0 g (yield: 69.9%) of product.

Compounds belonging to Sub 4 may be, but not limited to, the following compounds, and Table 5 shows FD-MS (Field Desorption-Mass Spectrometry) values of the following compounds.

TABLE 5

| Compound | FD-MS |
|---|---|
| Sub 4-1 | m/z = 452.17(C₃₀H₂₁BN₂O₂ = 452.32) |
| Sub 4-2 | m/z = 287.11(C₁₈H₁₄BNO₂ = 287.13) |
| Sub 4-3 | m/z = 449.16(C₃₁H₂₀BNO₂ = 449.32) |
| Sub 4-4 | m/z = 285.10(C₁₈H₁₂BNO₂ = 285.11) |
| Sub 4-5 | m/z = 413.16(C₂₈H₂₀BNO₂ = 413.28) |
| Sub 4-6 | m/z = 337.13(C₂₂H₁₆BNO₂ = 337.19) |
| Sub 4-7 | m/z = 554.22(C₃₈H₂₇BN₂O₂ = 554.46) |
| Sub 4-8 | m/z = 528.20(C₃₆H₂₅BN₂O₂ = 528.42) |
| Sub 4-9 | m/z = 362.15(C₂₅H₁₉BO₂ = 362.24) |
| Sub 4-10 | m/z = 362.15(C₂₅H₁₉BO₂ = 362.24) |
| Sub 4-11 | m/z = 360.13(C₂₅H₁₇BO₂ = 360.22) |
| Sub 4-12 | m/z = 362.15(C₂₅H₁₉BO₂ = 362.24) |
| Sub 4-13 | m/z = 389.16(C₂₆H₂₀BNO₂ = 389.26) |
| Sub 4-14 | m/z = 390.18(C₂₇H₂₃BO₂ = 390.29) |
| Sub 4-15 | m/z = 488.19(C₃₅H₂₅BO₂ = 488.39) |
| Sub 4-16 | m/z = 539.21(C₃₈H₂₆BNO₂ = 539.44) |
| Sub 4-17 | m/z = 364.13(C₂₄H₁₇BO₃ = 364.21) |
| Sub 4-18 | m/z = 228.04(C₁₂H₉BO₂S = 228.07) |
| Sub 4-19 | m/z = 212.06(C₁₂H₉BO₃ = 212.01) |
| Sub 4-20 | m/z = 212.06(C₁₂H₉BO₃ = 212.01) |
| Sub 4-21 | m/z = 465.15(C₃₁H₂₀BNO₃ = 465.32) |
| Sub 4-22 | m/z = 304.07(C₁₈H₁₃BO₂S = 304.17) |
| Sub 4-23 | m/z = 304.07(C₁₈H₁₃BO₂S = 304.17) |
| Sub 4-24 | m/z = 414.14(C₂₈H₁₉BO₃ = 414.27) |
| Sub 4-25 | m/z = 392.10(C₂₅H₁₇BO₂S = 392.28) |
| Sub 4-26 | m/z = 376.13(C₂₅H₁₇BO₃ = 376.22) |
| Sub 4-27 | m/z = 477.15(C₃₂H₂₀BNO₃ = 477.33) |
| Sub 4-28 | m/z = 376.13(C₂₅H₁₇BO₃ = 376.22) |
| Sub 4-29 | m/z = 452.16(C₃₁H₂₁BO₃ = 452.32) |
| Sub 4-30 | m/z = 468.14(C₃₁H₂₁BO₂S = 468.38) |
| Sub 4-31 | m/z = 392.10(C₂₅H₁₇BO₂S = 392.28) |
| Sub 4-32 | m/z = 417.10(C₂₆H₁₆BNO₂S = 417.29) |
| Sub 4-33 | m/z = 376.13(C₂₅H₁₇BO₃ = 376.22) |
| Sub 4-34 | m/z = 417.10(C₂₆H₁₆BNO₂S = 417.29) |
| Sub 4-35 | m/z = 452.16(C₃₁H₂₁BO₃ = 452.32) |
| Sub 4-36 | m/z = 392.10(C₂₅H₁₇BO₂S = 392.28) |
| Sub 4-37 | m/z = 375.14(C₂₅H₁₈BNO₂ = 375.23) |
| Sub 4-38 | m/z = 403.17(C₂₇H₂₂BNO₂ = 403.29) |
| Sub 4-39 | m/z = 350.12(C₂₂H₁₅BN₂O₂ = 350.18) |
| Sub 4-40 | m/z = 425.16(C₂₉H₂₀BNO₂ = 425.29) |
| Sub 4-41 | m/z = 425.16(C₂₉H₂₀BNO₂ = 425.29) |
| Sub 4-42 | m/z = 400.14(C₂₆H₁₇BN₂O₂ = 400.24) |
| Sub 4-43 | m/z = 276.11(C₁₆H₁₃BN₂O₂ = 276.10) |
| Sub 4-44 | m/z = 249.10(C₁₅H₁₂BNO₂ = 249.08) |
| Sub 4-45 | m/z = 199.08(C₁₁H₁₀BNO₂ = 199.02) |
| Sub 4-46 | m/z = 368.14(C₂₂H₁₈B₂O₄ = 368.00) |
| Sub 4-47 | m/z = 418.15(C₂₆H₂₀B₂O₄ = 418.06) |
| Sub 4-48 | m/z = 368.14(C₂₂H₁₈B₂O₄ = 368.00) |
| Sub 4-49 | m/z = 216.08(C₁₀H₁₀B₂O₄ = 215.81) |
| Sub 4-50 | m/z = 418.15(C₂₆H₂₀B₂O₄ = 418.06) |
| Sub 4-51 | m/z = 368.14(C₂₂H₁₈B₂O₄ = 368.00) |
| Sub 4-52 | m/z = 406.15(C₂₅H₂₀B₂O₄ = 406.05) |
| Sub 4-53 | m/z = 399.14(C₂₇H₁₈BNO₂ = 399.26) |
| Sub 4-54 | m/z = 474.18(C₃₄H₂₃BO₂ = 474.37) |
| Sub 4-55 | m/z = 450.18(C₃₂H₂₃BO₂ = 450.34) |
| Sub 4-56 | m/z = 439.17(C₃₀H₂₂BNO₂ = 439.32) |
| Sub 4-57 | m/z = 324.13(C₂₂H₁₇BO₂ = 324.19) |
| Sub 4-58 | m/z = 349.13(C₂₃H₁₆BNO₂ = 349.20) |
| Sub 4-59 | m/z = 349.13(C₂₃H₁₆BNO₂ = 349.20) |
| Sub 4-60 | m/z = 528.19(C₃₇H₂₅BO₃ = 528.41) |
| Sub 4-61 | m/z = 502.17(C₃₅H₂₃BO₃ = 502.38) |
| Sub 4-62 | m/z = 477.15(C₃₂H₂₀BNO₃ = 477.33) |
| Sub 4-63 | m/z = 288.11(C₁₇H₁₃BN₂O₂ = 288.11) |
| Sub 4-64 | m/z = 289.10(C₁₆H₁₂BN₃O₂ = 289.10) |
| Sub 4-65 | m/z = 515.21(C₃₆H₂₆BNO₂ = 515.42) |
| Sub 4-66 | m/z = 515.21(C₃₆H₂₆BNO₂ = 515.42) |
| Sub 4-67 | m/z = 412.14(C₂₇H₁₇BN₂O₂ = 412.26) |
| Sub 4-68 | m/z = 461.16(C₃₂H₂₀BNO₂ = 461.33) |
| Sub 4-69 | m/z = 364.14(C₂₃H₁₇BN₂O₂ = 364.21) |
| Sub 4-70 | m/z = 436.16(C₃₁H₂₁BO₂ = 436.32) |

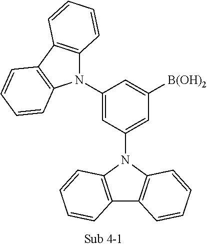 [SMILES: OB(O)c1cc(-n2c3ccccc3c3ccccc32)cc(-n2c3ccccc3c3ccccc32)c1]

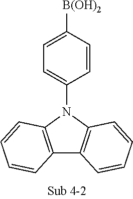 [SMILES: OB(O)c1ccc(-n2c3ccccc3c3ccccc32)cc1]

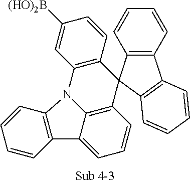 [SMILES: OB(O)c1ccc2c(c1)-n1c3ccccc3c3cccc(c31)C21c2ccccc2-c2ccccc21]

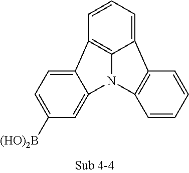 [SMILES: OB(O)c1ccc2c3cccc4c5ccccc5n(c2c1)c43]

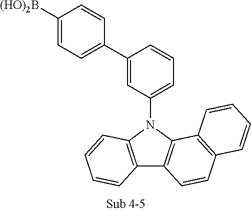 [SMILES: OB(O)c1ccc(-c2cccc(-n3c4ccccc4c4ccc5ccccc5c43)c2)cc1]

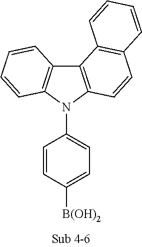 [SMILES: OB(O)c1ccc(-n2c3ccccc3c3c4ccccc4ccc32)cc1]

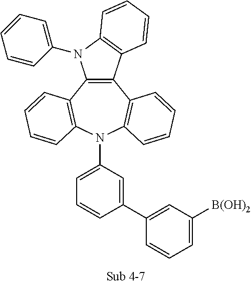 [SMILES: OB(O)c1cccc(-c2cccc(N3c4ccccc4-c4c(n(-c5ccccc5)c5ccccc45)-c4ccccc43)c2)c1]

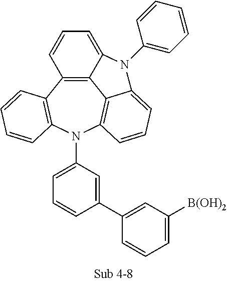 [SMILES: OB(O)c1cccc(-c2cccc(-n3c4ccccc4c4cccc5c4c4c3cccc4n5-c3ccccc3)c2)c1]

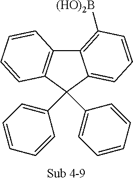 [SMILES: OB(O)c1cccc2c1-c1ccccc1C2(c1ccccc1)c1ccccc1]

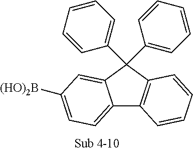 [SMILES: OB(O)c1ccc2c(c1)C(c1ccccc1)(c1ccccc1)c1ccccc1-2]

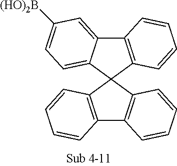 [SMILES: OB(O)c1ccc2c(c1)-c1ccccc1C21c2ccccc2-c2ccccc21]

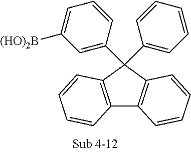 [SMILES: OB(O)c1cccc(C2(c3ccccc3)c3ccccc3-c3ccccc32)c1]

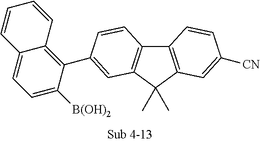 [SMILES: CC1(C)c2cc(C#N)ccc2-c2ccc(-c3c(B(O)O)ccc4ccccc34)cc21]

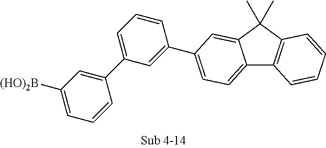 [SMILES: CC1(C)c2ccccc2-c2ccc(-c3cccc(-c4cccc(B(O)O)c4)c3)cc21]

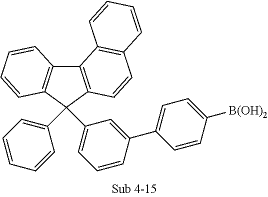 [SMILES: OB(O)c1ccc(-c2cccc(C3(c4ccccc4)c4ccccc4-c4c3ccc3ccccc43)c2)cc1]

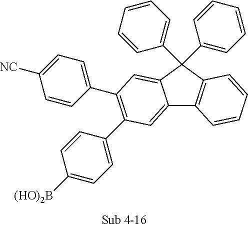 [SMILES: N#Cc1ccc(-c2cc3c(cc2-c2ccc(B(O)O)cc2)-c2ccccc2C3(c2ccccc2)c2ccccc2)cc1]

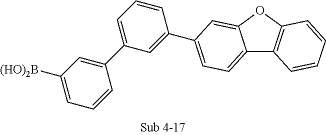 [SMILES: OB(O)c1cccc(-c2cccc(-c3ccc4c(c3)oc3ccccc34)c2)c1]

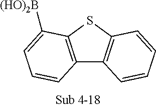 [SMILES: OB(O)c1cccc2c1sc1ccccc12]

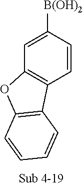 [SMILES: OB(O)c1ccc2c(c1)oc1ccccc12]

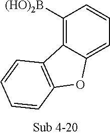 [SMILES: OB(O)c1cccc2oc3ccccc3c12]

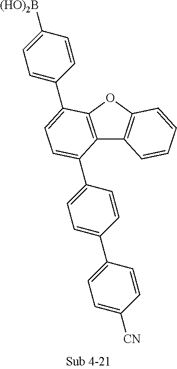 [SMILES: N#Cc1ccc(-c2ccc(-c3ccc(-c4ccc(B(O)O)cc4)c4oc5ccccc5c34)cc2)cc1]

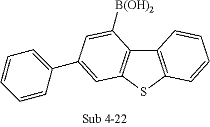 [SMILES: OB(O)c1cc(-c2ccccc2)cc2sc3ccccc3c12]

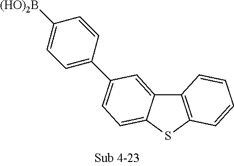 [SMILES: OB(O)c1ccc(-c2ccc3sc4ccccc4c3c2)cc1]

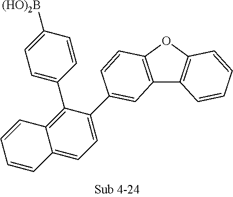 [SMILES: OB(O)c1ccc(-c2c(-c3ccc4oc5ccccc5c4c3)ccc3ccccc23)cc1]

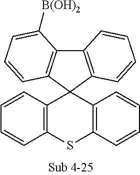 [SMILES: OB(O)c1cccc2c1-c1ccccc1C21c2ccccc2Sc2ccccc21]

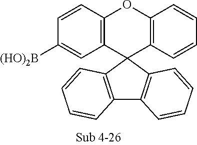 [SMILES: OB(O)c1ccc2c(c1)C1(c3ccccc3O2)c2ccccc2-c2ccccc21]

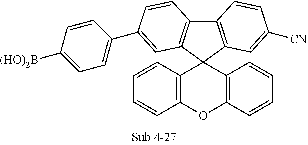 [SMILES: N#Cc1ccc2c(c1)C1(c3ccccc3Oc3ccccc31)c1cc(-c3ccc(B(O)O)cc3)ccc1-2]

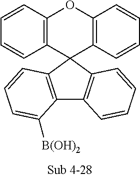 [SMILES: OB(O)c1cccc2c1-c1ccccc1C21c2ccccc2Oc2ccccc21]

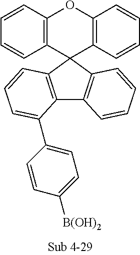 [SMILES: OB(O)c1ccc(-c2cccc3c2-c2ccccc2C32c3ccccc3Oc3ccccc32)cc1]

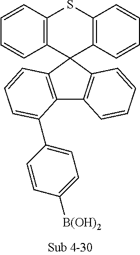 [SMILES: OB(O)c1ccc(-c2cccc3c2-c2ccccc2C32c3ccccc3Sc3ccccc32)cc1]

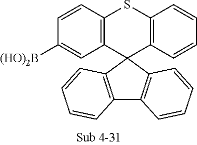 [SMILES: OB(O)c1ccc2c(c1)C1(c3ccccc3S2)c2ccccc2-c2ccccc21]

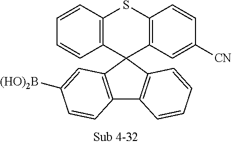 [SMILES: N#Cc1ccc2c(c1)C1(c3ccccc3S2)c2ccccc2-c2ccc(B(O)O)cc21]

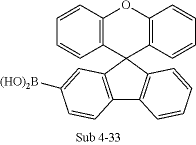 [SMILES: OB(O)c1ccc2c(c1)C1(c3ccccc3Oc3ccccc31)c1ccccc1-2]

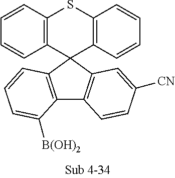 [SMILES: N#Cc1ccc2c(c1)C1(c3ccccc3Sc3ccccc31)c1cccc(B(O)O)c1-2]

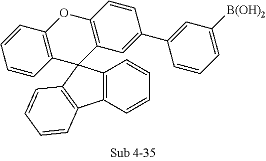 [SMILES: OB(O)c1cccc(-c2ccc3c(c2)C2(c4ccccc4O3)c3ccccc3-c3ccccc32)c1]

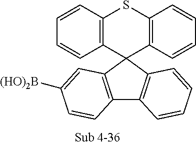 [SMILES: OB(O)c1ccc2c(c1)C1(c3ccccc3Sc3ccccc31)c1ccccc1-2]

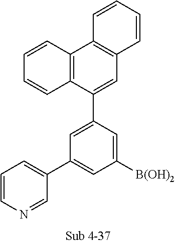 [SMILES: OB(O)c1cc(-c2cccnc2)cc(-c2cc3ccccc3c3ccccc23)c1]

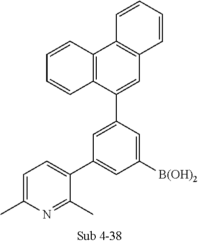 [SMILES: Cc1ccc(-c2cc(B(O)O)cc(-c3cc4ccccc4c4ccccc34)c2)c(C)n1]

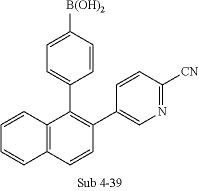 [SMILES: N#Cc1ccc(-c2ccc3ccccc3c2-c2ccc(B(O)O)cc2)cn1]

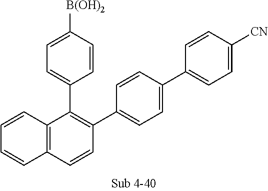 [SMILES: N#Cc1ccc(-c2ccc(-c3ccc4ccccc4c3-c3ccc(B(O)O)cc3)cc2)cc1]

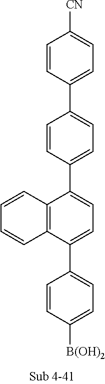 [SMILES: N#Cc1ccc(-c2ccc(-c3ccc(-c4ccc(B(O)O)cc4)c4ccccc34)cc2)cc1]

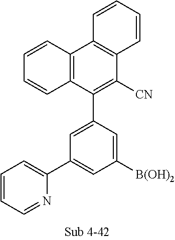 [SMILES: N#Cc1c(-c2cc(B(O)O)cc(-c3ccccn3)c2)c2ccccc2c2ccccc12]

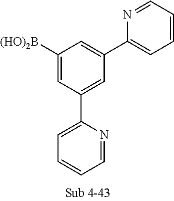 [SMILES: OB(O)c1cc(-c2ccccn2)cc(-c2ccccn2)c1]

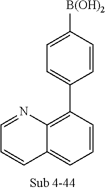 [SMILES: OB(O)c1ccc(-c2cccc3cccnc23)cc1]

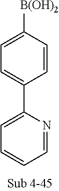 [SMILES: OB(O)c1ccc(-c2ccccn2)cc1]

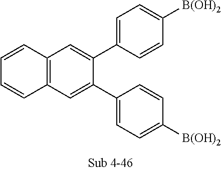 [SMILES: OB(O)c1ccc(-c2cc3ccccc3cc2-c2ccc(B(O)O)cc2)cc1]

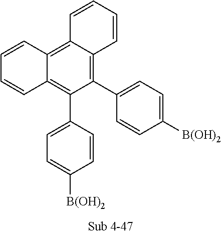 [SMILES: OB(O)c1ccc(-c2c(-c3ccc(B(O)O)cc3)c3ccccc3c3ccccc23)cc1]

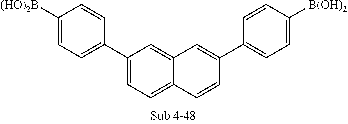 [SMILES: OB(O)c1ccc(-c2ccc3ccc(-c4ccc(B(O)O)cc4)cc3c2)cc1]

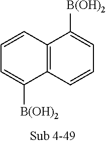 [SMILES: OB(O)c1cccc2c(B(O)O)cccc12]

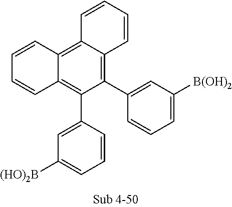 [SMILES: OB(O)c1cccc(-c2c(-c3cccc(B(O)O)c3)c3ccccc3c3ccccc23)c1]

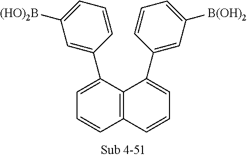 [SMILES: OB(O)c1cccc(-c2cccc3cccc(-c4cccc(B(O)O)c4)c23)c1]

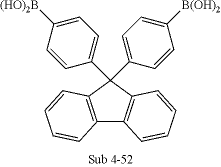 [SMILES: OB(O)c1ccc(C2(c3ccc(B(O)O)cc3)c3ccccc3-c3ccccc32)cc1]

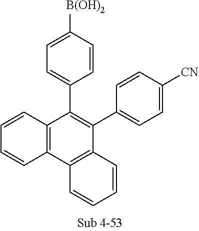 [SMILES: N#Cc1ccc(-c2c(-c3ccc(B(O)O)cc3)c3ccccc3c3ccccc23)cc1]

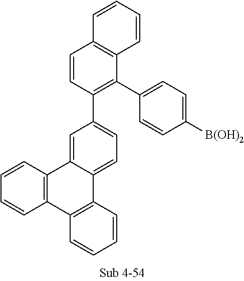 [SMILES: OB(O)c1ccc(-c2c(-c3ccc4c5ccccc5c5ccccc5c4c3)ccc3ccccc23)cc1]

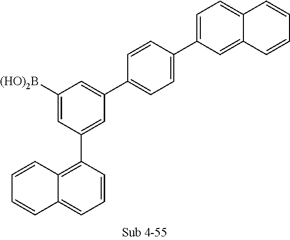 [SMILES: OB(O)c1cc(-c2ccc(-c3ccc4ccccc4c3)cc2)cc(-c2cccc3ccccc23)c1]

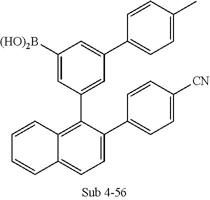 [SMILES: Cc1ccc(-c2cc(B(O)O)cc(-c3c(-c4ccc(C#N)cc4)ccc4ccccc34)c2)cc1]

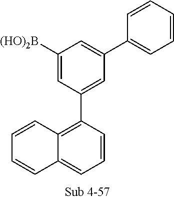 [SMILES: OB(O)c1cc(-c2ccccc2)cc(-c2cccc3ccccc23)c1]

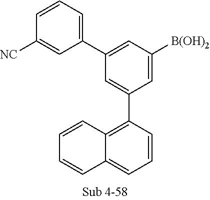 [SMILES: N#Cc1cccc(-c2cc(B(O)O)cc(-c3cccc4ccccc34)c2)c1]

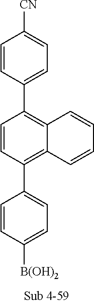 [SMILES: N#Cc1ccc(-c2ccc(-c3ccc(B(O)O)cc3)c3ccccc23)cc1]

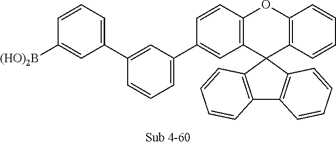 [SMILES: OB(O)c1cccc(-c2cccc(-c3ccc4c(c3)C3(c5ccccc5O4)c4ccccc4-c4ccccc43)c2)c1]

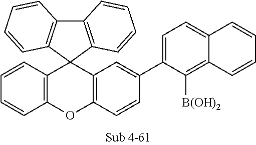 [SMILES: OB(O)c1c(-c2ccc3c(c2)C2(c4ccccc4O3)c3ccccc3-c3ccccc32)ccc2ccccc12]

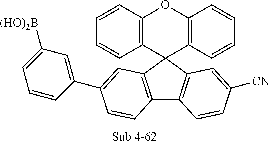 [SMILES: N#Cc1ccc2c(c1)C1(c3ccccc3Oc3ccccc31)c1cc(-c3cccc(B(O)O)c3)ccc1-2]

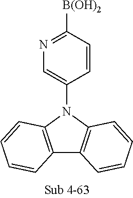 [SMILES: OB(O)c1ccc(-n2c3ccccc3c3ccccc32)cn1]

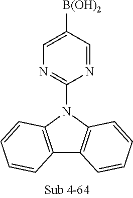 [SMILES: OB(O)c1cnc(-n2c3ccccc3c3ccccc32)nc1]

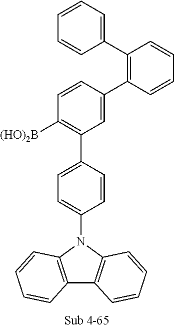 [SMILES: OB(O)c1ccc(-c2ccccc2-c2ccccc2)cc1-c1ccc(-n2c3ccccc3c3ccccc32)cc1]

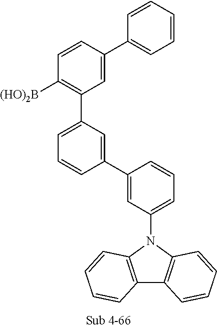 [SMILES: OB(O)c1ccc(-c2ccccc2)cc1-c1cccc(-c2cccc(-n3c4ccccc4c4ccccc43)c2)c1]

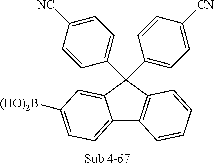 [SMILES: N#Cc1ccc(C2(c3ccc(C#N)cc3)c3ccccc3-c3ccc(B(O)O)cc32)cc1]

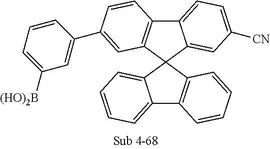 [SMILES: N#Cc1ccc2c(c1)C1(c3ccccc3-c3ccccc31)c1cc(-c3cccc(B(O)O)c3)ccc1-2]

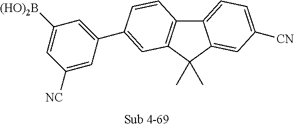 [SMILES: CC1(C)c2cc(C#N)ccc2-c2ccc(-c3cc(C#N)cc(B(O)O)c3)cc21]

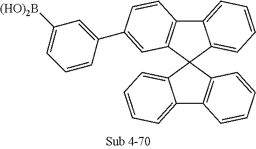 [SMILES: OB(O)c1cccc(-c2ccc3c(c2)C2(c4ccccc4-c4ccccc42)c2ccccc2-3)c1]

### Synthesis Example of the Final Compound

### 1. Synthesis Example of E-1

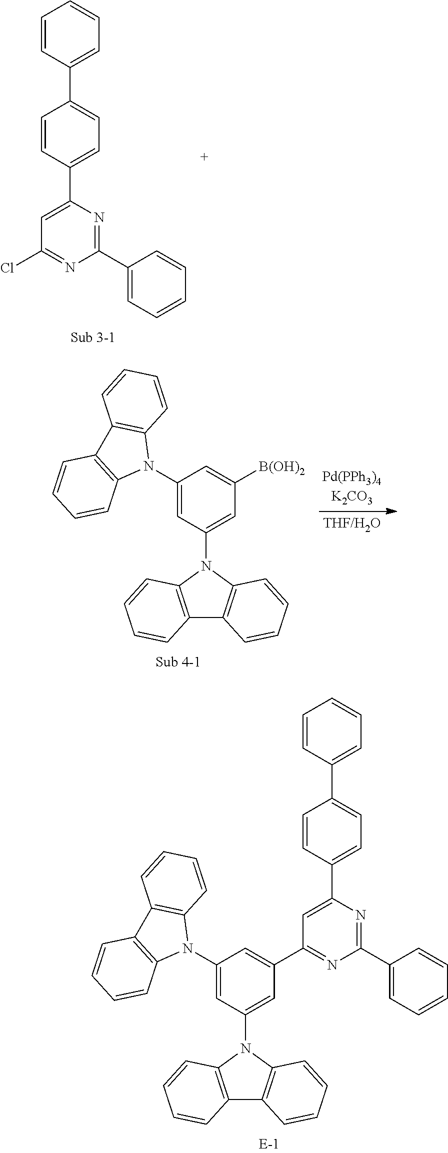 [SMILES: Clc1cc(-c2ccc(-c3ccccc3)cc2)nc(-c2ccccc2)n1 ; OB(O)c1cc(-n2c3ccccc3c3ccccc32)cc(-n2c3ccccc3c3ccccc32)c1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

After dissolving Sub 3-1(10.0 g, 29.2 mmol) in THF (146 ml), Sub 4-1 (13.2 g, 29.2 mmol), Pd(PPh₃)₄ (2.0 g, 1.8 mmol), NaOH (3.5 g, 87.5 mmol) and water (73 ml) are added thereto and the reaction is carried out at 80° C. When the reaction is completed, the reaction product is extracted with CH₂Cl₂ and water, and then the organic layer is dried with MgSO₄ and concentrated. Then, the concentrate is separated through a silica gel column and recrystallized to obtain 17.4 g (yield: 83.4%).

### 2. Synthesis Example of E-14

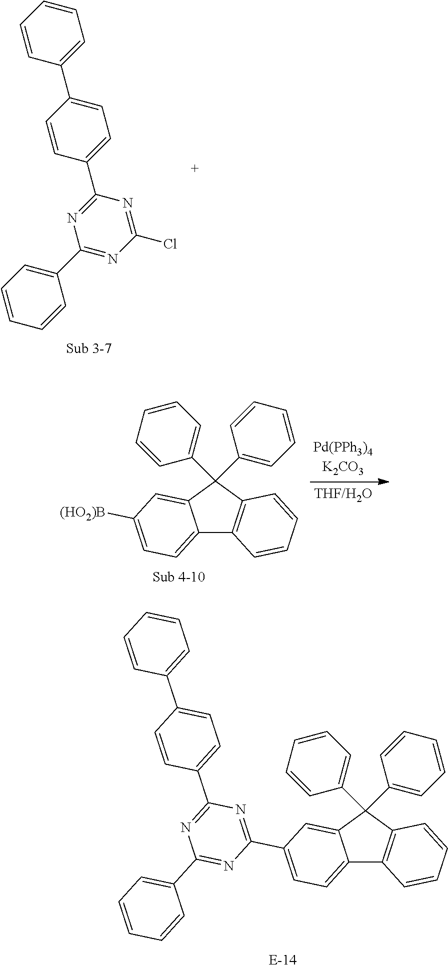

After dissolving Sub 3-7(10.0 g, 29.1 mmol) in THF (145 ml), Sub 4-10 (10.5 g, 29.1 mmol), Pd(PPh₃)₄ (2.0 g, 1.8 mmol), NaOH (3.5 g, 87.3 mmol) and water (73 ml) are thereto, and the synthesis is carried out in the same manner as the synthesis of E-1 to obtain 15.6 g (yield: 85.6%) of product.

### 3. Synthesis Example of E-31

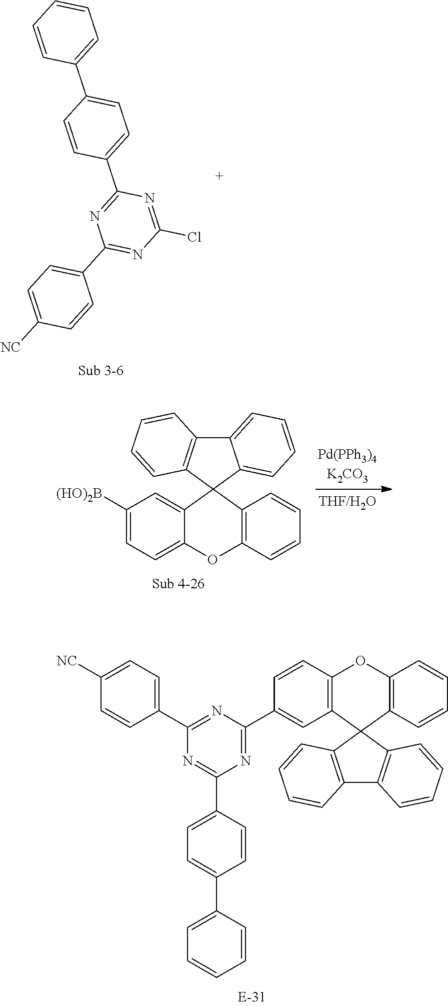 [SMILES: N#Cc1ccc(-c2nc(Cl)nc(-c3ccc(-c4ccccc4)cc3)n2)cc1 ; OB(O)c1ccc2c(c1)C1(c3ccccc3O2)c2ccccc2-c2ccccc21 ; N#Cc1ccc(-c2nc(-c3ccc(-c4ccccc4)cc3)nc(-c3ccc4c(c3)C3(c5ccccc5O4)c4ccccc4-c4ccccc43)n2)cc1]

After dissolving Sub 3-6(10.0 g, 27.1 mmol) in THF (136 ml), Sub 4-26 (10.2 g, 27.1 mmol), Pd(PPh₃)₄ (1.9 g, 1.6 mmol), NaOH (3.3 g, 81.3 mmol) and water (68 ml) are thereto, and the synthesis is carried out in the same manner as the synthesis of E-1 to obtain 14.6 g (yield: 80.9%) of product.

### 4. Synthesis Example of E-54

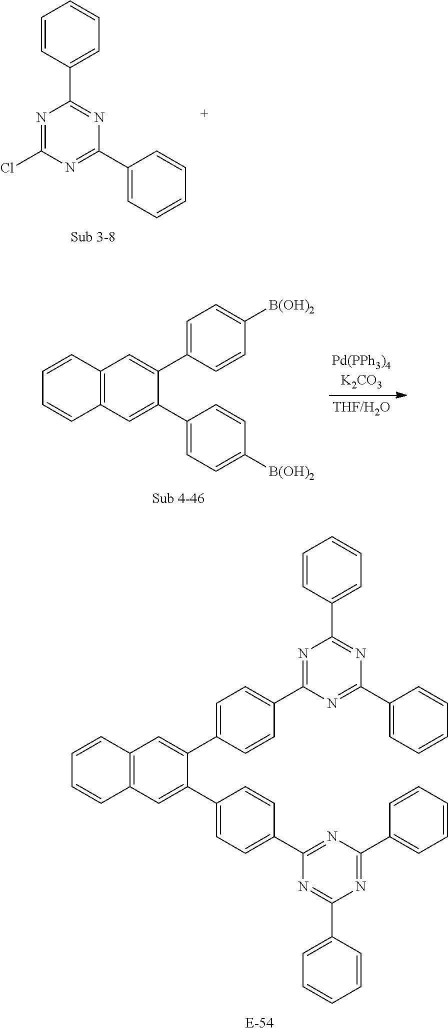 [SMILES: Clc1nc(-c2ccccc2)nc(-c2ccccc2)n1 ; OB(O)c1ccc(-c2cc3ccccc3cc2-c2ccc(B(O)O)cc2)cc1 ; c1ccc(-c2nc(-c3ccccc3)nc(-c3ccc(-c4cc5ccccc5cc4-c4ccc(-c5nc(-c6ccccc6)nc(-c6ccccc6)n5)cc4)cc3)n2)cc1]

After dissolving Sub 3-8(10.0 g, 37.4 mmol) in THF (187 ml), Sub 4-46 (6.9 g, 18.7 mmol), Pd(PPh₃)₄ (2.6 g, 2.2 mmol), NaOH (4.5 g, 112.1 mmol) and water (93 ml) are thereto, and the synthesis is carried out in the same manner as the synthesis of E-1 to obtain 21.0 g (yield: 75.8%) of product.

### 5. Synthesis Example of E-61

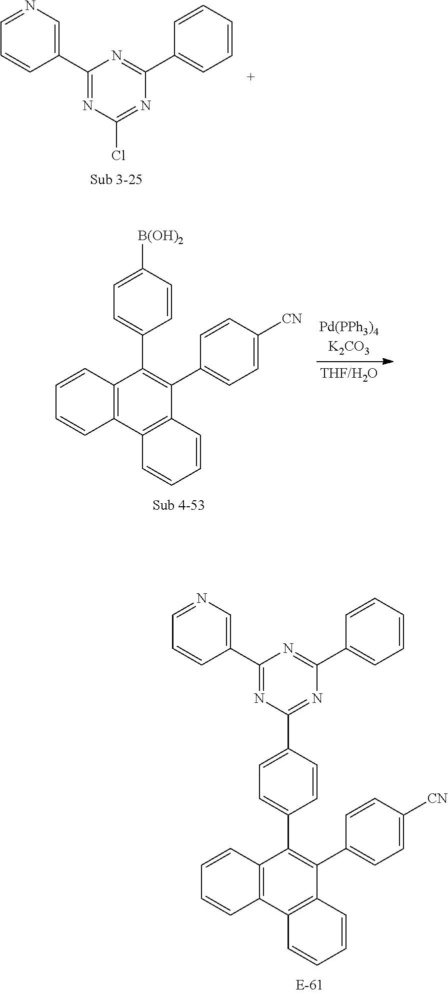 [SMILES: Clc1nc(-c2ccccc2)nc(-c2cccnc2)n1 ; N#Cc1ccc(-c2c(-c3ccc(B(O)O)cc3)c3ccccc3c3ccccc23)cc1 ; N#Cc1ccc(-c2c(-c3ccc(-c4nc(-c5ccccc5)nc(-c5cccnc5)n4)cc3)c3ccccc3c3ccccc23)cc1]

After dissolving Sub 3-25(10.0 g, 37.2 mmol) in THF (186 ml), Sub 4-53(14.9 g, 37.2 mmol), Pd(PPh₃)₄ (2.6 g, 2.2 mmol), NaOH (4.5 g, 111.6 mmol) and water (93 ml) are thereto, and the synthesis is carried out in the same manner as the synthesis of E-1 to obtain 17.7 g (yield: 81.1%) of product.

The FD-MS values of the compounds E-1 to E-80 of the present invention prepared according to the above synthesis examples are shown in Table 6 below.

TABLE 6

| Compound | FD-MS | Compound | FD-MS |
|---|---|---|---|
| E-1 | m/z = 714.28(C₅₂H₃₄N₄ = 714.87) | E-2 | m/z = 638.25(C₄₆H₃₀N₄ = 638.77) |
| E-3 | m/z = 714.28(C₅₂H₃₄N₄ = 714.87) | E-4 | m/z = 549.22(C₄₀H₂₇N₃ = 549.68) |
| E-5 | m/z = 804.30(C₅₇H₃₆N₆ = 804.96) | E-6 | m/z = 575.21(C₄₀H₂₅N₅ = 575.68) |
| E-7 | m/z = 712.26(C₅₂H₃₂N₄ = 712.86) | E-8 | m/z = 548.20(C₃₉H₂₄N₄ = 548.65) |
| E-9 | m/z = 600.23(C₄₃H₂₈N₄ = 600.73) | E-10 | m/z = 815.30(C₅₉H₃₇N₅ = 815.98) |
| E-11 | m/z = 741.29(C₅₃H₃₅N₅ = 741.90) | E-12 | m/z = 715.27(C₅₁H₃₃N₅ = 715.86) |
| E-13 | m/z = 625.25(C₄₆H₃₁N₃ = 625.78) | E-14 | m/z = 625.25(C₄₆H₃₁N₃ = 625.78) |
| E-15 | m/z = 572.20(C₄₁H₂₄N₄ = 572.67) | E-16 | m/z = 549.22(C₄₀H₂₇N₃ = 549.68) |
| E-17 | m/z = 576.23(C₄₁H₂₈N₄ = 576.70) | E-18 | m/z = 577.25(C₄₂H₃₁N₃ = 577.73) |
| E-19 | m/z = 776.29(C₅₇H₃₆N₄ = 776.94) | E-20 | m/z = 726.28(C₅₃H₃₄N₄ = 726.88) |
| E-21 | m/z = 628.23(C₄₄H₂₈N₄O = 628.74) | E-22 | m/z = 618.19(C₄₂H₂₆N₄S = 618.76) |
| E-23 | m/z = 528.20(C₃₆H₂₄N₄O = 528.62) | E-24 | m/z = 650.21(C₄₆H₂₆N₄O = 650.74) |
| E-25 | m/z = 652.23(C₄₆H₂₈N₄O = 652.76) | E-26 | m/z = 593.17(C₃₉H₂₃N₅S = 593.71) |
| E-27 | m/z = 517.14(C₃₃H₁₉N₅S = 517.61) | E-28 | m/z = 601.22(C₄₃H₂₇N₃O = 601.71) |
| E-29 | m/z = 579.18(C₄₀H₂₅N₃S = 579.72) | E-30 | m/z = 563.20(C₄₀H₂₅N₃O = 563.66) |
| E-31 | m/z = 664.23(C₄₇H₂₈N₄O = 664.77) | E-32 | m/z = 664.23(C₄₇H₂₈N₄O = 664.77) |
| E-33 | m/z = 563.20(C₄₀H₂₅N₃O = 563.66) | E-34 | m/z = 664.23(C₄₇H₂₈N₄O = 664.77) |
| E-35 | m/z = 639.23(C₄₆H₂₉N₃O = 639.76) | E-36 | m/z = 655.21(C₄₆H₂₉N₃S = 655.82) |
| E-37 | m/z = 579.18(C₄₀H₂₅N₃S = 579.72) | E-38 | m/z = 655.21(C₄₆H₂₉N₃S = 655.82) |
| E-39 | m/z = 765.28(C₅₆H₃₅N₃O = 765.92) | E-40 | m/z = 705.20(C₄₈H₂₇N₅S = 705.84) |
| E-41 | m/z = 728.26(C₅₂H₃₂N₄O = 728.86) | E-42 | m/z = 720.23(C₅₀H₃₂N₄S = 720.89) |
| E-43 | m/z = 779.26(C₅₆H₃₃N₃O₂ = 779.90) | E-44 | m/z = 730.22(C₅₁H₃₀N₄S = 730.89) |
| E-45 | m/z = 562.22(C₄₀H₂₆N₄ = 562.68) | E-46 | m/z = 590.25(C₄₂H₃₀N₄ = 590.73) |
| E-47 | m/z = 537.20(C₃₇H₂₃N₅ = 537.63) | E-48 | m/z = 612.23(C₄₄H₂₈N₄ = 612.74) |
| E-49 | m/z = 612.23(C₄₄H₂₈N₄ = 612.74) | E-50 | m/z = 587.21(C₄₁H₂₅N₅ = 587.69) |
| E-51 | m/z = 463.18(C₃₁H₂₁N₅ = 463.54) | E-52 | m/z = 536.20(C₃₈H₂₄N₄ = 536.64) |
| E-53 | m/z = 586.22(C₄₂H₂₆N₄ = 586.70) | E-54 | m/z = 742.28(C₅₂H₃₄N₆ = 742.89) |
| E-55 | m/z = 792.30(C₅₆H₃₆N₆ = 792.95) | E-56 | m/z = 742.28(C₅₂H₃₄N₆ = 742.89) |
| E-57 | m/z = 590.22(C₄₀H₂₆N₆ = 590.69) | E-58 | m/z = 792.30(C₅₆H₃₆N₆ = 792.95) |
| E-59 | m/z = 742.28(C₅₂H₃₄N₆ = 742.89) | E-60 | m/z = 780.30(C₅₅H₃₆N₆ = 780.94) |
| E-61 | m/z = 587.21(C₄₁H₂₅N₅ = 587.69) | E-62 | m/z = 661.25(C₄₉H₃₁N₃ = 661.81) |
| E-63 | m/z = 713.28(C₅₃H₃₅N₃ = 713.88) | E-64 | m/z = 637.25(C₄₇H₃₁N₃ = 637.79) |
| E-65 | m/z = 702.28(C₅₁H₃₄N₄ = 702.86) | E-66 | m/z = 612.23(C₄₄H₂₈N₄ = 612.74) |
| E-67 | m/z = 790.28(C₅₆H₃₄N₆ = 790.93) | E-68 | m/z = 713.26(C₅₁H₃₁N₅ = 713.84) |
| E-69 | m/z = 796.32(C₅₇H₄₀N₄O = 796.97) | E-70 | m/z = 815.29(C₆₀H₃₇N₃O = 815.98) |
| E-71 | m/z = 1005.37(C₇₅H₄₇N₃O = 1006.22) | E-72 | m/z = 816.29(C₅₉H₃₆N₄O = 816.96) |
| E-73 | m/z = 575.21(C₄₀H₂₅N₅ = 575.68) | E-74 | m/z = 704.27(C₄₉H₃₂N₆ = 704.84) |
| E-75 | m/z = 702.28(C₅₁H₃₄N₄ = 702.86) | E-76 | m/z = 727.27(C₅₂H₃₃N₅ = 727.87) |
| E-77 | m/z = 675.24(C₄₈H₂₉N₅ = 675.80) | E-78 | m/z = 648.23(C₄₇H₂₈N₄ = 648.77) |
| E-79 | m/z = 551.21(C₃₈H₂₅N₅ = 551.65) | E-80 | m/z = 926.34(C₆₉H₄₂N₄ = 927.12) |

### Fabrication and Evaluation of Organic Electric Element

### [Test Example 1] Green Organic Electroluminescent Element

After vacuum-depositing 4,4′,4″-tris[2-naphthyl(phenyl)amino]triphenylamine (hereinafter abbreviated as “2-TNATA”) on an ITO layer (anode) formed on a glass substrate to form a hole injection layer of 60 nm thickness, a hole transport layer of 60 nm thickness was formed by vacuum-depositing N,N′ -bis(1-naphthalenyl)-N,N′ -bis-phenyl-(1,1′-biphenyl)-4,4′-diamine (hereinafter abbreviated as “NPB”) on the hole injection layer.

Subsequently, an emission-auxiliary layer of 20 nm thickness was formed by vacuum-depositing the compound P-6 of the present invention on the hole transport layer and 4,4′-N,N′-dicarbazole-biphenyl(hereinafter abbreviated as “CBP”) as a host material and tris(2-phenylpyridine)-iridium (hereinafter abbreviated as “Ir(ppy)₃) as a dopant material in a weight ratio of 95:5 were deposited on the emission-auxiliary layer to form a light emitting layer of 30 nm thickness.

Next, (1,1′-bisphenyl-4-olato)bis(2-methyl-8-quinolinolato)aluminum (hereinafter abbreviated as “BAlq”) was vacuum-deposited to form a hole blocking layer of 10 nm thickness on the light emitting layer, and the compound E-14 of the present invention was vacuum-deposited to form an electron transport layer of 40 nm thickness on the hole blocking layer. Thereafter, LiF was deposited to form an electron injection layer of 0.2 nm thickness, and then Al was deposited to form a cathode of 150 nm thickness.

### [Test Example 2] to [Test Example 28]

The organic electroluminescent elements were fabricated in the same manner as described in Test Example 1 except that the compounds of the present invention described in the following Table 7 were used as material of an emission-auxiliary layer and an electron injection layer.

### [Comparative Example 1] to [Comparative Example 4]

The organic electroluminescent elements were fabricated in the same manner as described in Test Example 1 except that the compounds of the present invention described in the following Table 7 were used as material of an emission-auxiliary layer and an electron injection layer.

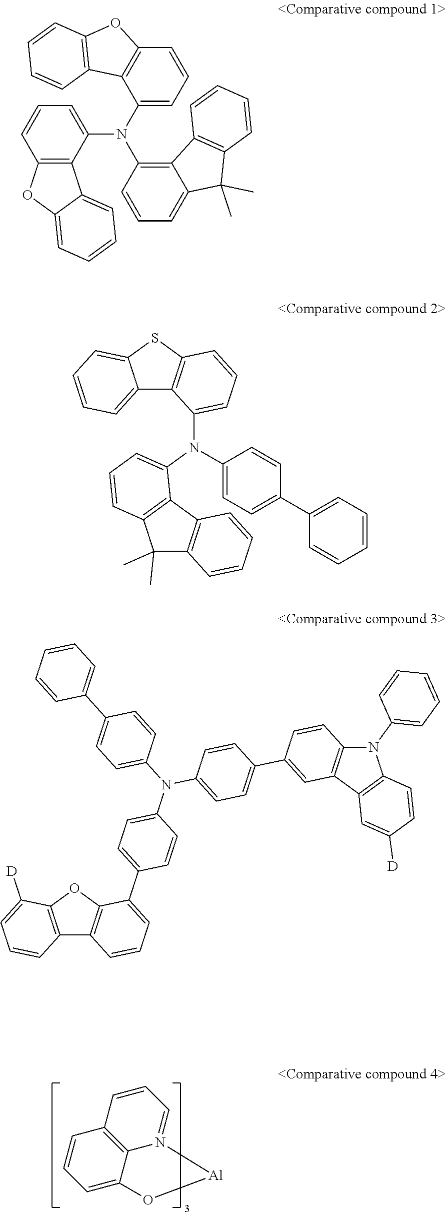

Electroluminescence (EL) characteristics were measured with PR-650 (Photoresearch) by applying a forward bias DC voltage to the organic electroluminescent elements prepared in Test Examples 1 to 28 of the present invention and Comparative Examples 1 to 7. T95 life time was measured using a life time measuring apparatus manufactured by mc science Inc. at reference brightness of 5000 cd/m². The measurement results are shown in Tables 7 below.

TABLE 7

| | Emission | Electron | | Current | | | |
|---|---|---|---|---|---|---|---|
| | auxiliary | transport | Voltage | Density | Brightness | Efficiency | Lifetime |
| | layer | layer | (V) | (mA/cm²) | (cd/m²) | (cd/A) | T(95) |
| comp. Ex(1) | Comp. compd 1 | Comp. compd 4 | 6.0 | 20.4 | 5000.0 | 24.5 | 66.8 |
| comp. Ex(2) | Comp. compd 1 | E-14 | 5.6 | 14.0 | 5000.0 | 35.7 | 79.9 |
| comp. Ex(3) | Comp. compd 2 | | 5.5 | 13.9 | 5000.0 | 36.1 | 77.2 |
| comp. Ex(4) | Comp. compd 3 | | 5.9 | 15.2 | 5000.0 | 32.8 | 73.8 |
| comp. Ex(5) | P-6 | Comp. compd 4 | 5.5 | 14.0 | 5000.0 | 35.8 | 85.1 |
| comp. Ex(6) | P-37 | | 5.6 | 13.3 | 5000.0 | 37.7 | 82.8 |
| comp. Ex(7) | P-101 | | 5.7 | 14.7 | 5000.0 | 34.0 | 79.9 |
| Test Ex. (1) | P-6 | E-14 | 5.2 | 10.2 | 5000.0 | 49.1 | 102.2 |
| Test Ex. (2) | P-9 | | 5.1 | 10.2 | 5000.0 | 48.9 | 102.4 |
| Test Ex. (3) | P-26 | | 5.2 | 9.9 | 5000.0 | 50.7 | 104.7 |
| Test Ex. (4) | P-37 | | 5.1 | 10.0 | 5000.0 | 49.8 | 103.3 |
| Test Ex. (5) | P-57 | | 5.1 | 9.9 | 5000.0 | 50.6 | 103.0 |
| Test Ex. (6) | P-69 | | 5.1 | 10.0 | 5000.0 | 50.2 | 102.4 |
| Test Ex. (7) | P-101 | | 5.2 | 10.3 | 5000.0 | 48.6 | 95.2 |
| Test Ex. (8) | P-6 | E-25 | 5.1 | 9.6 | 5000.0 | 52.2 | 106.9 |
| Test Ex. (9) | P-9 | | 5.0 | 9.4 | 5000.0 | 53.1 | 106.3 |
| Test Ex. (10) | P-26 | | 5.1 | 9.2 | 5000.0 | 54.2 | 109.3 |
| Test Ex. (11) | P-37 | | 5.0 | 9.3 | 5000.0 | 54.1 | 105.9 |
| Test Ex. (12) | P-57 | | 5.1 | 9.8 | 5000.0 | 51.0 | 104.7 |
| Test Ex. (13) | P-69 | | 5.0 | 9.8 | 5000.0 | 51.1 | 107.9 |
| Test Ex. (14) | P-101 | | 5.1 | 9.8 | 5000.0 | 50.8 | 100.5 |
| Test Ex. (15) | P-6 | E-31 | 4.8 | 9.1 | 5000.0 | 54.9 | 111.2 |
| Test Ex. (16) | P-9 | | 4.8 | 9.1 | 5000.0 | 54.9 | 110.0 |
| Test Ex. (17) | P-26 | | 4.8 | 9.0 | 5000.0 | 55.7 | 112.6 |
| Test Ex. (18) | P-37 | | 4.9 | 9.1 | 5000.0 | 54.7 | 110.4 |
| Test Ex. (19) | P-57 | | 4.8 | 9.1 | 5000.0 | 54.8 | 111.0 |
| Test Ex. (20) | P-69 | | 4.9 | 9.3 | 5000.0 | 54.0 | 110.7 |
| Test Ex. (21) | P-101 | | 4.9 | 9.3 | 5000.0 | 53.8 | 104.2 |
| Test Ex. (22) | P-6 | E-61 | 5.1 | 9.5 | 5000.0 | 52.6 | 106.7 |
| Test Ex. (23) | P-9 | | 5.2 | 9.6 | 5000.0 | 52.1 | 106.9 |
| Test Ex. (24) | P-26 | E-61 | 5.1 | 9.2 | 5000.0 | 54.1 | 105.6 |
| Test Ex. (25) | P-37 | | 5.1 | 9.7 | 5000.0 | 51.6 | 107.3 |
| Test Ex. (26) | P-57 | | 5.1 | 9.5 | 5000.0 | 52.7 | 106.5 |
| Test Ex. (27) | P-69 | | 5.2 | 9.4 | 5000.0 | 53.4 | 106.5 |
| Test Ex. (28) | P-101 | | 5.2 | 9.8 | 5000.0 | 51.0 | 99.7 |

As can be seen from the result in Table 7, when the compound represented by Formula 1 is used as an emission auxiliary layer material and the compound represented by Formula 2 is used as the electron transport layer material, compared to the case of using the compounds of Comparative Examples 1 to 7, the driving voltage of the organic electroluminescent element is lowered, and the luminous efficiency and lifespan are significantly improved.

Comparative Example 1 is a case where Comparative compound 1 is used as an emission auxiliary layer material and Comparative compound 4 is used as the electron transport layer material. Compared to Comparative Example 1, in Comparative Examples 2 to 7 in which the compound of the present invention is used as either an emission auxiliary layer material or the electron transport layer material, the driving voltage, efficiency and lifespan are improved.

Comparative Compound 1 and Comparative Compound 2 are similar to compound of the present invention in that an amine group is substituted at position 1 of the dibenzofuran or dibenzothiophene moiety, but compound of Formula 1 of the present invention differs from Comparative compound 1 or 2 in that dibenzofuran or dibenzothiophene moiety are substituted with one or more deuteriums.

When using compound represented by Formula 1 of the present invention in a hole transport band, wherein the compound is necessarily substituted with deuterium, the volume of molecular core is reduced. This is because the zero-point energy, that is, the energy of the ground state, is lower compared to compounds in which deuterium is not substituted, and the bond length of carbon and deuterium is shorter than that of carbon and hydrogen. Therefore, electrical polarizability can be reduced, and intermolecular interaction can be weakened, thereby increasing the thin film volume. These characteristics have the effect of lowering the crystallinity of the thin film, that is, creating an amorphous state.

This characteristic has the effect of lowering the crystallinity of the thin film, that is, creating an amorphous state. This amorphous state can let the flow of charge, that is, hole mobility fast by reducing grain boundaries due to isotropic and homogeneous characteristics

Additionally, when compound represented by Formula 2 is used in the electron transport band, electrons are injected into the light-emitting layer more quickly than Comparative compound 4.

Therefore, when compound represented by Formula 1 is used in the hole transport band and compound represented by Formula 2 is used in the electron transport band, holes and electrons are injected into the light-emitting layer more quickly, thereby improving the charge balance in the light-emitting layer. As a result, the characteristics of an organic electric element can be improved.

In addition, some electrons may pass from the emitting layer to the hole transport region. Stability of the compound increases by increasing the bond dissociation energy (BDE) of Compound represented by Formula 1, wherein the compound is substituted with deuterium. As a result, it appears that the lifespan of an organic electric element is improved.

Comparative compound 3 is similar to the compound of the present invention in that it is a tertiary amine substituted with deuterium-substituted dibenzofuran. However, Comparative compound 3 differs from the present invention in the position where the amine group is substituted. In compound of the present invention, an amine group is substituted at position 1 of dibenzofuran, whereas in Comparative compound 3, an amine group is substituted at position 4 of dibenzofuran.

When an amine group is substituted at position 1 of dibenzofuran or dibenzothiophene, the band gap between the HOMO and LUMO of the molecule is widened due to steric hindrance, and T1 energy level is higher and an amine group has a better shielding effect than when substituted at other positions. As a result, it appears that the efficiency and lifespan of compound of the present invention are significantly improved compared to Comparative compound 3.

In conclusion, when compound of the present invention represented by Formula 1 is used as an emission auxiliary layer and compound of the present invention represented by Formula 2 is used as an electron transport layer, more holes and electrons move quickly and easily into the light-emitting layer. As a result, the charge balance in the light-emitting layer increases and light is emitted from within the light-emitting layer rather than at the interface of an emission auxiliary layer, and deterioration at the interface between ITO and a hole transport layer is also reduced. As a result, it can be seen that the driving voltage, efficiency, and lifespan of the entire element are improved.

Although the exemplary embodiments of the present invention have been described for illustrative purposes, those skilled in the art to which the present invention pertains will be capable of various modifications without departing from the essential characteristics of the present invention. Therefore, the embodiment disclosed herein is intended to illustrate the present invention rather than to limit the present invention and the scope of the present invention is not limited by the embodiments. The scope of the present invention shall be construed on the basis of the accompanying claims, and it shall be construed that all of the technical ideas included within the scope equivalent to the claims belong to the present invention.

1. An organic electric element comprising:
a first electrode;
a second electrode; and
an organic material layer formed between the first electrode and the second electrode, the organic material layer comprising:
a light-emitting layer;
a hole transport band layer formed between the light-emitting layer and the first electrodes; and
an electron transport band layer formed between the light-emitting layer and the second electrode,
wherein the hole transport band layer includes compound represented by the following Formula 1, and the electron transport band layer includes compound represented by the following Formula 2: 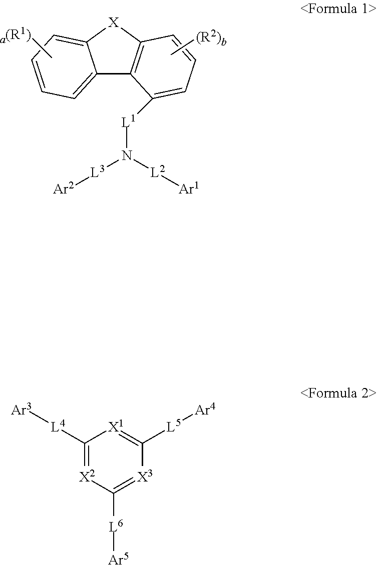
wherein:
X is O or S,
X¹ to X³ are N or C(R₁), and at least one of X¹ to X³ is N,
R¹, R² and R₁ are each independently selected from the group consisting of hydrogen, deuterium, halogen, a cyano group, a nitro group, a C₆-C₆₀ aryl group, a fluorenyl group, a C₂-C₆₀ heterocyclic group comprising at least one heteroatom selected from the group consisting of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, a C₆-C₂₀ aryloxy group and -L′-N(Rₐ)(R_{b}), and adjacent groups may be bonded to each other to form a ring, with a proviso that at least one of R¹ and R² is deuterium,
a is an integer of 1 to 4, b is an integer of 1 to 3, and when each of these is an integer of 2 or more, each of R¹s, each of R²s are the same as or different from each other, and when R₁ is multiple, each of these are the same or different from the other,
L¹ to L⁶ are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P,
Ar¹ to Ar⁵ are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group,
L′ is selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P,
R^{a} and R^{b} are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group, and
the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent groups may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a Cl₁C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

2. The organic electric element of claim 1, wherein at least one of Ar¹ to Ar⁵ is represented by one of Formulas Ar-1 to Ar-5 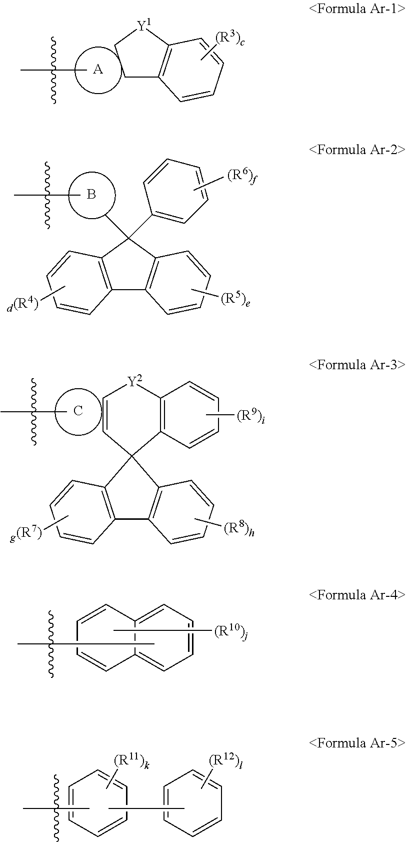
wherein:
Y¹ is O, S, C(R′)(R″) or N(Ar₁),
Y² is a single bond, O, S, C(R′)(R″) or N(Ar₂),
rings A to C are each independently selected from the group consisting of a C₆-C₆₀ aromatic ring group, a fluorenylene group, a C₃-C₆₀ aliphatic ring group, and a C₂-C₆₀ heterocyclic group containing at least one heteroatom of O, N, S, Si and P, the rings A to C may be each optionally substituted with R¹³,
R³ to R¹³, R′ and R″ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and adjacent groups may be bonded to each other to form a ring, R′ and R″ may be bonded to each other to form a ring, and multiple R¹³s are the same or different from each other,
c to e, g to i, and k are each an integer of 0 to 4, f and 1 are each an integer of 0 to 5, j is an integer of 0 to 7, and when each of these is an integer of 2 or more, each of R³, each of R⁴, each of R⁵, each of R⁶, each of R⁷, each of R⁸, each of R⁹, each of R¹⁰, each of R¹¹ and each of R¹² are the same as or different from each other, and
Ar₁ and Ar₂ are each independently selected from the group consisting of a C₆-C₂₀ aryl group, a fluorenyl group, a C₂-C₂₀ heterocyclic group containing at least one heteroatom of O, N, S, Si and P, and a C₃-C₂₀ aliphatic ring group.

3. The organic electric element of claim 2, wherein at least one of the rings A to C is selected from the group consisting of Formula F-1 to Formula F-7 below: 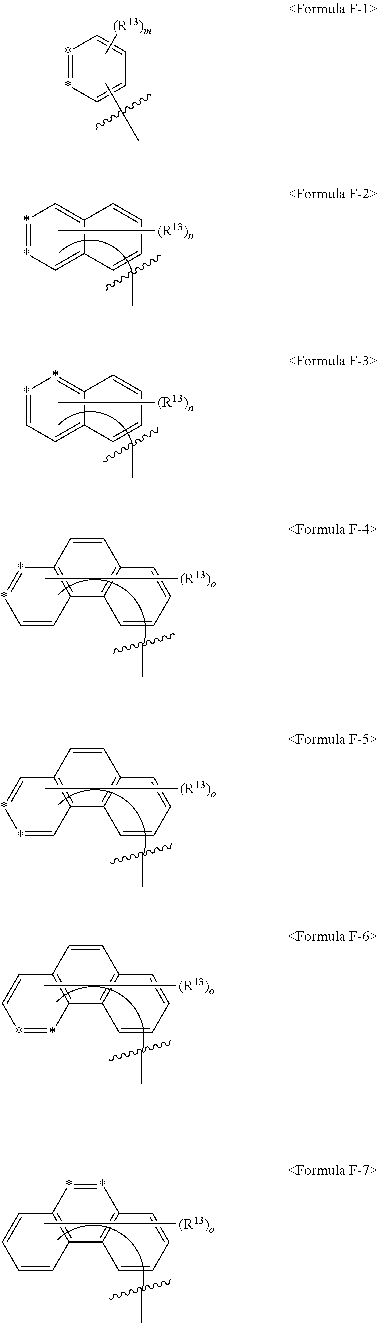
wherein * indicates the condensation position,
R¹³ is the same as defined in claim 2,
m is an integer from 0 to 3,
n is an integer from 0 to 5, and
o is an integer from 0 to 7.

4. The organic electric element of claim 1, wherein at least one of L¹ to L⁶ is selected from the group consisting of Formulas b-1 to b-13 below: 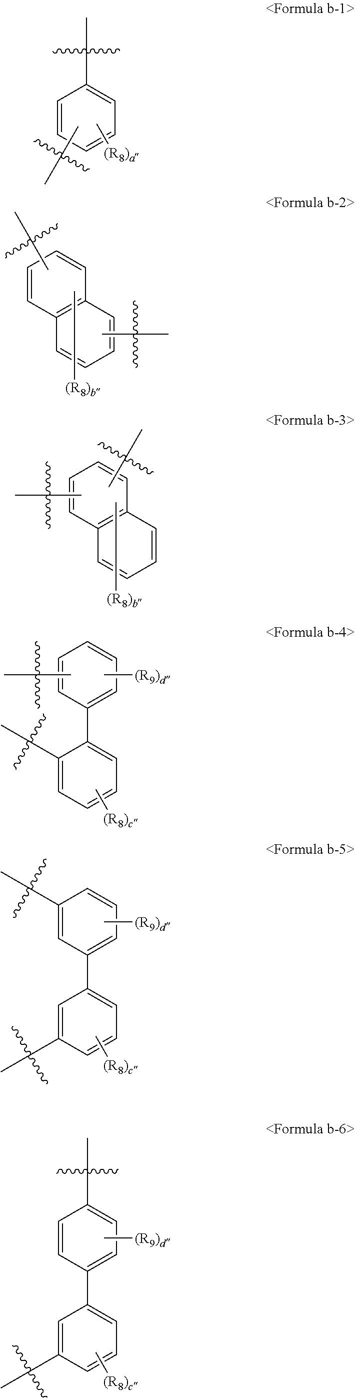 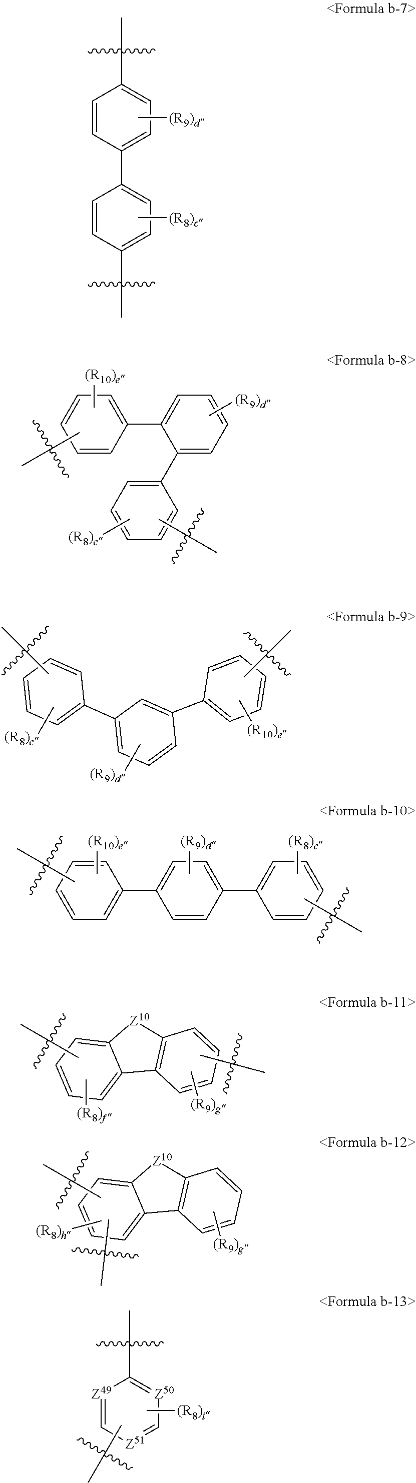
in Formula b-1 to Formula b-13,
Z¹⁰ is O, S, C(R′)(R″) or N(Ar₃),
Z⁴⁹ to Z⁵¹ are each independently N or C(R′), and at least one of Z⁴⁹ to Z⁵¹ is N,
R₈ to R₁₀, R′ and R″ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and adjacent groups may be bonded to each other to form a ring, and R′ and R″ may be bonded to each other to form a ring,
a″, c″, d″, e″ and g″ in Formula b-12 are each integer of 0 to 4, f′ and g″ in Formula b-11 are integers of 0 to 3, h″ is 0 is an integer of 0 to 2, and i″ is an integer of 0 or 1, and when each of these is an integer of 2 or more, each of R₈, each of R₉, and each of R₁₀ are the same as or different from each other, and
Ar₃ is selected from the group consisting of a C₆-C₂₀ aryl group, a fluorenyl group, a C₂-C₂₀ heterocyclic group containing at least one heteroatom of O, N, S, Si and P, and a C₃-C₂₀ aliphatic ring group.

5. The organic electric element of claim 1, wherein the compound represented by Formula 1 is represented by Formula 1-1 or Formula 1-2 below: 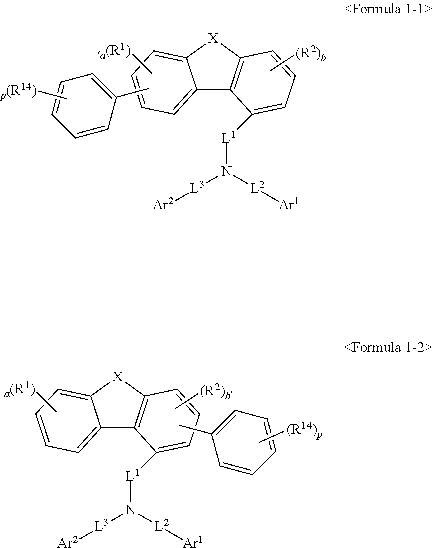
in Formula 1-1 and Formula 1-2, R¹, R², a, b, L¹ to L³, Ar¹ and Ar² are the same as defined in claim 1,
R¹⁴ is selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group, and adjacent groups may be bonded to each other to form a ring, and
a′ is an integer of 0 to 3, b′ is an integer of 0 to 2, and p is an integer of 0 to 5, and when each of these is an integer of 2 or more, each of R¹, each of R² and each of R¹⁴ are the same or different from each other.

6. The organic electric element of claim 1, wherein at least one of Ar¹ and Ar⁶ is a heterocyclic group.

7. The organic electric element of claim 1, wherein at least one of Ar⁴ to Ar⁶ is an aryl group substituted with a cyano group or a heterocyclic group substituted with a cyano group.

8. The organic electric element of claim 1, wherein the compound represented by Formula 1 is one of the following compounds:

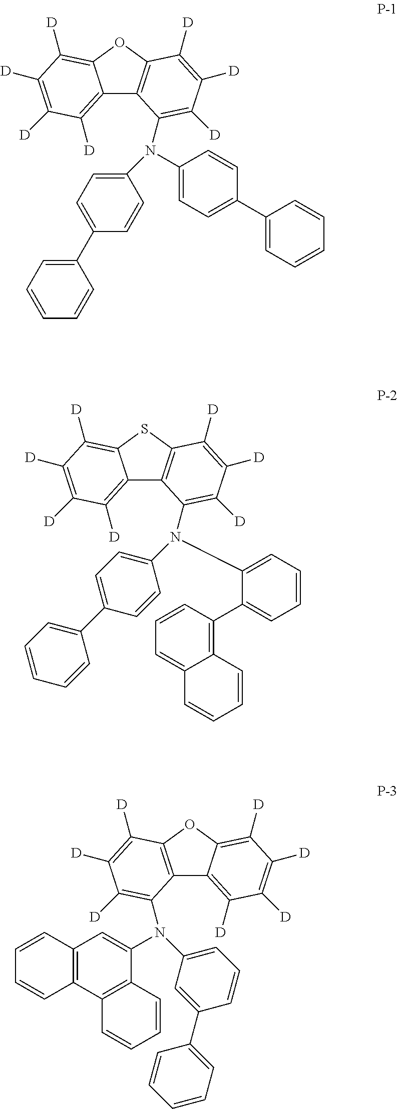 [SMILES: [2H]c1c([2H])c([2H])c2c(oc3c([2H])c([2H])c([2H])c(N(c4ccc(-c5ccccc5)cc4)c4ccc(-c5ccccc5)cc4)c32)c1[2H] ; [2H]c1c([2H])c([2H])c2c(sc3c([2H])c([2H])c([2H])c(N(c4ccc(-c5ccccc5)cc4)c4ccccc4-c4cccc5ccccc45)c32)c1[2H] ; [2H]c1c([2H])c([2H])c2c(oc3c([2H])c([2H])c([2H])c(N(c4cccc(-c5ccccc5)c4)c4cc5ccccc5c5ccccc45)c32)c1[2H]]

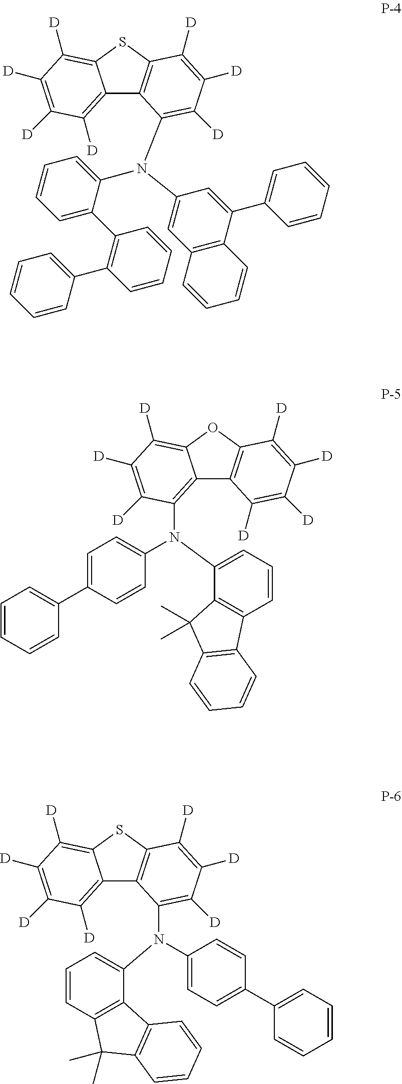 [SMILES: [2H]c1c([2H])c([2H])c2c(sc3c([2H])c([2H])c([2H])c(N(c4cc(-c5ccccc5)c5ccccc5c4)c4ccccc4-c4ccccc4-c4ccccc4)c32)c1[2H] ; [2H]c1c([2H])c([2H])c2c(oc3c([2H])c([2H])c([2H])c(N(c4ccc(-c5ccccc5)cc4)c4cccc5c4C(C)(C)c4ccccc4-5)c32)c1[2H] ; [2H]c1c([2H])c([2H])c2c(sc3c([2H])c([2H])c([2H])c(N(c4ccc(-c5ccccc5)cc4)c4cccc5c4-c4ccccc4C5(C)C)c32)c1[2H]]

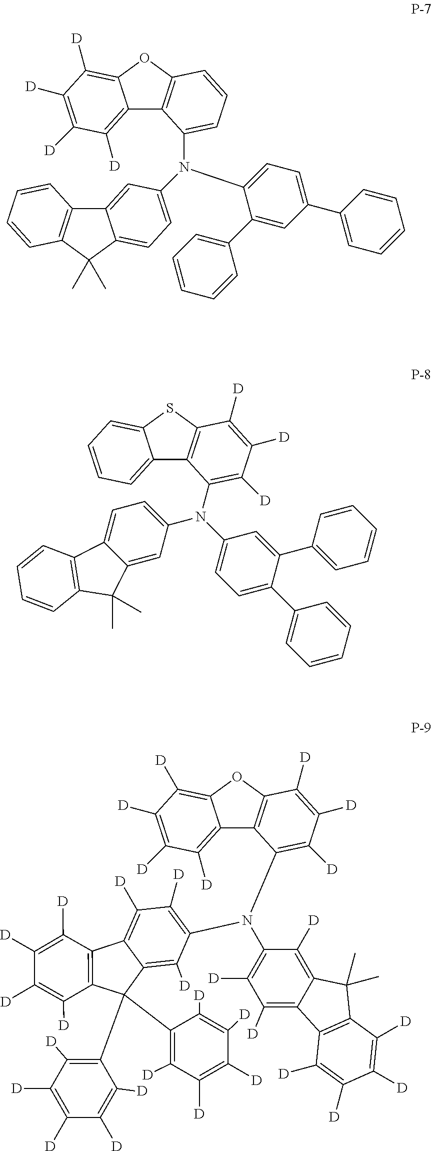 [SMILES: [2H]c1c([2H])c([2H])c2c(oc3cccc(N(c4ccc5c(c4)-c4ccccc4C5(C)C)c4ccc(-c5ccccc5)cc4-c4ccccc4)c32)c1[2H] ; [2H]c1c([2H])c(N(c2ccc(-c3ccccc3)c(-c3ccccc3)c2)c2ccc3c(c2)C(C)(C)c2ccccc2-3)c2c(sc3ccccc32)c1[2H] ; [2H]c1c([2H])c([2H])c(C2(c3c([2H])c([2H])c([2H])c([2H])c3[2H])c3c([2H])c([2H])c([2H])c([2H])c3-c3c([2H])c([2H])c(N(c4c([2H])c([2H])c5c(c4[2H])C(C)(C)c4c([2H])c([2H])c([2H])c([2H])c4-5)c4c([2H])c([2H])c([2H])c5oc6c([2H])c([2H])c([2H])c([2H])c6c45)c([2H])c32)c([2H])c1[2H]]

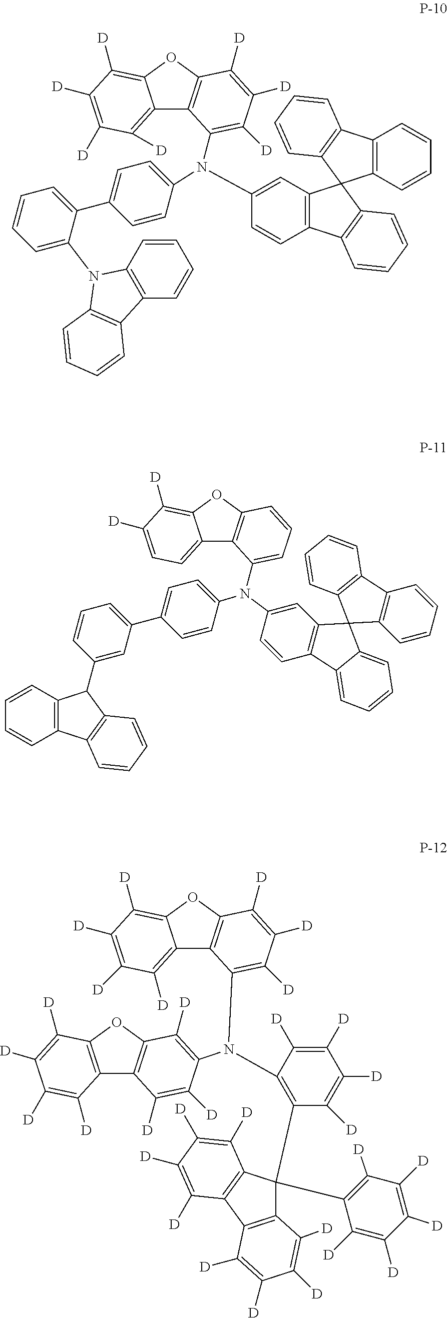

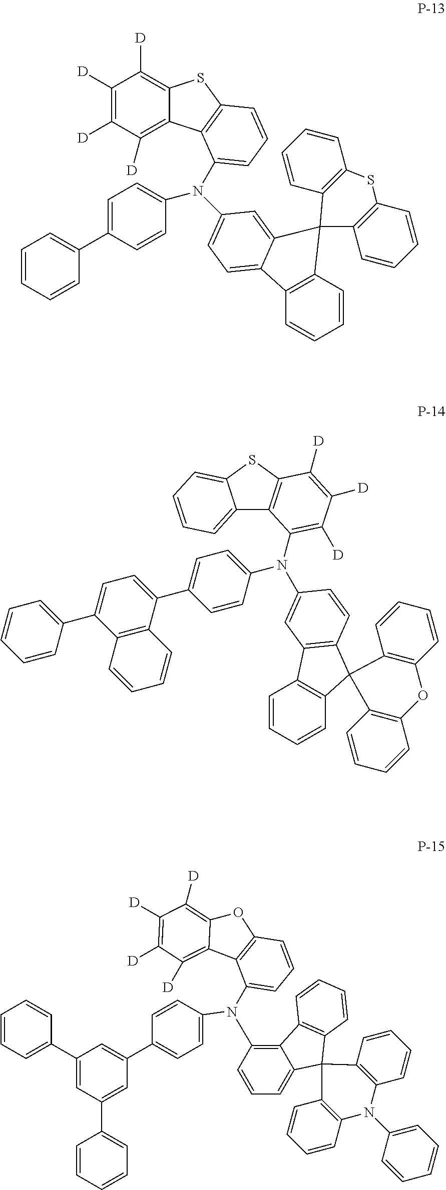 [SMILES: [2H]c1c([2H])c([2H])c2c(sc3cccc(N(c4ccc(-c5ccccc5)cc4)c4ccc5c(c4)C4(c6ccccc6Sc6ccccc64)c4ccccc4-5)c32)c1[2H] ; [2H]c1c([2H])c(N(c2ccc(-c3ccc(-c4ccccc4)c4ccccc34)cc2)c2ccc3c(c2)-c2ccccc2C32c3ccccc3Oc3ccccc32)c2c(sc3ccccc32)c1[2H] ; [2H]c1c([2H])c([2H])c2c(oc3cccc(N(c4ccc(-c5cc(-c6ccccc6)cc(-c6ccccc6)c5)cc4)c4cccc5c4-c4ccccc4C54c5ccccc5N(c5ccccc5)c5ccccc54)c32)c1[2H]]

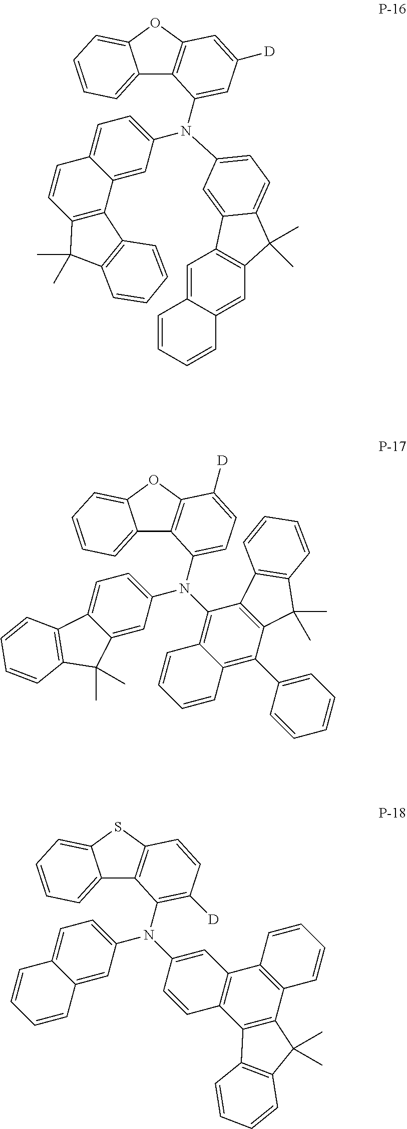

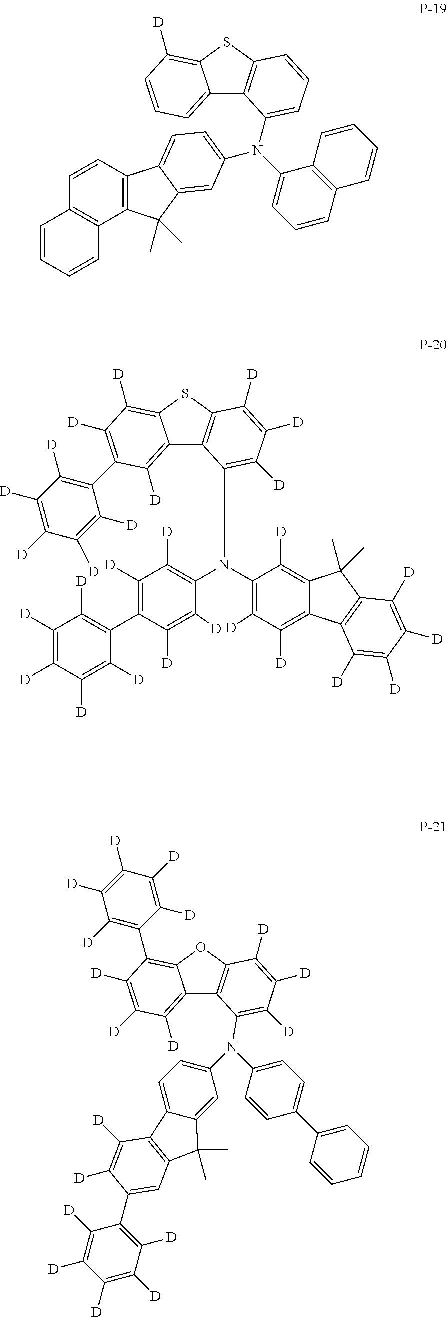

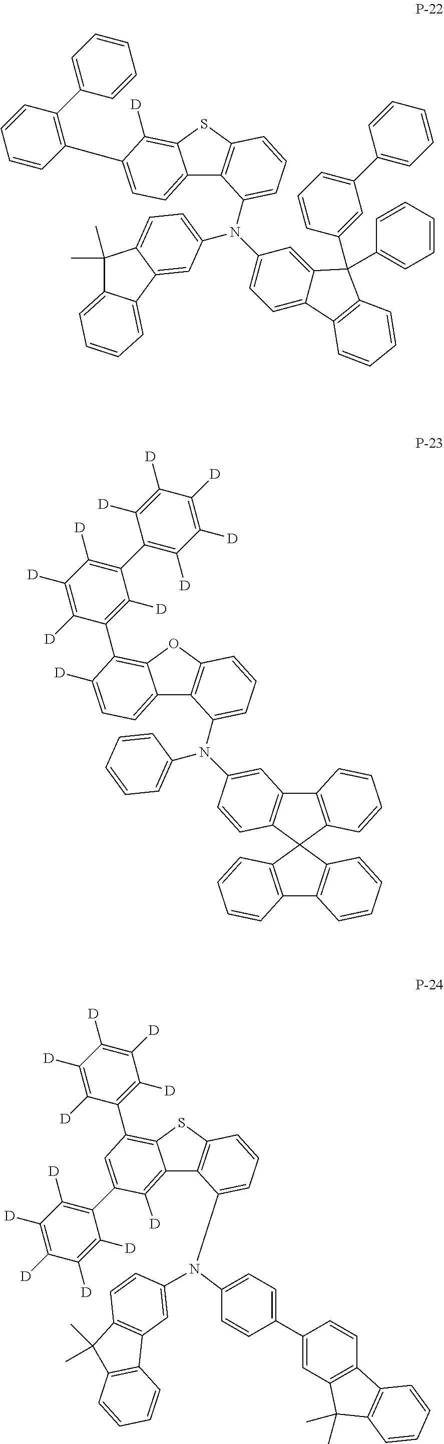 [SMILES: [2H]c1c(-c2ccccc2-c2ccccc2)ccc2c1sc1cccc(N(c3ccc4c(c3)-c3ccccc3C4(C)C)c3ccc4c(c3)C(c3ccccc3)(c3cccc(-c5ccccc5)c3)c3ccccc3-4)c12 ; [2H]c1ccc2c(oc3cccc(N(c4ccccc4)c4ccc5c(c4)-c4ccccc4C54c5ccccc5-c5ccccc54)c32)c1-c1c([2H])c([2H])c([2H])c(-c2c([2H])c([2H])c([2H])c([2H])c2[2H])c1[2H] ; [2H]c1c([2H])c([2H])c(-c2cc(-c3c([2H])c([2H])c([2H])c([2H])c3[2H])c3sc4cccc(N(c5ccc(-c6ccc7c(c6)C(C)(C)c6ccccc6-7)cc5)c5ccc6c(c5)-c5ccccc5C6(C)C)c4c3c2[2H])c([2H])c1[2H]]

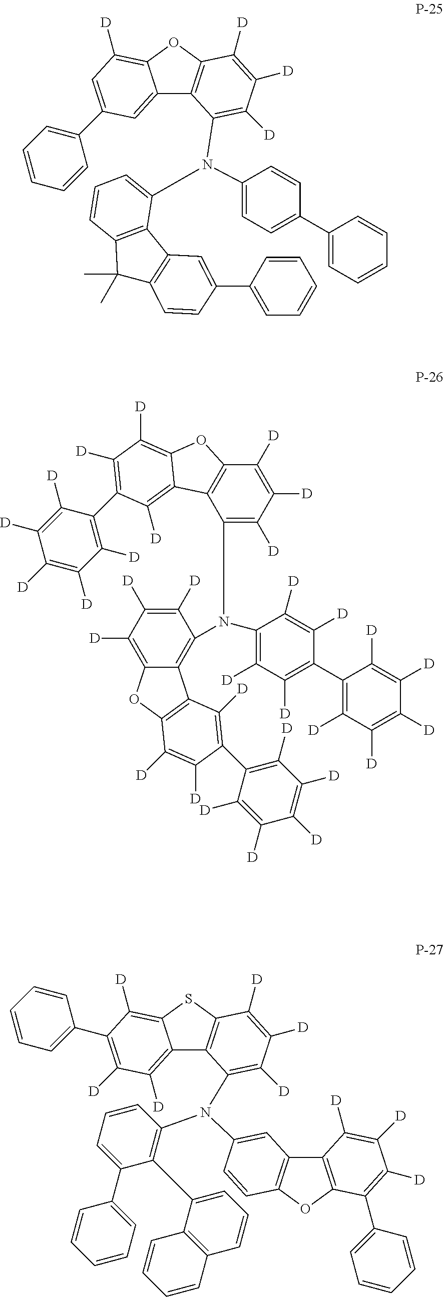

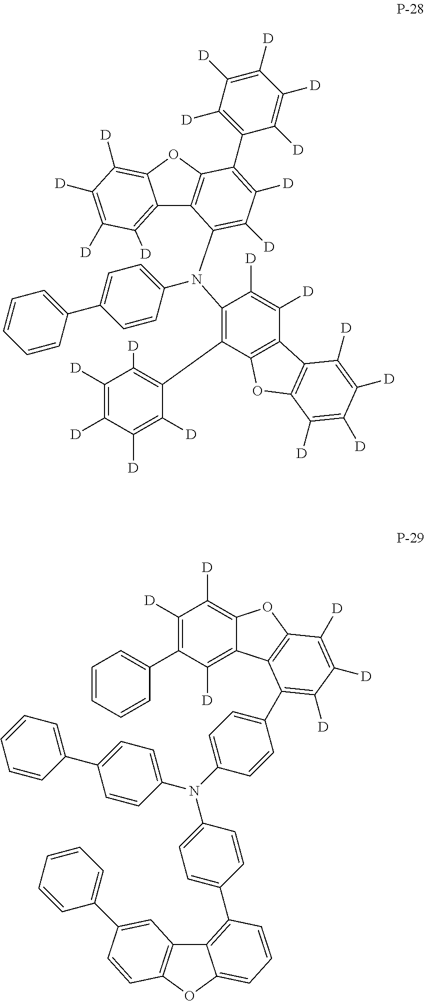 [SMILES: [2H]c1c([2H])c([2H])c(-c2c(N(c3ccc(-c4ccccc4)cc3)c3c([2H])c([2H])c(-c4c([2H])c([2H])c([2H])c([2H])c4[2H])c4oc5c([2H])c([2H])c([2H])c([2H])c5c34)c([2H])c([2H])c3c2oc2c([2H])c([2H])c([2H])c([2H])c23)c([2H])c1[2H] ; [2H]c1c(-c2ccccc2)c([2H])c2c(oc3c([2H])c([2H])c([2H])c(-c4ccc(N(c5ccc(-c6ccccc6)cc5)c5ccc(-c6cccc7oc8ccc(-c9ccccc9)cc8c67)cc5)cc4)c32)c1[2H]]

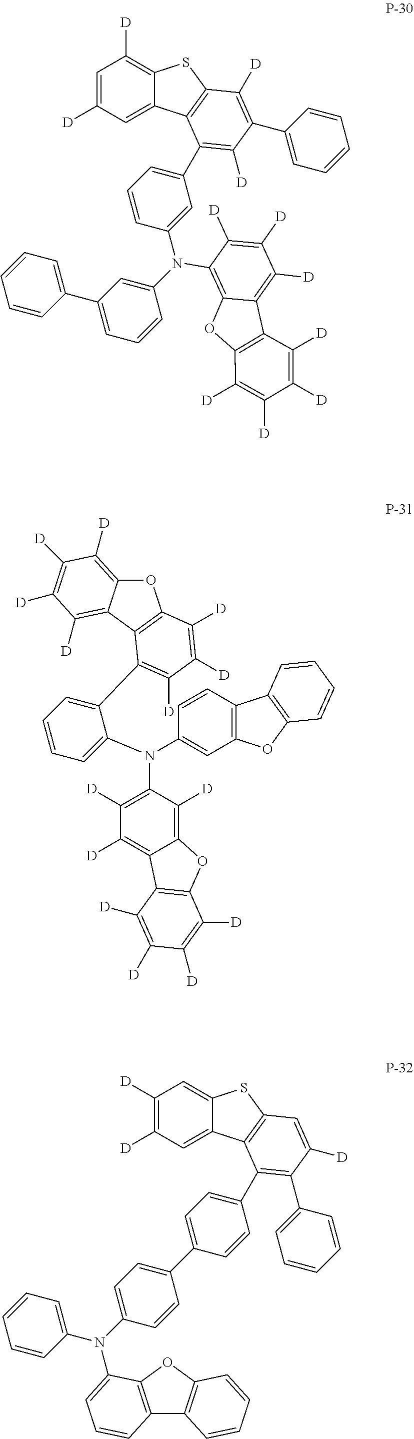 [SMILES: [2H]c1cc([2H])c2sc3c([2H])c(-c4ccccc4)c([2H])c(-c4cccc(N(c5cccc(-c6ccccc6)c5)c5c([2H])c([2H])c([2H])c6c5oc5c([2H])c([2H])c([2H])c([2H])c56)c4)c3c2c1 ; [2H]c1c([2H])c([2H])c2c(oc3c([2H])c(N(c4ccc5c(c4)oc4ccccc45)c4ccccc4-c4c([2H])c([2H])c([2H])c5oc6c([2H])c([2H])c([2H])c([2H])c6c45)c([2H])c([2H])c32)c1[2H] ; [2H]c1cc2sc3cc([2H])c(-c4ccccc4)c(-c4ccc(-c5ccc(N(c6ccccc6)c6cccc7c6oc6ccccc67)cc5)cc4)c3c2cc1[2H]]

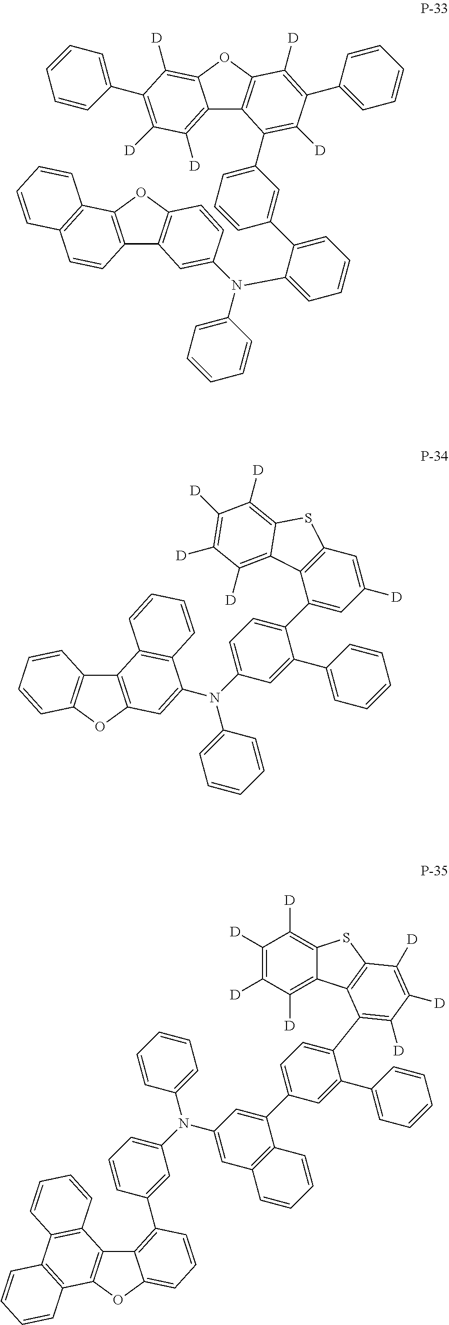 [SMILES: [2H]c1c(-c2ccccc2)c([2H])c2oc3c([2H])c(-c4ccccc4)c([2H])c(-c4cccc(-c5ccccc5N(c5ccccc5)c5ccc6oc7c8ccccc8ccc7c6c5)c4)c3c2c1[2H] ; [2H]c1cc(-c2ccc(N(c3ccccc3)c3cc4oc5ccccc5c4c4ccccc34)cc2-c2ccccc2)c2c(c1)sc1c([2H])c([2H])c([2H])c([2H])c12 ; [2H]c1c([2H])c([2H])c2c(sc3c([2H])c([2H])c([2H])c(-c4ccc(-c5cc(N(c6ccccc6)c6cccc(-c7cccc8oc9c%10ccccc%10c%10ccccc%10c9c78)c6)cc6ccccc56)cc4-c4ccccc4)c32)c1[2H]]

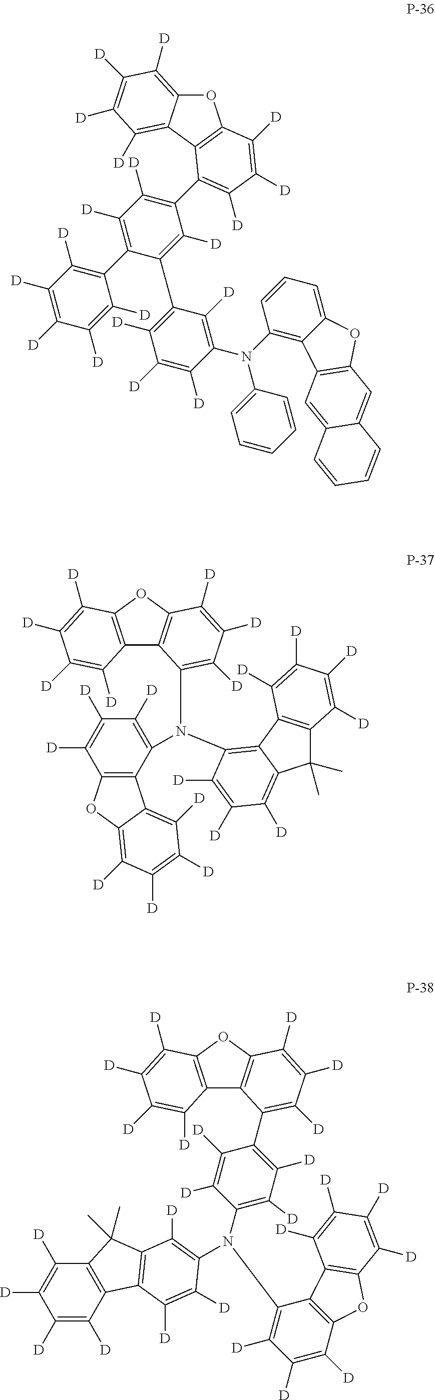

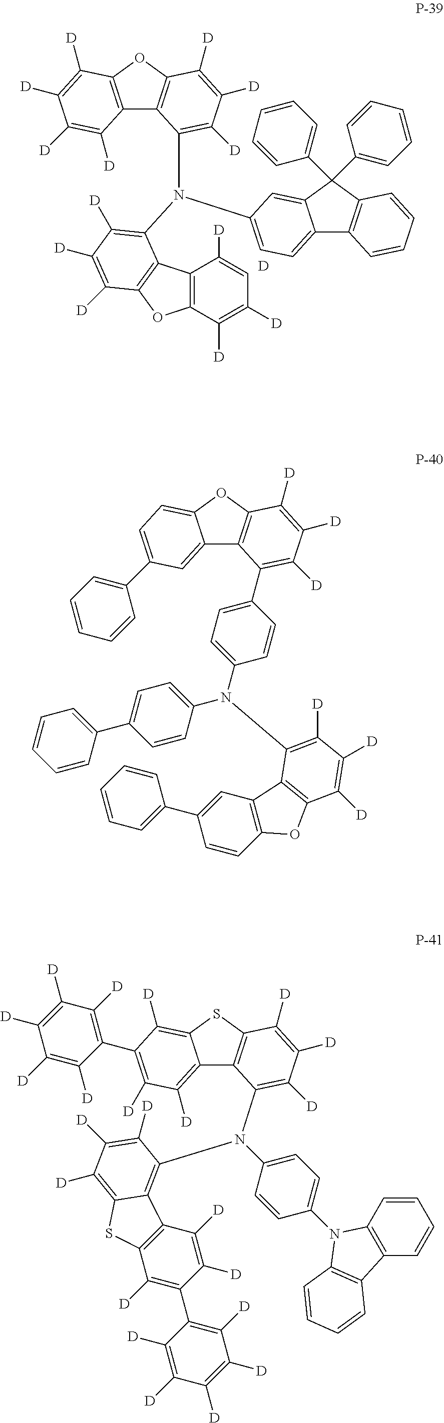

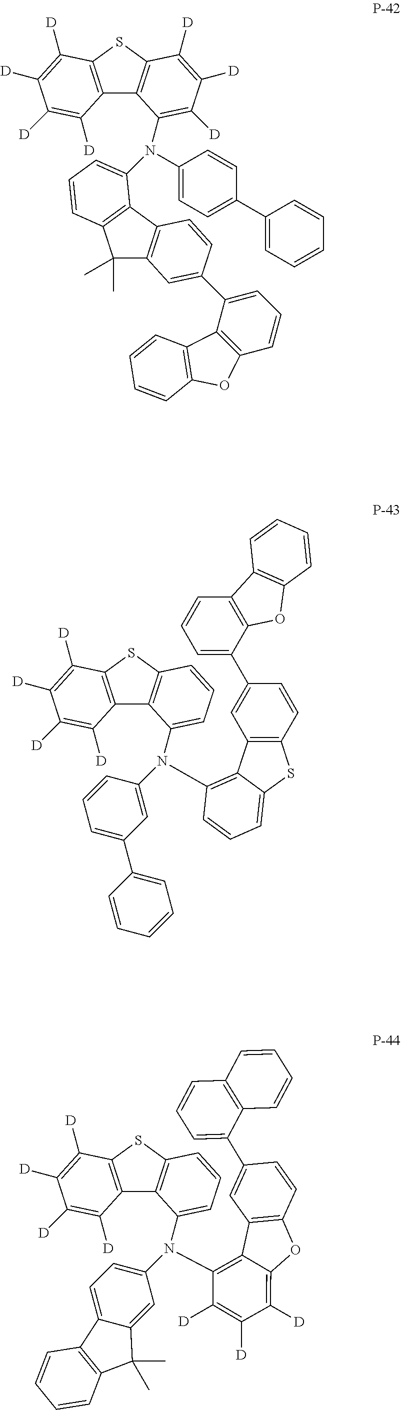 [SMILES: [2H]c1c([2H])c([2H])c2c(sc3c([2H])c([2H])c([2H])c(N(c4ccc(-c5ccccc5)cc4)c4cccc5c4-c4ccc(-c6cccc7oc8ccccc8c67)cc4C5(C)C)c32)c1[2H] ; [2H]c1c([2H])c([2H])c2c(sc3cccc(N(c4cccc(-c5ccccc5)c4)c4cccc5sc6ccc(-c7cccc8c7oc7ccccc78)cc6c45)c32)c1[2H] ; [2H]c1c([2H])c(N(c2ccc3c(c2)C(C)(C)c2ccccc2-3)c2cccc3sc4c([2H])c([2H])c([2H])c([2H])c4c23)c2c(oc3ccc(-c4cccc5ccccc45)cc32)c1[2H]]

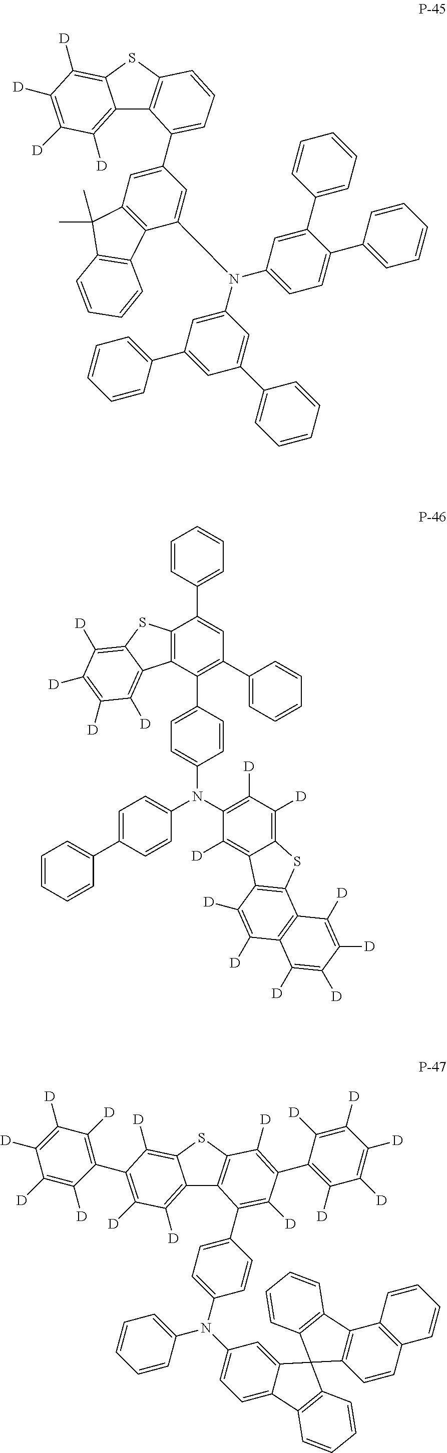

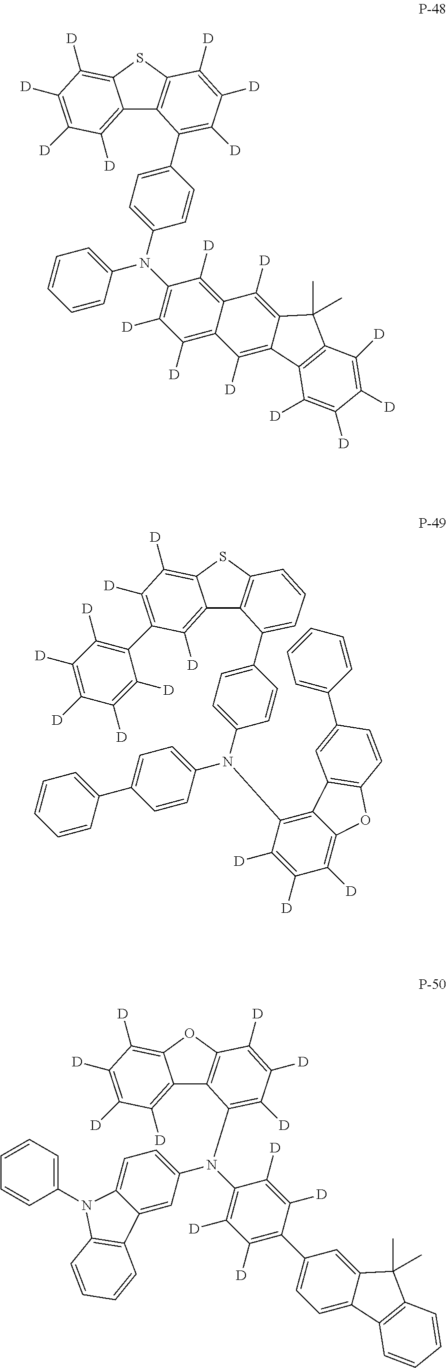 [SMILES: [2H]c1c([2H])c([2H])c2c(c1[2H])-c1c(c([2H])c3c([2H])c(N(c4ccccc4)c4ccc(-c5c([2H])c([2H])c([2H])c6sc7c([2H])c([2H])c([2H])c([2H])c7c56)cc4)c([2H])c([2H])c3c1[2H])C2(C)C ; [2H]c1c([2H])c([2H])c(-c2c([2H])c([2H])c3sc4cccc(-c5ccc(N(c6ccc(-c7ccccc7)cc6)c6c([2H])c([2H])c([2H])c7oc8ccc(-c9ccccc9)cc8c67)cc5)c4c3c2[2H])c([2H])c1[2H] ; [2H]c1c([2H])c(N(c2ccc3c(c2)c2ccccc2n3-c2ccccc2)c2c([2H])c([2H])c([2H])c3oc4c([2H])c([2H])c([2H])c([2H])c4c23)c([2H])c([2H])c1-c1ccc2c(c1)C(C)(C)c1ccccc1-2]

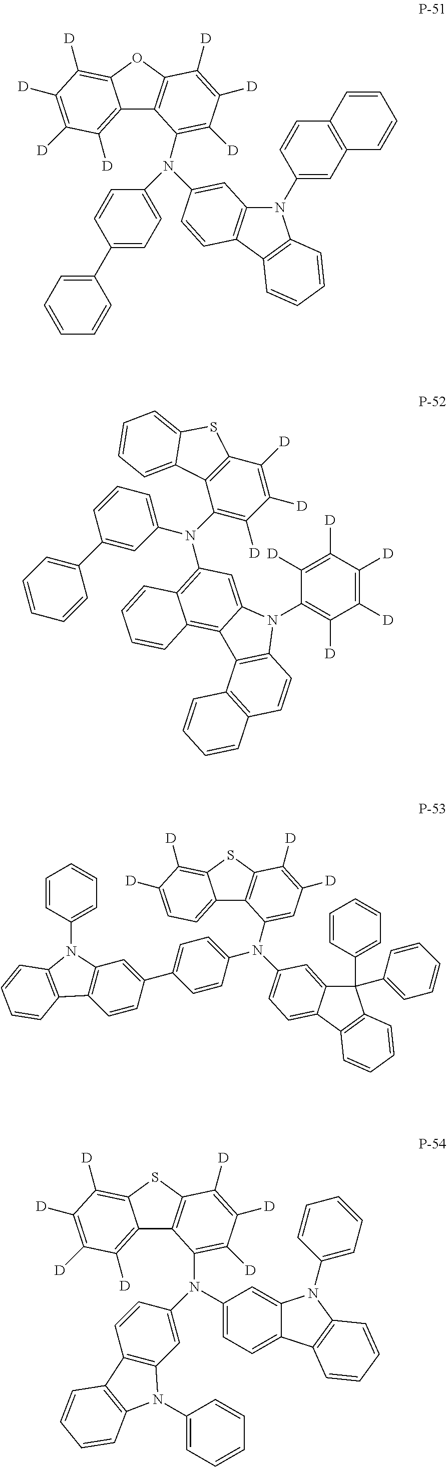

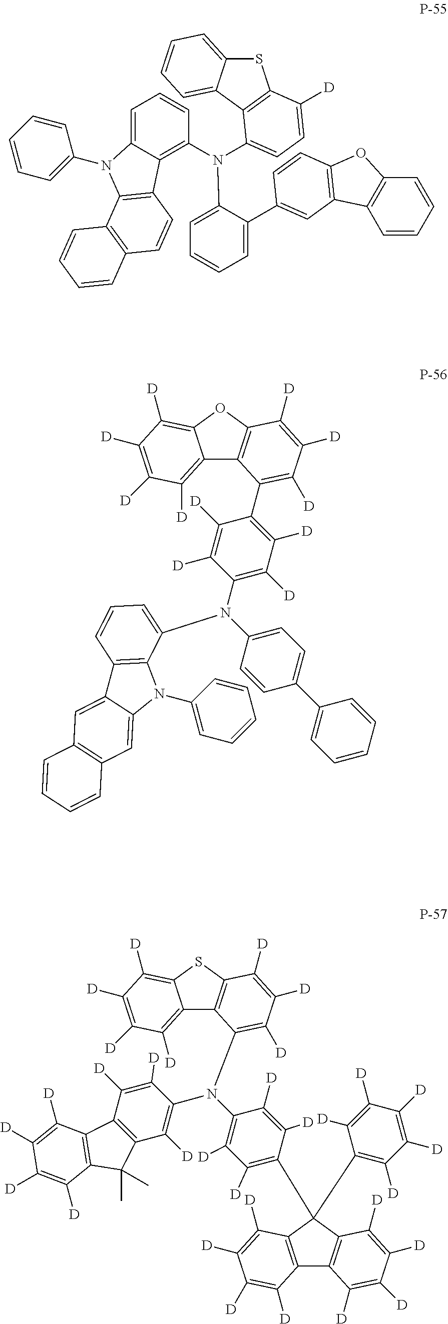

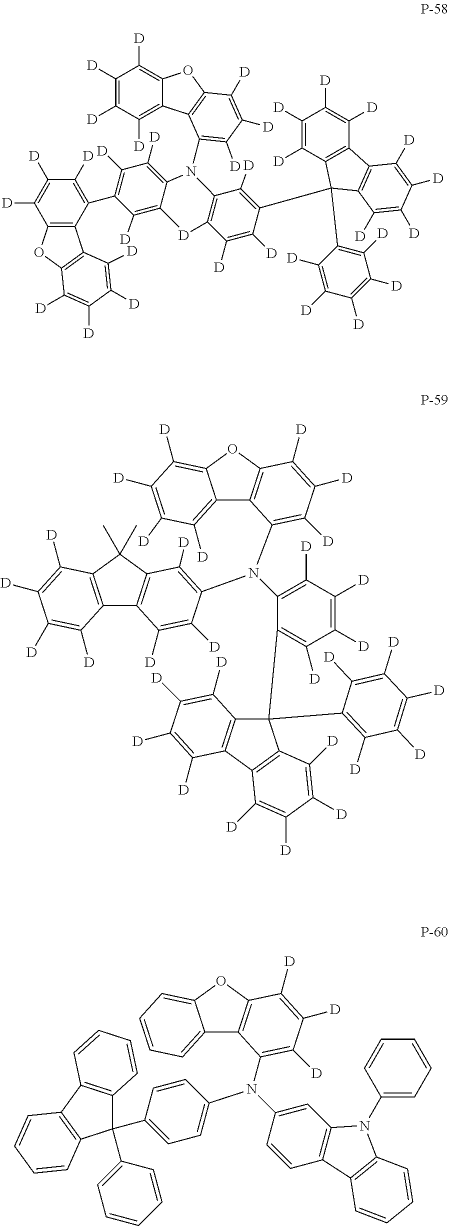

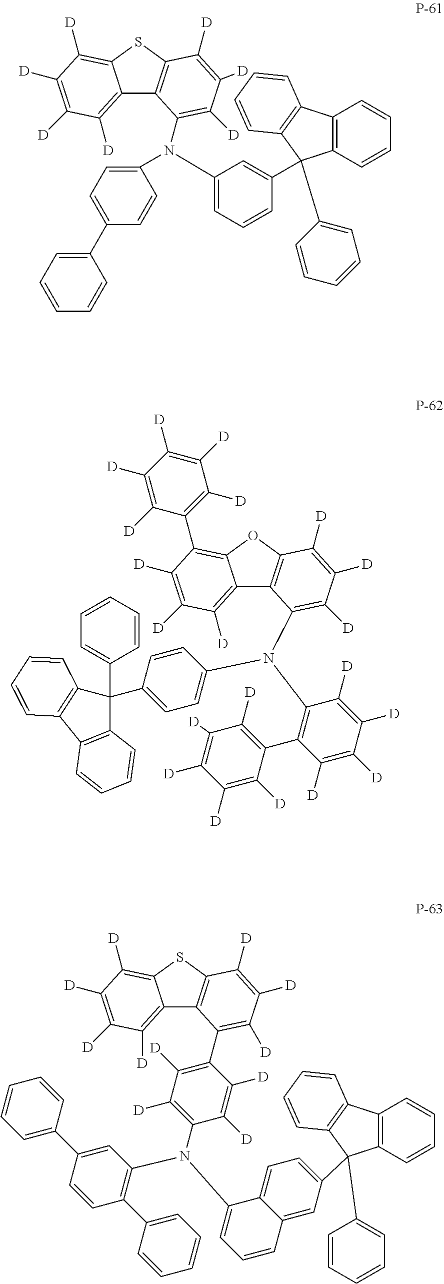

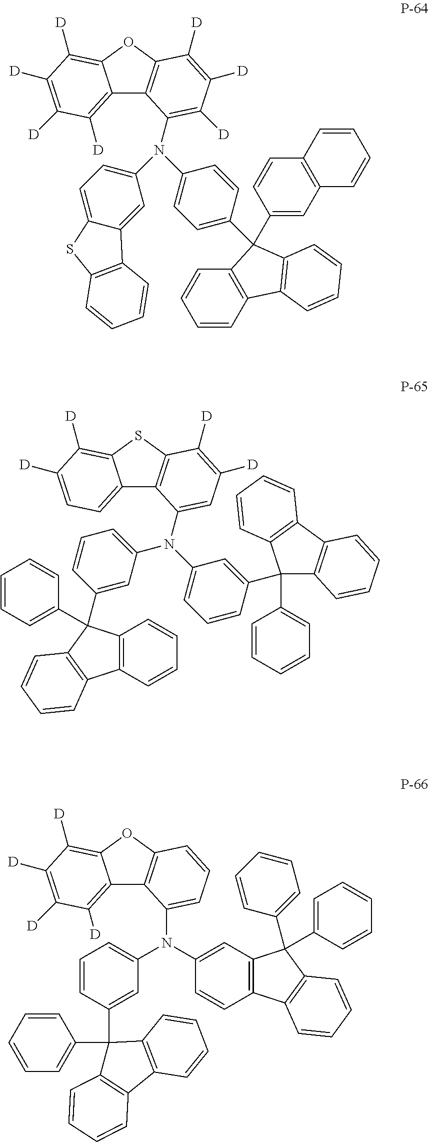 [SMILES: [2H]c1c([2H])c([2H])c2c(oc3c([2H])c([2H])c([2H])c(N(c4ccc(C5(c6ccc7ccccc7c6)c6ccccc6-c6ccccc65)cc4)c4ccc5sc6ccccc6c5c4)c32)c1[2H] ; [2H]c1ccc2c(sc3c([2H])c([2H])cc(N(c4cccc(C5(c6ccccc6)c6ccccc6-c6ccccc65)c4)c4cccc(C5(c6ccccc6)c6ccccc6-c6ccccc65)c4)c32)c1[2H] ; [2H]c1c([2H])c([2H])c2c(oc3cccc(N(c4cccc(C5(c6ccccc6)c6ccccc6-c6ccccc65)c4)c4ccc5c(c4)C(c4ccccc4)(c4ccccc4)c4ccccc4-5)c32)c1[2H]]

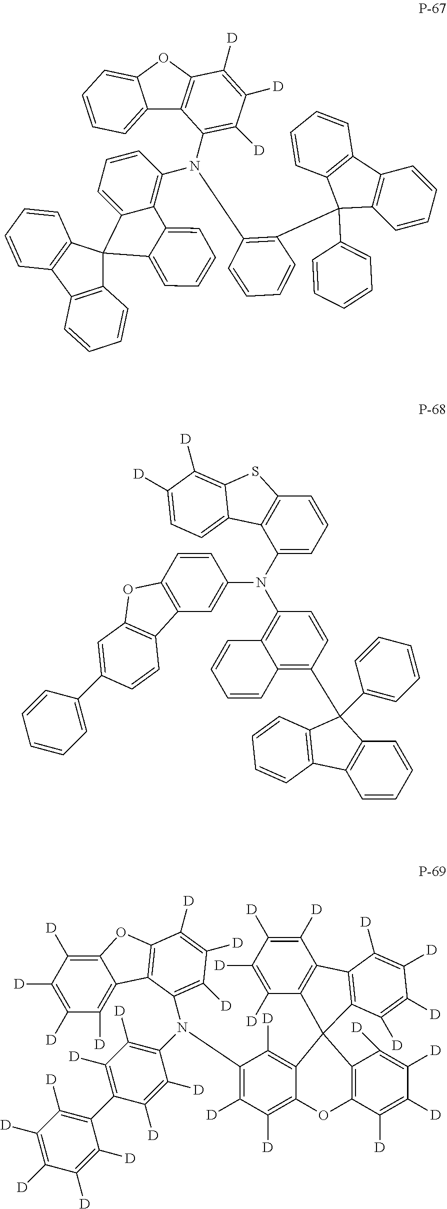 [SMILES: [2H]c1c([2H])c(N(c2ccccc2C2(c3ccccc3)c3ccccc3-c3ccccc32)c2cccc3c2-c2ccccc2C32c3ccccc3-c3ccccc32)c2c(oc3ccccc32)c1[2H] ; [2H]c1ccc2c(sc3cccc(N(c4ccc5oc6cc(-c7ccccc7)ccc6c5c4)c4ccc(C5(c6ccccc6)c6ccccc6-c6ccccc65)c5ccccc45)c32)c1[2H] ; [2H]c1c([2H])c([2H])c(-c2c([2H])c([2H])c(N(c3c([2H])c([2H])c4c(c3[2H])C3(c5c([2H])c([2H])c([2H])c([2H])c5O4)c4c([2H])c([2H])c([2H])c([2H])c4-c4c([2H])c([2H])c([2H])c([2H])c43)c3c([2H])c([2H])c([2H])c4oc5c([2H])c([2H])c([2H])c([2H])c5c34)c([2H])c2[2H])c([2H])c1[2H]]

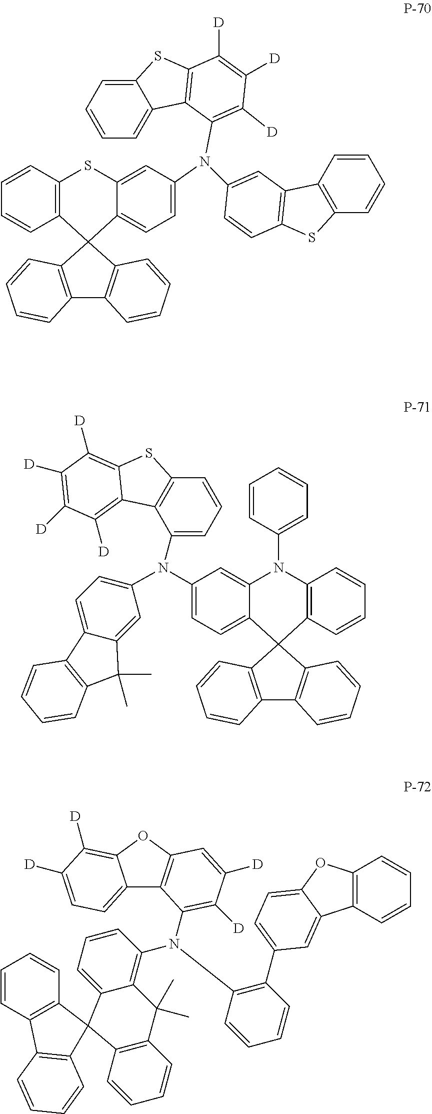 [SMILES: [2H]c1c([2H])c(N(c2ccc3c(c2)Sc2ccccc2C32c3ccccc3-c3ccccc32)c2ccc3sc4ccccc4c3c2)c2c(sc3ccccc32)c1[2H] ; [2H]c1c([2H])c([2H])c2c(sc3cccc(N(c4ccc5c(c4)N(c4ccccc4)c4ccccc4C54c5ccccc5-c5ccccc54)c4ccc5c(c4)C(C)(C)c4ccccc4-5)c32)c1[2H] ; [2H]c1cc2oc3c([2H])c([2H])ccc3c2c(N(c2ccccc2-c2ccc3oc4ccccc4c3c2)c2cccc3c2C(C)(C)c2ccccc2C32c3ccccc3-c3ccccc32)c1[2H]]

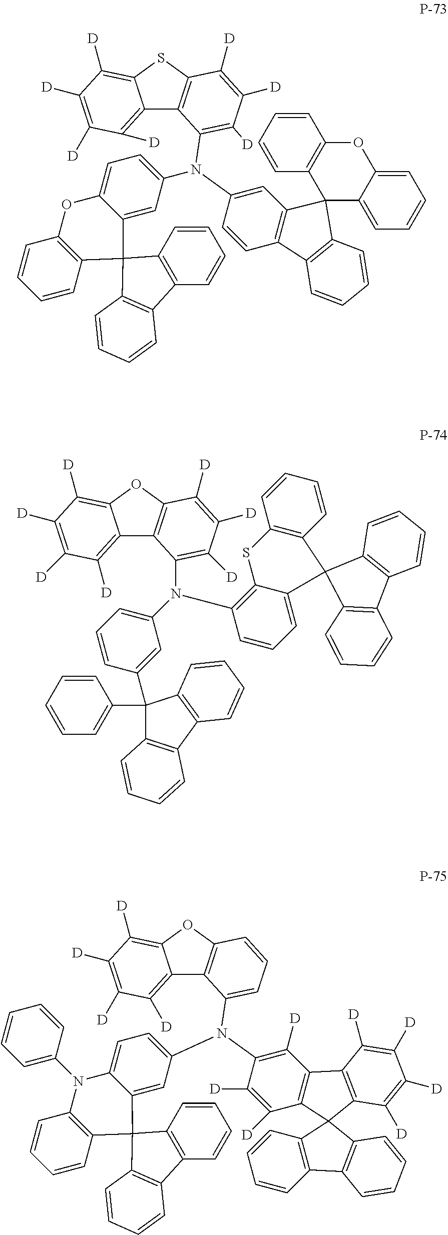 [SMILES: [2H]c1c([2H])c([2H])c2c(sc3c([2H])c([2H])c([2H])c(N(c4ccc5c(c4)C4(c6ccccc6O5)c5ccccc5-c5ccccc54)c4ccc5c(c4)C4(c6ccccc6Oc6ccccc64)c4ccccc4-5)c32)c1[2H] ; [2H]c1c([2H])c([2H])c2c(oc3c([2H])c([2H])c([2H])c(N(c4cccc(C5(c6ccccc6)c6ccccc6-c6ccccc65)c4)c4cccc5c4Sc4ccccc4C54c5ccccc5-c5ccccc54)c32)c1[2H] ; [2H]c1c([2H])c([2H])c2c(c1[2H])-c1c([2H])c(N(c3ccc4c(c3)C3(c5ccccc5-c5ccccc53)c3ccccc3N4c3ccccc3)c3cccc4oc5c([2H])c([2H])c([2H])c([2H])c5c34)c([2H])c([2H])c1C21c2ccccc2-c2ccccc21]

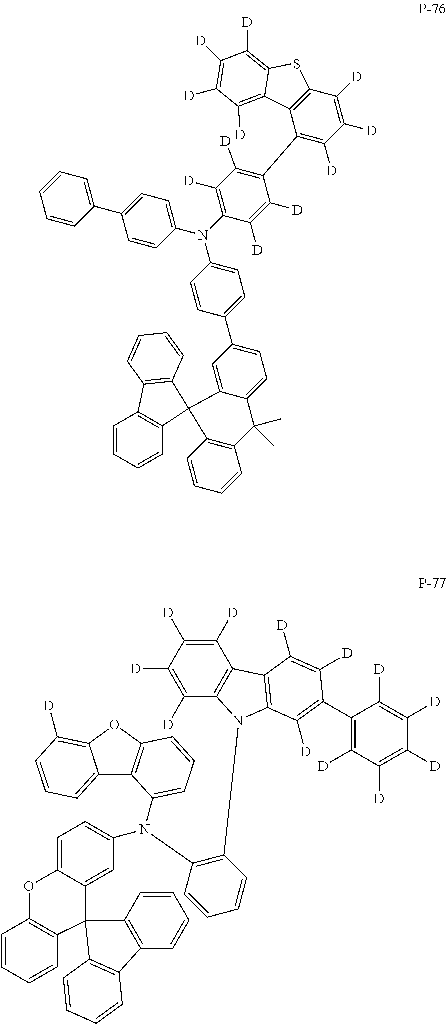 [SMILES: [2H]c1c([2H])c(N(c2ccc(-c3ccccc3)cc2)c2ccc(-c3ccc4c(c3)C3(c5ccccc5-c5ccccc53)c3ccccc3C4(C)C)cc2)c([2H])c([2H])c1-c1c([2H])c([2H])c([2H])c2sc3c([2H])c([2H])c([2H])c([2H])c3c12 ; [2H]c1c([2H])c([2H])c(-c2c([2H])c([2H])c3c4c([2H])c([2H])c([2H])c([2H])c4n(-c4ccccc4N(c4ccc5c(c4)C4(c6ccccc6O5)c5ccccc5-c5ccccc54)c4cccc5oc6c([2H])cccc6c45)c3c2[2H])c([2H])c1[2H]]

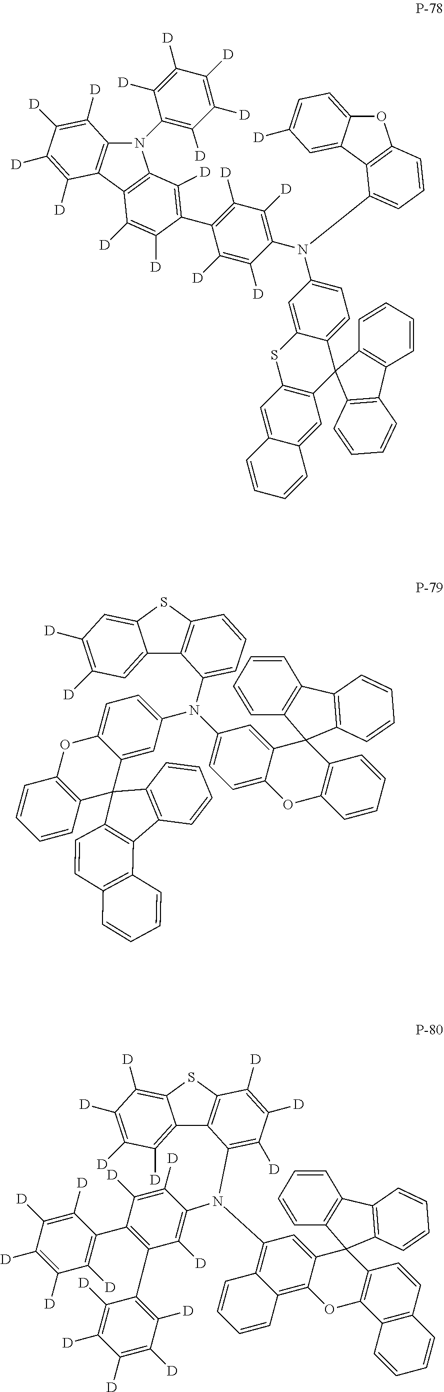

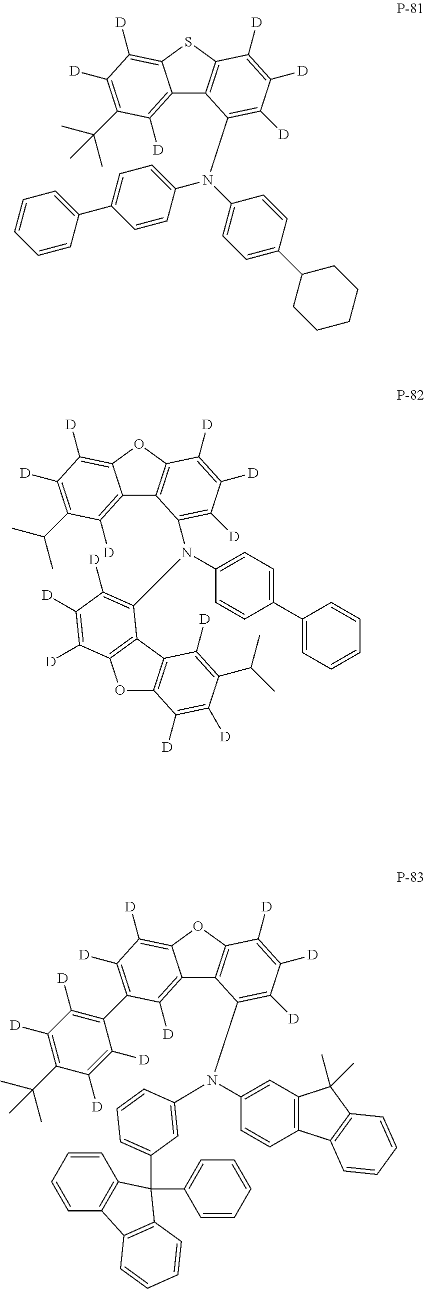 [SMILES: [2H]c1c(C(C)(C)C)c([2H])c2c(sc3c([2H])c([2H])c([2H])c(N(c4ccc(-c5ccccc5)cc4)c4ccc(C5CCCCC5)cc4)c32)c1[2H] ; [2H]c1c(C(C)C)c([2H])c2c(oc3c([2H])c([2H])c([2H])c(N(c4ccc(-c5ccccc5)cc4)c4c([2H])c([2H])c([2H])c5oc6c([2H])c([2H])c(C(C)C)c([2H])c6c45)c32)c1[2H] ; [2H]c1c([2H])c(C(C)(C)C)c([2H])c([2H])c1-c1c([2H])c([2H])c2oc3c([2H])c([2H])c([2H])c(N(c4cccc(C5(c6ccccc6)c6ccccc6-c6ccccc65)c4)c4ccc5c(c4)C(C)(C)c4ccccc4-5)c3c2c1[2H]]

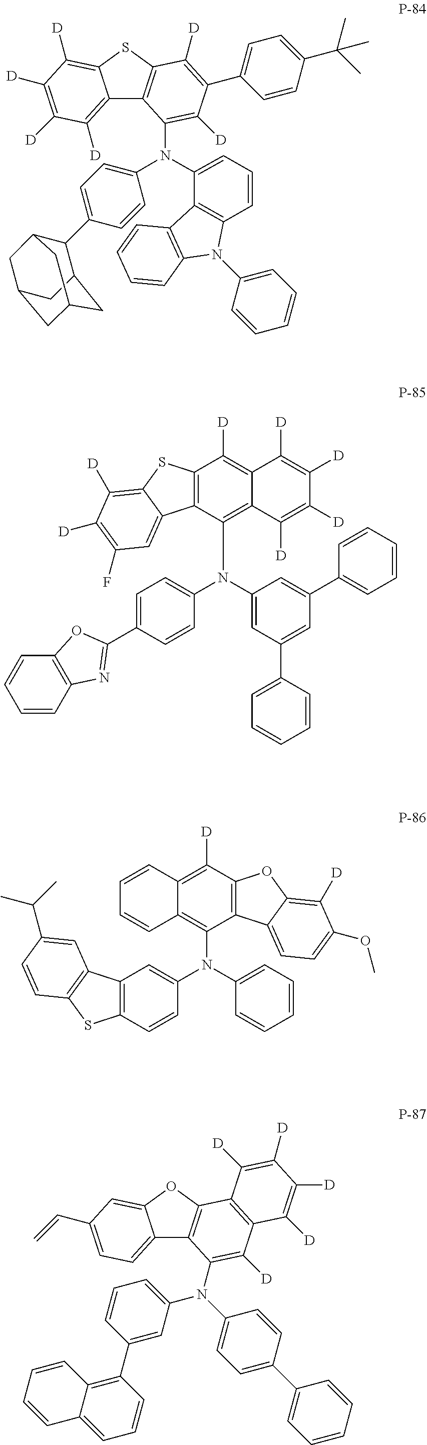

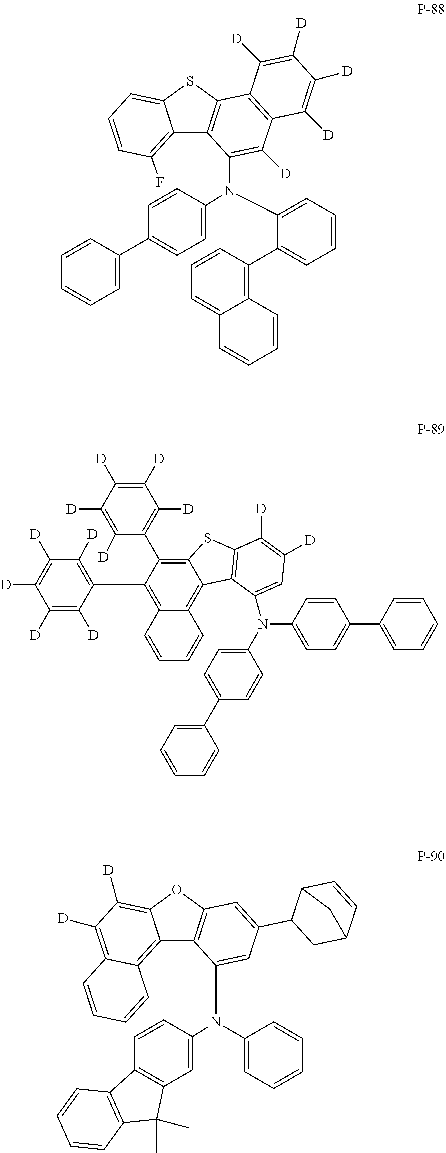 [SMILES: [2H]c1c([2H])c([2H])c2c(c1[2H])c([2H])c(N(c1ccc(-c3ccccc3)cc1)c1ccccc1-c1cccc3ccccc13)c1c2sc2cccc(F)c21 ; [2H]c1cc(N(c2ccc(-c3ccccc3)cc2)c2ccc(-c3ccccc3)cc2)c2c(sc3c(-c4c([2H])c([2H])c([2H])c([2H])c4[2H])c(-c4c([2H])c([2H])c([2H])c([2H])c4[2H])c4ccccc4c32)c1[2H] ; [2H]c1c([2H])c2oc3cc(C4CC5C=CC4C5)cc(N(c4ccccc4)c4ccc5c(c4)C(C)(C)c4ccccc4-5)c3c2c2ccccc12]

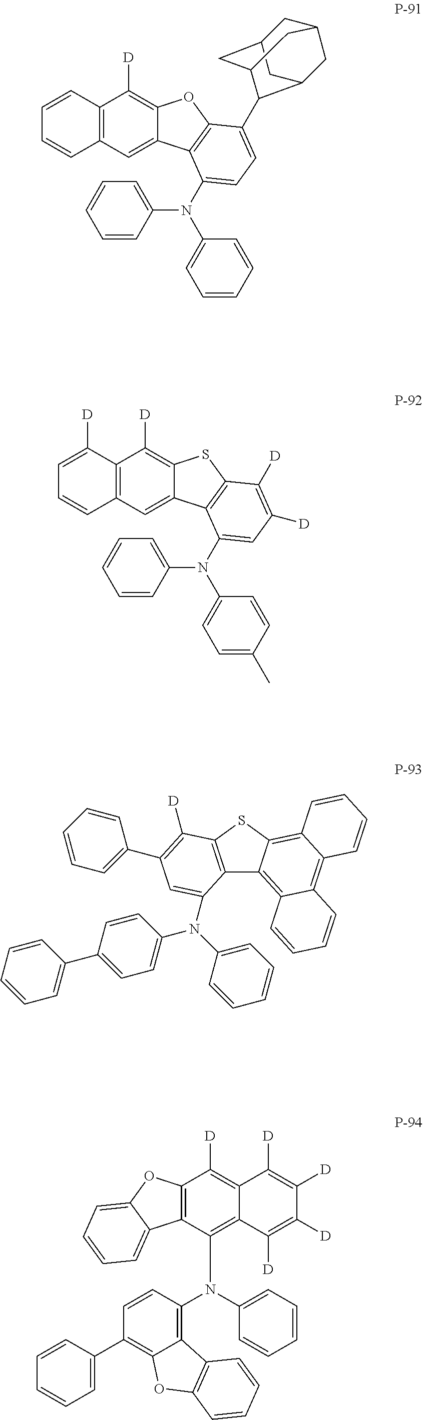 [SMILES: [2H]c1c2ccccc2cc2c1oc1c(C3C4CC5CC(C4)CC3C5)ccc(N(c3ccccc3)c3ccccc3)c12 ; [2H]c1cc(N(c2ccccc2)c2ccc(C)cc2)c2c(sc3c([2H])c4c([2H])cccc4cc32)c1[2H] ; [2H]c1c(-c2ccccc2)cc(N(c2ccccc2)c2ccc(-c3ccccc3)cc2)c2c1sc1c3ccccc3c3ccccc3c12 ; [2H]c1c([2H])c([2H])c2c(N(c3ccccc3)c3ccc(-c4ccccc4)c4oc5ccccc5c34)c3c(oc4ccccc43)c([2H])c2c1[2H]]

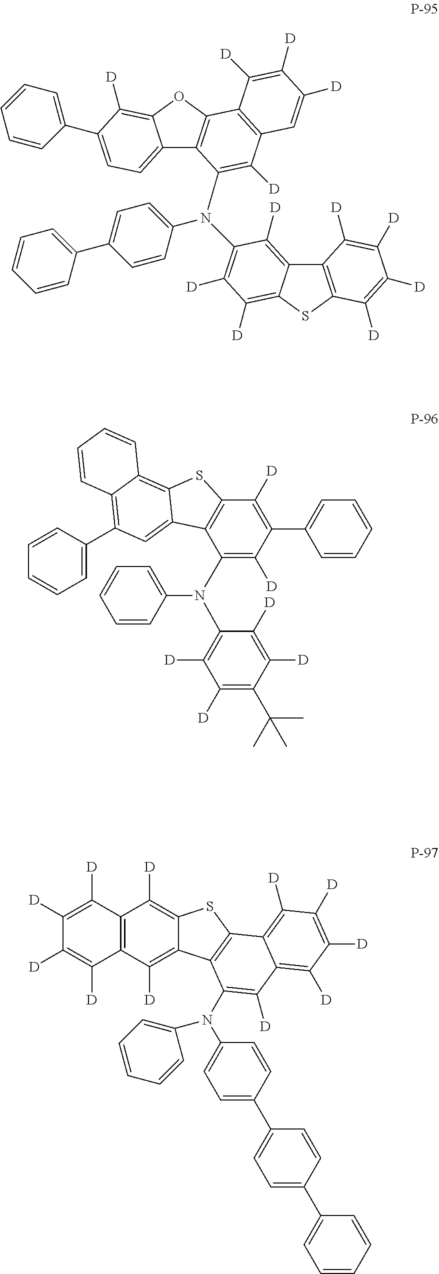 [SMILES: [2H]c1cc2c([2H])c(N(c3ccc(-c4ccccc4)cc3)c3c([2H])c([2H])c4sc5c([2H])c([2H])c([2H])c([2H])c5c4c3[2H])c3c4ccc(-c5ccccc5)c([2H])c4oc3c2c([2H])c1[2H] ; [2H]c1c([2H])c(C(C)(C)C)c([2H])c([2H])c1N(c1ccccc1)c1c([2H])c(-c2ccccc2)c([2H])c2sc3c4ccccc4c(-c4ccccc4)cc3c12 ; [2H]c1c([2H])c([2H])c2c([2H])c3c(sc4c5c([2H])c([2H])c([2H])c([2H])c5c([2H])c(N(c5ccccc5)c5ccc(-c6ccc(-c7ccccc7)cc6)cc5)c43)c([2H])c2c1[2H]]

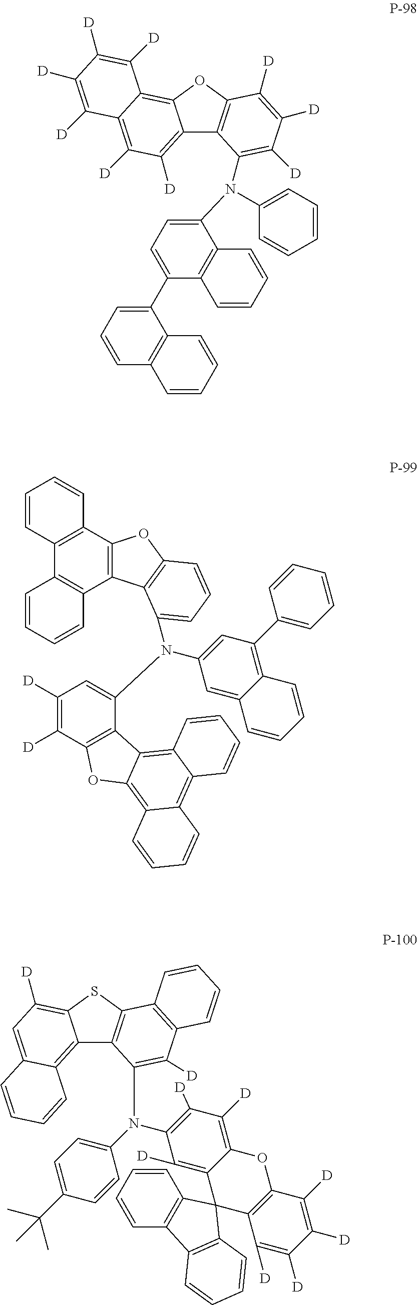 [SMILES: [2H]c1c([2H])c(N(c2ccccc2)c2ccc(-c3cccc4ccccc34)c3ccccc23)c2c(oc3c4c([2H])c([2H])c([2H])c([2H])c4c([2H])c([2H])c32)c1[2H] ; [2H]c1cc(N(c2cc(-c3ccccc3)c3ccccc3c2)c2cccc3oc4c5ccccc5c5ccccc5c4c23)c2c(oc3c4ccccc4c4ccccc4c32)c1[2H] ; [2H]c1c([2H])c([2H])c2c(c1[2H])Oc1c([2H])c([2H])c(N(c3ccc(C(C)(C)C)cc3)c3c([2H])c4ccccc4c4sc5c([2H])cc6ccccc6c5c34)c([2H])c1C21c2ccccc2-c2ccccc21]

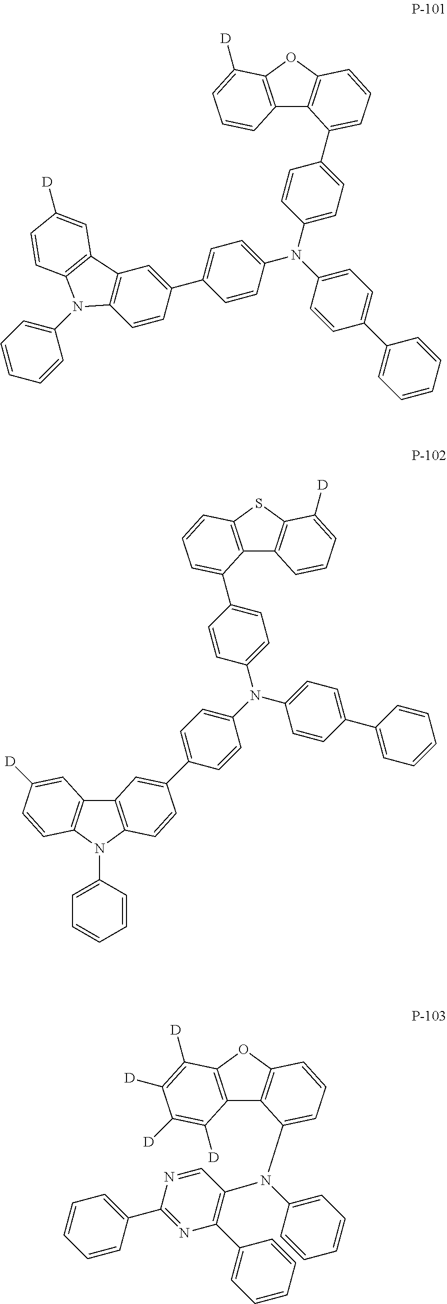 [SMILES: [2H]c1ccc2c(c1)c1cc(-c3ccc(N(c4ccc(-c5ccccc5)cc4)c4ccc(-c5cccc6oc7c([2H])cccc7c56)cc4)cc3)ccc1n2-c1ccccc1 ; [2H]c1ccc2c(c1)c1cc(-c3ccc(N(c4ccc(-c5ccccc5)cc4)c4ccc(-c5cccc6sc7c([2H])cccc7c56)cc4)cc3)ccc1n2-c1ccccc1 ; [2H]c1c([2H])c([2H])c2c(oc3cccc(N(c4ccccc4)c4cnc(-c5ccccc5)nc4-c4ccccc4)c32)c1[2H]]

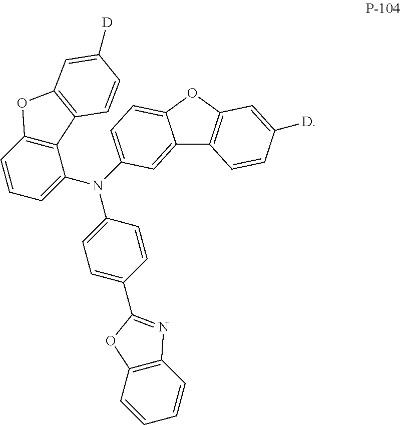

9. The organic electric element of claim 1, wherein the compound represented by Formula 2 is one of the following compounds:

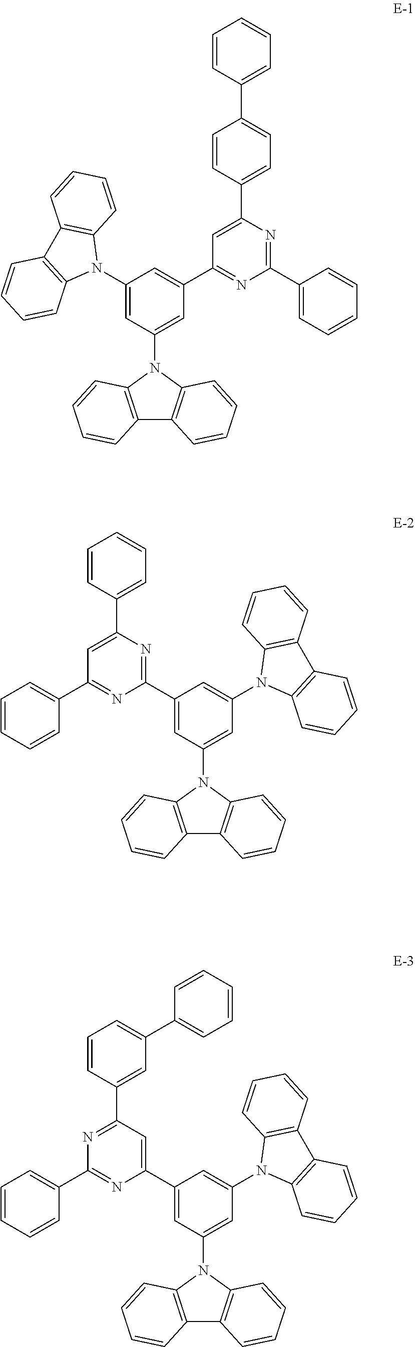 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cc(-c3ccccc3)nc(-c3cc(-n4c5ccccc5c5ccccc54)cc(-n4c5ccccc5c5ccccc54)c3)n2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

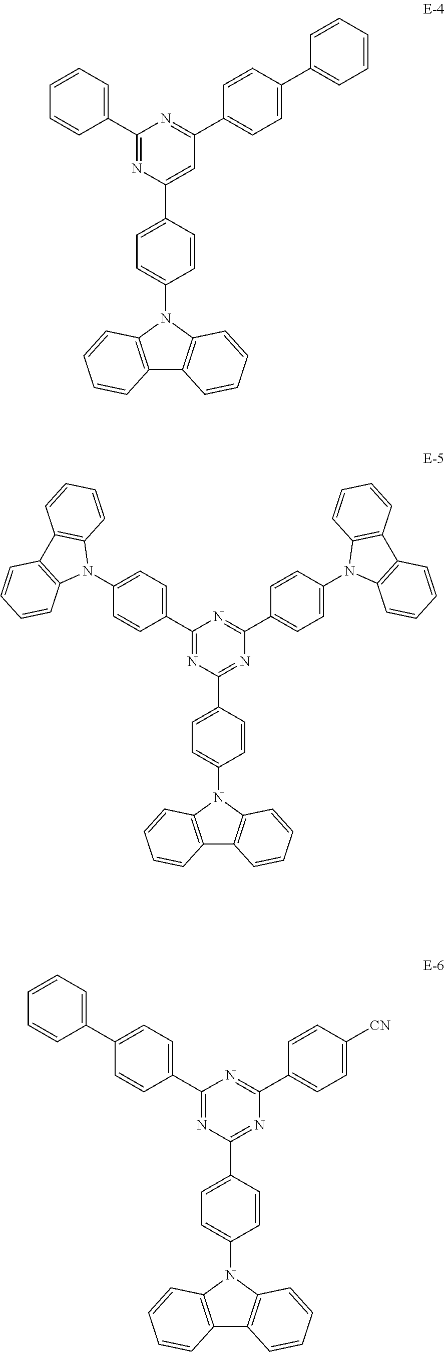 [SMILES: c1ccc(-c2ccc(-c3cc(-c4ccc(-n5c6ccccc6c6ccccc65)cc4)nc(-c4ccccc4)n3)cc2)cc1 ; c1ccc2c(c1)c1ccccc1n2-c1ccc(-c2nc(-c3ccc(-n4c5ccccc5c5ccccc54)cc3)nc(-c3ccc(-n4c5ccccc5c5ccccc54)cc3)n2)cc1 ; N#Cc1ccc(-c2nc(-c3ccc(-c4ccccc4)cc3)nc(-c3ccc(-n4c5ccccc5c5ccccc54)cc3)n2)cc1]

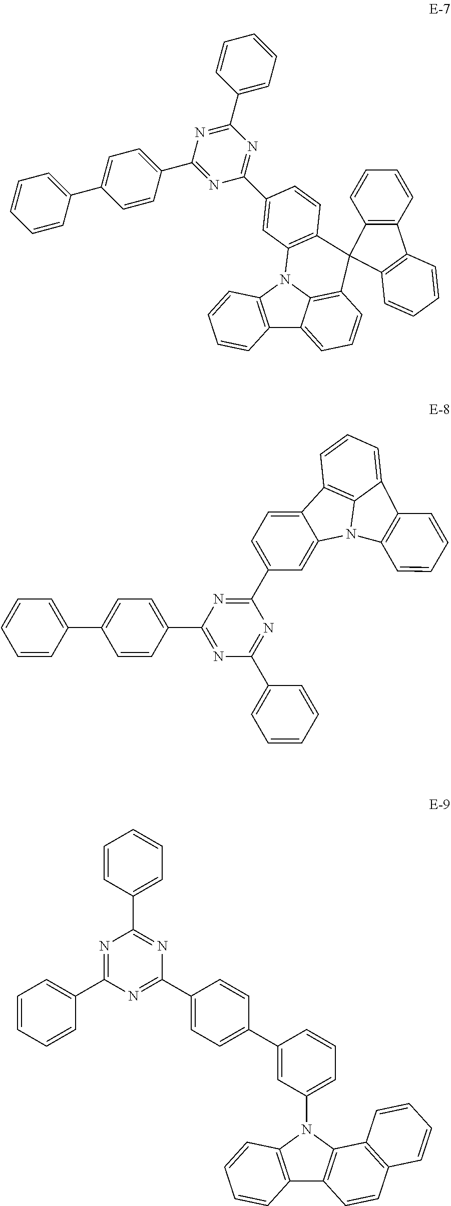 [SMILES: c1ccc(-c2ccc(-c3nc(-c4ccccc4)nc(-c4ccc5c(c4)-n4c6ccccc6c6cccc(c64)C54c5ccccc5-c5ccccc54)n3)cc2)cc1 ; c1ccc(-c2ccc(-c3nc(-c4ccccc4)nc(-c4ccc5c6cccc7c8ccccc8n(c5c4)c76)n3)cc2)cc1 ; c1ccc(-c2nc(-c3ccccc3)nc(-c3ccc(-c4cccc(-n5c6ccccc6c6ccc7ccccc7c65)c4)cc3)n2)cc1]

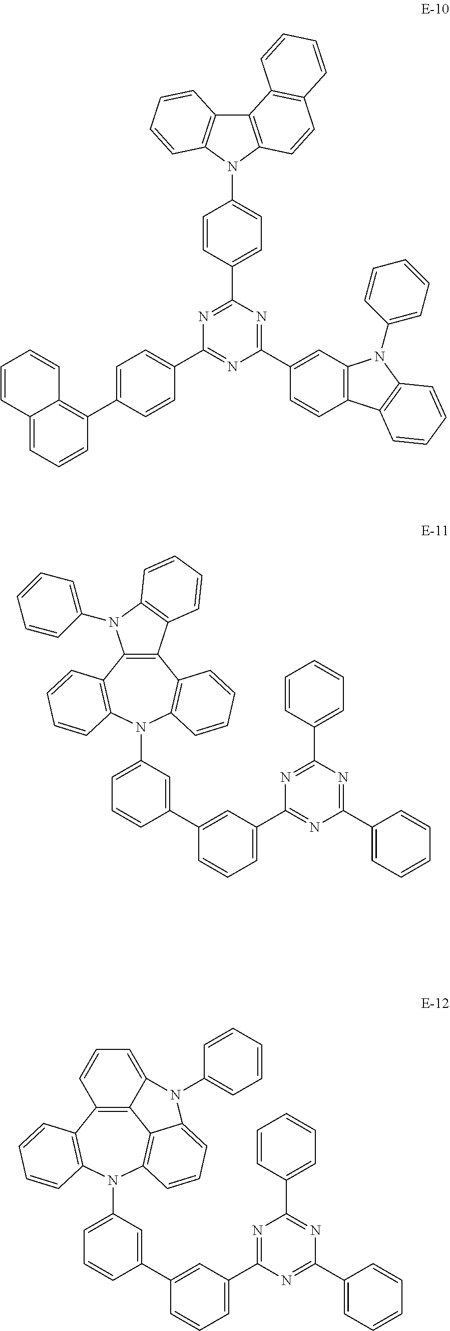 [SMILES: c1ccc(-n2c3ccccc3c3ccc(-c4nc(-c5ccc(-c6cccc7ccccc67)cc5)nc(-c5ccc(-n6c7ccccc7c7c8ccccc8ccc76)cc5)n4)cc32)cc1 ; c1ccc(-c2nc(-c3ccccc3)nc(-c3cccc(-c4cccc(N5c6ccccc6-c6c(n(-c7ccccc7)c7ccccc67)-c6ccccc65)c4)c3)n2)cc1 ; c1ccc(-c2nc(-c3ccccc3)nc(-c3cccc(-c4cccc(-n5c6ccccc6c6cccc7c6c6c5cccc6n7-c5ccccc5)c4)c3)n2)cc1]

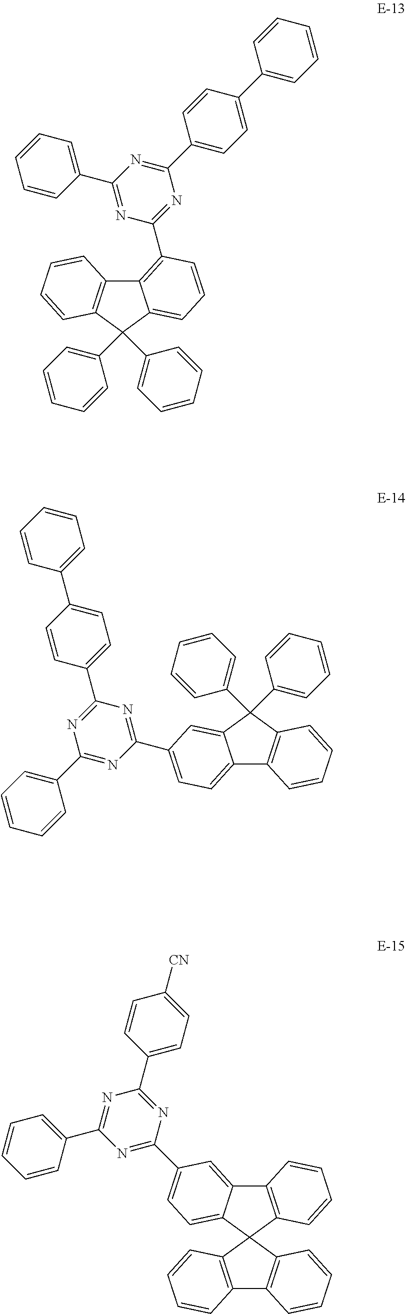 [SMILES: c1ccc(-c2ccc(-c3nc(-c4ccccc4)nc(-c4cccc5c4-c4ccccc4C5(c4ccccc4)c4ccccc4)n3)cc2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1 ; N#Cc1ccc(-c2nc(-c3ccccc3)nc(-c3ccc4c(c3)-c3ccccc3C43c4ccccc4-c4ccccc43)n2)cc1]

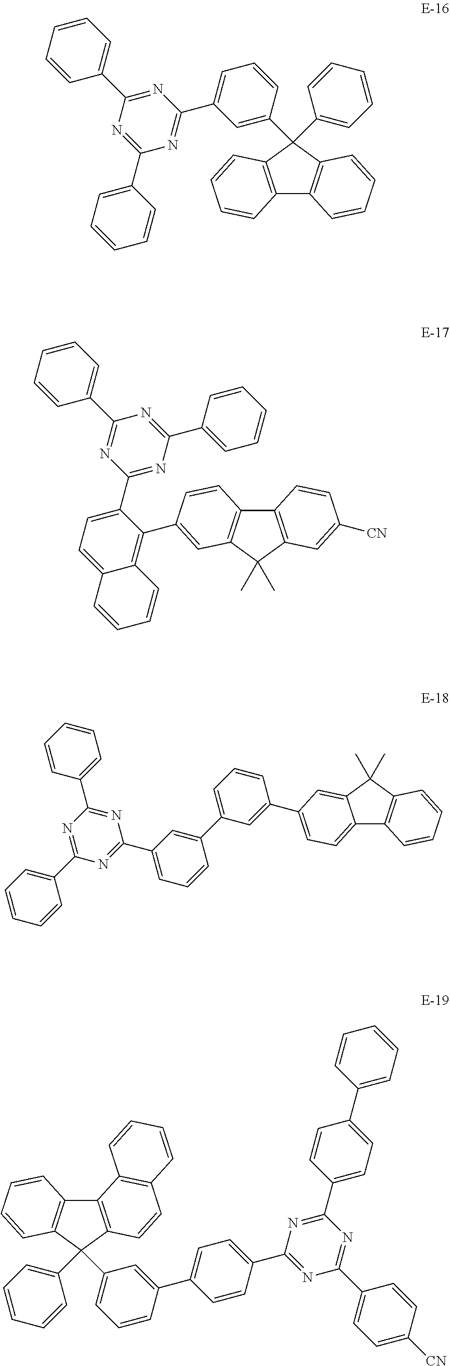 [SMILES: c1ccc(-c2nc(-c3ccccc3)nc(-c3cccc(C4(c5ccccc5)c5ccccc5-c5ccccc54)c3)n2)cc1 ; CC1(C)c2cc(C#N)ccc2-c2ccc(-c3c(-c4nc(-c5ccccc5)nc(-c5ccccc5)n4)ccc4ccccc34)cc21 ; CC1(C)c2ccccc2-c2ccc(-c3cccc(-c4cccc(-c5nc(-c6ccccc6)nc(-c6ccccc6)n5)c4)c3)cc21 ; N#Cc1ccc(-c2nc(-c3ccc(-c4ccccc4)cc3)nc(-c3ccc(-c4cccc(C5(c6ccccc6)c6ccccc6-c6c5ccc5ccccc65)c4)cc3)n2)cc1]

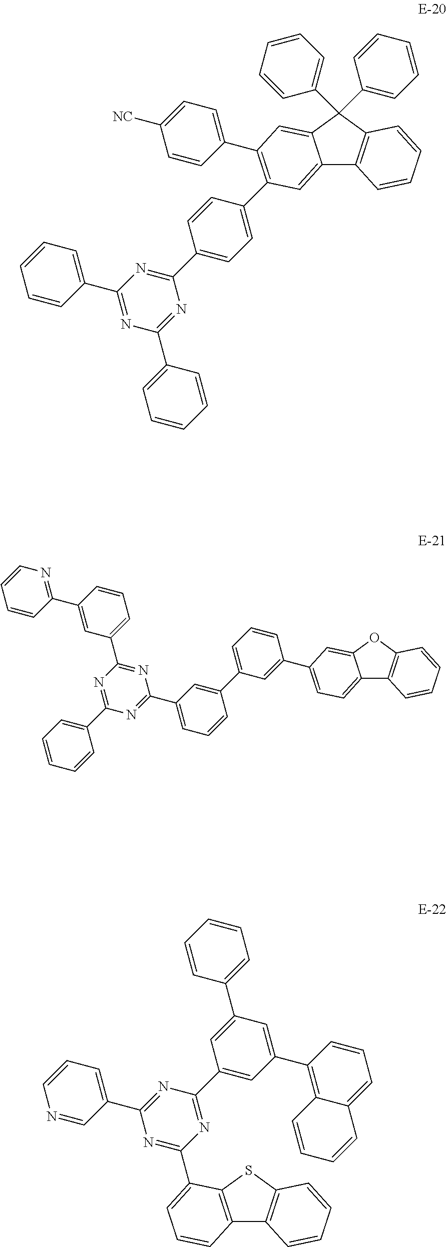 [SMILES: N#Cc1ccc(-c2cc3c(cc2-c2ccc(-c4nc(-c5ccccc5)nc(-c5ccccc5)n4)cc2)-c2ccccc2C3(c2ccccc2)c2ccccc2)cc1 ; c1ccc(-c2nc(-c3cccc(-c4cccc(-c5ccc6c(c5)oc5ccccc56)c4)c3)nc(-c3cccc(-c4ccccn4)c3)n2)cc1 ; c1ccc(-c2cc(-c3nc(-c4cccnc4)nc(-c4cccc5c4sc4ccccc45)n3)cc(-c3cccc4ccccc34)c2)cc1]

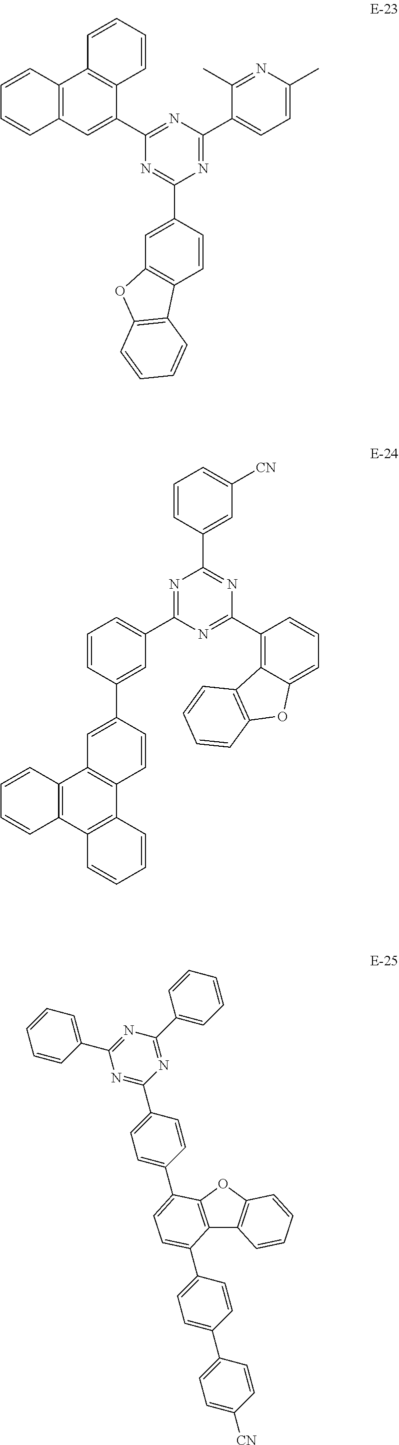 [SMILES: Cc1ccc(-c2nc(-c3ccc4c(c3)oc3ccccc34)nc(-c3cc4ccccc4c4ccccc34)n2)c(C)n1 ; N#Cc1cccc(-c2nc(-c3cccc(-c4ccc5c6ccccc6c6ccccc6c5c4)c3)nc(-c3cccc4oc5ccccc5c34)n2)c1 ; N#Cc1ccc(-c2ccc(-c3ccc(-c4ccc(-c5nc(-c6ccccc6)nc(-c6ccccc6)n5)cc4)c4oc5ccccc5c34)cc2)cc1]

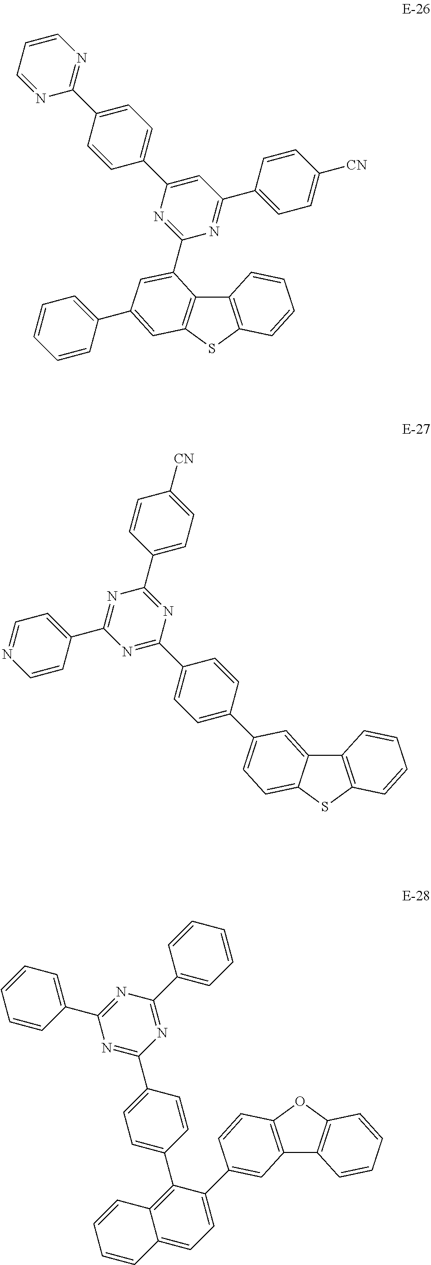 [SMILES: N#Cc1ccc(-c2cc(-c3ccc(-c4ncccn4)cc3)nc(-c3cc(-c4ccccc4)cc4sc5ccccc5c34)n2)cc1 ; N#Cc1ccc(-c2nc(-c3ccncc3)nc(-c3ccc(-c4ccc5sc6ccccc6c5c4)cc3)n2)cc1 ; c1ccc(-c2nc(-c3ccccc3)nc(-c3ccc(-c4c(-c5ccc6oc7ccccc7c6c5)ccc5ccccc45)cc3)n2)cc1]

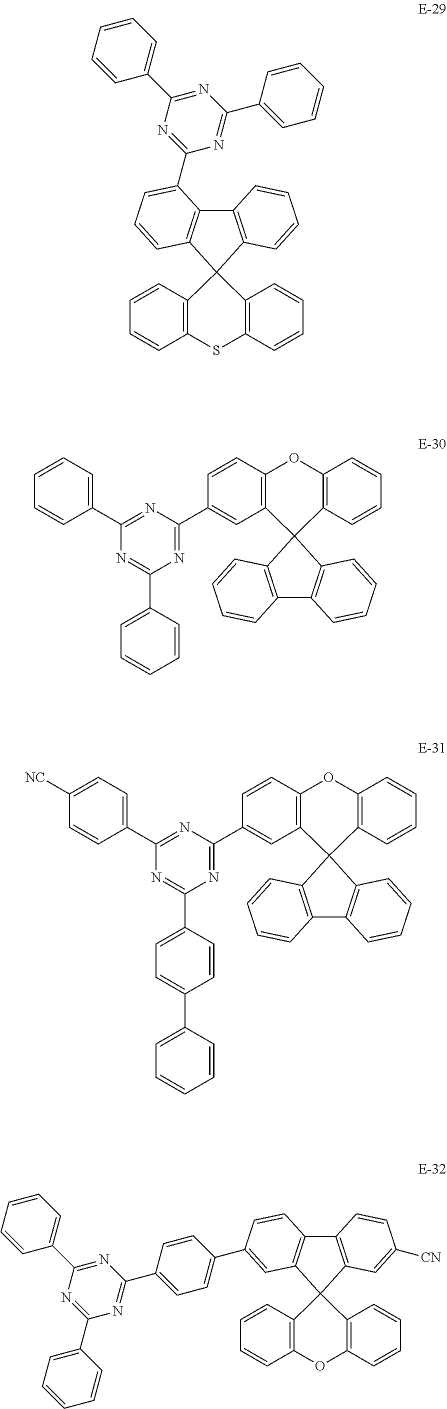 [SMILES: c1ccc(-c2nc(-c3ccccc3)nc(-c3cccc4c3-c3ccccc3C43c4ccccc4Sc4ccccc43)n2)cc1 ; c1ccc(-c2nc(-c3ccccc3)nc(-c3ccc4c(c3)C3(c5ccccc5O4)c4ccccc4-c4ccccc43)n2)cc1 ; N#Cc1ccc(-c2nc(-c3ccc(-c4ccccc4)cc3)nc(-c3ccc4c(c3)C3(c5ccccc5O4)c4ccccc4-c4ccccc43)n2)cc1 ; N#Cc1ccc2c(c1)C1(c3ccccc3Oc3ccccc31)c1cc(-c3ccc(-c4nc(-c5ccccc5)nc(-c5ccccc5)n4)cc3)ccc1-2]

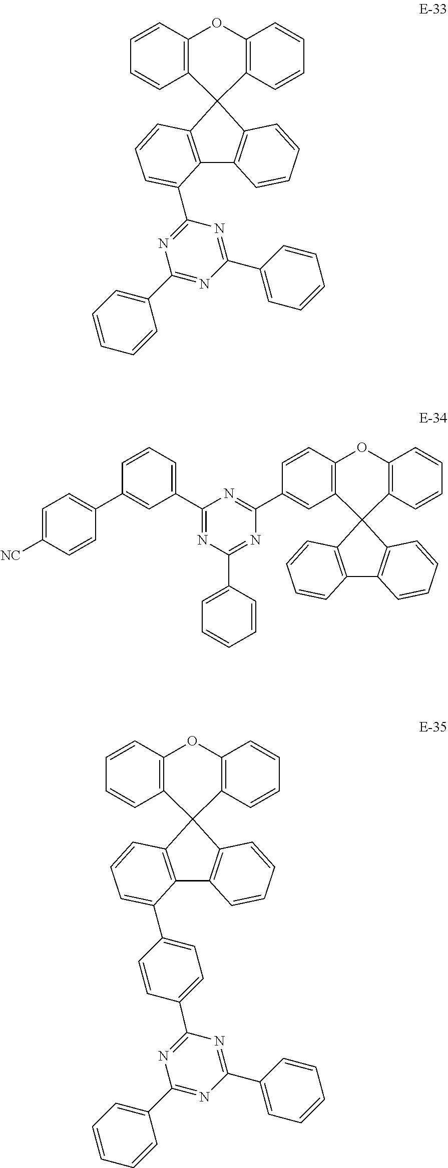 [SMILES: c1ccc(-c2nc(-c3ccccc3)nc(-c3cccc4c3-c3ccccc3C43c4ccccc4Oc4ccccc43)n2)cc1 ; N#Cc1ccc(-c2cccc(-c3nc(-c4ccccc4)nc(-c4ccc5c(c4)C4(c6ccccc6O5)c5ccccc5-c5ccccc54)n3)c2)cc1 ; c1ccc(-c2nc(-c3ccccc3)nc(-c3ccc(-c4cccc5c4-c4ccccc4C54c5ccccc5Oc5ccccc54)cc3)n2)cc1]

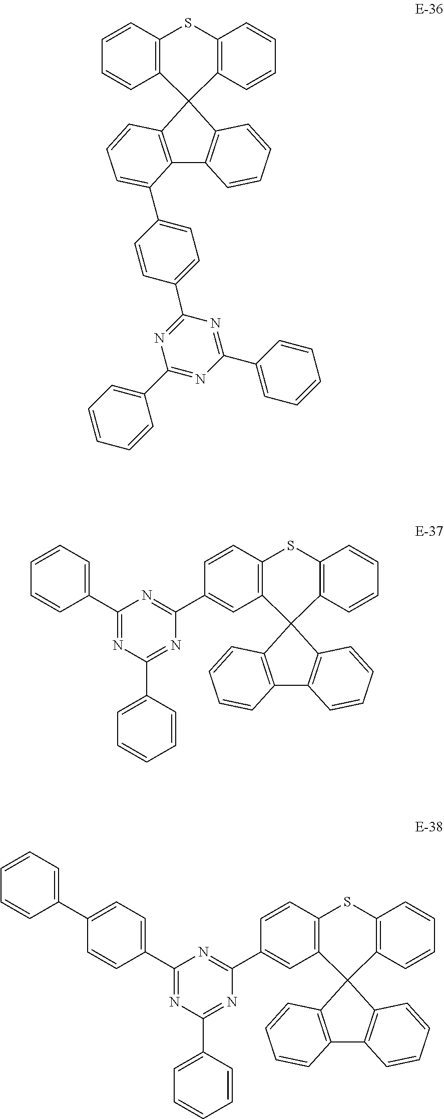 [SMILES: c1ccc(-c2nc(-c3ccccc3)nc(-c3ccc(-c4cccc5c4-c4ccccc4C54c5ccccc5Sc5ccccc54)cc3)n2)cc1 ; c1ccc(-c2nc(-c3ccccc3)nc(-c3ccc4c(c3)C3(c5ccccc5S4)c4ccccc4-c4ccccc43)n2)cc1 ; c1ccc(-c2ccc(-c3nc(-c4ccccc4)nc(-c4ccc5c(c4)C4(c6ccccc6S5)c5ccccc5-c5ccccc54)n3)cc2)cc1]

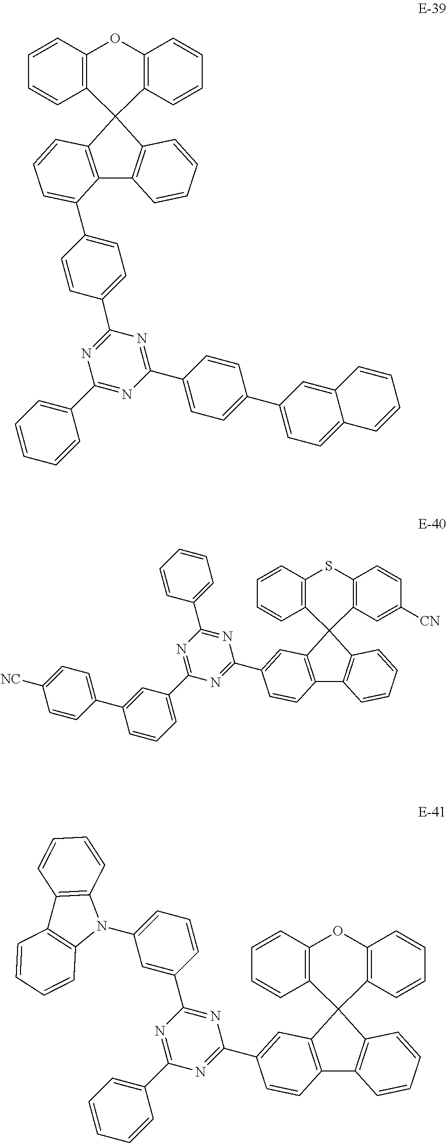 [SMILES: c1ccc(-c2nc(-c3ccc(-c4ccc5ccccc5c4)cc3)nc(-c3ccc(-c4cccc5c4-c4ccccc4C54c5ccccc5Oc5ccccc54)cc3)n2)cc1 ; N#Cc1ccc(-c2cccc(-c3nc(-c4ccccc4)nc(-c4ccc5c(c4)C4(c6ccccc6Sc6ccc(C#N)cc64)c4ccccc4-5)n3)c2)cc1 ; c1ccc(-c2nc(-c3cccc(-n4c5ccccc5c5ccccc54)c3)nc(-c3ccc4c(c3)C3(c5ccccc5Oc5ccccc53)c3ccccc3-4)n2)cc1]

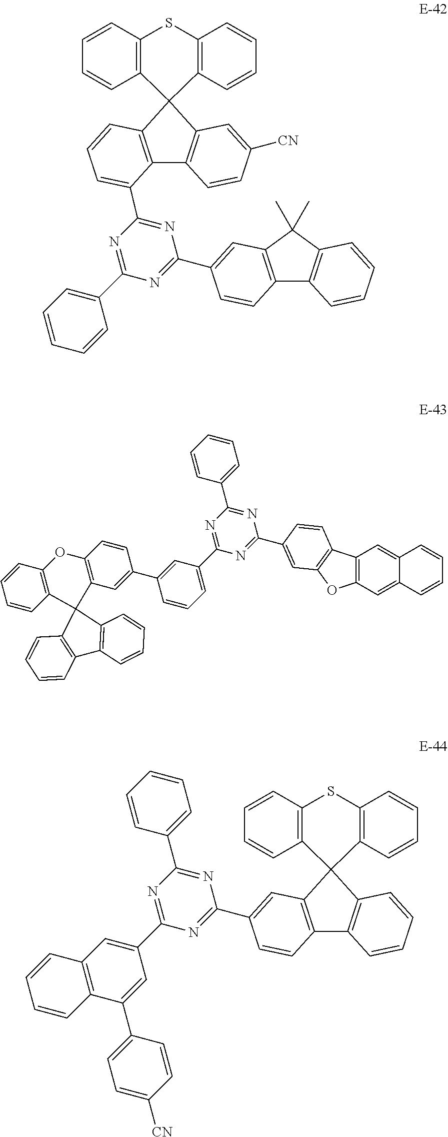 [SMILES: CC1(C)c2ccccc2-c2ccc(-c3nc(-c4ccccc4)nc(-c4cccc5c4-c4ccc(C#N)cc4C54c5ccccc5Sc5ccccc54)n3)cc21 ; c1ccc(-c2nc(-c3cccc(-c4ccc5c(c4)C4(c6ccccc6O5)c5ccccc5-c5ccccc54)c3)nc(-c3ccc4c(c3)oc3cc5ccccc5cc34)n2)cc1 ; N#Cc1ccc(-c2cc(-c3nc(-c4ccccc4)nc(-c4ccc5c(c4)C4(c6ccccc6Sc6ccccc64)c4ccccc4-5)n3)cc3ccccc23)cc1]

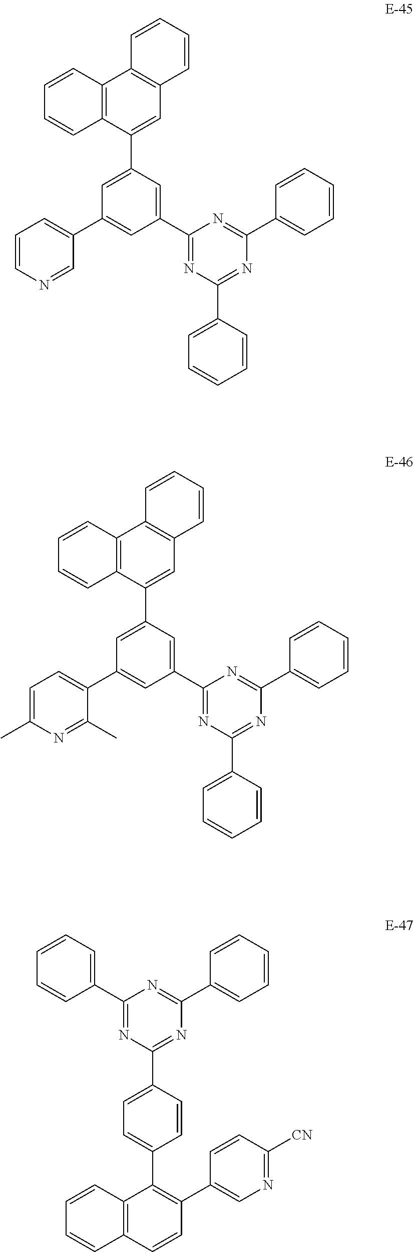 [SMILES: c1ccc(-c2nc(-c3ccccc3)nc(-c3cc(-c4cccnc4)cc(-c4cc5ccccc5c5ccccc45)c3)n2)cc1 ; Cc1ccc(-c2cc(-c3nc(-c4ccccc4)nc(-c4ccccc4)n3)cc(-c3cc4ccccc4c4ccccc34)c2)c(C)n1 ; N#Cc1ccc(-c2ccc3ccccc3c2-c2ccc(-c3nc(-c4ccccc4)nc(-c4ccccc4)n3)cc2)cn1]

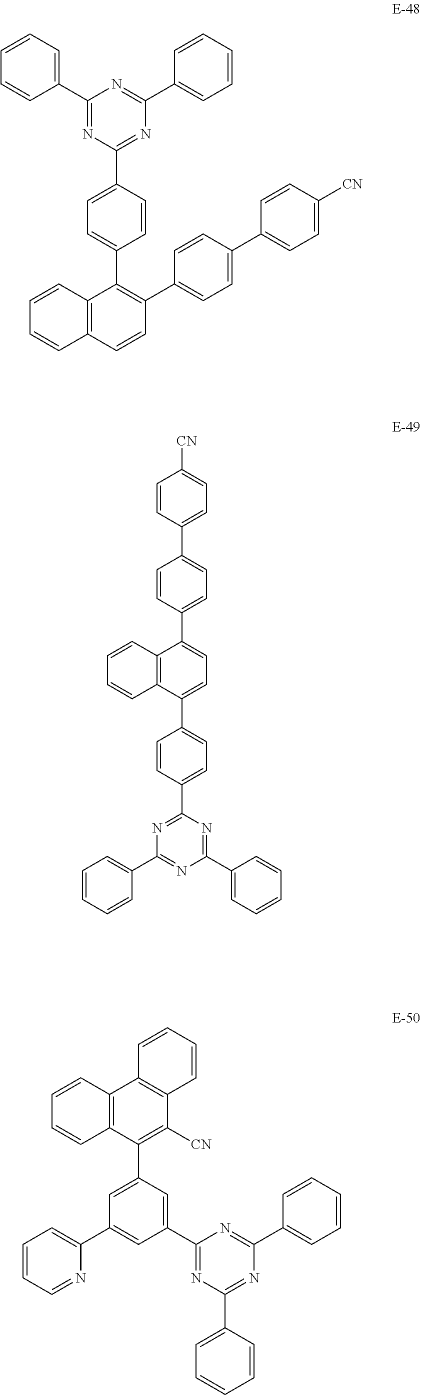 [SMILES: N#Cc1ccc(-c2ccc(-c3ccc4ccccc4c3-c3ccc(-c4nc(-c5ccccc5)nc(-c5ccccc5)n4)cc3)cc2)cc1 ; N#Cc1ccc(-c2ccc(-c3ccc(-c4ccc(-c5nc(-c6ccccc6)nc(-c6ccccc6)n5)cc4)c4ccccc34)cc2)cc1 ; N#Cc1c(-c2cc(-c3ccccn3)cc(-c3nc(-c4ccccc4)nc(-c4ccccc4)n3)c2)c2ccccc2c2ccccc12]

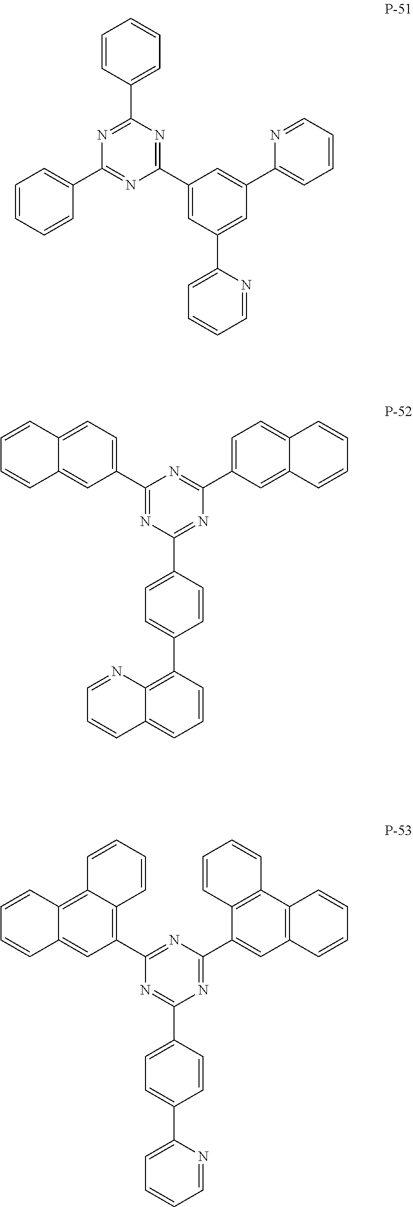 [SMILES: c1ccc(-c2nc(-c3ccccc3)nc(-c3cc(-c4ccccn4)cc(-c4ccccn4)c3)n2)cc1 ; c1ccc2cc(-c3nc(-c4ccc(-c5cccc6cccnc56)cc4)nc(-c4ccc5ccccc5c4)n3)ccc2c1 ; c1ccc(-c2ccc(-c3nc(-c4cc5ccccc5c5ccccc45)nc(-c4cc5ccccc5c5ccccc45)n3)cc2)nc1]

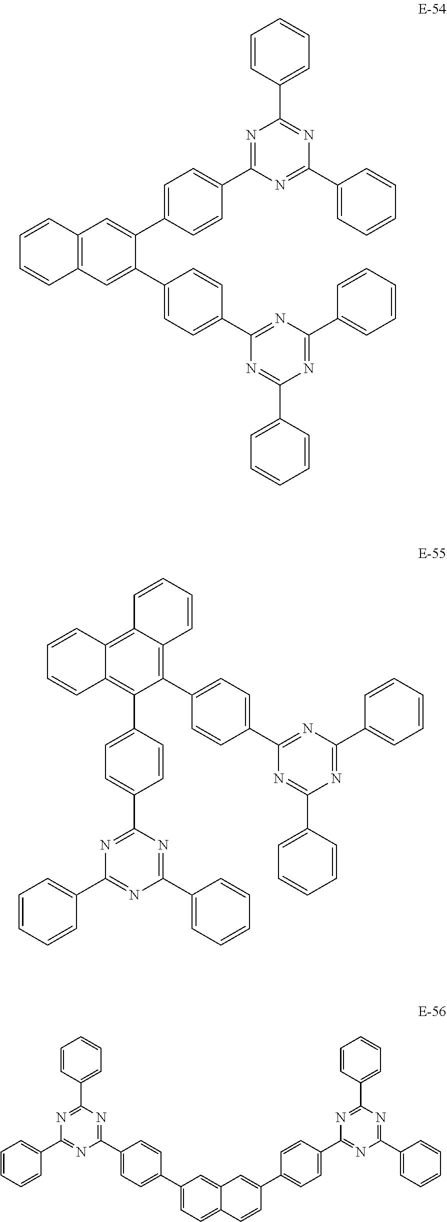 [SMILES: c1ccc(-c2nc(-c3ccccc3)nc(-c3ccc(-c4cc5ccccc5cc4-c4ccc(-c5nc(-c6ccccc6)nc(-c6ccccc6)n5)cc4)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

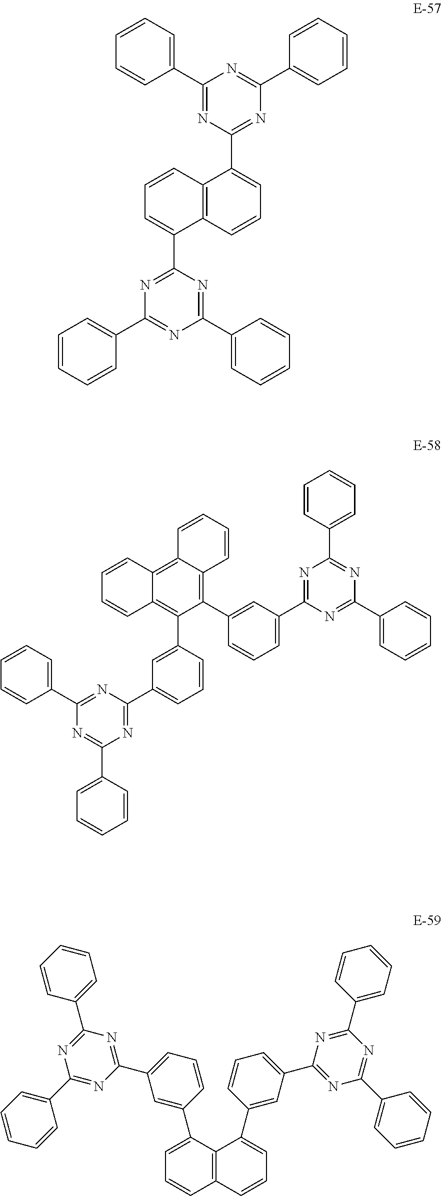 [SMILES: c1ccc(-c2nc(-c3ccccc3)nc(-c3cccc4c(-c5nc(-c6ccccc6)nc(-c6ccccc6)n5)cccc34)n2)cc1 ; c1ccc(-c2nc(-c3ccccc3)nc(-c3cccc(-c4c(-c5cccc(-c6nc(-c7ccccc7)nc(-c7ccccc7)n6)c5)c5ccccc5c5ccccc45)c3)n2)cc1 ; c1ccc(-c2nc(-c3ccccc3)nc(-c3cccc(-c4cccc5cccc(-c6cccc(-c7nc(-c8ccccc8)nc(-c8ccccc8)n7)c6)c45)c3)n2)cc1]

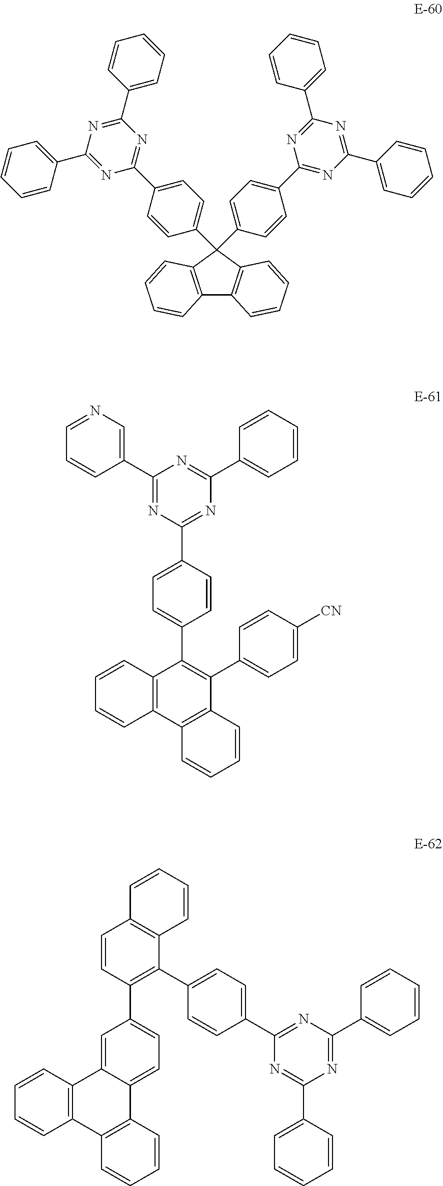 [SMILES: c1ccc(-c2nc(-c3ccccc3)nc(-c3ccc(C4(c5ccc(-c6nc(-c7ccccc7)nc(-c7ccccc7)n6)cc5)c5ccccc5-c5ccccc54)cc3)n2)cc1 ; N#Cc1ccc(-c2c(-c3ccc(-c4nc(-c5ccccc5)nc(-c5cccnc5)n4)cc3)c3ccccc3c3ccccc23)cc1 ; c1ccc(-c2nc(-c3ccccc3)nc(-c3ccc(-c4c(-c5ccc6c7ccccc7c7ccccc7c6c5)ccc5ccccc45)cc3)n2)cc1]

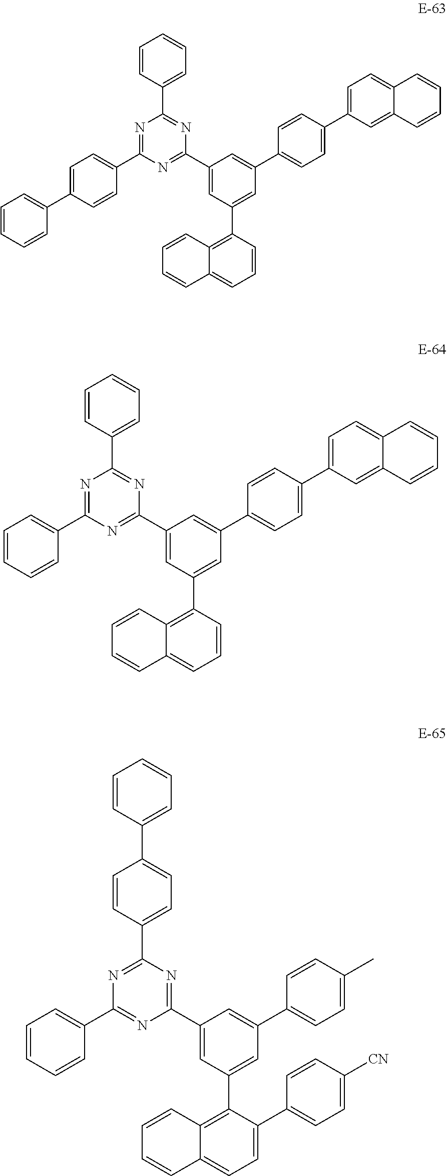 [SMILES: c1ccc(-c2ccc(-c3nc(-c4ccccc4)nc(-c4cc(-c5ccc(-c6ccc7ccccc7c6)cc5)cc(-c5cccc6ccccc56)c4)n3)cc2)cc1 ; c1ccc(-c2nc(-c3ccccc3)nc(-c3cc(-c4ccc(-c5ccc6ccccc6c5)cc4)cc(-c4cccc5ccccc45)c3)n2)cc1 ; Cc1ccc(-c2cc(-c3nc(-c4ccccc4)nc(-c4ccc(-c5ccccc5)cc4)n3)cc(-c3c(-c4ccc(C#N)cc4)ccc4ccccc34)c2)cc1]

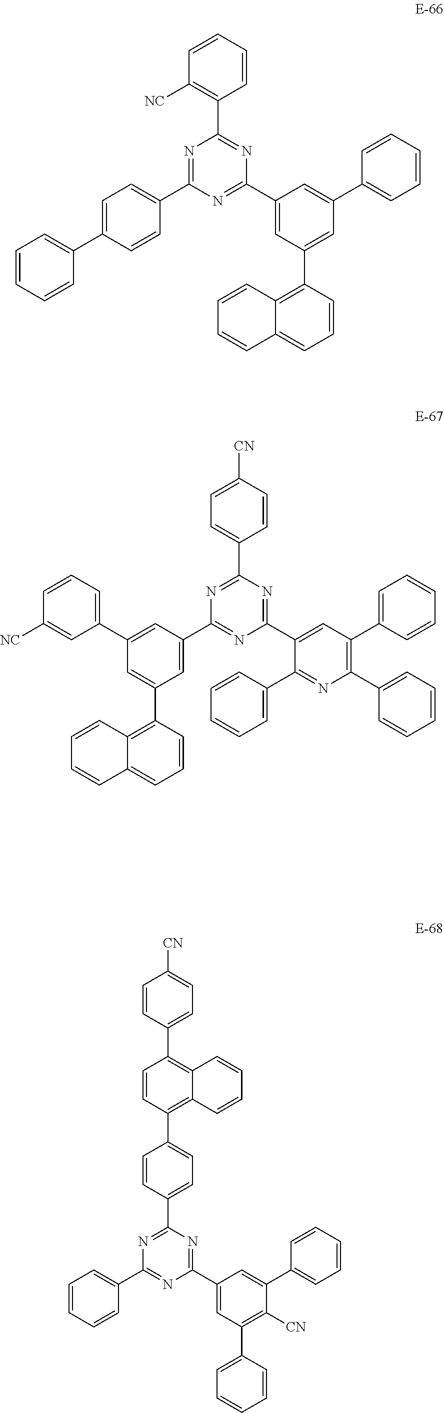 [SMILES: N#Cc1ccccc1-c1nc(-c2ccc(-c3ccccc3)cc2)nc(-c2cc(-c3ccccc3)cc(-c3cccc4ccccc34)c2)n1 ; N#Cc1ccc(-c2nc(-c3cc(-c4cccc(C#N)c4)cc(-c4cccc5ccccc45)c3)nc(-c3cc(-c4ccccc4)c(-c4ccccc4)nc3-c3ccccc3)n2)cc1 ; N#Cc1ccc(-c2ccc(-c3ccc(-c4nc(-c5ccccc5)nc(-c5cc(-c6ccccc6)c(C#N)c(-c6ccccc6)c5)n4)cc3)c3ccccc23)cc1]

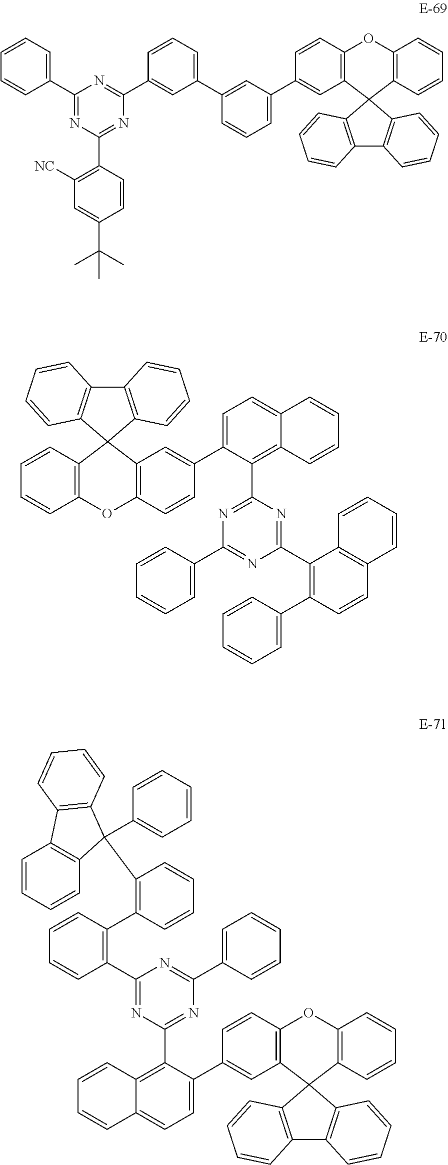 [SMILES: CC(C)(C)c1ccc(-c2nc(-c3ccccc3)nc(-c3cccc(-c4cccc(-c5ccc6c(c5)C5(c7ccccc7O6)c6ccccc6-c6ccccc65)c4)c3)n2)c(C#N)c1 ; c1ccc(-c2nc(-c3c(-c4ccccc4)ccc4ccccc34)nc(-c3c(-c4ccc5c(c4)C4(c6ccccc6O5)c5ccccc5-c5ccccc54)ccc4ccccc34)n2)cc1 ; c1ccc(-c2nc(-c3ccccc3-c3ccccc3C3(c4ccccc4)c4ccccc4-c4ccccc43)nc(-c3c(-c4ccc5c(c4)C4(c6ccccc6O5)c5ccccc5-c5ccccc54)ccc4ccccc34)n2)cc1]

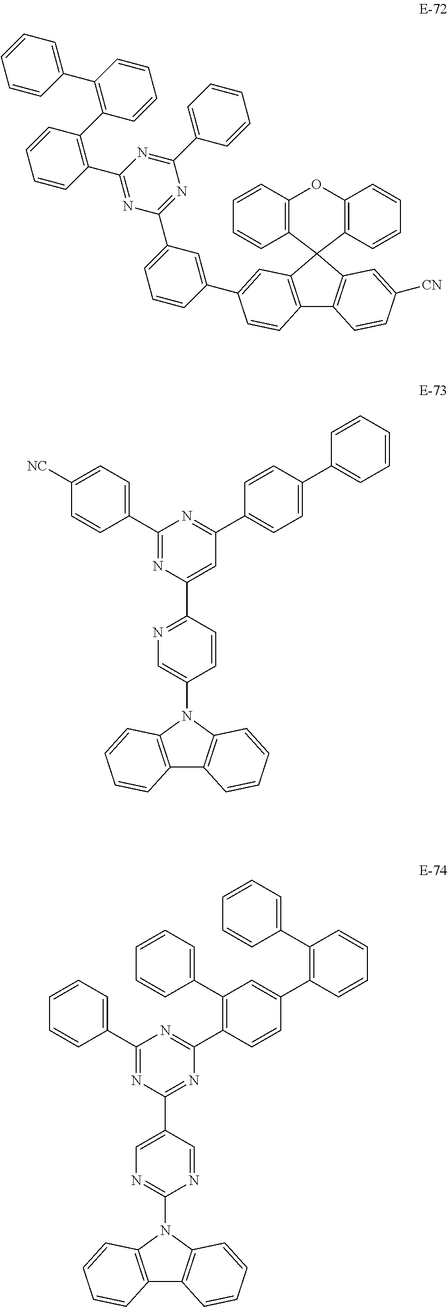 [SMILES: N#Cc1ccc2c(c1)C1(c3ccccc3Oc3ccccc31)c1cc(-c3cccc(-c4nc(-c5ccccc5)nc(-c5ccccc5-c5ccccc5-c5ccccc5)n4)c3)ccc1-2 ; N#Cc1ccc(-c2nc(-c3ccc(-c4ccccc4)cc3)cc(-c3ccc(-n4c5ccccc5c5ccccc54)cn3)n2)cc1 ; c1ccc(-c2nc(-c3cnc(-n4c5ccccc5c5ccccc54)nc3)nc(-c3ccc(-c4ccccc4-c4ccccc4)cc3-c3ccccc3)n2)cc1]

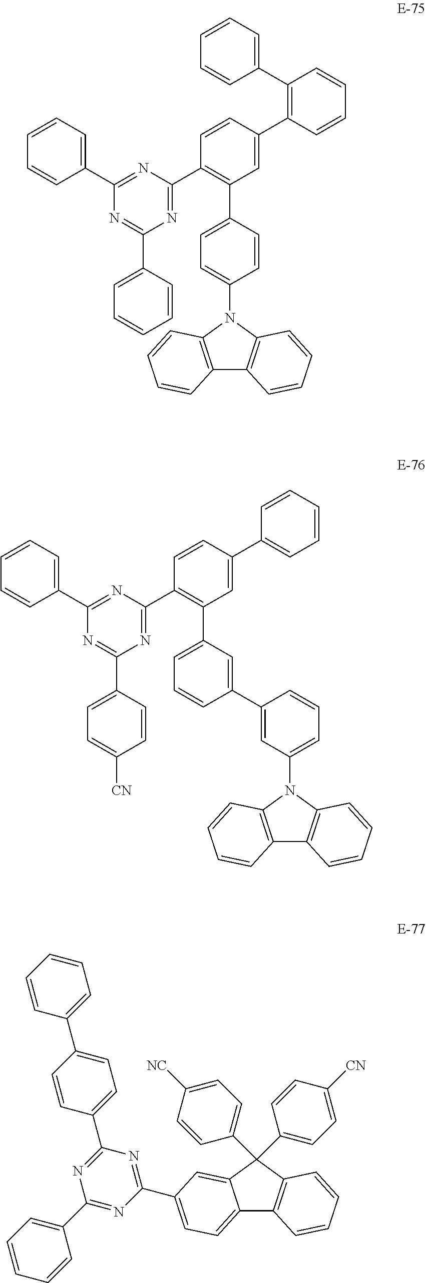 [SMILES: c1ccc(-c2nc(-c3ccccc3)nc(-c3ccc(-c4ccccc4-c4ccccc4)cc3-c3ccc(-n4c5ccccc5c5ccccc54)cc3)n2)cc1 ; N#Cc1ccc(-c2nc(-c3ccccc3)nc(-c3ccc(-c4ccccc4)cc3-c3cccc(-c4cccc(-n5c6ccccc6c6ccccc65)c4)c3)n2)cc1 ; N#Cc1ccc(C2(c3ccc(C#N)cc3)c3ccccc3-c3ccc(-c4nc(-c5ccccc5)nc(-c5ccc(-c6ccccc6)cc5)n4)cc32)cc1]

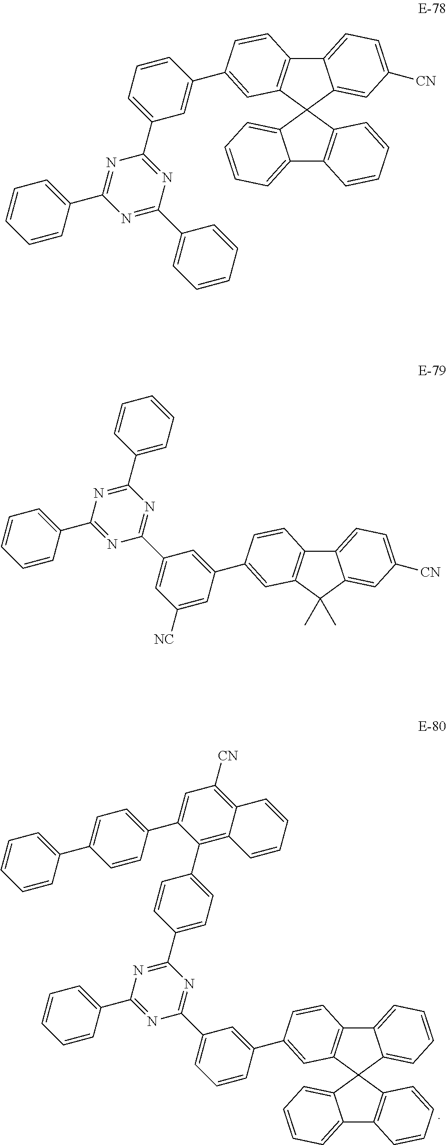 [SMILES: N#Cc1ccc2c(c1)C1(c3ccccc3-c3ccccc31)c1cc(-c3cccc(-c4nc(-c5ccccc5)nc(-c5ccccc5)n4)c3)ccc1-2 ; CC1(C)c2cc(C#N)ccc2-c2ccc(-c3cc(C#N)cc(-c4nc(-c5ccccc5)nc(-c5ccccc5)n4)c3)cc21 ; N#Cc1cc(-c2ccc(-c3ccccc3)cc2)c(-c2ccc(-c3nc(-c4ccccc4)nc(-c4cccc(-c5ccc6c(c5)C5(c7ccccc7-c7ccccc75)c5ccccc5-6)c4)n3)cc2)c2ccccc12]

10. The organic electric element of claim 1, wherein the hole transport band layer includes an emission auxiliary layer adjacent to the light-emitting layer, and the compound represented by Formula 1 is included in the emission auxiliary layer.

11. The organic electric element of claim 1, wherein the electron transport band layer includes an electron transport layer, and the compound represented by Formula 2 is included in the electron transport layer.

12. The organic electric element of claim 1, wherein the organic material layer includes two or more stacks including a hole transport layer, a light-emitting layer, and an electron transport layer sequentially formed on the first electrode.

13. The organic electric element of claim 12, wherein two or more stacks further includes an emission auxiliary layer formed between the hole transport layer and the light-emitting layer.

14. The organic electric element of claim 12, wherein the organic material layer further includes a charge generation layer formed between the two or more stacks.

15. An electronic device including a display device and a control unit for driving the display device, wherein the display device includes the organic electric element of claim 1.

16. The electronic device of claim 15, wherein the organic electric element is selected from the group consisting of an organic electroluminescent element, an organic solar cell, an organic photo conductor, an organic transistor, an element for monochromatic illumination and a quantum dot display.
